SEQUENCE LISTING (64 sequences)

SEQ ID NO: 1 - DNA, 1062 bp, Tuber melanosporum
atgactgctttgtccatgctcaatatggccgttgaagccgggcctcttattcaccaacgttatgatgggatcggtctcgccgagtatattcccttgctcgccaatggaacttttttctttgatgggtcggatggagccccgctccaggtccgtcatctccttccagcagactggaagcccgtcaatggaccacttacggtcttgttcgattccgccccccataccattattgggactcacttctctatccacaatcatcccatttacaagcttaacgatggttctatctttgttgtcttggacctggccaccaagcacggttttgagtttaccaatcaatctttcatggacccccagcttgaggtctccggggaaaaccgtttcttgcgtctcgttaccaatgaccaagacttcgcctaccatggctctagtcttagcatgattcatgacaagggcgttaagcgccctgccaatgcgtaagtacaattctaccaatgtaccagtttcgtgtgctcatatagtcataggtttatcttgttcagaacggacaaaatgaagcccctcaaggctcagtacccggagatgggcaacaacgatatctgtacgtggctctcattttccctttatttagatctctattcttgctaccatttgcctaacgttttaatagccaagattctcggtcgcatgtggcagaattcctcggatgaggtcaaaaatgtttataaggtaaaacatctcacctaactgttagttagaatcactaacacattaataggcaagggcccatcagcttgcggtttcccacaagttgatcaacccggattacaaatactccccgcgccgctctcgcgaaatcatgaggaggggctccgggaataaaggtccttcggcccgtatcgaccttcatcgtgctggtgttgcgaagcgtggaaaaaagaggatcattacccaccttgggttcctcactcaacgtttcgcgactcaccccactgctaacaatctcggtgatatcatcgaggaagccgaaaaggtcttgaaggcctttggtatggtcaattg

SEQ ID NO: 2 - DNA, 1069 bp, Tuber melanosporum
atgcgtaatgttaccttgggatgcgccttgtacctttcggaccgtgggtacattctcatgcaagacaggatcgggtgtatctgggtccagcacgagaactcccccattatcgttcaaccccccggtggtgtcactttcactgtcagcggcgaactcttgccctacgacaagtggctcggtgcaattcaatcaaccgtccgtcatcaagggcgagtccccctcgaccccgaagtcccttatgttgtgtcggttctgcgtttgtattgcggatactataaacctggacagccgtggacggcccaggatgccactattccggctgttatcgtcaatggtgatgaggccttggcccgtattcccccctctcagcaccttcgtgcattgaatccctacgttgcgcagagatgtaagtaaatcacatccctattttgcctctatttctaaccatctattagcctggatttctaagtactgtaacggttatgggctcactcaagccgaaatttccaatctcactcgtgatgtctgggtcgctgagaccaataagtatatgtggcaaaacatcgcctctctttatactgctgctagggatcgtggtgaccccggcctcgtgctcgaagaatttatcgagaccgaattggcgaaacacggtcaccctactacccctgagaagctcttgcgtgaagctggctttgtccccaaggcccccatcgacaaggaagcggccgataaggctgctgcaagaaggaaggttcgtgccaagcccgcacccggggtgtccagtttcactatcacccgtgtttacgtttccagcaaccgtcagctcgatctcaccgaggaggaagccgccaacatcctccatgctttcgacatcgctagcgctgaggagaatggagactctcaactcgacccgcacctccagcccgagatcgagatgcacatgaacactttcacttctggtggatacagcgtttttgtgagaagttccccgaacagcatattccgccactgcccaattctaacattatctaaacagcctactattggagaatcctccactggattgtga

SEQ ID NO: 3 - DNA, 5486 bp, Tuber melanosporum
tgagcgattgcttgcctgcaggcaagtgatccggacccaccactaggtgggcccggtacggggagcactgtctgtctgttcgcttcacttgtgcggttcgaatgctttacctggttgtgcgctctgagtacgagaaatgggcgggcaagctagtctgtctattcacgaagggcgcaatgaactatcacaaaccaatcccggattcttctactcttttgtccctttctcacggaggttctttccagtgagcttcgcccggtctcgtctcgtccgccattgcgtggttatttacagcggatggcttaatccgcacccttgtctatcatcgcaatacggcattggctctggcgaaactatatcgtgtcgttccgagggagaaaaaaaaatcccttcattctactttcccttcgtccactccgattcactggcagatgaaaccgtaccgcgcgctcgggctctttggtccctattttctcaaccggcgagggcgtcgttagtatgggggttggaaggctcctttggcttgtttgtcgtgagtagggagggcgctgtcattgctgagtagcctttcagcggggcctgcgggggtgcttctttccgatcggtcttgcgaggggtggtattgctattgggcggtgttggccattcggcggcgttgatgaaatttctatcgggggttctgtggcctctggatacgtggtgtcgtatatcttctggcggtttctctgctgctaagtatgatatcgcactattcgtggggacctgtcattaagtctttacatagttgctcactatggtcttaggaatccttgcgctatcgtacaccactttccttctatctgaacaacactctatatgtttatctatcgtacagggggagaaaggtaacgcgtgggcaagaatgcacggctggagaaaataaaatatgataggatgaaaaccgcaaggtataataaactagacttttttgccacagcgactagttccaaactctacagtactcagggggcaaacccacgcccgactataaaagcaaatctccaggggggctcgcatcagcacggtgacataagcgcattccgggctcgtctcatgggatctccttcgactggaaggggtgatgcctaaggacgactttcgtgggtagggtaccccggaaaagaaggaacggtgatgccttcgatgaggtcctgttttataagccttaaatcgcgctaggggttaatgctttgtgggactgtagcagcgctaccaccttctgatggccgtgcctagatgcgaacgagagaggtgtttgaccgtcattgtttggcctgtcggggctgacatcttccccgccgagtagtaccttcaccacctccgcatgcccatcccaagcagcgagcgagagcggtgtttggccgcaatcatctggcctgtcagggttgacctcttccctcatgagtaacattttcaccacttcttcatgtccatgaaaggcagcatgagagagaggtgattggccgtggctgtctggcttgtcgggattgacctcttcccgcatgagtagcattctcaccacctcttcatatccggaccagacggcatgcgagagcggtgttcggcaatagatatctggcttgtcagggttgacttcttcccgcccgagaaagattttcactactccctcatgtccagaaccagcggcatgcgagagtggcgttcggccgagattgtctggcttgtcggggttgacctcttcccgctcgagtaataatttcgcaactccctcataccccctccaagcagcattcgagagcggtgtttggccgtcattatctggctcgttgggattaacatcctcccgcccgagcagtattctcaccacttcctcatgtccaaaccaagcagcgagcgagagcggtgtttggccgtcattgtccggcttgtcggggttgacctcttcccgcccgagcagtattttcaccacttcctcatgtccacaggcagtagcatgtgagagcggtgtctaggccttctccgttcgttatattactataccatcctactgatctcagcatggcctaacttagccaagaacccctctgaaaccaccaggagtaacctctagcaatgtcagccacaatctcttaatgtccagacccttaccgagaccggttgtcgcattggatgactgcacctttgttctggagttccatcttttaaacagctttcccgagagctgcatacgccacatcaaacgtcgtagtcctccttgtccattcacctggatgcacaagcggatagccgaacaggggacattcattggatcggctggtagtgtttccaaggcatttggctttcctggttggccgggcagtgggtgaagctaccatacttctgaattcacatttcgcctaacggcctcctggccattggatatccccccaggacggaaagaacggcttctggatggccaggattaaggtatggtttcccagtccgccacacagatttatggagatcatattaaattcctcaatcagcaaactaccttgattcacatatctgatcacaaagaattattgttcagggctcggataccatacctctctatcaaagaagcaatgtacaagagaggtggggttggaggacggatgctgtaacagaacccacatttcccagctcaccctgtttcattaattcctttgttcggaacttctccgacagtaaaaaaaaattcgcggatcttttcgccaggaatcttgggaacctaagggctctctcttcctgccctgggggaaatcttattgtgtcataatattattatgtgtgataatattatgacattttcaagataccggtaatactattctcgggccctagtagggttgagttgtacgtgacggcttcttggcgtcactctattttttgggtcttggcatttcccctcgtgacaggaggcccactagggggatccatcctttcctccttctttcctccgccgacaaccgtcaagctccaccgacaaccgtcaagttccaccgacaaccgtcaagttcgacagaaacattcggcctttctacttgcacctctccactcacgtcgtttctttttttctgccactatgactgctttgtccatgctcaatatggccgttgaagccgggcctcttattcaccaacgttatgatgggatcggtctcgccgagtatattcccttgctcgccaatggaacttttttctttgatgggtcggatggagccccgctccaggtccgtcatctccttccagcagactggaagcccgtcaatggaccacttacggtcttgttcgattccgccccccataccattattgggactcacttctctatccacaatcatcccatttacaagcttaacgatggttctatctttgttgtcttggacctggccaccaagcacggttttgagtttaccaatcaatctttcatggacccccagcttgaggtctccggggaaaaccgtttcttgcgtctcgttaccaatgaccaagacttcgcctaccatggctctagtcttagcatgattcatgacaagggcgttaagcgccctgccaatgcgtaagtacaattctaccaatgtaccagtttcgtgtgctcatatagtcataggtttatcttgttcagaacggacaaaatgaagcccctcaaggctcagtacccggagatgggcaacaacgatatctgtacgtggctctcattttccctttatttagatctctattcttgctaccatttgcctaacgttttaatagccaagattctcggtcgcatgtggcagaattcctcggatgaggtcaaaaatgtttataaggtaaaacatctcacctaactgttagttagaatcactaacacattaataggcaagggcccatcagcttgcggtttcccacaagttgatcaacccggattacaaatactccccgcgccgctctcgcgaaatcatgaggaggggctccgggaataaaggtccttcggcccgtatcgaccttcatcgtgctggtgttgcgaagcgtggaaaaaagaggatcattacccaccttgggttcctcactcaacgtttcgcgactcaccccactgctaacaatctcggtgatatcatcgaggaagccgaaaaggtcttgaaggcctttggtatggtcaattgagtgatctctacggagggactcatcggtacctgggtggtatacttttaatgggagggatgatcttgtctatcaaaggtcattcattatctcagtcgactaaatttatgacctaaatagataaaaaatatcttttaaaaccaaaatgaggggtccgaacactacatcatacagtaaatatcaagttgtgaagcggaagagagccgactatgggaagaggcagtaagaatttaattctgactaagtctggaaagtttacacagcacttgcacagaagttatgctatggtggtgccatcaccggaaaaacacgccaaaaattcgggacctatggtgccgtgacttagtttgagcgcaagtacatggttcgacacaaaagagtgttacggagtaggggtgttggggttgattttggccattagcgttatcgtatagattgtttgaaaacacgactcgtattgcttaacaagaatatggagagagctatttttttaattttttttaattttgatagatgtattggactggaggccaagcatctgatcaattggttatactatactcctatttgttcatagttgactgaggccctgtttggctacaacggttcctttctgtttccccatacaaagcgaccgttgcagaacgggagggcaatagcttttatatgagaaacggttccaaaatgaaacggaaccgaggaaattgtgatctagtttcttggatgtgccgctgctactgaaatttttgttataaaggtatccgttttatatctgactcccttcacacagaccccaatggcgcgaatccccggagtactttgctcatgaaagtcgtcctgtcgtccttgggcatcaccccttccagtcgaaggagatcccatgagacgagcccggaatgcacttatatgtcaccatatggacatgaacctccgttgagatttgcttttacagtcgggcgttgttttatttcccgagtactttagagtttggaaccagttctattgctttgacaaagtctagtttattataccttgcagttttcatcttatcacattttattttctctagccgtacattcttgccctcgcgtttttttctcttgcacaactgatagacatataagtgttcttcagatagaaggaaatggcgtacgatagcgcaaggattcctaagaccattgtgagaaactatgtaaagacttctgacaggtccccacgaatagtgcaatatcatacttagcagcagagaaaccatcaagaaggtagactacagcacatagtcagaggccacggaactcctgagacataagggtagggata

SEQ ID NO: 4 - DNA, 7509 bp, Tuber melanosporum
ggcttgcatgatatcagcgaatgtagctcgaggacgtaaatctgatagttacactaaacgttggataccacgctggttcagagctagctccttctccgctcgaggtagaggattaagttcaccgccctctgaatggcatctgatgatgttccgggcaacttctctcggggcacctgtgtcgctagcaattgtatgataacttttgcctttctgcatgaagctcaatcattcgwactctctcatcgcttgaggccattcgccgcttgcctgtttcttgaccttgaggtatggtgtatgatgggaggtgttagctcctataaggaaatcgtggccaagatactagtgtcgggttactaaaataacctctgctgccgagaccgttgcgaaagctgggtagattctctctacatattctagtagatcttgacgctgatttacggatggataaaaaggaattttatgttttttttggttttaaagataccgagggcgtttcattgatcctagatacataagtttaatttaagtctacacgatctataatacgctgagaatgcctcccaactatgcccttgattcccgccgctcccacacggtcataccgtaatacagtaacttcattttttttttgtacccactagcctctttaggccttcatgggaggtcatatcggctcacaatccagtggaggattctccaatagtaggctgtttagataatgttagaattgggcagtggcggaatatgctgttcggggaacttctcacaaaaacgctgtatccaccagaagtgaaagtgttcatgtgcatctcgatctcgggctggaggtgcgggtcgagttgagagtctccattctcctcagcgctagcgatgtcgaaagcatggaggatgttggcggcttcctcctcggtgagatcgagctgacggttgctggaaacgtaaacacgggtgatagtgaaactggacaccccgggtgcgggcttggcacgaaccttccttcttgcagcagccttatcggccgcttccttgtcgatgggggccttggggacaaagccagcttcacgcaagagcttctcaggggtagtagggtgaccgtgtttcgccaattcggtctcgataaattcttcgagcacgaggccggggtcaccacgatccctagcagcagtataaagagaggcgatgttttgccacatatacttattggtctcagcgacccagacatcacgagtgagattggaaatttcggcttgagtgagcccataaccgttacagtacttagaaatccaggctaatagatggttagaaatagaggcaaaatagggatgtgatttacttacatctctgcgcaacgtagggattcaatgcacgaaggtgctgagaggggggaatacgggccaaggcctcatcaccattgacgataacagccggaatagtggcatcctgggccgtccacggctgtccaggtttatagtatccgcaatacaaacgcagaaccgacacaacataagggacttcggggtcgagggggactcgcccttgatgacggacggttgattgaattgcaccgagccacttgtcgtagggcaagagttcgccgctgacagtgaaagtgacaccaccggggggttgaacgataatgggggagttctcgtgctggacccagatacacccgatcctgtcttgcatgagaatgtacccacggtccgaaaggtacaaggcgcatcccaaggtaacattacgcataatgaggcgtctctcgagcctgttctcccactccgaaaccttaggggggggggagaggttaacgaccatgctcagattgacaggacgagacgacattatgaaaggtgggggggcggttggagacgggaaaagaaatttggaccgacaaaaggaggattgcgtatgggcttttcgatgagtggagattggcctcagtggctgttggtggtggaagaaaggagggtggaaggtctggcaacttttgaaggctacccgtcacgaagtgggatgccaaggcccaaaaaaaagagtgacgccaagaagctgtcacgtacaacttagccctactagggtctatcataatacatactatacgataataaattattatgatattatcataagattttccccagagcaggtagagagcccttaggtttccaagattcctggcggaaagatcggggaacatttttctttttttacggtcagagttgctcgaatagaaaaatgagtcaaggtatttgatgaagatctgatcgacattcgggagatgtttagaacaaaggaatttagtggggatgagctgggaaatgtgggttctgttacagtatctgtcccccaatcttaactagccctcttgtacattgcctctttggtaaaggggtatggtatctgagccttgaacagtaattccttgcgataggatatatcgatcaggatagtttgctggctggaaaatttaatatagtcttcataaaatcgtgtgacggactgggaaatcataccttgagtttggccatccggaagcctctctttctgctatgagggaatatcaaatagccaaaaggccatggggtgaaatgagaatttagaaatatgatagcggtcccttgccaccaaaagacaaatgcttcggcaataccatcacccgatccggtggatgtctccatgttcggctatccgcttgagcatccaggggaatagacaaggaagactaccacatttgatgcggtgtgtacggctgccgagaaaactgtttgaaagatggaactccagaacaaaggattaactggtcaacgcaatacctggttgccgcaagagtctgaagattaaaggattgtaactagtattgttggaggttacttctagcaattctagaggggtttttggtaaagatagattatactgatatcagtcggatgatatggcactacaacgaaagaaagtggtctagaccatgagcgcaaaaactccaaaaagaatttcctgccgggaaattagaatatttaaccggtttaaaaccgtaagatttgctggtccaggaccagcggcaaattgagagccattgtgagagcatctaagaactcaaccttcagaactccaggataagtaggatgataaaagggaaatggggtttagaatgggtgcataaagccttttgattctggattaagatatggccagataagggagtctgaagcaatgtgcatttaatctccgctcggactcctaggataaaaacggacatgacaagtaaattataatgaggaaaaaagcttgattggttgtaggggtcgattgtatgatcagaggtgggggtactacaagtttatcgatataatattgggaataactgtgaaaatggttccagaatatagctgagtattttacaaacatgcattagttgcatatgagatatcatagatactagagtcgccttgttcagtctcttggctgtctttgaatatctagatataccgggaaagaaaatgggaaagaaaatatcatcggcttctttagtaaactcttcaaagatagcaaaaacccaacctctacttacagcctgagcctatagcttggtatggtactcatttaccctttcttccacagagctatgcacttgcaatatttcttccaactgtttgtttaaaatccatgaggttcgacatcaaatgaaaggggtaatttccacatgcacaaaacatatattgtgtgaatacctacaattgagcctgcataaacacatgaacccaaatcaattcagtaaattaaaacccatcaagtagttggtgatttagctcatgaaatttgtgaagtttttttgagctaaagggaaaagagaaatgttacgcaaatttttttgtcacccttagctggtcggctgtcaatgcttgatattgtgatataaattactacaggttgtattgcgggaatgttgacatattgcagtacgacatcttctaaggccatatgcatataagagattactttatcgtactaagaaagccaagacaatgatttctactacttttttctatattctcttacctcatattccactatgatttacaagagtcatgccaaatcaagtattgtgtgatacctggtgaaaaatatccatcaaagcctcagagctgtaaatatcgcaaattgtaaggtgagaggggaatcacaatgcataatattatataaacaccacaactgctctactgggggtgccttatgctaggactttcctacatgcgacccattaacagattttaaaaaagaaaaaaagtcactgaggatgccccactgccacattaaaggagagagtagttcttagatcttgtctccctcagcttgcttagtgcacctgtggacagaaactcaggtgaagatagagaaagaggtatcatgaattttaatgcacagtattcattgcaatttaattccgggcacactaagttatttattcatttactcaatccctactgcaatccaagctactcttgtccttgcccaggctatatcttgagggcggggaacaaacttattctcattgcaccttctgggcgctacgagcgaaagattgactatttcatcaattttaaatcctaaatacaagtatcatacacatctactcaagtacaagaatccttgtaacgattggaacaaaacaaattctgggactcttgtcggacactacgttaacgctagtaagtaacaaaaaatctttttaatattagtagtgtgtgtgacatcctcttagattttgcgtgacacccctgccgctactaggtcttagtactcgagtagtagaataataggagtagcgaccttcaacgacagccaagcgatcaaaaagagtatcgtactcgagtattcaaatggaaaatctaccttcctatgataccaggtgaggtcattgcggtggttcaatacgacgatcaaatcctgcgattacgcataacaagtgtgagtgctcgagaagagcgcgaatgcacagctgcctggcctgcgtgggtctgacagatctgtccttgctctatttatttgttttgtttttcctgagcgcagcactttttaccctatgaggtatttataataatattatggatattctggaggaggttccaaaaaatatatcggaaatgcacttaggggcaatggtactaggattccatcgcacactatccgctaatagatatatctaggtttctagtttccataaatatcagacgagaaaggcttaccaaaatgaccaagggggttgggtctgggagatgtctacggtgcgacgatagaacggataaaagcgcagggcggggataaatcgagactcgggatggcagctttgatgtggatcagtcatgcgcagcggctactcgggatggacgagctctcccacgccctggcagttgagctgaactccaccgacttcaacgctagcaacatctcttcgatgtcgacactggtgggttgcttccaagggcttattaaggtggacaaggaagcatcagccgtgcaactggcccactttactctccaagagtacctttccgctcatcccgatattttctgcagacgccatgcggcaatggcagagatctgcttaacctatctgaattcccggcaaatcaaggacctttcgaccgaccatctcctgacaccccggatgcaccctttctagaatactgttccgtgcactggggaatccatgcgaaaacggaactctcggatggcaggagatcgcttgcgctgaagctattctaggagtttgatggccacatatccacagaatttcttttagcgcaagtggagaatttaagttcccaggggcgtgatacctgacctccgtccagtgggttgtactgcgcatcgttctttggaatcgtcgaggttgtggctgctttgacatccgtgggatgttgggatgccaaagaagggggttgtttggggtaaccgccacttgcacgggttcataacgggtatggggaggtggtggaaatgctactcgggttggaacaggtccatcccgacgagccggataacgcgaccggacaccgctctcacatgttactgcctgtgggcatgaagaagtggtgaaaatactgctcctgtttgagcggggagaggctagccacgaaaagccggacaatgacggtcaaacaccactctctttcgctgcttggtttggacatggggaagtggtgagaatactacttgggcggaagatgttaatcccaatgagccagataatgacggccaaacaccgctctcgaatgctgcttggaggggtcatgagagagttgcgaaaatattactcgggcgggaagaggtcaacctcgaagagccagacgagctcggccgggcgccgctcttgcatgccgctggtcatggacatgagggagtagtgaaaattttctcgggcgggaaggggtcaacctcgaaaagctagacaattactcccgaacaccgcttttgcatgccgtttggtctagatatgaagaggtggtgagtgtgctactcatagggggggtgtcgatctcggcgaaccggacagcagcggccaaataccgctctcgcatgctgccgctcatgggcacgaggaggtggtgaaaatgttactcatgtgggaagaggtcaaacccgacaggccagatgatttcggccaaacactgcgctcgcttgctgcttgggatggacatgggggatgatgaaagtactactcggccgggaggatgtcaaaccggacaggccagacgatagcggcaaaaccctgctctagttggccgccgggatgggcatgaggtagtggtgaaaatactactcgggcgcgaagttgtcagtcctgacaaaccagataatggcgaccaaacacccctctcgttcgcagctgggaatgaccatcagaaggtggtggccctgctacagtcccacaaagcgttaacccctagcgcggtttaaggtttataaaaaacaacctcttcgaaggcatcaccttccttttcttccagagtactttgctcgatgggggtcatcctagggcatcatcctttccagtcgaaggagatcccatgagacgagcccggaatgcgcttatatgccaccatgcggatgtgaacctccctggggatttgcttttatagtcgggcgtggatttttttttctgagtacggtagagtttggaactagttctatcgccgtgacaaagtttagtttagtttattacaccttgcagtcctcatcctatcatatttgatttcctccagccgtacattcttgccctcacgttttccctctcccttgcgcaattgataaacatatagagtgttcatatagaaggaaagtggtgtacgagaggggaagggttcttaagaccaatgtgagcaactatgtaaggactgaatgataggtccccacgaatagtgcgatatcatactcagcagcagagaaaccatcaagaaggtaactacagcacatatccagaggccccagacctgtgacataaggatagggata

SEQ ID NO: 5 - DNA, 12757 bp, Tuber melanosporum
gtgtgcgtgcttgaagtgaattggagtaaagtgaaggtggtggtggtgaaagtggcgaatatgagagtatcaaaggaggaggtgagcagagcattgagcttattagtaggttcagagcgagttgcgtgcgctgtctctagggttagagttacagttaggatgcggcgttgtgtgtgtgtgggcgtgggtgcgtgggtggatatgtgggtgtgtgtgttattcttatcatctgagtttgttgtcgtcttttcaatttcactcgagtcgctgtaggaggcaggagggagagttgtacgagtactgtagatgtagagcacctgtatcatattatcatactacacacagtacagtactgcacgagcacgagcacgaatacggtttcagtgaatctggagcttcactcgtttgtgttttttagcttcgtgcacttttgtcaatagagtaattatttaatatcatgatggttattataatatattatcatacaggtggtggagctgtatgatacccttcgtatcatatccttatgcatcacacatcctttgcataacaaagtagctggttttaggcctacagtgcccacgcgcacgggtactaccggtaccactctactaccaggatagtcaatgaagcttacctaacttgaacctgtctttttaccctcaattaccatccagctataaattttcctctcccacgaatccgatgctcgccactcgccgtgctcttcgctatttacggctgtcgaaacggctctcgctttcaaatcccccctcccaattccgccatattcacaccacacttcaaaaccgttcgagggatgaggaggaggcagctccttctcgtcccttgttgttgcccccccccgcgataacgataaaaccgcctgtggaggaatatgttcgacagatacgcgagcagtatggcgactacctccccgccggtctactggaggagggcgaatacagaatctacgagagatactatggccgtccaatccgactgctgcagccgggcgagacgctccaccattttccacctccaaagacgcccgcggaggaggccttatacgcacaggttatccagcgaatcgcccacccccaaggggtagggatggaagaaggagaagaaggggaagggggaggaggagggggaggaggaggtgaggaccttcgtgccaatggttctccaccattctttcggacacacccgctcacttacgccggtcgattcgagtcgtctcagattcgacttccgccatcggtaagggacgggacagagtctgtgatcgtggatgtcgccgcctcgcatctagcggaatcggcgcataagactctcggcggtccacacctccaattctcgcccgtatactcgaggaagcggagtaagcacatgttgactattcccttgaacccgattaacagtcgcatgtcaagtatggatgccgacgtgtttctcactgccgtgatgccggggctgtatgcgcagtgcttgagttcgttgaccgagctgcggaaacggttgggcggcgattgggtgcttggcggggatggggaaccgggtgttaggaatgtgcttgatgtggggggtgggggcgctggggtacttgcttggcggagtgttatcgaagtggaagaggaggtaaggaaggataggctttccggaagagccgggaagccgccacctggttcggatgggggggctctgcctgcggcgggggagaggagtgggatgcgagctgttgttgtcgccgggtccatcgctcttcgggggaaggtttcgaggctcttggacgatactctgtttattccccgaatgccggatatccatttcgaaaacctaaaccacacaccgggtggcctggtggagaagaataaggcccagccgagaaaactctacgatctgatcattgccacaaacaatctcctcccgatagtgaagtcgttttaccgccgccaagccattgaacaactctggtcgcatctaaatccgaacggcggcgtccttttgatgatcgaaagaggcacgccaatgggcttcgaggccatcgcctacgcccgctccgttattctagaggactacatcaaggacgttggcggagcctttcaatccctcccgaccacgaatgcccccggttccgccagcccccctgtcgaaaagggacccggcgctataatcgccccgtgcacgaaccacgagcaatgccccatgttcgtctctggccctacggatggcacccaacgcaaagattattgccgcttctcacagcgctatgagcgccccggctatctgcagaaactcatggaggagtcatccaaaaaccacgaggacctcgagtactcatacgtcgccttccgtcgtggtgtcgaccaccgcaccgacacgaagaacaagatcagcccaacaatagacgactttgacataatccccgtcgacggcgaaccccacacgacagttcaaagcccatatacagtgtcacaactccgtaattacagtctaaccctcccgcgcatcatcctcccacccatcaaacgcgacgggcacgtcatactcgacgtgtgcactccgacaggcttcatcgaacgctggattgtccctaagagcgtcggaaaattggagtttagagacgcgaggaagagcggatggggtgatctctgggcccttggagcgaaaagtcgcacaaggcggaacataaagattggcggtggcgacaagagatttaggtcaaagttagttgccatcaaggaccctcatagcgatagagtgagggctattgtcaaggacaagacgaccgccttcttgaaggctcggaagaaggagacaagggaggcgaggagggccgcaaggcttcagaagaagctagaggctaggatgaggcaagacgaaatggttcgtcagcaaaaggaggagaaggaaaggaagcagcgtgagaagttaatggcggagaagggggccagggaggtgcacgaaggggttgttcagatgcatggggagaggaagggaaagaaacggctggagaagcaaatgcgggaaagggagtaggaggcgacgaagggggggtgcaatcgcagttttgcgtaatgtaaccgtactttcaggccgccggtgggtggtgtgtatatatgaggcttcccgacttttgtaaattatatttctagctccgaaaaagcgttatacataacagccattgatggtggttcatgagttggtgcgtcattcgaaaggtcgagtgattgttggtgaaaggataccacgcccccccaccattcccgaatccgccaatctcttccatcgcccgtccagcagttgtgagggtggggggacgtgtatcggtgtatttgtggtatactcgtgtgcaggcaagtgctgtcggtggcggggttggcaggagatggcatgatatcttgagaaattttctgctagggggtgtggtggcatatcaacgtggcattctctattggaggattctgtcgtataccgttggatgggctttccatacattcctgccttaggccaatccgcttttcaccctccgcccggccgagccggcgcggtgtccttcaaggcggggcgtgggctcggagaacgggtgtttgtctatcgagtgatgttttgtggtcttactgattatagttatcaatacatccacatataccaggagcttctgagcgattgcttgcctgcaggcaagtgatccggacccaccactaggtgggcccggtacggggagcactgtctgtctgttcgcttcacttgtgcggttcgaatgctttacctggttgtgcgctctgagtacgagaaatgggcgggcaagctagtctgtctattcacgaagggcgcaatgaactatcacaaaccaatcccggattcttctactcttttgtccctttctcacggaggttctttccagtgagcttcgcccggtctcgtctcgtccgccattgcgtggttatttacagcggatggcttaatccgcacccttgtctatcatcgcaatacggcattggctctggcgaaactatatcgtgtcgttccgagggagaaaaaaaaatcccttcattctactttcccttcgtccactccgattcactggcagatgaaaccgtaccgcgcgctcgggctctttggtccctattttctcaaccggcgagggcgtcgttagtatgggggttggaaggctcctttggcttgtttgtcgtgagtagggagggcgctgtcattgctgagtagcctttcagcggggcctgcgggggtgcttctttccgatcggtcttgcgaggggtggtattgctattgggcggtgttggccattcggcggcgttgatgaaatttctatcgggggttctgtggcctctggatacgtggtgtcgtatatcttctggcggtttctctgctgctaagtatgatatcgcactattcgtggggacctgtcattaagtctttacatagttgctcactatggtcttaggaatccttgcgctatcgtacaccactttccttctatctgaacaacactctatatgtttatctatcgtacagggggagaaaggtaacgcgtgggcaagaatgcacggctggagaaaataaaatatgataggatgaaaaccgcaaggtataataaactagacttttttgccacagcgactagttccaaactctacagtactcagggggcaaacccacgcccgactataaaagcaaatctccaggggggctcgcatcagcacggtgacataagcgcattccgggctcgtctcatgggatctccttcgactggaaggggtgatgcctaaggacgactttcgtgggtagggtaccccggaaaagaaggaacggtgatgccttcgatgaggtcctgttttataagccttaaatcgcgctaggggttaatgctttgtgggactgtagcagcgctaccaccttctgatggccgtgcctagatgcgaacgagagaggtgtttgaccgtcattgtttggcctgtcggggctgacatcttccccgccgagtagtaccttcaccacctccgcatgcccatcccaagcagcgagcgagagcggtgtttggccgcaatcatctggcctgtcagggttgacctcttccctcatgagtaacattttcaccacttcttcatgtccatgaaaggcagcatgagagagaggtgattggccgtggctgtctggcttgtcgggattgacctcttcccgcatgagtagcattctcaccacctcttcatatccggaccagacggcatgcgagagcggtgttcggcaatagatatctggcttgtcagggttgacttcttcccgcccgagaaagattttcactactccctcatgtccagaaccagcggcatgcgagagtggcgttcggccgagattgtctggcttgtcggggttgacctcttcccgctcgagtaataatttcgcaactccctcataccccctccaagcagcattcgagagcggtgtttggccgtcattatctggctcgttgggattaacatcctcccgcccgagcagtattctcaccacttcctcatgtccaaaccaagcagcgagcgagagcggtgtttggccgtcattgtccggcttgtcggggttgacctcttcccgcccgagcagtattttcaccacttcctcatgtccacaggcagtagcatgtgagagcggtgtctaggccttctccgttcgttatattactataccatcctactgatctcagcatggcctaacttagccaagaacccctctgaaaccaccaggagtaacctctagcaatgtcagccacaatctcttaatgtccagacccttaccgagaccggttgtcgcattggatgactgcacctttgttctggagttccatcttttaaacagctttcccgagagctgcatacgccacatcaaacgtcgtagtcctccttgtccattcacctggatgcacaagcggatagccgaacaggggacattcattggatcggctggtagtgtttccaaggcatttggctttcctggttggccgggcagtgggtgaagctaccatacttctgaattcacatttcgcctaacggcctcctggccattggatatccccccaggacggaaagaacggcttctggatggccaggattaaggtatggtttcccagtccgccacacagatttatggagatcatattaaattcctcaatcagcaaactaccttgattcacatatctgatcacaaagaattattgttcagggctcggataccatacctctctatcaaagaagcaatgtacaagagaggtggggttggaggacggatgctgtaacagaacccacatttcccagctcaccctgtttcattaattcctttgttcggaacttctccgacagtaaaaaaaaattcgcggatcttttcgccaggaatcttgggaacctaagggctctctcttcctgccctgggggaaatcttattgtgtcataatattattatgtgtgataatattatgacattttcaagataccggtaatactattctcgggccctagtagggttgagttgtacgtgacggcttcttggcgtcactctattttttgggtcttggcatttcccctcgtgacaggaggcccactagggggatccatcctttcctccttctttcctccgccgacaaccgtcaagctccaccgacaaccgtcaagttccaccgacaaccgtcaagttcgacagaaacattcggcctttctacttgcacctctccactcacgtcgtttctttttttctgccactatgactgctttgtccatgctcaatatggccgttgaagccgggcctcttattcaccaacgttatgatgggatcggtctcgccgagtatattcccttgctcgccaatggaacttttttctttgatgggtcggatggagccccgctccaggtccgtcatctccttccagcagactggaagcccgtcaatggaccacttacggtcttgttcgattccgccccccataccattattgggactcacttctctatccacaatcatcccatttacaagcttaacgatggttctatctttgttgtcttggacctggccaccaagcacggttttgagtttaccaatcaatctttcatggacccccagcttgaggtctccggggaaaaccgtttcttgcgtctcgttaccaatgaccaagacttcgcctaccatggctctagtcttagcatgattcatgacaagggcgttaagcgccctgccaatgcgtaagtacaattctaccaatgtaccagtttcgtgtgctcatatagtcataggtttatcttgttcagaacggacaaaatgaagcccctcaaggctcagtacccggagatgggcaacaacgatatctgtacgtggctctcattttccctttatttagatctctattcttgctaccatttgcctaacgttttaatagccaagattctcggtcgcatgtggcagaattcctcggatgaggtcaaaaatgtttataaggtaaaacatctcacctaactgttagttagaatcactaacacattaataggcaagggcccatcagcttgcggtttcccacaagttgatcaacccggattacaaatactccccgcgccgctctcgcgaaatcatgaggaggggctccgggaataaaggtccttcggcccgtatcgaccttcatcgtgctggtgttgcgaagcgtggaaaaaagaggatcattacccaccttgggttcctcactcaacgtttcgcgactcaccccactgctaacaatctcggtgatatcatcgaggaagccgaaaaggtcttgaaggcctttggtatggtcaattgagtgatctctacggagggactcatcggtacctgggtggtatacttttaatgggagggatgatcttgtctatcaaaggtcattcattatctcagtcgactaaatttatgacctaaatagataaaaaatatcttttaaaaccaaaatgaggggtccgaacactacatcatacagtaaatatcaagttgtgaagcggaagagagccgactatgggaagaggcagtaagaatttaattctgactaagtctggaaagtttacacagcacttgcacagaagttatgctatggtggtgccatcaccggaaaaacacgccaaaaattcgggacctatggtgccgtgacttagtttgagcgcaagtacatggttcgacacaaaagagtgttacggagtaggggtgttggggttgattttggccattagcgttatcgtatagattgtttgaaaacacgactcgtattgcttaacaagaatatggagagagctatttttttaattttttttaattttgatagatgtattggactggaggccaagcatctgatcaattggttatactatactcctatttgttcatagttgactgaggccctgtttggctacaacggttcctttctgtttccccatacaaagcgaccgttgcagaacgggagggcaatagcttttatatgagaaacggttccaaaatgaaacggaaccgaggaaattgtgatctagtttcttggatgtgccgctgctactgaaatttttgttataaaggtatccgttttatatctgactcccttcacacagaccccaatggcgcgaatccccggagtactttgctcatgaaagtcgtcctgtcgtccttgggcatcaccccttccagtcgaaggagatcccatgagacgagcccggaatgcacttatatgtcaccatatggacatgaacctccgttgagatttgcttttacagtcgggcgttgttttatttcccgagtactttagagtttggaaccagttctattgctttgacaaagtctagtttattataccttgcagttttcatcttatcacattttattttctctagccgtacattcttgccctcgcgtttttttctcttgcacaactgatagacatataagtgttcttcagatagaaggaaatggcgtacgatagcgcaaggattcctaagaccattgtgagaaactatgtaaagacttctgacaggtccccacgaatagtgcaatatcatacttagcagcagagaaaccatcaagaaggtagactacagcacatagtcagaggccacggaactcctgagacataagggtagggataaccaccgtacacgagaagaacaattagaataaaacagagccattttataacaaatactacatgacaaagggctaccagaatatattgatgcgcagaggcccatgagagagagcataggatccctatttcggtacccgtagtttaagagggtgaaacatgccctagatattagtatattttctagggaactgctcttatatgatatgaaaaatatacttgtcctctggcgtatgtcacatccaccctgcaagcttaacctctttgggtgctcatcataggcaaggcccactagtttcaggagccagtcagatattatacaatagaatctatagatcggaggtcctaccgatttggatgtgattcgggtaagtctgggacattctgttaagattatacccactaccgaagccggaaagagtccagatggctggccaatatccgaaggggcataggccaaagctatcaggtgctctggcacagttgcacatgctcaggcaatcaaactttgaagataccgtaaaaacccttgccgttcactaccccaccgggttgttaatttcctaatctccgccgtattcagacataagcgcttaagcagaaatagtgctttcctgacacggtacagcctaagccctgtggaattgttggttctcatgaactagttttcgcaaccctgttaacatgtcggacgcatgtcgtgcgctgatgcggcgaactacagggatattggagacctacggagatatcgagtggaaaggatttttacagtattggtacatggctgttagactctcttgccctccctccttttgctacgcacgatgaagaatagaacaataccggtaggtcagatcagacgacgactctgcttggaatagagagatagagaaatagcatggctcgggcccctgcgcccgtcagccacccggaccgggccagggttgagatttgaaagaagatttcactgggtcagcccggcagcactggtacccagagccgcggatgtgccagcctggggttactcattcggcgctcgaacttcccccaataacccctcccttacggcgtctcgcccaagactagtggcatagggccaaaatactctgtatcatatgatatcatacgatatcgttgtccaatgcaaaaagaccgcaaacccaaacctaattgaaggttgtgataccactacaaaagtccgaggctcaatattcccacccaatcccatgtcaaccccccacaaaaaattctcctctcagcgcccactccagtgtaccaaattcaacgatatcttccacacaatacacccccgccttccttcctaaaatgcccccaagtatccccccaactaaaaaaaaccgaattcaatggcgatggctaacttggaattgtaaaacgaagatatcatgatccgccgcgaggtgatgttccgggctggaatgatgcttgtcaatgaaatggaagccactgcctggaaccctggcaaccttatggttgggattgccatccataagaatccaagcgttcgtatggttaaagcggttgagtcggccaaggacttgctccgtgaagtcgctgtgagcgaccataaactgcgcaaaatggatcgcatcaatgatcttcagccactggcgaatatccataatacacttttacagcttggaggattaatctctttgaaacaacatttctacgaggaacttcaaggtatcaaattacgccttcaggaactgcagagacacatgacagacaacattggccaggccgttgcagaagttcatgacttcatctgtttcgtcacttttgtcaacttgatccacccgcaaaactgcgtattaacctccaaatccatcaaggatgcggagattctcgcctctaagaactcatcccccgaggcactccaggcgtttgttgaactccgtcaggtttacgatctgcagactcccgttccagggtaccgtctaagcaaagccagggagctacctgcaatcactaggtgcgcaatggagcaatggcacaaaggttacttttctcatgcagccgcaactggaatagtaagtaaaaaaacatctcttccatctcagcactcgtacctaataagcgccatcccacttgatactaatatcaacccacagcaagttatcccattccagatcatattcggatggtggagtgagttcaatttcctcatttcaatttgatggtgttattagctgacatcaagagatgttgtcttatagttaattatcaacgcaagtttgttagtagctctcaagagaaaatcactccgattgtgcaagaacttatcaactgcgcatatacctcggaggccagtgagccaaaagataaggacgagaaactatggatgcaagaagataaggaggcttttaaggctgttactaacctagctatgtctatgactgaattgtgcaaaaatatccctctacccgagcatgtcaaaccgaaccctcagaaagagaagcagttgccgatttacacccagttcaaagtcgagcctcatgttcctccgatcttccaagaattccagaagctggtggctcaaatttacggactttttgaagaaaactcgtgctcaactttcactatcatgaccaaagcgtattatccaaaagtggagaactaccttaggttctctgaaaaagaccagacaacatcttctgtgaagcacttgaggtaagctatcttttaaggttccaggtttcttttgcactaaagctaattttcaacagcgccgcgacctactttttcagttcgctccgtgaggtcccagttgtggttctcatcaataagaatgtagggggtgtcgtgccacgcaaggctgaggttccactccttgatgctgttagcttggccggagtggatcccactattgccgatgaagagctcttggcggagattacttgcaagaatttgccacaagttggcatgcactgtaagtttttcattatggtactactggtttcggatatgttctgatctctgaacgtaacacagtccttgtcaggtatcgcggacaatctcaccagtgcccgcgcaactaaattctactatctctggtggcgtttgatcgcagcgctctatcagcgaaaagatgacaaatttcgctatccgggtagtctttagatagttccattcaaaatcaaactcttgtatcacaagatttgccatatctccatccacactttcattgtaaatttatatcaaaaaaaaaaagaccccatccagccccctggaagaacagaggaaggaggggactggttgagggcatttttcttcccttatcaccaggtacatggtgaacctcccagtgaggttggggagtttttattttacaaagggatctaattcgggatcatggcttcaggcccattcggggtagcgattgataatttagaggagtatgttattgtccattggttccactttggggaggataggagtttcttttattatttgggttttctaggccatctttccgggttcttaccgcgctttttcccgcagtttgcgaataaattcaaagacccaaagaaaacaaagtgatttagaccccatcaaaaagggcacctagctctgggaatgtaatta

SEQ ID NO: 6 - DNA, 15256 bp, Tuber melanosporum
  misc_feature (15085)..(15085): n is a, c, g, or t
gtgtgcgtgcttgaagtgaattggagtaaagtgaaggtggtggtggtgaaagtggcgaatatgagagtatcaaaggaggaggtgagcagagcattgagcttattagtaggttcagagcgagttgcgtgcgctgtctctagggttagagttacagttaggatgcggcgttgtgtgtgtgtgggcgtgggtgcgtgggtggatatgtgggtgtgtgtgttattcttatcatctgagtttgttgtcgtcttttcaatttcactcgagtcgctgtaggaggcaggagggagagttgtacgagtactgtagatgtagagcacctgtatcatattatcatactacacacagtacagtactgcacgagcacgagcacgaatacggtttcagtgaatctggagcttcactcgtttgtgttttttagcttcgtgcacttttgtcaatagagtaattatttaatatcatgatggttattataatatattatcatacaggtggtggagctgtatgatacccttcgtatcatatccttatgcatcacacatcctttgcataacaaagtagctggttttaggcctacagtgcccacgcgcacgggtactaccggtaccactctactaccaggatagtcaatgaagcttacctaacttgaacctgtctttttaccctccctcaattaccatccagctatcctctcccacgaatccgatgctcgccactcgccgtgctcttcgctatttacggctgtcgaaacggctctcgctttcaaatcccccctcccaattccgccatattcacaccacacttcaaaaccgttcgagggatgaggaggaggcagctccttctcgtcccttgttgttgcccccccccgcgataacgataaaaccgcctgtggaggaatatgttcgacagatacgcgagcagtatggcgactacctccccgccggtctactggaggagggcgaatacagaatctacgagagatactatggccgtccaatccgactgctgcagccgggcgagacgctccaccattttccacctccaaagacgcccgcggaggaggccttatacgcacaggttatccagcgaatcgcccacccccaaggggtagggatggaagaaggagaagaaggggaagggggaggaggagggggaggaggaggtgaggaccttcgtgccaatggttctccaccattctttcggacacacccgctcacttacgccggtcgattcgagtcgtctcagattcgacttccgccatcggtaagggacgggacagagtctgtgatcgtggatgtcgccgcctcgcatctagcggaatcggcgcataagactctcggcggtccacacctccaattctcgcccgtatactcgaggaagcggagtaagcacatgttgactattcccttgaacccgattaacagtcgcatgtcaagtatggatgccgacgtgtttctcactgccgtgatgccggggctgtatgcgcagtgcttgagttcgttgaccgagctgcggaaacggttgggcggcgattgggtgcttggcggggatggggaaccgggtgttaggaatgtgcttgatgtggggggtgggggcgctggggtacttgcttggcggagtgttatcgaagtggaagaggaggtaaggaaggataggctttccggaagagccgggaagccgccacctggttcggatgggggggctctgcctgcggcgggggagaggagtgggatgcgagctgttgttgtcgccgggtccatcgctcttcgggggaaggtttcgaggctcttggacgatactctgtttattccccgaatgccggatatccatttcgaaaacctaaaccacataccgggtggcctggtggagaagaataaggcccagccgagaaaactctacgatctgatcattgccacaaacaatctcctcccgatagtgaagtcgttttaccgccgccaagccattgaacaactctggtcgcatctaaatccgaacggcggcgtccttttgatgatcgaaagaggcacgccaatgggcttcgaggccatcgcctacgcccgctccgttattctaaaggactacatcaaggacgttggcggagcctttcaatccctcccgaccacgaatgcccccggttccgccagcccccctgtcgaaaagggacccggcgctataatcgccccgtgcacgaaccacgagcaatgccccatgttcgtctctggccctacggatggcacccaacgcaaagattattgccgcttctcacagcgctatgagcgccccggctatctgcagaaactcatggaggagtcatccaaaaaccacgaggacctcgagtactcatacgtcgccttccgtcgtggtgtcgaccaccgcaccgacacgaagaacaagatcagcccaacaatagacgactttgacataatccccgtcgacggcgaaccccacacgacagttcaaagcccatatacagtgtcacaactccgtaattacagtctaaccctcccgcgcatcatcctcccacccatcaaacgcgacgggcacgtcatactcgacgtgtgcactccgacaggcttcatcgaacgctggattgtccctaagagcgtcggaaaattggagtttagagacgcgaggaagagcggatggggtgatctctgggcccttggagcgaaaagtcgcacaaggcggaacataaagattggcggtggcgacaagagatttaggtcaaagttagttgccatcaaggaccctcatagcgatagagtgagggctattgtcaaggacaagacgaccgccttcttgaaggctcggaagaaggagacaagggaggcgaggagggccgcaaggcttcagaagaagctagaggctaggatgaggcaagacgaaatggttcgtcagcaaaaggaggagaaggaaaggaagcagcgtgagaagttaatggcggagaagggggccagggaggtgcwcgaaggggttgttcagatgcatggggagaggaagggaaagaaacggctggagaagcaaatgcgggaaagggagtaggaggcgacgaagggggggtgcaatcgcagttttgcgtaatgtarccgtactttcaggccgccggtgggtggtgtgtatatatgaggcttcccgacttttgtaaattatttctagctccgaaaaagcgttatacataacagccattgatggtggttcatgagttggtgcgtcattcgaaaggtcgagtgattgttggtgaaaggataccaccccccccaccattcccgaatccgccaatctcttccatcgcccgtccagcagttgtgagggtgggggacgtgtatcggtgtatttgtggtatactcgtgtgcaggcaagtgctgtcggtggcggggttggcaggagatggcatgatatcttgagaaattttctgctaggggtgtggtggcatatcaacgtggcattctctattggaggattctgtcgcatacccttggacgggctttccatacattcctgccttaggccaatccgcttttcgccctccgcccggctgagccggcgcagtgtccttcaaggcggggcgtgggctcggagaacacgtgtttgtctatcgagtgatgttttgtgatcttactgattatagttatcaatacatccacatataccaggagcttcggcttgcatgatatcagcgaatgtagctcgaggacgtaaatctgatagttacactaaacgttggataccacgctggttcagagctagctccttctccgctcgaggtagaggattaagttcaccgccctctgaatggcatctgatgatgttccgggcaacttctctcggggcacctgtgtcgctagcaattgtatgataacttttgcctttctgcatgaagctcaatcattcgwactctctcatcgcttgaggccattcgccgcttgcctgtttcttgaccttgaggtatggtgtatgatgggaggtgttagctcctataaggaaatcgtggccaagatactagtgtcgggttactaaaataacctctgctgccgagaccgttgcgaaagctgggtagattctctctacatattctagtagatcttgacgctgatttacggatggataaaaaggaattttatgttttttttggttttaaagataccgagggcgtttcattgatcctagatacataagtttaatttaagtctacacgatctataatacgctgagaatgcctcccaactatgcccttgattcccgccgctcccacacggtcataccgtaatacagtaacttcattttttttttgtacccactagcctctttaggccttcatgggaggtcatatcggctcacaatccagtggaggattctccaatagtaggctgtttagataatgttagaattgggcagtggcggaatatgctgttcggggaacttctcacaaaaacgctgtatccaccagaagtgaaagtgttcatgtgcatctcgatctcgggctggaggtgcgggtcgagttgagagtctccattctcctcagcgctagcgatgtcgaaagcatggaggatgttggcggcttcctcctcggtgagatcgagctgacggttgctggaaacgtaaacacgggtgatagtgaaactggacaccccgggtgcgggcttggcacgaaccttccttcttgcagcagccttatcggccgcttccttgtcgatgggggccttggggacaaagccagcttcacgcaagagcttctcaggggtagtagggtgaccgtgtttcgccaattcggtctcgataaattcttcgagcacgaggccggggtcaccacgatccctagcagcagtataaagagaggcgatgttttgccacatatacttattggtctcagcgacccagacatcacgagtgagattggaaatttcggcttgagtgagcccataaccgttacagtacttagaaatccaggctaatagatggttagaaatagaggcaaaatagggatgtgatttacttacatctctgcgcaacgtagggattcaatgcacgaaggtgctgagaggggggaatacgggccaaggcctcatcaccattgacgataacagccggaatagtggcatcctgggccgtccacggctgtccaggtttatagtatccgcaatacaaacgcagaaccgacacaacataagggacttcggggtcgagggggactcgcccttgatgacggacggttgattgaattgcaccgagccacttgtcgtagggcaagagttcgccgctgacagtgaaagtgacaccaccggggggttgaacgataatgggggagttctcgtgctggacccagatacacccgatcctgtcttgcatgagaatgtacccacggtccgaaaggtacaaggcgcatcccaaggtaacattacgcataatgaggcgtctctcgagcctgttctcccactccgaaaccttaggggggggggagaggttaacgaccatgctcagattgacaggacgagacgacattatgaaaggtgggggggcggttggagacgggaaaagaaatttggaccgacaaaaggaggattgcgtatgggcttttcgatgagtggagattggcctcagtggctgttggtggtggaagaaaggagggtggaaggtctggcaacttttgaaggctacccgtcacgaagtgggatgccaaggcccaaaaaaaagagtgacgccaagaagctgtcacgtacaacttagccctactagggtctatcataatacatactatacgataataaattattatgatattatcataagattttccccagagcaggtagagagcccttaggtttccaagattcctggcggaaagatcggggaacatttttctttttttacggtcagagttgctcgaatagaaaaatgagtcaaggtatttgatgaagatctgatcgacattcgggagatgtttagaacaaaggaatttagtggggatgagctgggaaatgtgggttctgttacagtatctgtcccccaatcttaactagccctcttgtacattgcctctttggtaaaggggtatggtatctgagccttgaacagtaattccttgcgataggatatatcgatcaggatagtttgctggctggaaaatttaatatagtcttcataaaatcgtgtgacggactgggaaatcataccttgagtttggccatccggaagcctctctttctgctatgagggaatatcaaatagccaaaaggccatggggtgaaatgagaatttagaaatatgatagcggtcccttgccaccaaaagacaaatgcttcggcaataccatcacccgatccggtggatgtctccatgttcggctatccgcttgagcatccaggggaatagacaaggaagactaccacatttgatgcggtgtgtacggctgccgagaaaactgtttgaaagatggaactccagaacaaaggattaactggtcaacgcaatacctggttgccgcaagagtctgaagattaaaggattgtaactagtattgttggaggttacttctagcaattctagaggggtttttggtaaagatagattatactgatatcagtcggatgatatggcactacaacgaaagaaagtggtctagaccatgagcgcaaaaactccaaaaagaatttcctgccgggaaattagaatatttaaccggtttaaaaccgtaagatttgctggtccaggaccagcggcaaattgagagccattgtgagagcatctaagaactcaaccttcagaactccaggataagtaggatgataaaagggaaatggggtttagaatgggtgcataaagccttttgattctggattaagatatggccagataagggagtctgaagcaatgtgcatttaatctccgctcggactcctaggataaaaacggacatgacaagtaaattataatgaggaaaaaagcttgattggttgtaggggtcgattgtatgatcagaggtgggggtactacaagtttatcgatataatattgggaataactgtgaaaatggttccagaatatagctgagtattttacaaacatgcattagttgcatatgagatatcatagatactagagtcgccttgttcagtctcttggctgtctttgaatatctagatataccgggaaagaaaatgggaaagaaaatatcatcggcttctttagtaaactcttcaaagatagcaaaaacccaacctctacttacagcctgagcctatagcttggtatggtactcatttaccctttcttccacagagctatgcacttgcaatatttcttccaactgtttgtttaaaatccatgaggttcgacatcaaatgaaaggggtaatttccacatgcacaaaacatatattgtgtgaatacctacaattgagcctgcataaacacatgaacccaaatcaattcagtaaattaaaacccatcaagtagttggtgatttagctcatgaaatttgtgaagtttttttgagctaaagggaaaagagaaatgttacgcaaatttttttgtcacccttagctggtcggctgtcaatgcttgatattgtgatataaattactacaggttgtattgcgggaatgttgacatattgcagtacgacatcttctaaggccatatgcatataagagattactttatcgtactaagaaagccaagacaatgatttctactacttttttctatattctcttacctcatattccactatgatttacaagagtcatgccaaatcaagtattgtgtgatacctggtgaaaaatatccatcaaagcctcagagctgtaaatatcgcaaattgtaaggtgagaggggaatcacaatgcataatattatataaacaccacaactgctctactgggggtgccttatgctaggactttcctacatgcgacccattaacagattttaaaaaagaaaaaaagtcactgaggatgccccactgccacattaaaggagagagtagttcttagatcttgtctccctcagcttgcttagtgcacctgtggacagaaactcaggtgaagatagagaaagaggtatcatgaattttaatgcacagtattcattgcaatttaattccgggcacactaagttatttattcatttactcaatccctactgcaatccaagctactcttgtccttgcccaggctatatcttgagggcggggaacaaacttattctcattgcaccttctgggcgctacgagcgaaagattgactatttcatcaattttaaatcctaaatacaagtatcatacacatctactcaagtacaagaatccttgtaacgattggaacaaaacaaattctgggactcttgtcggacactacgttaacgctagtaagtaacaaaaaatctttttaatattagtagtgtgtgtgacatcctcttagattttgcgtgacacccctgccgctactaggtcttagtactcgagtagtagaataataggagtagcgaccttcaacgacagccaagcgatcaaaaagagtatcgtactcgagtattcaaatggaaaatctaccttcctatgataccaggtgaggtcattgcggtggttcaatacgacgatcaaatcctgcgattacgcataacaagtgtgagtgctcgagaagagcgcgaatgcacagctgcctggcctgcgtgggtctgacagatctgtccttgctctatttatttgttttgtttttcctgagcgcagcactttttaccctatgaggtatttataataatattatggatattctggaggaggttccaaaaaatatatcggaaatgcacttaggggcaatggtactaggattccatcgcacactatccgctaatagatatatctaggtttctagtttccataaatatcagacgagaaaggcttaccaaaatgaccaagggggttgggtctgggagatgtctacggtgcgacgatagaacggataaaagcgcagggcggggataaatcgagactcgggatggcagctttgatgtggatcagtcatgcgcagcggctactcgggatggacgagctctcccacgccctggcagttgagctgaactccaccgacttcaacgctagcaacatctcttcgatgtcgacactggtgggttgcttccaagggcttattaaggtggacaaggaagcatcagccgtgcaactggcccactttactctccaagagtacctttccgctcatcccgatattttctgcagacgccatgcggcaatggcagagatctgcttaacctatctgaattcccggcaaatcaaggacctttcgaccgaccatctcctgacaccccggatgcaccctttctagaatactgttccgtgcactggggaatccatgcgaaaacggaactctcggatggcaggagatcgcttgcgctgaagctattctaggagtttgatggccacatatccacagaatttcttttagcgcaagtggagaatttaagttcccaggggcgtgatacctgacctccgtccagtgggttgtactgcgcatcgttctttggaatcgtcgaggttgtggctgctttgacatccgtgggatgttgggatgccaaagaagggggttgtttggggtaaccgccacttgcacgggttcataacgggtatggggaggtggtggaaatgctactcgggttggaacaggtccatcccgacgagccggataacgcgaccggacaccgctctcacatgttactgcctgtgggcatgaagaagtggtgaaaatactgctcctgtttgagcggggagaggctagccacgaaaagccggacaatgacggtcaaacaccactctctttcgctgcttggtttggacatggggaagtggtgagaatactacttgggcggaagatgttaatcccaatgagccagataatgacggccaaacaccgctctcgaatgctgcttggaggggtcatgagagagttgcgaaaatattactcgggcgggaagaggtcaacctcgaagagccagacgagctcggccgggcgccgctcttgcatgccgctggtcatggacatgagggagtagtgaaaattttctcgggcgggaaggggtcaacctcgaaaagctagacaattactcccgaacaccgcttttgcatgccgtttggtctagatatgaagaggtggtgagtgtgctactcatagggggggtgtcgatctcggcgaaccggacagcagcggccaaataccgctctcgcatgctgccgctcatgggcacgaggaggtggtgaaaatgttactcatgtgggaagaggtcaaacccgacaggccagatgatttcggccaaacactgcgctcgcttgctgcttgggatggacatgggggatgatgaaagtactactcggccgggaggatgtcaaaccggacaggccagacgatagcggcaaaaccctgctctagttggccgccgggatgggcatgaggtagtggtgaaaatactactcgggcgcgaagttgtcagtcctgacaaaccagataatggcgaccaaacacccctctcgttcgcagctgggaatgaccatcagaaggtggtggccctgctacagtcccacaaagcgttaacccctagcgcggtttaaggtttataaaaaacaacctcttcgaaggcatcaccttccttttcttccagagtactttgctcgatgggggtcatcctagggcatcatcctttccagtcgaaggagatcccatgagacgagcccggaatgcgcttatatgccaccatgcggatgtgaacctccctggggatttgcttttatagtcgggcgtggatttttttttctgagtacggtagagtttggaactagttctatcgccgtgacaaagtttagtttagtttattacaccttgcagtcctcatcctatcatatttgatttcctccagccgtacattcttgccctcacgttttccctctcccttgcgcaattgataaacatatagagtgttcatatagaaggaaagtggtgtacgagaggggaagggttcttaagaccaatgtgagcaactatgtaaggactgaatgataggtccccacgaatagtgcgatatcatactcagcagcagagaaaccatcaagaaggtaactacagcacatatccagaggccccagacctgtgacataaggatagggataaacaccgtacacgagaaggacaattagaataaaacagagccattttataacaaatactacatgacaaagggctaccagtatctattgatacgcagaggctcacgagagaaagtataagatcccgatttctgtacccgtagtttaagagggtgaaacatgccctagatcttagtatatttttctagggaaccgctcttataatattataataatatgaaaaatatacttgtgctctagcgtatgtcacatccaccctgcaagcttaacctctttgggtgctcatcataggcgaggcccactagttccaggagccagtcagatattatacaatagtatctatagatcggaggccctaccgatttggctgtgattcgggtgagtctgagacattctgttaagatattacccactactgaagccggacagagtccagatggcctcctggccgatatccgaaggggcataggccaaagctatcaggtgttctagcacagctgcacatgctgaggaaatcaaactctgaagataccgtaaaacaccttgccgttcgctaccccaccaggtagctaatctccatattcggacataagcgcttaagcaggaatagtgctttcctgacacggtacagcctaagccctgtggcattgttggttctcatgaactagttttcgcaaccctgttaacttgtcggacgtatgatagcatgtcgtgcgcgacctacagggatattggagacctatagtacagagatatcgaatcgaaaggatttttacagtattggtacatggccgttagactctcttgcccaccctccttttgctacccacgatgaaaaatagggtgccggtgggtcagatcaaacaactttgcttggaatagagagatagagaaacagcatggctcgggccactgctcccgtcagccacccggaccggtccagagttgggatttgaaaggggatttccctggattagtccggcagcactgtacctaaagccacacgctgtgccagcctggggttactcactcggcactcgaacctcccccgataacccctcccttacagctaaggatttcraatatacgtatattattacaatatactgtataatattattatacgttgcttttataccatacataccatacgtgtactaaaataagtaaccctaaattaatgaccctaaaaagtaaacgtcctgaacaaatatcccaactagaggttggcatagcaacttctatacttaaatttaatgtagattttgtaattttcactttacaccttcgacaaacttaagatattatttgggagggtattcagaaaactagttactcaaaattgacttgaatttgtaactattgtttttgcaataaaaagtattcttccccatttacgactcctactagtgattgttatccaatcgtggatttttgtggaaacaattttcttctatcaaaatttaataatatccttatctcgtaatattaaaccattaaaagaccagattttacggtattctacaaaattgtataaatacgaaattaatcctaacttacagcatctcgcctaaaactagtagcatagggctaaaatactatgtatcatatgacaccacacgatatcgttgtcccatgcaaaaagaccgcaaacccaaacctaattgaaggttgtgatcccactacaaaggtcagaggatcaatattcccacccgatcccatgtccacccccacgaaaaaatcctctcagcacccactccagtgtatctaagttgacaatatcttccacacatacacccccaccttccttgctaaaatgcccccaagtatccccccaactaaaaatctgaattcaataacgatagctaacttggaattgtagacgaagatatcatgatccgccgcgagacgatgttccgggccggaataatgcttgtcaatgaaatggaagccactgcctggaaccctggcaacctgatggttgggattgccatccataagaatcctagcgttcgtatggttaaagcggtcgaatcagccaaggacttgctccgtgaagttgctgtgagcgacaataaactgcgcaagatggatcgcatcaatgatcttcagccactggcgaatatccataatacacttttacagcttggaggattaatctctttgaaaaaacatttctacgaggaacttcgaggtatcaagttacgccttcaggaactgcagagacacatgacagacaacattggccaggccgttgcagaggttcatgacttcatctgtttcgtcacttttgtcaacttgatccacccgcaaaactgcatattaacctccaaatccatcaaggatgccgagattctcgcctctaagaacccatcccccgaggcacatcaggtgtttgttgaactccgtcaggtttacgatctgcagacccccgttccagggtaccgtctaagcaaggccagggagttacttgcaatcactaggtgcgcaatggagcaatggcacaaaggttacttttctcatgcggccgcaactggaatcgtaagtaaaaaaacatcaacactcgtacctaataagcgccatcccacatggtactaacatcaacccacagcaagttatcccattccagatcatattcggatggtggagtgagttcaatctccttattccggtttgatggtgttattagctgacatcaagagatgttgtcttatagttaattatcaacgcaagtttgttagtagctctcaagagaaaattactccgattgtgcaagaacttatcaaccgcgcatatacctcggaggccagtgagccagaggatgaggacgagaaaccatggatgcaagaagataaggaggcttttaaagctgttactaatctagctatgtctatgaccgaattgtgcaaaaatatccctctacccgagcatgtcaaaccaaaccctcagaaagagaagcagttgccgatttacacccagttcaaagttgagcctcaagttcccccgctcttccatgaattccagaagctggtggctcaaatctacggactttttgaggaaaactcgtgctcaactttcactatcatgaccaaagcgtattatccaaaagtggagaactaccttaggttctctgaacaagaccagataacatcttctgtgaagcacttgaggtaagctatcttttaaggtggtaggtttttttttacaccaaagctaattttcaacagcgctgcgacctacttttttacttcgctccgggaggtcccagttgtggttctcgtcaataagaatgtacagggtgttggcccatgcaaggttgaggttcccctccttgatgctattagtttggcctgctcggatcccaccattgccgatgaagagctcttggcggaaattacttgcaagaatttgccacaagttggcatgcactgtaagtttttcattatgatattactggtttcggatatgttctaatctctgaacctaacacagtccttatcaggtatcgcggacaagctcacccgtgcccgcgcaactaaattctactatctctggtggcgtttgatctcagcgctctatcagcgaaaaggtgacaaattttactatctgggtagtctttagatagttctatttaaaatcaaactcttgtatcacaagatttgcaatatctccatccacacattcgttgtgaatttatatcaaaaaaagaattgctctccagaccccatccagccccctggaagaacagaggaaggagggggctggttgagagcattttctttccttatcaccaggtacatggtgaacctcccagtgaggttagggagtttttattttacaaagggatctaattcgggatcatggcttcaggcccattcggggtagcgattgataatttagaggagtatgttattgtccattngttccactttggggaggataggantttcttttattntttgggttttctaggccatctttccgggttcttaccgcgctttttcccgcagtttgcgaataaatcaaagacccaaagaaaacaaagtgatttagaccccatcaaaaagggcacctagctctgggaatgtaatta

SEQ ID NO: 7 - DNA, 25 bp, Tuber melanosporum
caggtccgtcatctccttccagcag

SEQ ID NO: 8 - DNA, 26 bp, Tuber melanosporum
ccacatgcgaccgagaatcttggcta

SEQ ID NO: 9 - DNA, 25 bp, Tuber melanosporum
caatctcactcgtgatgtctgggtc

SEQ ID NO: 10 - DNA, 26 bp, Tuber melanosporum
tctcgggctggaggtgcgggtcgagt

SEQ ID NO: 11 - DNA, 31 bp, Tuber melanosporum
tggtatatgtggatgtattgataactataat

SEQ ID NO: 12 - DNA, 26 bp, Tuber melanosporum
agagatagagaaatagcatggctcgg

SEQ ID NO: 13 - DNA, 29 bp, Tuber melanosporum
gtgtgcgtgcttgaagtgaattggagtaa

SEQ ID NO: 14 - DNA, 27 bp, Tuber melanosporum
taattacattcccagagctaggtgccc

SEQ ID NO: 15 - DNA, 25 bp, Tuber melanosporum
aagtaacctttgtgccattgctcca

SEQ ID NO: 16 - DNA, 27 bp, Tuber melanosporum
cagcatggtctggttggacgttttgat

SEQ ID NO: 17 - DNA, 26 bp, Tuber melanosporum
ctatgcacccaagtgtgattgaggac

SEQ ID NO: 18 - DNA, 26 bp, Tuber melanosporum
ccattcgttgagatgcgccgttgcta

SEQ ID NO: 19 - DNA, 27 bp, Tuber melanosporum
aggcggtatcagagcttttccatgcag

SEQ ID NO: 20 - DNA, 32 bp, Tuber melanosporum
gccaacctctagttgggatatttgttcaggac

SEQ ID NO: 21 - DNA, 25 bp, Tuber melanosporum
gtgaatagacagactagcttgcccg

SEQ ID NO: 22 - DNA, 26 bp, Tuber melanosporum
aatctcttaatgtccagacccttacc

SEQ ID NO: 23 - DNA, 27 bp, Tuber melanosporum
tgaatcaaggtagtttgctgattgagg

SEQ ID NO: 24 - DNA, 27 bp, Tuber melanosporum
tatcgtacaccactttccttctatctg

SEQ ID NO: 25 - DNA, 25 bp, Tuber melanosporum
gatcttcagccactggcgaatatcc

SEQ ID NO: 26 - DNA, 27 bp, Tuber melanosporum
gttcctccgatcttccaagaattccag

SEQ ID NO: 27 - DNA, 27 bp, Tuber melanosporum
gccggaatagtggcatcctgggccgtc

SEQ ID NO: 28 - DNA, 28 bp, Tuber melanosporum
gacggacggttgattgaattgcaccgag

SEQ ID NO: 29 - DNA, 26 bp, Tuber melanosporum
gatcgtggtgaccccggcctcgtgct

SEQ ID NO: 30 - DNA, 28 bp, Tuber melanosporum
cttgcgtgaagctggctttgtccccaag

SEQ ID NO: 31 - DNA, 24 bp, Tuber melanosporum
atcctaacttacagcatctcgcct

SEQ ID NO: 32 - DNA, 28 bp, Tuber melanosporum
tatcggctcacaatccagtggaggattc

SEQ ID NO: 33 - DNA, 22 bp, Tuber melanosporum
acggcccaggatgccactattc

SEQ ID NO: 34 - DNA, 12121 bp, Tuber indicum
tgggcgattgcttgcctgaaaacaagtgatccgggggagcactgtctgtctgttcgcttcacttgtgctgttcgaatgctttacctggttgtgcgctctgagtacgagaaatgggcgagcaagctagtctgtctgttcacgaagggcgcaatgaactatcacaaaccaattccagattcttctactcttttatccccttctcacggaggttctttccagtgagcttcgcccggtcttgtctcgtctaccattgcatagttatttacagcggatggcgtaatccgcacccttgtctatgatcgcaatacggcattggctctaacaaaactatatcgtgtcgttccgagggagaaaaaaagtcccttcattctactttcccttcctccactccgattcactggcagatgaaaccgtaccgcgcgctcgggctctttggtccctattttctcaaccggcgagggcgtcgttagtatgggggttggaaggctcctttggcttgtttgccgtgagtaggaagggcgctgtcattgctgagtagcctttcagcgggacctgcgggggtgcttctttccgatcggtcttgcgaggggtggtattggtattgggcggtgttggccattcggcggcgttgatgaaatttctatcaggagttctgtggcctctggatacgtggtgtagtatatcttcttggcggtttctctgctgctgctaagtatgatatcgcactattcgtggggacctgtcattaaatctttacatagttgctcacaatggtcttaggaatcctcgcgctaccgtacaccactttccttctatctgaacaacgctctatatgtttatgtatcgtacagggggagaaaggtaacgcgtgggcaagaaatgcacggctggagaaaataaaatatgataggatgaaaactgcaaggtataataaactagacttttttgccacagcgatagagctagttccaaactctacagtactcagggggcaaacccacgcccgactataaaagcaaatctccggaggggctcacatccgcacggtgacatataagcgcattccgggctcgtctcatgggatctccttcgactggaaggggtgatgcccaaagacgactttcatgagtaaagtaccccggaaaagaaggaacggtgatgccttcgatgaggtcctgttttataagccttaaatcgcgctaggggttaacgctttgtgggactgtagcagcgctaccaccttctgatggccgtgcctagatgcgaacgagagaggtgtttgaccgtcattgtttggcctgtcggggctgacatcttcccggccgagtagtaccttcaccacctccgcatgcccaccccaagcagcgagcgagagcggtgtttggccgcaatcatctggcctgtcggggttgacctcttcccgcatgagtaaaattttcaccacttcttcaagtccatgagcggcagcatgagagagcggtgattggccgtggctgtctgacttgtcgggattgacctcttcccgcatgagtagcattctcaccacctcttcatatccggaccaaacggcgtgcgagagcggtgttcggcagtagatatctggcttgtcggggttgacctcttcccgcccgagaaagattttcactactccctcatgtccataaccagcggcatgcgagagtggcgttcgaccgagattgtctggcttgtcggggttgacctcttcccgctcgagtaataatttcgcaactccctcataccccctccaagcagcattcgagagcggtgtttggccgtcattatctggttcgttgggattaacatcctcccgcccgagcagtattctcaccacttcctcatgtccaaaccaagcagcgagcgagagcggtgtttggccgtcattgtccggcttgtcggggttgacctcttccctcccgagtagtattttcaccacttcctcatgtccgcaggcagtagcatgtgagagcggtgtttggccgcgattatcgggctcgccgggatcgacctgttccagcccaagtagcattttcaccacttcctcatgtccgttatgagcagcccatgcaagtggcgtatacccccaacaatctccttcgttgatatcccaacatcccatctctattaaagcagccacaacctcgacaattccaaagaacgatgcgcagtgcagcccactgaatgggggccaggtatcgtgccctccgggacctaaattccccacttgcgctaaaagaaattcggtggatatgtggccatcaaacccctggaatagcttcagcgcaagtgatctcccgcaatccgagagttccgttttcgcatggactccccaatacatagaacaatattctagaaagggtgcatccgaggtgtcaggagatggatcggtcgaaaggtccttgacttgctgggaattcagataggtcaagcatatctctgccattgccgcatggggtctactgaaaatatcgggatgagcggaaaggtactcttggagagtaaagtggactagtcgcacggctgatgcctccttccccaccgtaataagcccttgacagcaacctatcagtgtcgacatcgaagggatgttgttagcattgaagtcggtggagcccagctcgactgccagggcgcagcagagctcgtccgtcctgagcggccgctgcgcatgactgatccacatcaaggctgccatcccgagtctcgatttatccccgccctgcgcctttatccgtcctatcgtcgcaccgtaggcatctcccagacccaaaccgtcggtcattttgctaagcctttctcgtctccgataggttgtcgattcttgtaagattgcttcgatgtttagggaaactagtataaacctggatatatctattagcggatagtgtgcaatgggaatccctctgtcattgccctacaaacatttccgagatcttcccagggatctgctctagaacgtctgcctcaagaccctcgtccatggcatctggggtttcgtcctcgtctagcctaacacgaaggtatctgataatatcatctcttctagggcctatagacacacttgccactcgcccggcaagacgcttttcgatctcggcccgaaatataggaccttccagtcaggaatattcgtatccccggggacttcccaaggatttttttcagcgaatcaaggagcttggctcgatgcactgcggcacattcgtccagggcgtcaatgcacataaatgtgcgctgtgaggacgtgatggtctgtagcatctttacgatatcaggaagttgcggtccccgcccgccaatggccctcctctgctcttgaaaagcccgcgatatttcctctggaatcctttccattccgctaacgatctgtttaagcaaggaacccagcatattcgtcgcggactgctcctttcgggccgcaaagtcaaagtaaaaacaggtaacggtagtgttttgtcctctagcctgatcgcacagcctgtccactaccatcgaactgtcatcgcggcttattagcactggcgcacttctctccccagagaataactcttgttacctgataaatgtcttgcccacacctggatctccatggcaaaacaacactgccttattgccttcaccttccaaactccagtcataccagcttctgaattcgtcagtttggataagccattcccccacattgtccacacggtgctcttgaatgtcgcgatgccgtaacctaggttccagcggcgacaaccaagctaggagttgagaccgatcatcggcaactgtgaagttgttccagacattgctaaaagagctcgtattattgaagcagttgttattgctattgattgtatattgagacatcttctgctctttactgcttgcactattgaagggaagtatttttctggtttggttggttgacgatatccttgggaggtcaagaaaagcggggtctagatggggaaagcaagggtctgctggtgttaaagcagcatcgcagataccgctactcgtacatgcagtactaactcaagagttttcgtccggtaccctttttacaccaaaagtatctaacttcaccaccctctctcacagcacaatgtcaaggggaatatgccaaaatttcgcggtgtattggcaaatctgtagctaatatgggagataatattattacagaattgttgagtaatagagccccataccggtacaaaaatctaggttaatttcacacaggccaacgttattgtaaacaatgggcacaactttgatcctctacagttctacttctagagatttaaatgaataacagctatctagccgagggagggatttggagtgttttcaacagggtattgtttggattgttaggtatgttgcggtagtctgcgacatgataaacaccatacttcgtggagaatgatataaagcgctgcgcccgggaaaagtaaggacaaaataaatagacactgttaaatacgtagaacaaggtctttggcattcgagtgcaatttgaaaacttgcggtcgatattcgtgatcgagagattgtggctgttgcatcgaactaccatatgactttcctaaataggaaggcagactcttgattcttttagatggcctggcagttgttgaaggttctgaactatcccttagagagctgaatcaacgaaattcagtgccgccaagatattgcgcaactgtgctgtctaagacatagggtgaataagcactaagcgacgtcgtgagaagctgaatctcgtgagaagattaagaaaacaaatcccaatcgttcagaattctactagaaatgccacgaacaagaaaatcaagtgcacaaacggctgctagaggagtggtgtcccagtcggttcaccagagagagcctttagctgtcctatcagtgaacggtaggaagccaagccgaatatgttcatagaatgctaaacgtttcacagcaggtgcagaatctcgaatgggaccagtggggccatcacatctgcatatggataccataatgcctatgggtgagatgggaaagggaaagggaaaagcaaaggagtcgggtggggagtcactctctgcggccgaaaagattcgaaatcttcctcctccgccacaccacataccattccaaggactatttccaccgcaccaagctagagccataggacctgcaactggagtaactgacccgtatcggttattctcactgtttttttcgccggagcaactggagattctggcgaggcataccaatatttatgcaagtatgcatgatgctggcctgcaagacagtaggcaccctcttgttcggaagtggtatccaacaactccctctgagcttcgaatattgttagcgatcttgattcatctgggtatatcaagaggtgctagtccaaaactcttttggagaaaggttggaggtgttgttccggaaccgatgtgcagaatgagatatatacgctttcagcagctaaagcgctatttgcatatctcagagccatctgagtccccaattcctactcaaaagtggtggaaaaagctcgaacctttaaatagcagtattcgaaaaacctcaaaaagagtgtttccttccgtccaccaatgtggctgtcgatgagatgatgatacgcttccttggccgctcagcccacacaattaaaatgccaaataaacctattagtttaggatataaggtactggcattgtgtgatgcaggatacacctatgactgggaacttacttcacgtattgcgagctttgcaacctccgaacagcaaaaaatgccatatcctctttctcctacctcctccgctgtacttcagatgcttaccacactaccctaccgtacccacttttttaccgcctatatggacaattacttctcaaatatacgcctatttgcacgtttactggattatggaattggagcctgtggtacagtgcggtgcagcagcaaagattttcctccctctctcaatatcagcaaagataaggctgctggtatcttgaaatggaattttgctactggagttgttgtacgcgaaatagttgatacagcaaaaaagataaagggagacagcccacccaagctacataggtgggggattcctccggttatggcttttttgtggcaggataataacacggtgcatttattatctactgttcatgatcttgagccatcaacttcctggataaagaaacttcgcagaaggccacgggaaacaagtaccaatgctgctgctgctcggaagcccttttctgctggggagcatcgcaagctattggcaattcccaagatcgacaatgactataaccaattcatgggtagtgtggatattgcagatcagcttcgatcatatttttctacgcaacgtattgtgcgtcggaactggcaaccttttttttactggttacttgatactgcaatcattaatgcctaccgaatcgcgcgaaccaatggctctaaaaccacccaccgtacatttcgctcctcccttacaagttcccttcttactgccggtcagaaacactcctcccctgagccaaaatttacattcctctaccgaactcgacgacggtatcgcctacgcaacccagtacgtcaaatatatattacgaagaactcacagaagccagaaccggtaggtggagggggaactttaaggggccatgtactggtacggaaagctactcgagcatggtgcctatggtgccgctggcgtcacaagcaaggtggctcggaggtgaaaatccaccaagtgcgatctcagtgtgaacggtgtcatgtagccctttgtcattcatgttttcccttatatcatggtgtaggaagctctttggtataggaagctcttcggtctaggttctcttttttttttttttttatatcactttgtctatttagtaatccagatccctgctctggcttgctgtcgattgcgcaatatcttggcggcactgaatttcgttgtttcagctctcgaagggataggaactattggtacttgtactaaatactaattcctaacaacgtacagggatgtcacgcaaaatcaaatcaaacattacacactaatggtattaaagaatgttttacttgctagcgccgtcatcgttcctgatcacctggaatttgtcagttccaatagacagacagctataagggctcttgtagatgagtgtttgaggatttacattgctatctcagcttcagggagaacaagaaaggtatgtcattttttgcccccacagcccaatatgtgcagcgaaaatggttgttttttctcccgtcaaaatacaatttttgccgggataaggggaccctaggtcgggtggggttaaatggcttggggtatcatatctagaaggaaattgacaattagcgcgcattaaactcacagctctttggtgagctttcctcgttgtctgtctgtaggtgcacgtggagcgatccaggtatctggggggacctgggaagcattcccctcttattgtggtggtagggcacctgcagtggggaaactccagcatataaataaagttataatataatattattacgattcccgcctatctcataattaaattctgcgatattcccagctctggggtattattggtcgttatctccacgatgtatcacccgatgtttgatttagcgtgactgttgtaaatcacagtggaatgagagataagagtatgtggggacaggtagaagtgacttgcttgacactctgggtggggtgaatacatacatataactcatagagatgtcatgctaaggtatatagacaactctataacacaacttggatatggtggataaagtgtgacggcctactaactgggaccacaaaggattcaattgtgtaccttaagaacatggaatacttatgctgtaatgttttatgcatgcaggaatcacccattcagtagatgtcgaattttacagctttcgaactaacactccattaccaaataagtatcgcgggtgcgcatttgtgggaagagatgggcattataccataaaccacaggttctaataagtgtttagatattggctgcttccagcgattccatcaggacttaagatatttcctttctagtacaactactaaatagagaaagacagcgaagagatggaacagagaggccctagtgcccgcctcatgtgcaattacggtatgtttgtgagagacactccgctatgatctactaccttcacagatgttcctaacattatataaacaaacgtgcaatatctcggtctctgatcatacaatcccctgtcctacatccaatcaagttcctttcctccgtgtaatccacttgacatcttagcttctatcccgggagtcggagcgggaccgggggcacattgccccaggctctcttatctgacgatatcttcgtccagaatcaagagactttgtctgtcccttctaaactccgcccccttccatcatcctgccttgcatgaagctcaggaggttgagtgcttagatgctccacaatggtttgctcaatcggccgctgctcctgggccagaagctctgacggcttagagttggttaaagattctaatttccaagctgaaaatccttttgggggcgggctttgctcgctcgtgatccagaccttctccgttcgttatattactataccgtcctactgatttcagaatggcctaacttagccaaaaacccctctgaaaccactaggagtaactgctagcaatgttagccacaatcccttaatttccagacccttaccgagaccggttgtcgcattggatggctgctcctttgttctggagttccatcttttaaacagctttcccgagagctgcatacgccgcatcaaacgtcgtagtcctccttgtccattcacctggatgcacaagcggatagccgaatatgggacatccaccggatcggctggtagtgttgcaaaagcatttggctttcctggttggccgggcagcgggtgaagctaccatacttctgaattcacatttcgcctaacggcctcctggccattggatatccccccaggacggaaaggacagcttctggatggtcaggcttaaggtatggtttcccagtccgccacacagatttatggagatcatattaaattccgcaatcagaaaaaactatcttgaaccacatacggtatctgatcgcaaagaattattgttcagggctcggataccatacctctctatcaaagaagcaatgtacgagtacaagagaggtggggttgggggacggatgctgtaacagaacccacatttcctagctcaccctgtttcattaattcctttgttcggaacttctcccgaatgtcgatcggatcttcatcaataccttgactcgttttctaccatactccagcatctccgaccgtaaaaaaaaaatttcgcggatcttttcgccaggaatcttggaaacctaagggctctcccttcctgccctgggggaaatcttatcgtgtcatatgtgtgataataatattatgacattttcatgagaatactattctcgggccctaatagggttgagttgtacgtgacggcttcttggcgtcactctattttttgggtcttggcagtccccctcgtgactggaggctacaaaggggatccatcctttccccttctttcctccaccgacaaccgtcaagttcgacagaaacattcggcctttctacttgcacctctccactcacgtcgtttcctttttttctgccactatgacttcttctttgtccaacctcaatatggcccttgaagccgggcctcttattcaccaacgttatgatggaatcggtctcgccgagtatattcccttgctcgccaacggaacctttttctttgatgggtcggatggagtctccctccaggtccgccacctccttccagcagactggaagcccgtcaatggaccacttgcggtcttgttcgattccgtcccacataccattattgggactcacttttctatccgcaatcaccccatttataagcttaacgatggttctatcttggtcgtctttgacccggccgctaagcacggtttcgagtttaccaatcaatctttcgtggacccccagcttgaggtctccggagagaaccgtttcttgcgtctcgttaccaatgaccaagacttcgcctaccatggttctagtcttagcatgattcatgacaagggcgttaagcgccctgccaatgcgtaagtacaattctaccaatgtaccagtttcgtgtgctcatctagtcataggtttatcttattcagaacggacaaaatgaagcccctcaaggctcagtacccggagatgggcaacaacgatatctgtatgtggctcttattttccctttattttgatctctattcttggtaccatttgcctaacgttttaatagccaagattctcggtcgcatgtggcagaattcctcggatgaggtcaaagatgtttataaggtaaaacatctcacctaactgttagttagaatcactaacacattaataggcgagggcccatcagcttgcggtttcccataagttgaccaacccggattacaaatactccccgcgccgctctcgcgaaatcatgaggagggactccgggaataaaggtccttctccccgtatcgaccttcatcgtgctggtgttgcgaagcgtgaaaaaaagaggatcattacccaccttgggttcctcactcaacgtttcgcaactcaccccactgctaacaatctcggtgatatcatcgaggaagccgaaaaggtcttgaaggcctttggtatggtcaattgagtgatctcaacggagggactcatcagtgcctgggtgatatactttcaatgggagggatgatcttgtctatcaaaggtcattatctcagtcgaataaatttatatgacctaaataaataaaaaatatgtttaaaaaaaaaaaaatgaggggtctgaacactatatcatacagtaaaatatcaagttgtgaagcagaagagagtcgactaccggtatgggaagaggcagtgagaatctaattctgactgtcttagtctggaaagttcgtacagcacttgcacacaagttatgctatggtggtgccatcaccggaaaaacacgccaaaaattcgggacctatggtgccgtgacttagtttgcgcgcaagtatgtactgtacatggtccgacacaaaagagtgttatggagcaggggtggtggggttgattttggccatcagcgttatcgcacagattgtttgaaaacacgactcgcattgctcaacaaggacatggagagctattttttttaatttttttttaattttgatagatgtattggactagaggccaagcatctgatcaattggttgtactatatttgctcatagttgactgaggccctgtttggatacaatggttcctttctgtttccccgtacgaggtgaccgttgcagaacgggagggcaatcgcttttatatgagaaacagttccaaaacgaaacggaaccgttccatccaaacaaggcctgagagaagaggaaattgtgatcttctcagggttaagtttcttggatgtgtcgctgctactgaaacttttgttatataggtatccattttatatctgactccctccacacagaccccagtggagcgaatccccggagcactttgctcatgaaagtcgcccttgggcattactccttccagtcgaaggagatctcatgagacgagctcggaatgtgcttatatgtcaccagacggacgtgaacctccctggagatttgcttttacagtcgggcgttttttgtttcccgagtactttagagtttggaaccagttctattgctttgacaaggtctagtttattataccttcttgctctcgcgtttttttctctcgcacaactgatagacatataagtgttcttcagatagaaggaaatggtgtacgatagcgcaaggattcctaaggccattgtggacaactatgtaaggacttatgacaggtccccacgaatagtgcgatatcatacttagcagcagagaaaccatcaagaaggtagactatagcacatattcagaggcc

SEQ ID NO: 35 - DNA, 9770 bp, Tuber indicum
tgggcgattgcttgcctgaaaacaagtgatccgggggagcactgtctgtctgttcgcttcacttgtgctgttcgaatgctttacctggttgtgcgctctgagtacgagaaatgggcgagcaagctagtctgtctgttcacgaagggcgcaatgaactatcacaaaccaattccagattcttctactcttttatccccttctcacggaggttctttccagtgagcttcgcccggtcttgtctcgtctaccattgcatagttatttacagcggatggcgtaatccgcacccttgtctatgatcgcaatacggcattggctctaacaaaactatatcgtgtcgttccgagggagaaaaaaagtcccttcattctactttcccttcctccactccgattcactggcagatgaaaccgtaccgcgcgctcgggctctttggtccctattttctcaaccggcgagggcgtcgttagtatggggattggaaggctcctttggcttgtttgccgtgagtaggaagggcgctgtcattgctgagtagcctttcagcgggacctgcgggggtgcttctttccgatcggtcttgcgaggggtggtattggtattgggcggtgttggccattcggcggcgttgatgaaatttctatcaggagttctgtggcctctggatacgtggtgtagtatatcttcttggcggtttctctgctgctgctaagtatgatatcgcactattcgtggggacctgtcattaaatctttacatagttgctcacaatggtcttaggaatcctcgcgctaccgtacaccactttccttctatctgaacaacgctctatatgtttatgtatcgtacagggggagaaaggtaacgcgtgggcaagaatgcacggctggagaaaataaaatatgataggatgaaaactgcaaggtataataaactagacttttttgccacagcgatagagctagttccaaactctacagtactcagggggcaaacccacgcccgactataaaagcaaatctccggaggggctcacatccgcacggtgacatataagcgcattccgggctcgtctcatgggatctccttcgactggaaggggtgatgcccaaagacgactttcatgagtaaagtaccccggaaaagaaggaacggtgatgccttcgatgaggtcctgttttataagccttaaatcgcgctaggggttaacgctttgtgggactgtagcagcgctaccaccttctgatggtcgtgcctagatgcgaacgagagaggtgtttgaccgtcattgtttggcctgtcggggctgacatcttcccggccgagtagtaccttcaccacctccgcatgcccaccccaagcagcgagcgagagcggtgtttggccgcaatcatctggcctgtcggggttgacctcttcccgcatgagtaaaattttcaccacttcttcaagtccatgagcggcagcatgagagagcggtgattggccgtggctgtctgacttgtcgggattgacctcttcccgcatgagtagcattctcaccacctcttcatatccggaccaaacggcgtgcgagagcggtgttcggcagtagatatctggcttgtcggggttgacctcttcccgcccgagaaagattttcactactccctcatgtccataaccagcggcatgcgagagtggcgttcgaccgagattgtctggcttgtcggggttgacctcttcccgctcgagtaataatttcgcaactccctcataccccctccaagcagcattcgagagcggtgtttggccgtcattatctggttcgttgggattaacatcctcccgcccgagcagtattctcaccacttcctcatgtccaaaccaagcagcgagcgagagcggtgtttggccgtcattgtccggcttgtcggggttgacctcttccctcccgagtagtattttcaccacttcctcatgtccgcaggcagtagcatgtgagagcggtgtttggccgcgattatcgggctcgccgggatcgacctgttccagcccaagtagcattttcaccacttcctcatgtccgttatgagcagcccatgcaagtggcgtatacccccaacaatctccttcgttgatatcccaacatcccatctctattaaagcagccacaacctcgacaattccaaagaacgatgcgcagtgcagcccactgaatgggggccaggtatcgtgccctccgggacctaaattccccacttgcgctaaaagaaattcggtggatatgtggccatcaaacccctggaatagcttcagcgcaagtgatctcccgcaatccgagagttccgttttcgcatggactccccaatacatagaacaatattctagaaagggtgcatccgaggtgtcaggagatggatcggtcgaaaggtccttgacttgctgggaattcagataggtcaagcatatctctgccattgccgcatggggtctactgaaaatatcgggatgagcggaaaggtactcttggagagtaaagtggactagtcgcacggctgatgcctccttccccaccgtaataagcccttgacagcaacctatcagtgtcgacatcgaagggatgttgttagcattgaagtcggtggagcccagctcgactgccagggcgcagcagagctcgtccgtcctgagcggccgctgcgcatgactgatccacatcaaggctgccatcccgagtctcgatttatccccgccctgcgcctttatccgtcctatcgtcgcaccgtaggcatctcccagacccaaaccgtcggtcattttgctaagcctttctcgtctccgataggttgtcgattcttgtaagattgcttcgatgtttagggaaactagtataaacctggatatatctattagcggatagtgtgcaatgggaatccctctgtcattgccctacaaacatttccgagatcttcccagggatctgctctagaacgtctgcctcaagaccctcgtccatggcatctggggtttcgtcctcgtctagcctaacacgaaggtatctgataatatcatctcttctagggcctatagacacacttgccactcgcccggcaagacgcttttcgatctcggcccgaatataggaccttccagtcaggaatattcgtatccccggggacttcccaaggatttttttcagcgaatcaaggagcttggctcgatgcactgcggcacattcgtccagggcgtcaatgcacataaatgtgcgctgtgaggacgtgatggtctgtagcatctttacgatatcaggaagttgcggtccccgcccgccaatggccctcctctgctcttgaaaagcccgcgatatttcctctggaatcctttccattccgctaacgatctgtttaagcaaggaacccagcatattcgtcgcggactgctcctttcgggccgcaaagtcaaagtaaaaacaggtaacggtagtgttttgtcctctagcctgatcgcacagcctgtccactaccatcgaactgtcatcgcggcttattagcactggcgcacttctctccccagagaataactcttgttacctgataaatgtcttgcccacacctggatctccatggcaaaacaacactgccttattgccttcaccttccaaactccagtcataccagcttctgaattcgtcagtttggataagccattcccccacattgtccacacggtgctcttgaatgtcgcgatgccgtaacctaggttccagcggcgacaaccaagctaggagttgagaccgatcatcggcaactgtgaagttgttccagacattgctaaaagagctcgtattattgaagcagttgttattgctattgattgtatattgagacatcttctgctctttactgcttgcactattgaagggaagtatttttctggtttggttggttgacgatatccttgggaggtcaagaaaagcggggtctagatggggaaagcaagggtctgctggtgttaaagcagcatcgcagataccgctactcgtacatgcagtactaactcaagagttttcgtccggtaccctttttacaccaaaagtatctaacttcaccaccctctctcacagcacaatgtcaaggggaatatgccaaaatttcgcggtgtattggcaaatctgtagctaatatgggagataatattattacagaattgttgagtaatagagccccataccggtacaaaaatctaggttaatttcacacaggccaacgttattgtaaacaatgggcacaactttgatcctctacagttctacttctagagatttaaatgaataacagctatctagccgagggagggatttggagtgttttcaacagggtattgtttggattgttaggtatgttgcggtagtctgcgacatgataaacaccatacttcgtggagaatgatataaagcgctgcgcccgggaaaagtaaggacaaaataaatagacactgttaaatacgtagaacaaggtctttggcattcgagtgcaatttgaaaacttgcggtcgatattcgtgatcgagagattgtggctgttgcatcgaactaccatatgactttcctaaataggaaggcagactcttgattcttttagatggcctggcagttgttgaaggttctgaactataggaactattggtacttgtactaaatactaattcctaacaacgtacagggatgtcacgcaaaatcaaatcaaacattacacactaatggtattaaagaatgttttacttgctagcgccgtcatcgttcctgatcacctggaatttgtcagttccaatagacagacagctataagggctcttgtagatgagtgtttgaggatttacattgctatctcagcttcagggagaacaagaaaggtatgtcattttttgcccccacagcccaatatgtgcagcgaaaatggttgttttttctcccgtcaaaatacaatttttgccgggataaggggaccctaggtcgggtggggttaaatggcttggggtatcatatctagaaggaaattgacaattagcgcgcattaaactcacagctctttggtgagctttcctcgttgtctgtctgtaggtgcacgtggagcgatccaggtatctggggggacctgggaagcattcccctcttattgtggtggtagggcacctgcagtggggaaactccagcatataaataaagttataatataatattattacgattcccgcctatctcataattaaattctgcgatattcccagctctggggtattattggtcgttatctccacgatgtatcacccgatgtttgatttagcgtgactgttgtaaatcacagtggaatgagagataagagtatgtggggacaggtagaagtgacttgcttgacactctgggtggggtgaatacatacatataactcatagagatgtcatgctaaggtatatagacaactctataacacaacttggatatggtggataaagtgtgacggcctactaactgggaccacaaaggattcaattgtgtaccttaagaacatggaatacttatgctgtaatgttttatgcatgcaggaatcacccattcagtagatgtcgaattttacagctttcgaactaacactccattaccaaataagtatcgcgggtgcgcatttgtgggaagagatgggcattataccataaaccacaggttctaataagtgtttagatattggctgcttccagcgattccatcaggacttaagatatttcctttctagtacaactactaaatagagaaagacagcgaagagatggaacagagaggccctagtgcccgcctcatgtgcaattacggtatgtttgtgagagacactccgctatgatctactaccttcacagatgttcctaacattatataaacaaacgtgcaatatctcggtctctgatcatacaatcccctgtcctacatccaatcaagttcctttcctccgtgtaatccacttgacatcttagcttctatcccgggagtcggagcgggaccgggggcacattgccccaggctctcttatctgacgatatcttcgtccagaatcaagagactttgtctgtcccttctaaactccgcccccttccatcatcctgccttgcatgaagctcaggaggttgagtgcttagatgctccacaatggtttgctcaatcggccgctgctcctgggccagaagctctgacggcttagagttggttaaagattctaatttccaagctgaaaatccttttgggggcgggctttgctcgctcgtgatccagaccttctccgttcgttatattactataccgtcctactgatttcagaatggcctaacttagccaaaaacccctctgaaaccactaggagtaactgctagcaatgttagccacaatcccttaatttccagacccttaccgagaccggttgtcgcattggatggctgctcctttgttctggagttccatcttttaaacagctttcccgagagctgcatacgccgcatcaaacgtcgtagtcctccttgtccattcacctggatgcacaagcggatagccgaatatgggacatccaccggatcggctggtagtgttgcaaaagcatttggctttcctggttggccgggcagcgggtgaagctaccatacttctgaattcacatttcgcctaacggcctcctggccattggatatccccccaggacggaaaggacagcttctggatggtcaggcttaaggtatggtttcccagtccgccacacagatttatggagatcatattaaattccgcaatcagaaaaaactatcttgaaccacatacggtatctgatcgcaaagaattattgttcagggctcggataccatacctctctatcaaagaagcaatgtacgagtacaagagaggtggggttgggggacggatgctgtaacagaacccacatttcctagctcaccctgtttcattaattcctttgttcggaacttctcccgaatgtcgatcggatcttcatcaataccttgactcgttttctaccatactccagcatctccgaccgtaaaaaaaaaaattcgcggaccttttcgccaggaatcttggaaacctaagggctctcccttcctgccctgggggaaatcttatcgtgtcatatgtgtgataataatattatgacattttcatgagaatactattctcgggccctaatagggttgagttgtacgtgacggcttcttggcgtcactctattttttgggtcttggcagtccccctcgtgactggaggctacaaaggggatccatcctttccccttctttcctccaccgacaaccgtcaagttcgacagaaacattcggcctttctacttgcacctctccactcacgtcgtttcctttttttctgccactatgacttcttctttgtccaacctcaatatggcccttgaagccgggcctcttattcaccaacattatgatggaatcggtctcgccgagtatattcccttgctcgccaacggaacctttttctttgatgggtcggatggagtctccctccaggtccgccacctccttccagcagactggaagcccgtcaatggaccacttgcggtcttgttcgattccgtcccacataccattattgggactcacttttctatccgcaatcaccccatttataagcttaacgatggttctatcttggtcgtctttgacccggccgctaagcacggtttcgagtttaccaatcaatctttcgtggacccccagcttgaggtctccggagagaaccgtttcttgcgtctcgttaccaatgaccaagacttcgcctaccatggttctagtcttagcatgattcatgacaagggcgttaagcgccctgccaatgcgtaagtacaattctaccaatgtaccagtttcgtgtgctcatctagtcataggtttatcttattcagaacggacaaaatgaagcccctcaaggctcagtacccggagatgggcaacaacgatatctgtatgtggctcttattttccctttattttgatctctattcttggtaccatttgcctaacgttttaatagccaagattctcggtcgcatgtggcagaattcctcggatgaggtcaaagatgtttataaggtaaaacatctcacctaactgttagttagaatcactaacacattaataggcgagggcccatcagcttgcggtttcccataagttgaccaacccggattacaaatactccccgcgccgctctcgcgaaatcatgaggagggactccgggaataaaggtccttctccccgtatcgaccttcatcgtgctggtgttgcgaagcgtgaaaaaaagaggatcattacccaccttgggttcctcactcaacgtttcgcaactcaccccactgctaacaatctcggtgatatcatcgaggaagccgaaaaggtcttgaaggcctttggtatggtcaattgagtgatctcaacggagggactcatcagtgcctgggtgatatactttcaatgggagggatgatcttgtctatcaaaggtcattatctcagtcgaataaatttatatgacctaaataaataaaaaatatgtttaaaaaaaaaaaaatgaggggtctgaacactatatcatacagtaaaatatcaagttgtgaagcagaagagagtcgactaccggtatgggaagaggcagtgagaatctaattctgactgtcttagtctggaaagttcgtacagcacttgcacacaagttatgctatggtggtgccatcaccggaaaaacacgccaaaaattcgggacctatggtgccgtgacttagtttgcgcgcaagtatgtactgtacatggtccgacacaaaagagtgttatggagcaggggtggtggggttgattttggccatcagcgttatcgcacagattgtttgaaaacacgactcgcattgctcaacaaggacatggagagctattttttttaatttttttttaattttgatagatgtattggactagaggccaagcatctgatcaattggttgtactatatttgctcatagttgactgaggccctgtttggatacaatggttcctttctgtttccccgtacgaggtgaccgttgcagaacgggagggcaatcgcttttatatgagaaacagttccaaaacgaaacggaaccgttccatccaaacaaggcctgagagaagaggaaattgtgatcttctcagggttaagtttcttggatgtgtcgctgctactgaaacttttgttatataggtatccattttatatctgactccctccacacagaccccagtggagcgaatccccggagcactttgctcatgaaagtcgcccttgggcattactccttccagtcgaaggagatctcatgagacgagctcggaatgtgcttatatgtcaccagacggacgtgaacctccctggagatttgcttttacagtcgggcgttttttgtttcccgagtactttagagtttggaaccagttctattgctttgacaaggtctagtttattataccttcttgctctcgcgtttttttctctcgcacaactgatagacatataagtgttctttagatagaaggaaatggtgtacgatagcgcaaggattcctaaggccattgtggacaactatgtaaggacttatgacaggtccccacgaatagtgcgatatcatacttagcagcagagaaaccatcaagaaggtagactatagcacatattcagaggcc

SEQ ID NO: 36 - DNA, 10118 bp, Tuber indicum
tgggcgattgcttgcctgcagacaagtgatccggacccatccagtggtgggtccggcacggggggagcactgtctgtctgttcgcttcacttgtgctgttcgaatgctttacctggttgtgcgctctgagtacgagaaatgggcgagcaagctagtctgtctattcacgaagggcgcaatgaactatcacaaaccaatcgcagattcttctactcttttgtccctttctcacggaggttctttccagtgagcttcgcccggtcttgtctcgtctaccattgcatagttatttacagcggatggcgtaatccgcacccttgtctatcatcgcaatacggcattggctctaacaaaactatatcgtgtcgttctgagggagaaaaaaagtcccttcactctactttcccttcctccactccgattcactggcagatgaaaccgtaccgcgcgctcgggctctttggtccctattttctcaatgggcgagggcgtcgttagtatgggggttgttaggctcctttggcctatttgccgtgagtagggagggcgctgtcattgctgagtagcctttcagcggggcctgcgggggtgcttctttccgatcggtcttgcgaggggtggtattggtattgggcggtgttggccattcggcggcgttgatgaaatttctatcaggagttctgtggcctctggatacatggtgtagtatatcttattggcggtttctctgctgctgctaagtatgatatcgcactattcgtggggacctgtcattaagtctttacatagttgctcacaatggtcttaggaatcctcgcgccatcgtacatcactttccttctatctgaacaacgctctatatgtttatctatcgtacggggggagaagggtgacgcgtgggcaagaatgcacggctggagaaaataaaatatgataggatgaaaacttcaaggtataataaactagacttttttgccacggcgatagaactaattccaaactttacagtactcagggggcaagcccacgcccgactataaaagcaaatctccaggggggctcacatccgcacggtgacatataagcgcattccgggctcgtctcatgggatctccttcgactggaagggctgatgcccaagggcgactttcatgagtaaagtaccccggaaaagaaggaacggtgatgccttcgatgaggtcctgttttataagccttaaatcgcgctaggggttaacgctttgtgggactgtagcagcgctaccaccttctgatggccgtgcctagatgcgaacgagagaggtgtttgaccgtcattgtttggcctgtcggggctgacatcttcccggccgagtagcaccttcaccacctccgcatgcccatcccaagcagcgagcgagagcggtgtttggccgcaatcatctggcctgtcggggttgacctcttcccgcatgagtaacattttcaccacttcttcaagtccatgagccgcagcatgagagagtggcgttcggccgagattgtctggcttgtcggggttgacctcttcccgctcgagtaataatttcgcaactgcctcatgccccctccaagcagcattcgagagcggtgtttggccgtcattatctggctcgttgggattaacatcctcccgcccgagcagtattctcaccacttcctcatgtccaaaccaagcagcgagcgagagcggtgtttggccgtcattgtccggcttgtcggggttgacctcttcccgcccgagcagtattttcaccacttcctcatgtccacaggcagtagcatgtgagagcggtgtttggccgcgattatccggctcgtcgggatcgacctgttccagcccaagtagcattttcaccacttcctcatgtccgttatgagcagcccatgcaagtggcgtatacccccaacaatctccttcgttgatatcccaacatcccatctctattaaagcagccacaacctcgacaattccaaagaaagatgcacagtgcagcccactgaatggaggccaggtatcgtgcccttcgggacctaaattccccacttgcgctaaaagaaattcggtggatatatggccatcaaactcctggaatagcttcagcgcaagagatctcccgcaatccgagagttccgttttcgcatggactccccaatacatagaacaatattctagaaagggtgcatccgaggtgtcaggagatggatcggtcgaaaggtccttgacttgccgggagttcagataggtcaagcatatctctgccattgccgcatggggtctactgaaaatatcgggatgagcggaaaggtactcttggagggtaaagtggactagtcgcacggctgatgcctccttgcccaccgtaataagcccttgacagcaacctactagtgtcgatatcgaagggatgttgttagcattgaagtcggtggagcccagctcgactgccagggcgcggcagagctcgtccgtcctgagcggccgctgcgcatgactgatccacatcaaggctgccatcccgagtctcgatttatccccgccctgcgcctttatccgtcctatcgtcgcaccgtaggcatctcccagacccaaaccgtcggtcattttgctaagcctgtctcgtctccgataggttgttgattcttgtaagattgcttcgatgtttagggaaactagtataaacctggatatatctattagcggatagtgtgcaatgggaatccctctgtcattgccctacaaacatttccgagatcttccctgggatctgctctagaacgtctgcctcaagaccctcgtccatggcatccggggtttcgtcctcgcccagcctaacacgaaggtatctgataatatcatctcttctagggcttatagacacacttgccgctcgcccgggaagacgcttttcgatctcggcccgaatataggaccttccagtcaggaatattcgtatccccggggacttcccaaggatttgtttcagcgaatcaaggagcttggctcgatgcactgcggcacattcgtccagggcgtcaatgcacataaatgtgcgctgtgaggacgtgatggtctgtagcatctttacgatatcaggaagttgcggtccccgcccgccgatggccctcctctgctcctgaaaagcccgcgatatttcctctggaatcctttccattccgctaacgatctgtttaagcaaggaacccagcatattcgtcgcggactgctcctttcgggccgcaaagtcaaagtaaaaacaggtaacggtagtgttttgtcctctggcctgatcgcacagcctgtccactaccatcgaactgtcatcgcggcttattagcactggcgcacttctctccccagagaataactcttgttacctgataaatgtcttgcccacacctgggtctccatggcaaaacaacactgccttattgccttcaccttccaaactccagtcataccagcttctgaattcgtcagtttggataagccattcccccacattgtccacacggtgctcttggatgtcgcgatgccgtaacctaggttccagcggcgacaaccaagctaggagttgagaccgatcatcggcaactgtgaagttgttccagacattgctaaaagagctcgtattattgaagcagttgttattgctattgattgtatatcgagacatcttccgctctttactgcttgcactattgaagggaagtatttatctggtttggttggttgacgatatcctagggaggtcaagaaaagcggggtctagctggggaaagcaagggtctgctggtgttaaagcagcatcgcagataccgctactcgtacatgcagtactaactcaagagttttggtccggtaccctttttacaccaaaagtatctaacttcaccaccctctctcacagcacaatgccaaggggaatatgccaaaatttcgcggtgtattggcaaatctgtagataatatgggagataatattattacagaattgttgagtattatagaggggtggagaaaatgtcatttaagtgcgaggtattggttgtcatgtacttttttgtatagagccccatacaggtacaaaaatccaggttaatttcacacaggccaacgttattgtaaacaatgggcacaactttgatccctttgatcctctacagttctacttctagagatttaaataaataacagctatctagctgagagagggatttaaagtgttttcaacgggtattgttgggattgttaggtatgttgcggtagtctgcgacttgataaacaccatacttcgtggagaatgatataaagcgctgcgcccgggaaaagtaaggacgaaataaatagacactgttaaatacctagaacaaggtctctagcattagagtgcaatttgaaaacttgcggtcgataaattattcgtgatcgagagattgtggctgttgcatcgaactaccatatgactttcctaaataggaaggcagactcttggtgcctggcagttgaaggttctgaaataggaactattggtactcgtactaaatactaattcctagcaacgtacagggatgtcacgcaaaatcaaatcaaacataacacactaatggtattaaagaatgttttacttgctagcgccgtcatagttcctttcccaatcacctggaatttgttagttccaatagacagctataagggctcttgtagatgagtgtttaaggatttacattgctatctcagcttcagcgagaacaagaaaaatgtcattttttgcccccacagcccaatatgtgcagcgaaaatgtttgttttttctcccgtcaaaatacaatttttgccaggataaggggaccctaggtcagggtggggttaaacggcttggggtatcatatctagaaggaaattgtggatccctggacaattagcgcgcattaaactcacagctctttggtgagctttcctcgttgtctttctgtaggtgcacgtggagccagccaggtatctggggggacctgggaagcattccctcttattgtggtggtagggcctgcaatggggaaactccagcatataaaatttaataaagttatattataatattattattacgactcccgcctatctcataattaaattctgcgatattcccagctctggggtattattggttgttatctccatgatgtataacccgatgtttgatttagcgtgactattgtaaatcacagtggaatgaaagataagagtaaatggggacaggtggtagaagtgacttgcttgactttctgggtggggtggatacatacatatagctcatagagatgtcatgctaaggtatatagacaactctatgacacaacttggatatggtggataaagtgtgacggcctactaactgagaccacaaaggattcaattgtgtaccttaagaacatggaatacttatgctgtaatgttttgtgcatgcaggaatcaccctttcagtagatgtcgaattttacagctttcgaactagcaatccattaccaaataagtatcgcgggtgcgcatttgtgggaagagatgggcattataccataaaccacaggttgtaaaaagtgtttagattttggctgcttccagcgattccatcaggactcaagatatttcctttccagtacaactactaaatagcaaaagatagcgaagagatggaagagagaggccctagtgtccgcctcatgtgcaattacggtatgtttgtgagagacactccgctatgatctactaccttcacagatgttcctaacattatatatacaaacgtgcaatatcccgatctctgatcacacaatcccctgtcctacatccaatcaagttcctttcctccgtgtaatccacttgacatcttagcttctatcccgggagtcggagcgggaccgggggcacattgccccaggctctcttatctgacaatattttcgtccagaatcaagagactttgtccgtcccttctaaactccgcccccttcaatcatcctgccttgcatgaagctcaggaggttgagtgcttagatgctccacaatggtttgctcaatcggcagcggctcctgagccagaagctctgatggcttagagttggttaaagattttaatttcccagctgaaaatccttttggggtcgagctttgctcgctcgtgatccagaccttctccgttcgttatattactataccatcctactgatatcagaatggcctaacttagccaaaaaacccctctgagaccactaggagtaactgctagcaatgttagccacaatccttaatttccagacccttaccgagacccgttgtagcattggatggctgctcctttgttctggagttccatcttttaaacagctttcccgagagctgcatacgccgcatcaaacgccgtagtcctccttgtccattcacctggatgcacaagcggatagccgaacatgggacatccgccggatcggctggtagttttgcaaaagaatttggctttcctggttggcagggcagcgggtgaagctaccatacttctgaattcacatttcgcctaacggcctcctggccattggatatccccccaggacggaaaggacagcctcgggatggtcaggcttaaggtaccggtatggtttcccagcccgccacacagatttatggagatcatattaaattcccaatcagaaaaaactatcttgatccacatacggtatctgatcgcaaagaattattgttcagggctcagataccatacctctctatcaaagaagcaatgtacgagtacaagagaggtggggttgggggacgggcgctgtaacagaacccacatttcctagctcaccctgtttcattaatttctttgttcggaacttctcccgaatgtcaatcagatcttcatcaataccttgactcgttttctaccgtactccagcatctccgaccgtaaaaaaaaattcgcggatctttttcaagataatactattctcgggccctaatagggttgagttgtacgtgacggcttcttggcgtcactctattttttgggtcttggcagtccccctcgtgactagaggcctacaaaggagatccatcctttcccccttctttcctccaccgacaaccgtcaagttcaacagaaacattcggcctttcaacttgcacctctccactcacgtcgtttccttttttctgccactatgactatgacttcttctttgtccattctcaatatggccgttgaagccgggcctcttattcaccaacgttatgatggaatcggtcttgccgagtatattcccttgctcgccaacggaacctttttctttgatgggccggatggagtctccctccaagtccgccatctccttccagcagactggaagcccgtcaatggaccacttacggtcttgttcgattccgccccccataccattattgggactcatttttctatccgcaatcatcccatttataagcttaacgatggttctatctttgtcgtcttggacccggccaccaagcacggtttcgagtttaccgatcaatctttcgtggacccccagcttgaggtctccggagaaaaccgtttcttgcgtctcgttaccaatgaccaagacttcgcctaccatggttctagtcttagcatgattcatgacaagggcgttaagcgccctgccaatgcgtattgatacaattctaccaatgtaccagtttcgtgtgctcatctagtcataggttcattttattcagaacggacaaaatgaagcccctcaaggctcagtacccggagatgggcaacaacgatatctgtacgtggctctcatttcccccttatttagatctctattcttggtaccatttgcctaacgttttaatagccaagattctcggtcgcatgtggcagaattcctcggatgaggtcaaagatgtttataaggtaaaacatctcacctaactgttagttagaatcactaacacattaataggcaagggcccatcagcttgcggtttcccataagttgatcaacccggattacaaatactccccgcgccgctctcgcgaaatcatgaggagggactccgggaataaaggtccttccccccgtatcgaccttcaccgtgctggcgttgcgaagcgtggaaaaaagaggatcgttacccaccttgggttcctcactcaacgtttcgcgactcaccccactgttaacaatctcggtgatatcatcgaggaagccgaaaaggtcttgaaggcctttggtatggtcaattgagtgatctcaacggagggactcatcagtacctgggtggtatactttcaatgggagggatgatcttgtctatcaaaggtcattatctcagtcgaataaaaaaatttatatgacctaaataaataaaaaatatgtttaaaaaaaaaaaaaaaaataaggggtctgaacactatatcatacagtaaaatatcaagttgtgaagcagaagagagtcgactaccggtatgggaagaggcagtgagagtttaattctgactatcttagtctggaaagttcgtacagtacttgcacatatgttatgttatggtggtgctatcacctgagaaacacgccaaaaattcgggacctatggtgccgtgacttagtttgcgcgcaaatatgtactgtacatggtccgacataaaagagtgttacggagcaggggtggtggggttgattttggccatcagcgttatcgcacagattgtttgaaaacacggctcgcattgctcaacaagaacatggagagagctatttttttaattttgataaatgtattggactagaggccaagaatctgatcaattggttatactatatttgttcacagttgactgaggccctgtttggatacaacggtccctttctgtttccccgtacgaggtgaccgttgcagaacgggagggcaatcacttttatatgagaaacagttccaaaacgagacggaaccgttccatccaaacaaggcctgggagaagaggaaattgtgatcttctcaggtttaagtttcttggatgtgtcgctgctactgaaaattttggtatataggtatccgttttatatctgactccctccacacagaccccagtggagtgaatacccggagcactttgctcatgaaagtcgcccttgggcatcaccccttccagtcaaggagatctcatgagacgagctcggaatgtgcttattgtcaccatacggacatgaacctccctggagatttgcttttacagtctggcgtttttttgtttcccgagtactttagagtttggaactagttctattgctttgacaaggtctagtttattataacttcttgccctcgcgttttttctcttgcacaactaatagacatataagtgttcttcagatagaaggaaatggcgtacgatagcgcaaggattcctaagaccattgtgagcaactatgtaaagacttatgacaggtccccacgaatagtgcgatatcatacttagcagcagagaaaccatcaagaaggtagactacagtgttgaaaatcacgaagcttccactgtttagagtatgaggcgatggttgtttggcaatgttttagaaacacaatggtggaacatgctaaaggaacgagtcaacgaaagagtcaacgggtaagtaagagtacgtaggaactagcgttagtgcaaggagaagtgtttgggatgttttaggagttagcactagggatgccggaagtaacgggttacactccggaggtctagtcgacaaccaggtgtcgcactatgggagataggaaactggagaagatgcacgaaggtatcgggactgtctggaacagcgatagcctaggagaaacgtttcggaggatgttcgaaggagttgtgtggatggaactgtgtggcatcatgaagaaaagatggtatggaattctgcgaggcagatggacgaagaaaatgtttgcaaaggaacagcgcgtgtatatataggacacctccggcagcatagaacaaacaagaatcgcagacactctatacctatcatagccttattatttgatacagcacatattcagaggcc

SEQ ID NO: 37 - DNA, 7583 bp, Tuber indicum
  misc_feature (3894)..(3894): n is a, c, g, or t
ggcttgcatgatatcagcgaatgtagctcggggactaaatctgatagttacactaaacgttggataccacgctcgttcagagctagctccttctccgctcgaggtagaggattaagttcacaggcctctgaatggcatctgatgatgttccgggcaacttctctcggggcatctgtgtcgctagcaattgtatgataacttttgccttctgcatgaagctcaatcattcgtactctctcatcgcttgcggccattctccgcttgcctgttgcttgaccttgaggtatggtgtatgatgggaggtgttggctcccataaagaagtcgtggctaagatactagtgtcgggttactaaataacctctactgctgagaccgttgcgaaagctgggtaggttctctctacatattctagtagatattgacgctgatttgcgggaggaaaaaaaggaattttatgttttttttggttttaaagataccgagggcgtttcattgatcctagatacataagtttaatttaagtctacgcgatctataatacgctgagaatgcctcccaactatgcccttaatttccgccgctcccacacagtcatactgtgatacagtaacttcagtttttttttgtacccactagcctctttaggccttctcgggaggtcatatcggttcacaatccagtggaggattctccaataggaggctgtttagatcatgttagaattgggcgctggtggaatatgttgttcggggaacttctcacaaaaacgctgtatccaccagaagtgaaagtgttcatgtgcatctcggtctcgggctggaagtgcgggtcgagttgagagtctccattctcctcagcgctagcgatgtcgaaagcattgaggatgttggcggcttcctcctcggtgagatcgagctgatggttgctggaaacgtaaacacgggtgatggtgaatctggacaccgcggatgcgggcttggcacgagccttccttcttgcagcagccttatcggccgcttccttgtcgatgggggccttggggacaaagccagcttcacgcaagagcttctcaggggtagtagggtgaccgtgtttcgccaattcggtctcgataaattcttcgagcacgagaccagggtcaccacgatccctagcagcagtataaagagaggcaatgttttgccacatatacttattggtctcagcgacccagacatcacgagtgagattggaaatttcggcttgagtgagcccataaccgttacagtacttagaaatccaggctaatagatggttagaaatagaggcaaaatagggatgtgatttacttacatctctgcgcaacgtagggattcaatgcacgaaggtgctgagagggggggatgcgggccaaggcctcatcaccattgacgataacagccggaatagtggcatcctgggccgtccacggctgtccaggtttatagtatccgcaatacaaacgtagaaccgacacaacataagggacttcggggtcgagggggactcgccctcgatgacggacggttgattgaattgcaccaagccacttgtcgtagggcaagagttcgccgctgacagtgaaagtgacaccaccggggggttgaacgataatgggggagttctcgtgttggacccagatgcacccgatcctgtcttgcatgagaatgtacccacggtccgaaaggtacaaggcgcatcccaaggtagcattacgcataatgaggcgtctctcgagcctgttctcccactccaaaaccttaggggggggggagaggttaacgaccatgctcagattgacaggacgagacgacattatgaaaggcggggggggtggagacgggaaaagaaatttgaaccgacaaaaggaggattgcgtatgggcgtttcaatgagtggagattggcctcaatggctgttgatggaagaaaggagggtggaaggtctggcctcttttgtatgctacccgtcgaagtgggatgccaaggcccaaaaaaaagagtgacgccaagaagccgtcacgtacaactcagccctactagggtctatcataagatttttcccagagcaggtagagagcccttaggtttctgggattcctcgcggaaagatcggggaacatttttgtatttacggtcagagttgctcgaatagaaagacgagtcaaggtatttgatgaagatctgatcgacattcgggagaagtttaaaacaaaggaattagtgggggtgagtgggaaatgtgggttctgttaaagtatctgtcccccaatcttaactacccctcttgtacattgcctctttggtaaaggggtatggtatctgaaccttgaacaataattccttgcgataatatatatcgatcaggacagtttgctgactggaaaatttaatatagtctccataaaaccgtgtgaccgactcggactgggaaatcataccttaagtctggccatccggaagccgctctttctgctatggggtaatatcaaatagccaagaggccatcaggcgaaatgagaattcagaaatatgatagccctcccctgccaaccaagagacaaatacttcggcaataccaccacccgatctggtggatgtctccatgttcggctatccgcttgagtatccaggggaatagacaaggaagactaccacatttgatgcggtgtgtgcggctgccgagaaaactgtttgaaagatggaactccagaacaaaggattaactggtcaatgcaacacctggtcgctgcaagggtctgaagattaaaggattgtaactagtattgttggaggttactcctagcaatttcagaggggtttttggtaaagatagactacagtcgcaccccgctgtcccggacctcgctctcccggatcccccgttgtccgggacgaaattctctgagctctgaatttttttgcaccagaacgtaccccgctgtccaggacagattctgcggattttggccatccgggacagcgaggtgcgactgtatactgatatcagtaggatggtatggcactacaatgaaagaaagtggtctggaccatgagctcaaaaactccaaagagaatttccggccgggaaattataatatttaaccggtttaaaaccgtgagatcctctggcccaggaccagcggccgattgagagagctgttgtgagagcatcttagcactcaaccattagaactccatgataggtaggatgataaaaggggaatgggtttagaatgggtgcacaaagcttcttgattctggattaagatatgactacataagggagtctgaaacaatgtacactctatctccgcttggacttataggataaaaatggacatgttaagtgaattataatgaggaaaaaagcctgattggttgtagggatcgattgtatgatcaggggtgggagtactacaagtttgtctatatagtattgggaataactgtaaaaatagttccagaatatggctgagtatttcacaaacatgcattagttgaatatgaggtagatactagagtcaccttgttcagtcccttggctgtctttgaatatctggaaaatataccaggaaagaaagtgggaaaaacaatatcttcaacttctttggcaaactctttggaagtagcaaaacctaacctctccttacagcctgagcctgtagcttgatatggtactcatttactctttcatccgcatagctatgcacttgcaatatctcttccaactgtttgtttaaaaaccatgaggtttgacatcaaatgaaggagtaatttccaggtgcacaaaaaatatattgtgtgaatacctacaattgagcccaaatcgatttagtaaattgaaacccatcaagtagttggtaatctatctnatgaagttttttgagctgaagggaaaagagaaatgttaggcaaatttttttgtgacccttagctggtcggctgtcaatgcttgatattgtcatccaaaccaccacagattgtattgctgggatgttgacatgttgcagtacgacatctcctaaggcgatatgcatatcagtgattaccttatcgtactaagaaagacaagacagtcatttctactacttgcttttttattctcttacttcatattctaccatgatttacaggggtcatgctaaattaaatattgtgtgataccttgtgaaaaatgtccaccaaagccacagagctgtgaatatcgcaaattgtagggtgagagggaaatcacaatgcataactttataataattataaacaccacaactgctccactggggtgccttatgctaggactttcctccatgcgaccctttttgcatcataaaacaaataataacagattttacaaaagaaaaaataaaaaccaatgaggatgccccaccgccacattaaaggagaaagtagttcttagatcttgtctccctcaattcacttagtgcacctgtggatagaaacttaggtgaagatagagaaagaggtatcatgagttttaatgtacattattcattgcaatttaattccgggcacactaagttatttatttatttattcaatccctactgcaatccaagctactcttgttcttgccaagactatatcttgggggcggggaacaaactattttcattgcaccttttgggcgctacgagcaaaaaattgaccattaaattttcatcaatttaaaatcctaaatacaagtatcatacacatctactcaagtacaagaatccttgtaactattggaacaaaacaaattctgggactcttgtcggacactacgttaacgctagtaagtaacaaaaaatcttttaatattagaagtgtgtgtgacatccacttagattttgcgtgacatccctgccgctaccagaccttagtactcgagtagtagaataagaggagtagcgaccttcaacgacagccaagccatcaaaaagggtatcgtactcgagtgttcaaatagaaaatctaccttcctgtgataccaggtgaggtcattgtggtggttcaatacaacaatcaagtcctgcgattacgcataacaagtgtgagtactcgagaagagcgcgaatacacggctgcctggcccgcgtgggtctaacagatctgtccttgctctatttatttgttttgtttttcctaagcgcagcactttttaccctatgaggtatttataataatattatagatattctggaggaggttcccaaaaatatatcggaaatgcacctaggggcagtggtactaggattcccaccgcacactatctgctagtggatatgtctaggtttctagtttccataaacaccagacgagaaaggcttaccaaaacgacagagggggttgggtctgggagatgtctacggtgcgacgatagaacggataaaagcgcagggcggggataaatcgagactcgggatggcaactttgatgtggatcagtcatgcgcagaggctgctcgggacggacgagctctcccacgccctggcagttgagctgagctccaccaacttcaacgctagcaacatctcttcgatgtcgatactgctgggttgtttctaagggcttattacggtggacaaggaagcatcagccgtgcaactggcccactttactctccaagagtacctttccgctcatcccgatattttctgcagaccccatgcggcgatggcagagatctgcttgacctatctgaattcccggcaaatcaaggacctttcgaccgacccatctcctaacaccccgggtgcaccctttctagaatattgttctgtgcactggggaatccatgcgaaaacggaactctgggattgcaggagatcgcttgcgctgaagctattctaggagtttgatggccacatatccacagaatttcttttagcgcaagtggcgaatttaagtcccaaggggcgtgatacctggcctccgttcagtgggctgtactgcgcatcgttcttcggaatcgtggagattatggctgctttaacatacgtgggatgktgggataccggggaaggaggttgtttggggtaaccgccgcttgcacgggctgttcataacgggcatgggggggtggtggaaatgctattcgggttggaaaaggtccatccggacgagctggataaccgcgaccggacaccgctctcacatgttactgcctgtcggcatgaagaagtggtgaaaatattgctcctgtttgagcgggaagaggatagccacaaaaagccggacaatgacggtcaaacaacactctctttcgctgctcggtttggacatgaggaagtggtgggaatactacttgggtgggaagatgttaatcccaatgagctagataatgacggccaaacaccgctctcgaatgctgcttggaggggtcatgagagagttgcgaagatattactcgggcgggaagaggtcaacctcgaagagccagacgagctcggccgggcgccgctcttgcatgccgctggtcatggacatgagggagtagtgaaaatttttctcgggcgggaagaggtcaacctcgggaagctagacaattactcccaaacaccgcttttgcatgcgtttggtctggatatgaagaggtggtgagagtgctactcatacggggagtgtcgataccggcgaaccggacatcagcggccaaataccgctctcgcatgctgncgctcatgggcacgaggaggtggtagaaatgttactcatgtgggaagaggtcaaacccgacaggccagatgatttcggccaaacacttgctgcttgggatggacatggggggatgatgaagtactactcggccgggaagatgtcaaaccggacaggccagacgatagcggtaaaaccctgctcttgttggcctcttgggataggaatgagggagcggtgaaaatactacttgggcgcgaagttgtcggccccgacgaaccggataatgacgaccaaatacccctctcgttcgcaactgggaatgaccatcagaaggcggtggcccacaaagcgttaacccctagcgcggtttaaggtttataaaaagcaacctcttcgaagggatcaccttccttttcttacagagtactttgctcgtgggggtcgacctagagcatcaccctttccagtcgaaggagatcccatgagacgagtctggaatgcgcttatatgccaccatgcggacgtgaacctctctggggatttgcttttatagtcgggcgtgggttttttttctgagtacggtagagtttggaactagttctattgccgtgacaaagtctagtttagtttattacaccttgcagtcctcatcctatcatatttgatttcctccagccgtacattcttgccctcacgttttttctctcccttgcgcaattgataagcatatagagtgttgttcatatagaaggaaagtggtgtatgagaggggaagggttcttaagaccaatgtgagcagctatgtaaggactgaatgataggtccccacgaatagtgcgatatcatacttagcagcagagaaaccatcaggaaggtaaactacagcacatattcagatgcc

SEQ ID NO: 38 - DNA, 7742 bp, Tuber indicum
ggcttgcatgatatcagcgaatgtagctcggggactaaatctgatagttacactaaacgttggataccacgctcgttcagagctagctccttctccgctcgaggtagaggattaagttcacaggcctctgaatggcatctgatgatgttccgggcaacttctctcggggcatctgtgtcgctagcaattgtatgataacttttgccttctgcatgaagctcaatcattcgtactctctcatcgcttgcggccattctccgcttgcctgttgcttgaccttgaggtatggtgtatgatgggaggtgttggctcccataaagaagtcgtggctaagatactagtgtcgggttactaaataacctctactgctgagaccgttgcgaaagctgggtaggttctctctacatattctagtagatattgacgctgatttgcgggaggaaaaaaaggaattttatgttttttttggttttaaagataccgagggcgtttcattgatcctagatacataagtttaatttaagtctacgcgatctataatacgctgagaatgcctcccaactatgcccttaatttccgccgctcccacacagtcatactgtgatacagtaacttcagtttttttttgtacccactagcctctttaggccttctcgggaggtcatatcggttcacaatccagtggaggattctccaataggaggctgtttagatcatgttagaattgggcgctggtggaatatgttgttcggggaacttctcacaaaaacgctgtatccaccagaagtgaaagtgttcatgtgcatctcggtctcgggctggaagtgcgggtcgagttgagagtctccattctcctcagcgctagcgatgtcgaaagcattgaggatgttggcggcttcctcctcggtgagatcgagctgatggttgctggaaacgtaaacacgggtgatggtgaatctggacaccgcggatgcgggcttggcacgagccttccttcttgcagcagccttatcggccgcttccttgtcgatgggggccttggggacaaagccagcttcacgcaagagcttctcaggggtagtagggtgaccgtgtttcgccaattcggtctcgataaattcttcgagcacgagaccagggtcaccacgatccctagcagcagtataaagagaggcaatgttttgccacatatacttattggtctcagcgacccagacatcacgagtgagattggaaatttcggcttgagtgagcccataaccgttacagtacttagaaatccaggctaatagatggttagaaatagaggcaaaatagggatgtgatttacttacatctctgcgcaacgtagggattcaatgcacgaaggtgctgagagggggggatgcgggccaaggcctcatcaccattgacgataacagccggaatagtggcatcctgggccgtccacggttgtccaggtttatagtatccgcaatacaaacgtagaaccgacacaacataagggacttcggggtcgagggggactcgccctcgatgacggacggttgattgaattgcaccaagccacttgtcgtagggcaagagttcgccgctgacagtgaaagtgacaccaccggggggttgaacgatgatgggggagttctcgtgttggacccagatgcacccgatcctgtcttgcatgagaatgtacccacggtccgaaaggtacaaggcgcatcccaaggtagcattacgcataatgaggcgtctctcgagcctgttctcccactccaaaaccttaggggggggggagaggttaacgaccatgctcagattgacaggacgagacgacattatgaaaggcggggggggtggagacgggaaaagaaatttgaaccgacaaaaggaggattgcgtatgggcgtttcaatgagtggagattggcctcagtggctgttgatggaagaaaggagggtggaaggtctggcctcttttgtatgctacccgtcgaagtgggatgccaaggcccaaaaaaaagagtgacgccaagaagccgtcacgtacaactcagccctactagggtctatcataagatttttcccagagcaggtagagagcccttaggtttctaagattcctcgcggaaagatcggggaacatttttgtatttacggtcagagttgctcgaatagaaagacgagtcaaggtatttgatgaagatctgatcgacattcgggagaagtttaaaacaaaggaattagtgggggtgagtgggaaatgtgggttctgttaaagtatctgtcccccaatcttaactacccctcttgtacattgcctctttggtaaaggggtatggtatctgaaccttgaacaataattccttgcgataatatatatcgatcaggacagtttgctgactggaaaatttaatatagtctccataaaaccgtgtgaccgactcggactgggaaatcataccttaagtctggccatccggaagccgctctttctgctatggggtaatatcaaatagccaagaggccatcaggcgaaatgagaattcagaaatatgatagccctcccctgccaaccaagagacaaatacttcggcaataccaccacccgatctggtggatgtctccatgttcggctatccgcttgagtatccaggggaatagacaaggaagactaccacatttgatgcggtgtgtgcggctgccgagaaaactgtttgaaagatggaactccagaacaaaggattaactggtcaatgcaacacctggtcgctgcaagggtctgaagattaaaggattgtaactagtattgttggaggttactcctagcaatttcagaggggtttttggtaaagatagactacagtcgcaccccgctgtcccggacctcgctctcccggatcccccgttgtccgggacgaaattctctgagctctgaatttttttgcaccagaacgtaccccgctgtccagggcagattctgcggattttggccatccgggacagcgaggtgcgactgtatactgatatcagtaggatggtatggcactacaatgaaagaaagtggtctggaccatgagctcaaaaactccaaagagaatttccggccgggaaattataatatttaaccggtttaaaaccgtgagatcctctggcccaggaccagcggccgattgagagagctgttgtgagagcatcttagcactcaaccattagaactccatgataggtaggatgataaaaggggaatggggtttagaatgggtgcacaaagcttcttgattctggattaagatatgactacataagggagtctgaaacaatgtacactctatctccgcttggacttataggataaaaatagacatgttaagtgaattataatgaggaaaaaagcctgattggttgtagggatcgattgtatgatcaggggtgggagtactacaagtttgtctatatagtattgggaataactgtaaaaatagtttcagaatatggctgagtatttcacaaacatgcattagttgaatatgaggtagatactagagtcaccttgttcagtcccttggctgtctttgaatatctggaaaatataccaggaaagaaagtgggaaaaacaatatcttcaacttctttggcaaactgtttggaagtagcaaaacctaacctctccttacagcctgagcctgtagcttgatatggtactcatttactctttcataaataaataaacaaataaacccgtcggacggggggggggggctggctggctggctggctggactgggctggcggttgtgtatgtgtgagatttgctattttaccttccacagtaccatcatcatcatacttgcttacttttttacttttaacttcatccgcatagctatgcacttgcaatatctcttccaactgtttgtttaaaaaccatgaggtttgacatcaaatgaaggagtaatttccaggtgcacaaaaaatatattgtgtgaatacctacaattgagcccaaatcgatttagtaaattgaaacccatcaagtagttggtaatctatcttatgaagttttttgagctgaagggaaaagagaaatgttaggcaaatttttttgtgacccttagctggtcggctgtcaatgcttgatattgtcatccaaaccaccacagattgtattgctgggatgttgacatgttgcagtacgacatctcctaaggcgatatgcatatcagagattaccttatcgtactaagaaagacaagacagtcatttctactacttgcttttttattctcttacttcatattctaccatgatttacaggggtcatgctaaattaaatattgtgtgataccttgtgaaaaatgtccaccaaagctatagagctgtgaatatcgcaaattgtagggtgagagggaaatcacaatgcataactttataataattataaacaccacaactgctccactggggtgccttatgctaggactttcctccatgcgaccctttttgcatcataaaacaaataataacagattttacaaaagaaaaaataaaaaccaatgaggatgccccaccgccacattaaaggagaaagtagttcttagatcttgtctctctcaattcacttagtgcacctgtggatagaaacttaggtgaagatagagaaagaggtatcatgagttttaatgtacattattcattgcaatttaattccgggcacactaagttatttatttatttattcaatccctactgcaatccaagctactcttgttcttgccaagactatatcttgggggcggggaacaaactattttcattgcaccttttgggcgctacgagcaaaaaattgaccatcaaattttcgtcaatttaaaatcctaaatacaagtatcatacacatctactcaagtacaagaatccttgtaactattggaacaaaacaaattctgggactcttgtcggacactacgttaacgctagtaagtaacaaaaaatctttttaatattagaagtgtgtgtgacatccacttagattttgcgtgacatccctgccgctaccagaccttagtactcgagtagtagaataagaggagtagcgaccttcaacgacagccaagccatcaaaaagggtatcgtactcgagtgttcaaatagaaaatctaccttcctgtgataccaggtgaggtcattgtggtggttcaatacaacaatcaagtcctgcgattacgcataacaagtgtgagtactcgagaagagcgcgaatacacggctgcctggcccgcgtgggtctaacagatctgtccttgctctatttatttgttttgtttttcctaagcgcagcactttttaccctatgaggtatttataataatattatagatattctggaggaggttcccaaaaatatatcggaaatgcacctaggggcagtggtactaggattcccaccgcacactatctgctagtggatatgtctaggtttctagtttccataaacaccagacgagaaaggcttaccaaaacgacagagggggttgggtctgggagatgtctacggtgcgacgatagaacggataaaagcgcagggcggggataaatcgagactcgggatggcaactttgatgtggatcagtcatgcgcagaggctgctcgggacggacgagctctcccacgccctggcagttgagctgagctccaccaacttcaacgctagcaacatctcttcgatgtcgatactgctgggttgtttctaagggcttattacggtggacaaggaagcatcagccgtgcaactggcccactttactctccaagagtacctttccgctcatcccgatattttctgcagaccccatgcggcgatggtagagatctgcttgacctatctgaattcccggcaaatcaaggacctttcgaccgacccatctcctgacaccccgggtgcaccctttctagaatattgttctgtgcactggggaatccatgcgaaaacggaactctgggattgtaggagatcgcttgcgctgaagctattctaggagtttgatggccacatatccacagaatttcttttagcgcaagtggcgaatttaagtcccaaggggcgtgatacctggcctccgttcagtgggctgtactgcgcatcgttcttcggaatcgtcgagattatggctgctttaacatacgtgggatgttgggataccggggaaggaggttgtttggggtaaccgccgcttgcacgggctgttcataacgggcatggggaggtggtggaaatgctattcgggttggaaaaggtccatccggacgagctggataaccgcgaccggacaccgctctcacatgttactgcctgtcggcatgaagaagtggtgaaaatattgctcctgtttgagcgggaagaggatagccacaaaaatccggacaatgacggtcaaacaacactctctttcgctgctcggtttggacatgaggaagtggtgggaatactacttgggtgggaagatgttaatcccaatgagctagataatgacggccaaacaccgctctcgaatgctgcttggaggggtcatgagagagttgcgaagatattactcgggcgggaagaggtcaacctcgaagagccagacgagctcggccgggcgccgctcttgcatgccgctggtcatggacatgagggagtagtgaaaatttttctcgggcgggaagaggtcaacctcaggaagctagacaattactcccaaacaccgcttttgcatgcgtttggtctggatatgaagaggtggtgagagtgctactcatacggggagtgtcgataccggcgaaccggacatcagcggccaaataccgctctcgcatgctgccgctcatgggcacgaggaggtggtagaaatgttactcatgtgggaagaggtcaaacccgacaggccagatgatttcggccaaacacttgctgcttgggatggacatggggggatgatgaagtactactcggccgggaagatgtcaaaccggacaggccagacgatagcggtaaaaccctgctcttgttggcctcttgggataggaatgagggagcggtgaaaatactacttgggcgcgaagttgtcggccccgacgaaccggataatgacgaccaaatacccctctcgttcgcaactgggaatggccatcagaaggcggtggcccacaaagcgttaacccctagcgcggtttaaggtttataaaaaacaacctcttcgaaggcatcaccttccttttcttacagagtactttgctcgtaggggtcgacctagagcatcaccctttccagtcgaaggagatcccatgagacgagtctggaatgcgcttatatgccaccatgcggacgtgaacctctctggggatttgcttttatagtcgggcgtgggttttttttctgagtacggtagagtttggaactagttctattgccgtgacaaagtctagtttagtttattacaccttgcagtcctcatcctatcatatttgatttcctccagccgtacattcttgccctcacgttttttctctcccttgcgcaattgataagcatatagagtgttgttcatatagaaggaaagtggtgtatgagaggggaagggttcttaagaccaatgtgagcagctatgtaaggactgaatgataggtccccacgaatagtgcgatatcatacttagcagcagagaaaccatcaggaaggtaaactacagcacatattcagatgcc

SEQ ID NO: 39 - DNA, 7667 bp, Tuber indicum
ggcttgcatgatatcagcgaatgtagctcggggacgtaatctgataattacactaaacgttggataccacgcaggttcagagctagctccttctccgctcgaggtagaggattaagttcaccggcctctgaatgacatctgatgatgttccgggcaacttctctcggggcatctgtgtcgctagcaattgtatgataacttttgccttctgcatgaagctcaatcattcgtactctctcatcgcttgcggccattcgccgcttgcctgttgcttgaccttgaggtatggtgtataatgggaggtgttggctcccataaagaaatcgtggctaagatactagtgacgggttactaaatagcctctactgctgagaccgttgcgaaagctgagtaggttctctctacatattctagtagatcttgacgctgatttgtggaaggaaaaaaagaaattttatgttttaagagattttatgttttaagaaattttatgttttaagaaattttatgctttaagaaattttattttttaagaaattttatgctttaagaaattttatgttttaagaaattttatgtttttaagaaatttcatgttttaagaaattttatgttttttttggttttaaagatactgagggcgtttcattgatcctaggtacataagtttaatttaagtctacacgatctataatacgctgagaatgcctcccagctatgcccttgatttccgccgctcccacacagtcatactgtaatacagtaacttcagtttttttttgtacctgctagcctctttaggctgtctcgggaagtcatataggttcacaatccagtggaggattctccaatagtaggctgtttagatcatgttagaattgggggctggcggaatatgctgttcggggaacttctcacaaaaacgctgtatccaccagaagtgaaagtgttcatgtgcatctcgttctcgggctggaagtgcgggtcgagttgagagtctccattctcctcagcgctagtgatgtcaaaagcatggaggatgttggcggcttcctcctcggtgagatcgagctgacggttgctggaaacgtaaacacgggtgatagtgaatctggacactgcgggtgcgggcttggcacgagccttccttcttgcagcagccttatcggccgcttccttgtcgatgggggccttggggacaaagccagcttcacgcaagagcttctcaggggtagtagggtgaccatgtttcgccaattcggtctcgataaattcttcgagcacgagaccagggtcaccacgatccctagcagcagtataaagagaggcgatgttttgccacatatacttattggtctcagcgacccagacgccacgagtgagattggaaatttcggcttgagtgagcccataaccgttacagtacttagaaatccaggctaatagatggttagaaatagaggcaaaatagggatgtgatttacttacatctctgagcaacgtagggattcaatgcacgaaggtgctgagaggggggaatacgggccaaggcctcatcaccattgacgataacagccggaatagtggcatcctgggcggtccacggctgtccaggtctatagtatccgcaatacaaacgcagaaccgacacaacataagggacttcggggtcgagggggactcgcccttgatgacggacggttgattgaattgcaccgagccacttgtcatagggcaagagttcgccgctgacagtgaaagtgacaccaccggggggttgaacgataatgggggagttctcatgttggacccagatgcacccgatcctgtcttgcatgaggatgtacccacggtccgaaaggtacaaggcgcatcccaaggtagcattgcgcataatgaggcgtctctcgagcctgttctcccactccaaaaccttagggggggggagaggttaacgaccatgctcagattgacaggacgagacgacattatgaaaggcgggggggggggtggagacgggaaaagaaatttgaaccgacaaaaggaggattgcgtatgggcgtttcaatgagtggagattagcctcagtggctgttgatgttgatggaagaaaggagggtggaaggtctggcctcagttgtatgctacctcacgaagtgggatgccaaggcccaaaaaaaagagtgacgccaagaagccgtcatgtacaactcagccctactagggtctatcatagtacatacaataataataataatattattatgataaataatattattattatgatatcataatatttttcccagagcaggtagagaacccttaggtttctaagattcctggcggaaagattggggaacatttttgtatttacggtcagagttgctcgaatagaaaaacgagtaaaggtatttgatgaagatctgattgacattcgggagaagtttagaacaaaggaattagtggggatgagctgggaaatgtgggttctgttacagtatctgtcacccaatcttaactacccctcttgtacattgcctctttggtagggggggatggtatctgaggcttgaacaataattccttgcgataatatatatcgatcaggacagtttgctgactggaaagtctaatatatactatatagtcttcataaaaccgtgtgaccgactgggaaatcataccttaagtctggccatccggaagccgctctttctgctatgggggaatatcaaatagccaagaggccatcaggtgaaatgagaattcagaaatatgatagccttcccctgccaaccaagagacaaatgtttcggcaataccaccacctgatctggtggatgtctccatgttcggctatccgcttgagtatccaggggaatagacaaggaagactaccacattttatgcagtgtgtgcggctgccgagaaaactgtttgaaagatggaactccagaacaaaggattaactggtcaacgcaacacctggttgatgcaagggtctgaagattaaaggattgtaactagtattgttggacgttacttctagcaatttcagagtggttttgggtaaagatagactatactgatattggtaggatggtatagcactacaacgaaagaaagtggtctggacaatgagcacaaaaactctaaagagaatttccgggtgggaaattagaatatttaactagtttaaaaccataagattctctatcccaagacctgttgaaaatcacgaagcttccatggtttggatgatgaggtgtaggttgtttggccttggtttggaaacgcaatggcggaataagctaaggaaagggtcggcgtgtgggtgagaactagcagttggtgtaaggagaagtgtttgggatgttttaggagttagcactagggatactggaagtaacaggttatgctccggaggtccagtcgacaaccaggtgtcgcactataggagataggaaactggagaacaaccacaaaagtatcagggctgtctggaatagcgatagcttaggggaaacgttttggaggatgtttgaaggagttgtgtgaaaggaacggtgtggcaacatgaagaaaagatggtatggaattctgcgaggcagatggatgaagaaaatatttgcaaaggaatagcttctgtatatagaggacacctccggcagcatagaacaaacaagaatcgcagacactctctacctatcatagccttattatttgaagaccagcagctgattgagagagctgttgtgagagcatctaagcactcaacctgcagaactccatgataggtaggatgataaaaggggaatggggtttagaatgggtatataaagcctcttgattctggattaggatatggctagataatgttgttttttgtcacatctcttccaactatttatttaaaaaccacgagattggacattaaaatttaatgaaggagtgatttcctcatgcacaaaatatatattgtgtgaatacctacaattgagactgcataaacacatgagcctaaatcaatttagtaaattaaaatccatcaagtagttggtgatctagcttatgaagtttgtgaagttttttgagctgaagggaaaagagaaaggttaggcaaatttttttgtgacccttagctggttggctgtcaatgcttgatattgtcatccaaaccaccacagattgtattgtggggatgttgacatgttgcagtacgacatctcctaagaccgtatgcatataagagattactttatcctactaaaagagacaagacagtcgtttctactacttgcttctgtattctcttacctcatattcaaccataatttacaagagtcatgccaaatcaaatgttgtgtgatacctggtgaaaaatgtccacctaagcctcagctgtgaatatcgcaaattgtaaggtgagaggggaatcacaatgcataactttatattataaacaccacaactgctccactggggtaccttatactaggactttcctacatgcaactctttttgcatcataaaacaaataagaacagattttacaaaagaaaaaaaaaaaactagtgaggatgccctaccgcgacgttaaaggagaaagtagttctcagatcttgtctccctcagctcgcttagtgtacctgtggacagaaactcatgtgaagatagagaaagaggtatcatgaattttaatgcagattattcattgcaatttaattccgggcatactaatttatttattcatttactcaatccctactgcaatccaagctactcttgtccttgccaaggctatcttgagcgccgtgaacaaacttattttcattgcaccttttgggcgctatgagcaaaagattgactatttcatcaatttaaaatcctaaatacaaatatcatacacatctactcaagtactgtacaagaatccttgtaactattggaacaaaataaattctgggactcttgtcggacactacgttaacgctagtaagtaacaataaatcttttaaaattactagtgtgtgacatccacttagattttgcgtgacatccctgccgctactagaccttagtactcgagtaatagaataataggagtcgcgaccttcaacgacaagggtatcgtactcgagtattcaaatagaaaatctaccttcctatgataccaggtgaggtctttgctgtggttcaatacgacaatcaagtcccgcgattacgcataacaagtgtgagtactcgagaagagtgcgaatacacggctgcctggcctgcgtgggtctaacagatctatccttgctctatttatttgttttgtttttcctgagcgcagcactttttaccctatgaggtatttataataatattatagatattctggaggaggttcccaaaaatatatcggaaatgcacttaggggcaatggtactaggattcccaccgcacactatccgctaatagatatatctaggtttctagtttccataaacaccagatgagaaaggcttaccaaaacgacagagggggttgggtctgggggatgcctacggtgcgacgatagaacggataaaagcgcagggcggggataaatcgagactcgggatggcaactttgatgtggatcagtcatgcgcagaggctgctcgggacggacgagctctcccacgccctggcagttgagctgagctccaccgacttcaacgctagcaacatctcttcgatgtcgacactgctgggttacttctaagggcttattacggtggacaaggaagcatcagccgtgcaactggcccaatttactctccaagagtacttttccgctcatcccgatattttctgcagaccccatggggcaatggcagagatatacttgacctatctgaattcccggcaaatcaaggacctttcgaccgacccatctcctgacaccccggatgcaccctttctagaatattgttctgtgcactggggaatccatgcgaaaacggaactctcggattgcaggagatcgcttgcgctgaagctattctaggagtttgatggccacatatccacagaatttcttttagcgcaagtggagaatttaagtcccaaggggcgtgatacctttcctccgttcagtgggctgtactgcgcatcgttcttcggaaccgtcgaggttatggctgctttaacatacgtgggatgttgggatactcggggtggaacaggtccatcccgacgagctggataaccgcgaccggacaccgctctcacatgttgctgcctgtgggcatggggggggtggtgaaaatactgttcctgtttgagcaggaagaggatagccacgaaaagccggacaatgacggtcaagcaccactctctttcgctgcttggtttggacatgaagaagtggtgagaatactacttgggcgggaagatgtaaatcccaatgagccagataatgacggccaaacaccgctctcgaatgctacttggaggggtcatgagagagttgcgaagatattactcgggcgggatgaggtcaacctcgaagagccagatgagctcggccgggcgccgctcttgcatgccgctggtcatggacatgagggagtagtgaaaatttttctcgggcgggaaggggtcaacctcgaaaagctagacaattactcccgaacaccgcttttgcatgcgtttggtctggatatgaagaggtggtgagagtgctactcatacggagggtgtcgatcctggcgaaccggacagcagcggccaaataccgctctcgcgtgctgccgctcatgggcacgaggaggtggtaaaaatgttactcatgtgggaagaggtcaaacccgacacgccagatgatttcggccaaacacttgctgcttgggatggacatggggggataatgaagtactactcggccgggaagatgtcaaaccggacagcggtaaaaccctgctctcgtggcctcttgggatgggaatgagagagtggtgaatatactacttgggcgcgaagttatcggccccgacgaaccggataatgacgaccaaacacccctctcgttcgcagctgggaatgaccatcagaaggtggtggccctgctacagtcttacaaatcgttaactcctagcgcggtttaaggtttataaaaagcagcctcttcgaaggcatcaccttccttttcttacagagtactttgctcgtgggggtcgtcctagggcatcaccctttccagtcgaaggagatcccatgagacgagtccggaatgcgcttatatgccaccatgcggacgtgaacctccctggggatttgcttttatagtcgggtgtgggttttttttctgaatacggtagaggttggaactagttctattgccgtgacaaagcctagtttagtttattacaccttgcagtcctcatcctatcatatttgatttcctccagccgtacattcttgccttcacgttttttctctcccttgcgcaattgataagcatatagagtattgttcatatagaaggaaagtggtgtacgagaggggaagggttcttaaggccaatgtgagcagctatgtaagggctgaatgatagatcctcacgaatagtgcgatatcatacttagcagcagagaaaccatcaagaaggtaaactacagcacatattcagaggcc

SEQ ID NO: 40 - DNA, 1065 bp, Tuber indicum
atgacttcttctttgtccaacctcaatatggcccttgaagccgggcctcttattcaccaacgttatgatggaatcggtctcgccgagtatattcccttgctcgccaacggaacctttttctttgatgggtcggatggagtctccctccaggtccgccacctccttccagcagactggaagcccgtcaatggaccacttgcggtcttgttcgattccgtcccacataccattattgggactcacttttctatccgcaatcaccccatttataagcttaacgatggttctatcttggtcgtctttgacccggccgctaagcacggtttcgagtttaccaatcaatctttcgtggacccccagcttgaggtctccggagagaaccgtttcttgcgtctcgttaccaatgaccaagacttcgcctaccatggttctagtcttagcatgattcatgacaagggcgttaagcgccctgccaatgcgtaagtacaattctaccaatgtaccagtttcgtgtgctcatctagtcataggtttatcttattcagaacggacaaaatgaagcccctcaaggctcagtacccggagatgggcaacaacgatatctgtatgtggctcttattttccctttattttgatctctattcttggtaccatttgcctaacgttttaatagccaagattctcggtcgcatgtggcagaattcctcggatgaggtcaaagatgtttataaggtaaaacatctcacctaactgttagttagaatcactaacacattaataggcgagggcccatcagcttgcggtttcccataagttgaccaacccggattacaaatactccccgcgccgctctcgcgaaatcatgaggagggactccgggaataaaggtccttctccccgtatcgaccttcatcgtgctggtgttgcgaagcgtgaaaaaaagaggatcattacccaccttgggttcctcactcaacgtttcgcaactcaccccactgctaacaatctcggtgatatcatcgaggaagccgaaaaggtcttgaaggcctttggtatggtcaattg

SEQ ID NO: 41 - DNA, 732 bp, Tuber indicum
atgacttcttctttgtccaacctcaatatggcccttgaagccgggcctcttattcaccaacattatgatggaatcggtctcgccgagtatattcccttgctcgccaacggaacctttttctttgatgggtcggatggagtctccctccaggtccgccacctccttccagcagactggaagcccgtcaatggaccacttgcggtcttgttcgattccgtcccacataccattattgggactcacttttctatccgcaatcaccccatttataagcttaacgatggttctatcttggtcgtctttgacccggccgctaagcacggtttcgagtttaccaatcaatctttcgtggacccccagcttgaggtctccggagagaaccgtttcttgcgtctcgttaccaatgaccaagacttcgcctaccatggttctagtcttagcatgattcatgacaagggcgttaagcgccctgccaatgcgtaagtacaattctaccaatgtaccagtttcgtgtgctcatctagtcataggtttatcttattcagaacggacaaaatgaagcccctcaaggctcagtacccggagatgggcaacaacgatatctgtatgtggctcttattttccctttattttgatctctattcttggtaccatttgcctaacgttttaatagccaagattctcggtcgcatgtggcagaattcctcggatgaggtcaaagatgtttataag

SEQ ID NO: 42 - DNA, 1067 bp, Tuber indicum
atgacttcttctttgtccattctcaatatggccgttgaagccgggcctcttattcaccaacgttatgatggaatcggtcttgccgagtatattcccttgctcgccaacggaacctttttctttgatgggccggatggagtctccctccaagtccgccatctccttccagcagactggaagcccgtcaatggaccacttacggtcttgttcgattccgccccccataccattattgggactcatttttctatccgcaatcatcccatttataagcttaacgatggttctatctttgtcgtcttggacccggccaccaagcacggtttcgagtttaccgatcaatctttcgtggacccccagcttgaggtctccggagaaaaccgtttcttgcgtctcgttaccaatgaccaagacttcgcctaccatggttctagtcttagcatgattcatgacaagggcgttaagcgccctgccaatgcgtattgatacaattctaccaatgtaccagtttcgtgtgctcatctagtcataggttcattttattcagaacggacaaaatgaagcccctcaaggctcagtacccggagatgggcaacaacgatatctgtacgtggctctcatttcccccttatttagatctctattcttggtaccatttgcctaacgttttaatagccaagattctcggtcgcatgtggcagaattcctcggatgaggtcaaagatgtttataaggtaaaacatctcacctaactgttagttagaatcactaacacattaataggcaagggcccatcagcttgcggtttcccataagttgatcaacccggattacaaatactccccgcgccgctctcgcgaaatcatgaggagggactccgggaataaaggtccttccccccgtatcgaccttcaccgtgctggcgttgcgaagcgtggaaaaaagaggatcgttacccaccttgggttcctcactcaacgtttcgcgactcaccccactgttaacaatctcggtgatatcatcgaggaagccgaaaaggtcttgaaggcctttggtatggtcaattg

SEQ ID NO: 43 - DNA, 1069 bp, Tuber indicum
tcacaatccagtggaggattctccaataggaggctgtttagatcatgttagaattgggcgctggtggaatatgttgttcggggaacttctcacaaaaacgctgtatccaccagaagtgaaagtgttcatgtgcatctcggtctcgggctggaagtgcgggtcgagttgagagtctccattctcctcagcgctagcgatgtcgaaagcattgaggatgttggcggcttcctcctcggtgagatcgagctgatggttgctggaaacgtaaacacgggtgatggtgaatctggacaccgcggatgcgggcttggcacgagccttccttcttgcagcagccttatcggccgcttccttgtcgatgggggccttggggacaaagccagcttcacgcaagagcttctcaggggtagtagggtgaccgtgtttcgccaattcggtctcgataaattcttcgagcacgagaccagggtcaccacgatccctagcagcagtataaagagaggcaatgttttgccacatatacttattggtctcagcgacccagacatcacgagtgagattggaaatttcggcttgagtgagcccataaccgttacagtacttagaaatccaggctaatagatggttagaaatagaggcaaaatagggatgtgatttacttacatctctgcgcaacgtagggattcaatgcacgaaggtgctgagagggggggatgcgggccaaggcctcatcaccattgacgataacagccggaatagtggcatcctgggccgtccacggctgtccaggtttatagtatccgcaatacaaacgtagaaccgacacaacataagggacttcggggtcgagggggactcgccctcgatgacggacggttgattgaattgcaccaagccacttgtcgtagggcaagagttcgccgctgacagtgaaagtgacaccaccggggggttgaacgataatgggggagttctcgtgttggacccagatgcacccgatcctgtcttgcatgagaatgtacccacggtccgaaaggtacaaggcgcatcccaaggtagcattacgcat

SEQ ID NO: 44 - DNA, 1069 bp, Tuber indicum
tcacaatccagtggaggattctccaataggaggctgtttagatcatgttagaattgggcgctggtggaatatgttgttcggggaacttctcacaaaaacgctgtatccaccagaagtgaaagtgttcatgtgcatctcggtctcgggctggaagtgcgggtcgagttgagagtctccattctcctcagcgctagcgatgtcgaaagcattgaggatgttggcggcttcctcctcggtgagatcgagctgatggttgctggaaacgtaaacacgggtgatggtgaatctggacaccgcggatgcgggcttggcacgagccttccttcttgcagcagccttatcggccgcttccttgtcgatgggggccttggggacaaagccagcttcacgcaagagcttctcaggggtagtagggtgaccgtgtttcgccaattcggtctcgataaattcttcgagcacgagaccagggtcaccacgatccctagcagcagtataaagagaggcaatgttttgccacatatacttattggtctcagcgacccagacatcacgagtgagattggaaatttcggcttgagtgagcccataaccgttacagtacttagaaatccaggctaatagatggttagaaatagaggcaaaatagggatgtgatttacttacatctctgcgcaacgtagggattcaatgcacgaaggtgctgagagggggggatgcgggccaaggcctcatcaccattgacgataacagccggaatagtggcatcctgggccgtccacggttgtccaggtttatagtatccgcaatacaaacgtagaaccgacacaacataagggacttcggggtcgagggggactcgccctcgatgacggacggttgattgaattgcaccaagccacttgtcgtagggcaagagttcgccgctgacagtgaaagtgacaccaccggggggttgaacgatgatgggggagttctcgtgttggacccagatgcacccgatcctgtcttgcatgagaatgtacccacggtccgaaaggtacaaggcgcatcccaaggtagcattacgcat

SEQ ID NO: 45 - DNA, 1069 bp, Tuber indicum
tcacaatccagtggaggattctccaatagtaggctgtttagatcatgttagaattgggggctggcggaatatgctgttcggggaacttctcacaaaaacgctgtatccaccagaagtgaaagtgttcatgtgcatctcgttctcgggctggaagtgcgggtcgagttgagagtctccattctcctcagcgctagtgatgtcaaaagcatggaggatgttggcggcttcctcctcggtgagatcgagctgacggttgctggaaacgtaaacacgggtgatagtgaatctggacactgcgggtgcgggcttggcacgagccttccttcttgcagcagccttatcggccgcttccttgtcgatgggggccttggggacaaagccagcttcacgcaagagcttctcaggggtagtagggtgaccatgtttcgccaattcggtctcgataaattcttcgagcacgagaccagggtcaccacgatccctagcagcagtataaagagaggcgatgttttgccacatatacttattggtctcagcgacccagacgccacgagtgagattggaaatttcggcttgagtgagcccataaccgttacagtacttagaaatccaggctaatagatggttagaaatagaggcaaaatagggatgtgatttacttacatctctgagcaacgtagggattcaatgcacgaaggtgctgagaggggggaatacgggccaaggcctcatcaccattgacgataacagccggaatagtggcatcctgggcggtccacggctgtccaggtctatagtatccgcaatacaaacgcagaaccgacacaacataagggacttcggggtcgagggggactcgcccttgatgacggacggttgattgaattgcaccgagccacttgtcatagggcaagagttcgccgctgacagtgaaagtgacaccaccggggggttgaacgataatgggggagttctcatgttggacccagatgcacccgatcctgtcttgcatgaggatgtacccacggtccgaaaggtacaaggcgcatcccaaggtagcattgcgcat

SEQ ID NO: 46 - DNA, 13341 bp, Tuber indicum
cttcacggcggggcgtgggctcggagaacgggtgtttgtctatcgagtgatgttttgtgatcttactgattttagttatcaatacatccacatataccaggagcttctgggcgattgcttgcctgaaaacaagtgatccgggggagcactgtctgtctgttcgcttcacttgtgctgttcgaatgctttacctggttgtgcgctctgagtacgagaaatgggcgagcaagctagtctgtctgttcacgaagggcgcaatgaactatcacaaaccaattccagattcttctactcttttatccccttctcacggaggttctttccagtgagcttcgcccggtcttgtctcgtctaccattgcatagttatttacagcggatggcgtaatccgcacccttgtctatgatcgcaatacggcattggctctaacaaaactatatcgtgtcgttccgagggagaaaaaaagtcccttcattctactttcccttcctccactccgattcactggcagatgaaaccgtaccgcgcgctcgggctctttggtccctattttctcaaccggcgagggcgtcgttagtatgggggttggaaggctcctttggcttgtttgccgtgagtaggaagggcgctgtcattgctgagtagcctttcagcgggacctgcgggggtgcttctttccgatcggtcttgcgaggggtggtattggtattgggcggtgttggccattcggcggcgttgatgaaatttctatcaggagttctgtggcctctggatacgtggtgtagtatatcttcttggcggtttctctgctgctgctaagtatgatatcgcactattcgtggggacctgtcattaaatctttacatagttgctcacaatggtcttaggaatcctcgcgctaccgtacaccactttccttctatctgaacaacgctctatatgtttatgtatcgtacagggggagaaaggtaacgcgtgggcaagaaatgcacggctggagaaaataaaatatgataggatgaaaactgcaaggtataataaactagacttttttgccacagcgatagagctagttccaaactctacagtactcagggggcaaacccacgcccgactataaaagcaaatctccggaggggctcacatccgcacggtgacatataagcgcattccgggctcgtctcatgggatctccttcgactggaaggggtgatgcccaaagacgactttcatgagtaaagtaccccggaaaagaaggaacggtgatgccttcgatgaggtcctgttttataagccttaaatcgcgctaggggttaacgctttgtgggactgtagcagcgctaccaccttctgatggccgtgcctagatgcgaacgagagaggtgtttgaccgtcattgtttggcctgtcggggctgacatcttcccggccgagtagtaccttcaccacctccgcatgcccaccccaagcagcgagcgagagcggtgtttggccgcaatcatctggcctgtcggggttgacctcttcccgcatgagtaaaattttcaccacttcttcaagtccatgagcggcagcatgagagagcggtgattggccgtggctgtctgacttgtcgggattgacctcttcccgcatgagtagcattctcaccacctcttcatatccggaccaaacggcgtgcgagagcggtgttcggcagtagatatctggcttgtcggggttgacctcttcccgcccgagaaagattttcactactccctcatgtccataaccagcggcatgcgagagtggcgttcgaccgagattgtctggcttgtcggggttgacctcttcccgctcgagtaataatttcgcaactccctcataccccctccaagcagcattcgagagcggtgtttggccgtcattatctggttcgttgggattaacatcctcccgcccgagcagtattctcaccacttcctcatgtccaaaccaagcagcgagcgagagcggtgtttggccgtcattgtccggcttgtcggggttgacctcttccctcccgagtagtattttcaccacttcctcatgtccgcaggcagtagcatgtgagagcggtgtttggccgcgattatcgggctcgccgggatcgacctgttccagcccaagtagcattttcaccacttcctcatgtccgttatgagcagcccatgcaagtggcgtatacccccaacaatctccttcgttgatatcccaacatcccatctctattaaagcagccacaacctcgacaattccaaagaacgatgcgcagtgcagcccactgaatgggggccaggtatcgtgccctccgggacctaaattccccacttgcgctaaaagaaattcggtggatatgtggccatcaaacccctggaatagcttcagcgcaagtgatctcccgcaatccgagagttccgttttcgcatggactccccaatacatagaacaatattctagaaagggtgcatccgaggtgtcaggagatggatcggtcgaaaggtccttgacttgctgggaattcagataggtcaagcatatctctgccattgccgcatggggtctactgaaaatatcgggatgagcggaaaggtactcttggagagtaaagtggactagtcgcacggctgatgcctccttccccaccgtaataagcccttgacagcaacctatcagtgtcgacatcgaagggatgttgttagcattgaagtcggtggagcccagctcgactgccagggcgcagcagagctcgtccgtcctgagcggccgctgcgcatgactgatccacatcaaggctgccatcccgagtctcgatttatccccgccctgcgcctttatccgtcctatcgtcgcaccgtaggcatctcccagacccaaaccgtcggtcattttgctaagcctttctcgtctccgataggttgtcgattcttgtaagattgcttcgatgtttagggaaactagtataaacctggatatatctattagcggatagtgtgcaatgggaatccctctgtcattgccctacaaacatttccgagatcttcccagggatctgctctagaacgtctgcctcaagaccctcgtccatggcatctggggtttcgtcctcgtctagcctaacacgaaggtatctgataatatcatctcttctagggcctatagacacacttgccactcgcccggcaagacgcttttcgatctcggcccgaaatataggaccttccagtcaggaatattcgtatccccggggacttcccaaggatttttttcagcgaatcaaggagcttggctcgatgcactgcggcacattcgtccagggcgtcaatgcacataaatgtgcgctgtgaggacgtgatggtctgtagcatctttacgatatcaggaagttgcggtccccgcccgccaatggccctcctctgctcttgaaaagcccgcgatatttcctctggaatcctttccattccgctaacgatctgtttaagcaaggaacccagcatattcgtcgcggactgctcctttcgggccgcaaagtcaaagtaaaaacaggtaacggtagtgttttgtcctctagcctgatcgcacagcctgtccactaccatcgaactgtcatcgcggcttattagcactggcgcacttctctccccagagaataactcttgttacctgataaatgtcttgcccacacctggatctccatggcaaaacaacactgccttattgccttcaccttccaaactccagtcataccagcttctgaattcgtcagtttggataagccattcccccacattgtccacacggtgctcttgaatgtcgcgatgccgtaacctaggttccagcggcgacaaccaagctaggagttgagaccgatcatcggcaactgtgaagttgttccagacattgctaaaagagctcgtattattgaagcagttgttattgctattgattgtatattgagacatcttctgctctttactgcttgcactattgaagggaagtatttttctggtttggttggttgacgatatccttgggaggtcaagaaaagcggggtctagatggggaaagcaagggtctgctggtgttaaagcagcatcgcagataccgctactcgtacatgcagtactaactcaagagttttcgtccggtaccctttttacaccaaaagtatctaacttcaccaccctctctcacagcacaatgtcaaggggaatatgccaaaatttcgcggtgtattggcaaatctgtagctaatatgggagataatattattacagaattgttgagtaatagagccccataccggtacaaaaatctaggttaatttcacacaggccaacgttattgtaaacaatgggcacaactttgatcctctacagttctacttctagagatttaaatgaataacagctatctagccgagggagggatttggagtgttttcaacagggtattgtttggattgttaggtatgttgcggtagtctgcgacatgataaacaccatacttcgtggagaatgatataaagcgctgcgcccgggaaaagtaaggacaaaataaatagacactgttaaatacgtagaacaaggtctttggcattcgagtgcaatttgaaaacttgcggtcgatattcgtgatcgagagattgtggctgttgcatcgaactaccatatgactttcctaaataggaaggcagactcttgattcttttagatggcctggcagttgttgaaggttctgaactatcccttagagagctgaatcaacgaaattcagtgccgccaagatattgcgcaactgtgctgtctaagacatagggtgaataagcactaagcgacgtcgtgagaagctgaatctcgtgagaagattaagaaaacaaatcccaatcgttcagaattctactagaaatgccacgaacaagaaaatcaagtgcacaaacggctgctagaggagtggtgtcccagtcggttcaccagagagagcctttagctgtcctatcagtgaacggtaggaagccaagccgaatatgttcatagaatgctaaacgtttcacagcaggtgcagaatctcgaatgggaccagtggggccatcacatctgcatatggataccataatgcctatgggtgagatgggaaagggaaagggaaaagcaaaggagtcgggtggggagtcactctctgcggccgaaaagattcgaaatcttcctcctccgccacaccacataccattccaaggactatttccaccgcaccaagctagagccataggacctgcaactggagtaactgacccgtatcggttattctcactgtttttttcgccggagcaactggagattctggcgaggcataccaatatttatgcaagtatgcatgatgctggcctgcaagacagtaggcaccctcttgttcggaagtggtatccaacaactccctctgagcttcgaatattgttagcgatcttgattcatctgggtatatcaagaggtgctagtccaaaactcttttggagaaaggttggaggtgttgttccggaaccgatgtgcagaatgagatatatacgctttcagcagctaaagcgctatttgcatatctcagagccatctgagtccccaattcctactcaaaagtggtggaaaaagctcgaacctttaaatagcagtattcgaaaaacctcaaaaagagtgtttccttccgtccaccaatgtggctgtcgatgagatgatgatacgcttccttggccgctcagcccacacaattaaaatgccaaataaacctattagtttaggatataaggtactggcattgtgtgatgcaggatacacctatgactgggaacttacttcacgtattgcgagctttgcaacctccgaacagcaaaaaatgccatatcctctttctcctacctcctccgctgtacttcagatgcttaccacactaccctaccgtacccacttttttaccgcctatatggacaattacttctcaaatatacgcctatttgcacgtttactggattatggaattggagcctgtggtacagtgcggtgcagcagcaaagattttcctccctctctcaatatcagcaaagataaggctgctggtatcttgaaatggaattttgctactggagttgttgtacgcgaaatagttgatacagcaaaaaagataaagggagacagcccacccaagctacataggtgggggattcctccggttatggcttttttgtggcaggataataacacggtgcatttattatctactgttcatgatcttgagccatcaacttcctggataaagaaacttcgcagaaggccacgggaaacaagtaccaatgctgctgctgctcggaagcccttttctgctggggagcatcgcaagctattggcaattcccaagatcgacaatgactataaccaattcatgggtagtgtggatattgcagatcagcttcgatcatatttttctacgcaacgtattgtgcgtcggaactggcaaccttttttttactggttacttgatactgcaatcattaatgcctaccgaatcgcgcgaaccaatggctctaaaaccacccaccgtacatttcgctcctcccttacaagttcccttcttactgccggtcagaaacactcctcccctgagccaaaatttacattcctctaccgaactcgacgacggtatcgcctacgcaacccagtacgtcaaatatatattacgaagaactcacagaagccagaaccggtaggtggagggggaactttaaggggccatgtactggtacggaaagctactcgagcatggtgcctatggtgccgctggcgtcacaagcaaggtggctcggaggtgaaaatccaccaagtgcgatctcagtgtgaacggtgtcatgtagccctttgtcattcatgttttcccttatatcatggtgtaggaagctctttggtataggaagctcttcggtctaggttctcttttttttttttttttatatcactttgtctatttagtaatccagatccctgctctggcttgctgtcgattgcgcaatatcttggcggcactgaatttcgttgtttcagctctcgaagggataggaactattggtacttgtactaaatactaattcctaacaacgtacagggatgtcacgcaaaatcaaatcaaacattacacactaatggtattaaagaatgttttacttgctagcgccgtcatcgttcctgatcacctggaatttgtcagttccaatagacagacagctataagggctcttgtagatgagtgtttgaggatttacattgctatctcagcttcagggagaacaagaaaggtatgtcattttttgcccccacagcccaatatgtgcagcgaaaatggttgttttttctcccgtcaaaatacaatttttgccgggataaggggaccctaggtcgggtggggttaaatggcttggggtatcatatctagaaggaaattgacaattagcgcgcattaaactcacagctctttggtgagctttcctcgttgtctgtctgtaggtgcacgtggagcgatccaggtatctggggggacctgggaagcattcccctcttattgtggtggtagggcacctgcagtggggaaactccagcatataaataaagttataatataatattattacgattcccgcctatctcataattaaattctgcgatattcccagctctggggtattattggtcgttatctccacgatgtatcacccgatgtttgatttagcgtgactgttgtaaatcacagtggaatgagagataagagtatgtggggacaggtagaagtgacttgcttgacactctgggtggggtgaatacatacatataactcatagagatgtcatgctaaggtatatagacaactctataacacaacttggatatggtggataaagtgtgacggcctactaactgggaccacaaaggattcaattgtgtaccttaagaacatggaatacttatgctgtaatgttttatgcatgcaggaatcacccattcagtagatgtcgaattttacagctttcgaactaacactccattaccaaataagtatcgcgggtgcgcatttgtgggaagagatgggcattataccataaaccacaggttctaataagtgtttagatattggctgcttccagcgattccatcaggacttaagatatttcctttctagtacaactactaaatagagaaagacagcgaagagatggaacagagaggccctagtgcccgcctcatgtgcaattacggtatgtttgtgagagacactccgctatgatctactaccttcacagatgttcctaacattatataaacaaacgtgcaatatctcggtctctgatcatacaatcccctgtcctacatccaatcaagttcctttcctccgtgtaatccacttgacatcttagcttctatcccgggagtcggagcgggaccgggggcacattgccccaggctctcttatctgacgatatcttcgtccagaatcaagagactttgtctgtcccttctaaactccgcccccttccatcatcctgccttgcatgaagctcaggaggttgagtgcttagatgctccacaatggtttgctcaatcggccgctgctcctgggccagaagctctgacggcttagagttggttaaagattctaatttccaagctgaaaatccttttgggggcgggctttgctcgctcgtgatccagaccttctccgttcgttatattactataccgtcctactgatttcagaatggcctaacttagccaaaaacccctctgaaaccactaggagtaactgctagcaatgttagccacaatcccttaatttccagacccttaccgagaccggttgtcgcattggatggctgctcctttgttctggagttccatcttttaaacagctttcccgagagctgcatacgccgcatcaaacgtcgtagtcctccttgtccattcacctggatgcacaagcggatagccgaatatgggacatccaccggatcggctggtagtgttgcaaaagcatttggctttcctggttggccgggcagcgggtgaagctaccatacttctgaattcacatttcgcctaacggcctcctggccattggatatccccccaggacggaaaggacagcttctggatggtcaggcttaaggtatggtttcccagtccgccacacagatttatggagatcatattaaattccgcaatcagaaaaaactatcttgaaccacatacggtatctgatcgcaaagaattattgttcagggctcggataccatacctctctatcaaagaagcaatgtacgagtacaagagaggtggggttgggggacggatgctgtaacagaacccacatttcctagctcaccctgtttcattaattcctttgttcggaacttctcccgaatgtcgatcggatcttcatcaataccttgactcgttttctaccatactccagcatctccgaccgtaaaaaaaaaatttcgcggatcttttcgccaggaatcttggaaacctaagggctctcccttcctgccctgggggaaatcttatcgtgtcatatgtgtgataataatattatgacattttcatgagaatactattctcgggccctaatagggttgagttgtacgtgacggcttcttggcgtcactctattttttgggtcttggcagtccccctcgtgactggaggctacaaaggggatccatcctttccccttctttcctccaccgacaaccgtcaagttcgacagaaacattcggcctttctacttgcacctctccactcacgtcgtttcctttttttctgccactatgacttcttctttgtccaacctcaatatggcccttgaagccgggcctcttattcaccaacgttatgatggaatcggtctcgccgagtatattcccttgctcgccaacggaacctttttctttgatgggtcggatggagtctccctccaggtccgccacctccttccagcagactggaagcccgtcaatggaccacttgcggtcttgttcgattccgtcccacataccattattgggactcacttttctatccgcaatcaccccatttataagcttaacgatggttctatcttggtcgtctttgacccggccgctaagcacggtttcgagtttaccaatcaatctttcgtggacccccagcttgaggtctccggagagaaccgtttcttgcgtctcgttaccaatgaccaagacttcgcctaccatggttctagtcttagcatgattcatgacaagggcgttaagcgccctgccaatgcgtaagtacaattctaccaatgtaccagtttcgtgtgctcatctagtcataggtttatcttattcagaacggacaaaatgaagcccctcaaggctcagtacccggagatgggcaacaacgatatctgtatgtggctcttattttccctttattttgatctctattcttggtaccatttgcctaacgttttaatagccaagattctcggtcgcatgtggcagaattcctcggatgaggtcaaagatgtttataaggtaaaacatctcacctaactgttagttagaatcactaacacattaataggcgagggcccatcagcttgcggtttcccataagttgaccaacccggattacaaatactccccgcgccgctctcgcgaaatcatgaggagggactccgggaataaaggtccttctccccgtatcgaccttcatcgtgctggtgttgcgaagcgtgaaaaaaagaggatcattacccaccttgggttcctcactcaacgtttcgcaactcaccccactgctaacaatctcggtgatatcatcgaggaagccgaaaaggtcttgaaggcctttggtatggtcaattgagtgatctcaacggagggactcatcagtgcctgggtgatatactttcaatgggagggatgatcttgtctatcaaaggtcattatctcagtcgaataaatttatatgacctaaataaataaaaaatatgtttaaaaaaaaaaaaatgaggggtctgaacactatatcatacagtaaaatatcaagttgtgaagcagaagagagtcgactaccggtatgggaagaggcagtgagaatctaattctgactgtcttagtctggaaagttcgtacagcacttgcacacaagttatgctatggtggtgccatcaccggaaaaacacgccaaaaattcgggacctatggtgccgtgacttagtttgcgcgcaagtatgtactgtacatggtccgacacaaaagagtgttatggagcaggggtggtggggttgattttggccatcagcgttatcgcacagattgtttgaaaacacgactcgcattgctcaacaaggacatggagagctattttttttaatttttttttaattttgatagatgtattggactagaggccaagcatctgatcaattggttgtactatatttgctcatagttgactgaggccctgtttggatacaatggttcctttctgtttccccgtacgaggtgaccgttgcagaacgggagggcaatcgcttttatatgagaaacagttccaaaacgaaacggaaccgttccatccaaacaaggcctgagagaagaggaaattgtgatcttctcagggttaagtttcttggatgtgtcgctgctactgaaacttttgttatataggtatccattttatatctgactccctccacacagaccccagtggagcgaatccccggagcactttgctcatgaaagtcgcccttgggcattactccttccagtcgaaggagatctcatgagacgagctcggaatgtgcttatatgtcaccagacggacgtgaacctccctggagatttgcttttacagtcgggcgttttttgtttcccgagtactttagagtttggaaccagttctattgctttgacaaggtctagtttattataccttcttgctctcgcgtttttttctctcgcacaactgatagacatataagtgttcttcagatagaaggaaatggtgtacgatagcgcaaggattcctaaggccattgtggacaactatgtaaggacttatgacaggtccccacgaatagtgcgatatcatacttagcagcagagaaaccatcaagaaggtagactatagcacatattcagaggccacggaactcctgagacataagggtagggataaccactgcacacgagaagaacaatcagaataaaacagagccattttataacaaatactacatgacaaagggctaccagaatctattgatgagcagaggcgcatgagagaaagcataggatcccgatttcggtacccgtagtttaagggggtagaacatgccctagatcttagtatattttctagggaactgctattatatgataataatattattatgataaatatacttgtcctctagtgtgtgtaacatccaccctgcaagcttaaccgctttgggtgctcatcataggggaggtccactagttccaggagccagtcagatattttacaatagaatctatagatcggaggtcttaccaatttggctgcggttcgggtgagtctgggacattctgttaagattatacccactaccgaatccggacagagtccagatggccgcttaaccactatccgaagggccctaggccaaagctatcaggagtgtgctctagcacagttacacatgcttaggcaatcaaactttgaagataccgtaaaaacccttgtcgttcgctaccccaccgggttgctaatctccagattcagacatagcgcttaagcaggaatagtgctttcctgacacggtacagcctaagtcctgtggcattgttggttctcatgaactagttttcgcaaccctgttatcctgtcggatgtatgatagcatgtcgtgcgctgatgcgccgacccacagggatattggagacctacggagatatagaatcgaaaggatttttacagtattggtagatggctgttagactctcttgccctccctccttttgctacgcacgatgaagaatagaacaatatcggtaggtcagatcaaacgacgactttgcttggaattgagagatagagaaatagcttggctcgggcccctgcgcccgtcagccacccggaccgggtcagggttgggatttgaaagaagatttccctgggtcagtccggcagcactggtatcacagatgtgtcagcctggggttactgattcggcgctcgaacctcccccaataacccctcccttgcagcatcgccc

SEQ ID NO: 47 - DNA, 10992 bp, Tuber indicum
gtccttcacggcggggcgtgggctcggagaacgggtgtttgtctatcgagtgatgttttgtgatcttactgattttagttatcaatacatccacatataccaggagcttctgggcgattgcttgcctgaaaacaagtgatccgggggagcactgtctgtctgttcgcttcacttgtgctgttcgaatgctttacctggttgtgcgctctgagtacgagaaatgggcgagcaagctagtctgtctgttcacgaagggcgcaatgaactatcacaaaccaattccagattcttctactcttttatccccttctcacggaggttctttccagtgagcttcgcccggtcttgtctcgtctaccattgcatagttatttacagcggatggcgtaatccgcacccttgtctatgatcgcaatacggcattggctctaacaaaactatatcgtgtcgttccgagggagaaaaaaagtcccttcattctactttcccttcctccactccgattcactggcagatgaaaccgtaccgcgcgctcgggctctttggtccctattttctcaaccggcgagggcgtcgttagtatggggattggaaggctcctttggcttgtttgccgtgagtaggaagggcgctgtcattgctgagtagcctttcagcgggacctgcgggggtgcttctttccgatcggtcttgcgaggggtggtattggtattgggcggtgttggccattcggcggcgttgatgaaatttctatcaggagttctgtggcctctggatacgtggtgtagtatatcttcttggcggtttctctgctgctgctaagtatgatatcgcactattcgtggggacctgtcattaaatctttacatagttgctcacaatggtcttaggaatcctcgcgctaccgtacaccactttccttctatctgaacaacgctctatatgtttatgtatcgtacagggggagaaaggtaacgcgtgggcaagaatgcacggctggagaaaataaaatatgataggatgaaaactgcaaggtataataaactagacttttttgccacagcgatagagctagttccaaactctacagtactcagggggcaaacccacgcccgactataaaagcaaatctccggaggggctcacatccgcacggtgacatataagcgcattccgggctcgtctcatgggatctccttcgactggaaggggtgatgcccaaagacgactttcatgagtaaagtaccccggaaaagaaggaacggtgatgccttcgatgaggtcctgttttataagccttaaatcgcgctaggggttaacgctttgtgggactgtagcagcgctaccaccttctgatggtcgtgcctagatgcgaacgagagaggtgtttgaccgtcattgtttggcctgtcggggctgacatcttcccggccgagtagtaccttcaccacctccgcatgcccaccccaagcagcgagcgagagcggtgtttggccgcaatcatctggcctgtcggggttgacctcttcccgcatgagtaaaattttcaccacttcttcaagtccatgagcggcagcatgagagagcggtgattggccgtggctgtctgacttgtcgggattgacctcttcccgcatgagtagcattctcaccacctcttcatatccggaccaaacggcgtgcgagagcggtgttcggcagtagatatctggcttgtcggggttgacctcttcccgcccgagaaagattttcactactccctcatgtccataaccagcggcatgcgagagtggcgttcgaccgagattgtctggcttgtcggggttgacctcttcccgctcgagtaataatttcgcaactccctcataccccctccaagcagcattcgagagcggtgtttggccgtcattatctggttcgttgggattaacatcctcccgcccgagcagtattctcaccacttcctcatgtccaaaccaagcagcgagcgagagcggtgtttggccgtcattgtccggcttgtcggggttgacctcttccctcccgagtagtattttcaccacttcctcatgtccgcaggcagtagcatgtgagagcggtgtttggccgcgattatcgggctcgccgggatcgacctgttccagcccaagtagcattttcaccacttcctcatgtccgttatgagcagcccatgcaagtggcgtatacccccaacaatctccttcgttgatatcccaacatcccatctctattaaagcagccacaacctcgacaattccaaagaacgatgcgcagtgcagcccactgaatgggggccaggtatcgtgccctccgggacctaaattccccacttgcgctaaaagaaattcggtggatatgtggccatcaaacccctggaatagcttcagcgcaagtgatctcccgcaatccgagagttccgttttcgcatggactccccaatacatagaacaatattctagaaagggtgcatccgaggtgtcaggagatggatcggtcgaaaggtccttgacttgctgggaattcagataggtcaagcatatctctgccattgccgcatggggtctactgaaaatatcgggatgagcggaaaggtactcttggagagtaaagtggactagtcgcacggctgatgcctccttccccaccgtaataagcccttgacagcaacctatcagtgtcgacatcgaagggatgttgttagcattgaagtcggtggagcccagctcgactgccagggcgcagcagagctcgtccgtcctgagcggccgctgcgcatgactgatccacatcaaggctgccatcccgagtctcgatttatccccgccctgcgcctttatccgtcctatcgtcgcaccgtaggcatctcccagacccaaaccgtcggtcattttgctaagcctttctcgtctccgataggttgtcgattcttgtaagattgcttcgatgtttagggaaactagtataaacctggatatatctattagcggatagtgtgcaatgggaatccctctgtcattgccctacaaacatttccgagatcttcccagggatctgctctagaacgtctgcctcaagaccctcgtccatggcatctggggtttcgtcctcgtctagcctaacacgaaggtatctgataatatcatctcttctagggcctatagacacacttgccactcgcccggcaagacgcttttcgatctcggcccgaatataggaccttccagtcaggaatattcgtatccccggggacttcccaaggatttttttcagcgaatcaaggagcttggctcgatgcactgcggcacattcgtccagggcgtcaatgcacataaatgtgcgctgtgaggacgtgatggtctgtagcatctttacgatatcaggaagttgcggtccccgcccgccaatggccctcctctgctcttgaaaagcccgcgatatttcctctggaatcctttccattccgctaacgatctgtttaagcaaggaacccagcatattcgtcgcggactgctcctttcgggccgcaaagtcaaagtaaaaacaggtaacggtagtgttttgtcctctagcctgatcgcacagcctgtccactaccatcgaactgtcatcgcggcttattagcactggcgcacttctctccccagagaataactcttgttacctgataaatgtcttgcccacacctggatctccatggcaaaacaacactgccttattgccttcaccttccaaactccagtcataccagcttctgaattcgtcagtttggataagccattcccccacattgtccacacggtgctcttgaatgtcgcgatgccgtaacctaggttccagcggcgacaaccaagctaggagttgagaccgatcatcggcaactgtgaagttgttccagacattgctaaaagagctcgtattattgaagcagttgttattgctattgattgtatattgagacatcttctgctctttactgcttgcactattgaagggaagtatttttctggtttggttggttgacgatatccttgggaggtcaagaaaagcggggtctagatggggaaagcaagggtctgctggtgttaaagcagcatcgcagataccgctactcgtacatgcagtactaactcaagagttttcgtccggtaccctttttacaccaaaagtatctaacttcaccaccctctctcacagcacaatgtcaaggggaatatgccaaaatttcgcggtgtattggcaaatctgtagctaatatgggagataatattattacagaattgttgagtaatagagccccataccggtacaaaaatctaggttaatttcacacaggccaacgttattgtaaacaatgggcacaactttgatcctctacagttctacttctagagatttaaatgaataacagctatctagccgagggagggatttggagtgttttcaacagggtattgtttggattgttaggtatgttgcggtagtctgcgacatgataaacaccatacttcgtggagaatgatataaagcgctgcgcccgggaaaagtaaggacaaaataaatagacactgttaaatacgtagaacaaggtctttggcattcgagtgcaatttgaaaacttgcggtcgatattcgtgatcgagagattgtggctgttgcatcgaactaccatatgactttcctaaataggaaggcagactcttgattcttttagatggcctggcagttgttgaaggttctgaactataggaactattggtacttgtactaaatactaattcctaacaacgtacagggatgtcacgcaaaatcaaatcaaacattacacactaatggtattaaagaatgttttacttgctagcgccgtcatcgttcctgatcacctggaatttgtcagttccaatagacagacagctataagggctcttgtagatgagtgtttgaggatttacattgctatctcagcttcagggagaacaagaaaggtatgtcattttttgcccccacagcccaatatgtgcagcgaaaatggttgttttttctcccgtcaaaatacaatttttgccgggataaggggaccctaggtcgggtggggttaaatggcttggggtatcatatctagaaggaaattgacaattagcgcgcattaaactcacagctctttggtgagctttcctcgttgtctgtctgtaggtgcacgtggagcgatccaggtatctggggggacctgggaagcattcccctcttattgtggtggtagggcacctgcagtggggaaactccagcatataaataaagttataatataatattattacgattcccgcctatctcataattaaattctgcgatattcccagctctggggtattattggtcgttatctccacgatgtatcacccgatgtttgatttagcgtgactgttgtaaatcacagtggaatgagagataagagtatgtggggacaggtagaagtgacttgcttgacactctgggtggggtgaatacatacatataactcatagagatgtcatgctaaggtatatagacaactctataacacaacttggatatggtggataaagtgtgacggcctactaactgggaccacaaaggattcaattgtgtaccttaagaacatggaatacttatgctgtaatgttttatgcatgcaggaatcacccattcagtagatgtcgaattttacagctttcgaactaacactccattaccaaataagtatcgcgggtgcgcatttgtgggaagagatgggcattataccataaaccacaggttctaataagtgtttagatattggctgcttccagcgattccatcaggacttaagatatttcctttctagtacaactactaaatagagaaagacagcgaagagatggaacagagaggccctagtgcccgcctcatgtgcaattacggtatgtttgtgagagacactccgctatgatctactaccttcacagatgttcctaacattatataaacaaacgtgcaatatctcggtctctgatcatacaatcccctgtcctacatccaatcaagttcctttcctccgtgtaatccacttgacatcttagcttctatcccgggagtcggagcgggaccgggggcacattgccccaggctctcttatctgacgatatcttcgtccagaatcaagagactttgtctgtcccttctaaactccgcccccttccatcatcctgccttgcatgaagctcaggaggttgagtgcttagatgctccacaatggtttgctcaatcggccgctgctcctgggccagaagctctgacggcttagagttggttaaagattctaatttccaagctgaaaatccttttgggggcgggctttgctcgctcgtgatccagaccttctccgttcgttatattactataccgtcctactgatttcagaatggcctaacttagccaaaaacccctctgaaaccactaggagtaactgctagcaatgttagccacaatcccttaatttccagacccttaccgagaccggttgtcgcattggatggctgctcctttgttctggagttccatcttttaaacagctttcccgagagctgcatacgccgcatcaaacgtcgtagtcctccttgtccattcacctggatgcacaagcggatagccgaatatgggacatccaccggatcggctggtagtgttgcaaaagcatttggctttcctggttggccgggcagcgggtgaagctaccatacttctgaattcacatttcgcctaacggcctcctggccattggatatccccccaggacggaaaggacagcttctggatggtcaggcttaaggtatggtttcccagtccgccacacagatttatggagatcatattaaattccgcaatcagaaaaaactatcttgaaccacatacggtatctgatcgcaaagaattattgttcagggctcggataccatacctctctatcaaagaagcaatgtacgagtacaagagaggtggggttgggggacggatgctgtaacagaacccacatttcctagctcaccctgtttcattaattcctttgttcggaacttctcccgaatgtcgatcggatcttcatcaataccttgactcgttttctaccatactccagcatctccgaccgtaaaaaaaaaaattcgcggaccttttcgccaggaatcttggaaacctaagggctctcccttcctgccctgggggaaatcttatcgtgtcatatgtgtgataataatattatgacattttcatgagaatactattctcgggccctaatagggttgagttgtacgtgacggcttcttggcgtcactctattttttgggtcttggcagtccccctcgtgactggaggctacaaaggggatccatcctttccccttctttcctccaccgacaaccgtcaagttcgacagaaacattcggcctttctacttgcacctctccactcacgtcgtttcctttttttctgccactatgacttcttctttgtccaacctcaatatggcccttgaagccgggcctcttattcaccaacattatgatggaatcggtctcgccgagtatattcccttgctcgccaacggaacctttttctttgatgggtcggatggagtctccctccaggtccgccacctccttccagcagactggaagcccgtcaatggaccacttgcggtcttgttcgattccgtcccacataccattattgggactcacttttctatccgcaatcaccccatttataagcttaacgatggttctatcttggtcgtctttgacccggccgctaagcacggtttcgagtttaccaatcaatctttcgtggacccccagcttgaggtctccggagagaaccgtttcttgcgtctcgttaccaatgaccaagacttcgcctaccatggttctagtcttagcatgattcatgacaagggcgttaagcgccctgccaatgcgtaagtacaattctaccaatgtaccagtttcgtgtgctcatctagtcataggtttatcttattcagaacggacaaaatgaagcccctcaaggctcagtacccggagatgggcaacaacgatatctgtatgtggctcttattttccctttattttgatctctattcttggtaccatttgcctaacgttttaatagccaagattctcggtcgcatgtggcagaattcctcggatgaggtcaaagatgtttataaggtaaaacatctcacctaactgttagttagaatcactaacacattaataggcgagggcccatcagcttgcggtttcccataagttgaccaacccggattacaaatactccccgcgccgctctcgcgaaatcatgaggagggactccgggaataaaggtccttctccccgtatcgaccttcatcgtgctggtgttgcgaagcgtgaaaaaaagaggatcattacccaccttgggttcctcactcaacgtttcgcaactcaccccactgctaacaatctcggtgatatcatcgaggaagccgaaaaggtcttgaaggcctttggtatggtcaattgagtgatctcaacggagggactcatcagtgcctgggtgatatactttcaatgggagggatgatcttgtctatcaaaggtcattatctcagtcgaataaatttatatgacctaaataaataaaaaatatgtttaaaaaaaaaaaaatgaggggtctgaacactatatcatacagtaaaatatcaagttgtgaagcagaagagagtcgactaccggtatgggaagaggcagtgagaatctaattctgactgtcttagtctggaaagttcgtacagcacttgcacacaagttatgctatggtggtgccatcaccggaaaaacacgccaaaaattcgggacctatggtgccgtgacttagtttgcgcgcaagtatgtactgtacatggtccgacacaaaagagtgttatggagcaggggtggtggggttgattttggccatcagcgttatcgcacagattgtttgaaaacacgactcgcattgctcaacaaggacatggagagctattttttttaatttttttttaattttgatagatgtattggactagaggccaagcatctgatcaattggttgtactatatttgctcatagttgactgaggccctgtttggatacaatggttcctttctgtttccccgtacgaggtgaccgttgcagaacgggagggcaatcgcttttatatgagaaacagttccaaaacgaaacggaaccgttccatccaaacaaggcctgagagaagaggaaattgtgatcttctcagggttaagtttcttggatgtgtcgctgctactgaaacttttgttatataggtatccattttatatctgactccctccacacagaccccagtggagcgaatccccggagcactttgctcatgaaagtcgcccttgggcattactccttccagtcgaaggagatctcatgagacgagctcggaatgtgcttatatgtcaccagacggacgtgaacctccctggagatttgcttttacagtcgggcgttttttgtttcccgagtactttagagtttggaaccagttctattgctttgacaaggtctagtttattataccttcttgctctcgcgtttttttctctcgcacaactgatagacatataagtgttctttagatagaaggaaatggtgtacgatagcgcaaggattcctaaggccattgtggacaactatgtaaggacttatgacaggtccccacgaatagtgcgatatcatacttagcagcagagaaaccatcaagaaggtagactatagcacatattcagaggccacggaactcctgagacataagggtagggataaccactgcacacgagaagaacaatcagaataaaacagagccattttataacaaatactacatgacaaagggctaccagaatctattgatgagcagaggcgcatgagagaaagcataggatcccgatttcggtacccgtagtttaagggggtagaacatgccctagatcttagtatattttctagggaactgctattataatattatgataataatattattatgataaatatacttgtcctctagtgtgtgtaacatccaccctgcaagcttaaccgctttgggtgctcatcataggggaggtccactagttccaggagccagtcagatattttacaatagaatctatagatcggaggtcttaccaatttggctgcggttcgggtgagtctgggacattctgttaagattatacccactaccgaatccggacagagtccagatggccgcttaaccactatccgaagggccctaggccaaagctatcaggagtgtgctctagcacagttacacatgcttaggcaatcaaactttgaagataccgtaaaaacccttgtcgttcgctaccccaccgggttgctaatctccagattcagacatagcgcttaagcaggaatagtgctttcctgacacggtacagcctaagtcctgtggcattgttggttctcatgaactagttttcgcaaccctgttatcctgtcggatgtatgatagcatgtcgtgcgctgatgcgccgacccacagggatattggagacctacggagatatagaatcgaaaggatttttacagtattggtagatggctgttagactctcttgccctccctccttttgctacgcacgatgaagaatagaacaatatcggtaggtcagatcaaacgacgactttgcttggaattgagagatagagaaatagcttggctcgggcccctgcgcccgtcagccacccggaccgggtcagggttgggatttgaaagaagatttccctgggtcagtccggcagcactggtatcatacccaaagtcacagatgtgtcagcctggggttactgattcggcgctcgaacctcccccaataaccc

SEQ ID NO: 48 - DNA, 13263 bp, Tuber indicum
gggcgtggggctcggggaacgggtgtttttctatcgagtgattttttgtgatcttactggttttggctatcaatacatccacatataccaggagcttctgggcgattgcttgcctgcagacaagtgatccggacccatccagtggtgggtccggcacggggggagcactgtctgtctgttcgcttcacttgtgctgttcgaatgctttacctggttgtgcgctctgagtacgagaaatgggcgagcaagctagtctgtctattcacgaagggcgcaatgaactatcacaaaccaatcgcagattcttctactcttttgtccctttctcacggaggttctttccagtgagcttcgcccggtcttgtctcgtctaccattgcatagttatttacagcggatggcgtaatccgcacccttgtctatcatcgcaatacggcattggctctaacaaaactatatcgtgtcgttctgagggagaaaaaaagtcccttcactctactttcccttcctccactccgattcactggcagatgaaaccgtaccgcgcgctcgggctctttggtccctattttctcaatgggcgagggcgtcgttagtatgggggttgttaggctcctttggcctatttgccgtgagtagggagggcgctgtcattgctgagtagcctttcagcggggcctgcgggggtgcttctttccgatcggtcttgcgaggggtggtattggtattgggcggtgttggccattcggcggcgttgatgaaatttctatcaggagttctgtggcctctggatacatggtgtagtatatcttattggcggtttctctgctgctgctaagtatgatatcgcactattcgtggggacctgtcattaagtctttacatagttgctcacaatggtcttaggaatcctcgcgccatcgtacatcactttccttctatctgaacaacgctctatatgtttatctatcgtacggggggagaagggtgacgcgtgggcaagaatgcacggctggagaaaataaaatatgataggatgaaaacttcaaggtataataaactagacttttttgccacggcgatagaactaattccaaactttacagtactcagggggcaagcccacgcccgactataaaagcaaatctccaggggggctcacatccgcacggtgacatataagcgcattccgggctcgtctcatgggatctccttcgactggaagggctgatgcccaagggcgactttcatgagtaaagtaccccggaaaagaaggaacggtgatgccttcgatgaggtcctgttttataagccttaaatcgcgctaggggttaacgctttgtgggactgtagcagcgctaccaccttctgatggccgtgcctagatgcgaacgagagaggtgtttgaccgtcattgtttggcctgtcggggctgacatcttcccggccgagtagcaccttcaccacctccgcatgcccatcccaagcagcgagcgagagcggtgtttggccgcaatcatctggcctgtcggggttgacctcttcccgcatgagtaacattttcaccacttcttcaagtccatgagccgcagcatgagagagtggcgttcggccgagattgtctggcttgtcggggttgacctcttcccgctcgagtaataatttcgcaactgcctcatgccccctccaagcagcattcgagagcggtgtttggccgtcattatctggctcgttgggattaacatcctcccgcccgagcagtattctcaccacttcctcatgtccaaaccaagcagcgagcgagagcggtgtttggccgtcattgtccggcttgtcggggttgacctcttcccgcccgagcagtattttcaccacttcctcatgtccacaggcagtagcatgtgagagcggtgtttggccgcgattatccggctcgtcgggatcgacctgttccagcccaagtagcattttcaccacttcctcatgtccgttatgagcagcccatgcaagtggcgtatacccccaacaatctccttcgttgatatcccaacatcccatctctattaaagcagccacaacctcgacaattccaaagaaagatgcacagtgcagcccactgaatggaggccaggtatcgtgcccttcgggacctaaattccccacttgcgctaaaagaaattcggtggatatatggccatcaaactcctggaatagcttcagcgcaagagatctcccgcaatccgagagttccgttttcgcatggactccccaatacatagaacaatattctagaaagggtgcatccgaggtgtcaggagatggatcggtcgaaaggtccttgacttgccgggagttcagataggtcaagcatatctctgccattgccgcatggggtctactgaaaatatcgggatgagcggaaaggtactcttggagggtaaagtggactagtcgcacggctgatgcctccttgcccaccgtaataagcccttgacagcaacctactagtgtcgatatcgaagggatgttgttagcattgaagtcggtggagcccagctcgactgccagggcgcggcagagctcgtccgtcctgagcggccgctgcgcatgactgatccacatcaaggctgccatcccgagtctcgatttatccccgccctgcgcctttatccgtcctatcgtcgcaccgtaggcatctcccagacccaaaccgtcggtcattttgctaagcctgtctcgtctccgataggttgttgattcttgtaagattgcttcgatgtttagggaaactagtataaacctggatatatctattagcggatagtgtgcaatgggaatccctctgtcattgccctacaaacatttccgagatcttccctgggatctgctctagaacgtctgcctcaagaccctcgtccatggcatccggggtttcgtcctcgcccagcctaacacgaaggtatctgataatatcatctcttctagggcttatagacacacttgccgctcgcccgggaagacgcttttcgatctcggcccgaatataggaccttccagtcaggaatattcgtatccccggggacttcccaaggatttgtttcagcgaatcaaggagcttggctcgatgcactgcggcacattcgtccagggcgtcaatgcacataaatgtgcgctgtgaggacgtgatggtctgtagcatctttacgatatcaggaagttgcggtccccgcccgccgatggccctcctctgctcctgaaaagcccgcgatatttcctctggaatcctttccattccgctaacgatctgtttaagcaaggaacccagcatattcgtcgcggactgctcctttcgggccgcaaagtcaaagtaaaaacaggtaacggtagtgttttgtcctctggcctgatcgcacagcctgtccactaccatcgaactgtcatcgcggcttattagcactggcgcacttctctccccagagaataactcttgttacctgataaatgtcttgcccacacctgggtctccatggcaaaacaacactgccttattgccttcaccttccaaactccagtcataccagcttctgaattcgtcagtttggataagccattcccccacattgtccacacggtgctcttggatgtcgcgatgccgtaacctaggttccagcggcgacaaccaagctaggagttgagaccgatcatcggcaactgtgaagttgttccagacattgctaaaagagctcgtattattgaagcagttgttattgctattgattgtatatcgagacatcttccgctctttactgcttgcactattgaagggaagtatttatctggtttggttggttgacgatatcctagggaggtcaagaaaagcggggtctagctggggaaagcaagggtctgctggtgttaaagcagcatcgcagataccgctactcgtacatgcagtactaactcaagagttttggtccggtaccctttttacaccaaaagtatctaacttcaccaccctctctcacagcacaatgccaaggggaatatgccaaaatttcgcggtgtattggcaaatctgtagataatatgggagataatattattacagaattgttgagtattatagaggggtggagaaaatgtcatttaagtgcgaggtattggttgtcatgtacttttttgtatagagccccatacaggtacaaaaatccaggttaatttcacacaggccaacgttattgtaaacaatgggcacaactttgatccctttgatcctctacagttctacttctagagatttaaataaataacagctatctagctgagagagggatttaaagtgttttcaacgggtattgttgggattgttaggtatgttgcggtagtctgcgacttgataaacaccatacttcgtggagaatgatataaagcgctgcgcccgggaaaagtaaggacgaaataaatagacactgttaaatacctagaacaaggtctctagcattagagtgcaatttgaaaacttgcggtcgataaattattcgtgatcgagagattgtggctgttgcatcgaactaccatatgactttcctaaataggaaggcagactcttggtgcctggcagttgaaggttctgaaataggaactattggtactcgtactaaatactaattcctagcaacgtacagggatgtcacgcaaaatcaaatcaaacataacacactaatggtattaaagaatgttttacttgctagcgccgtcatagttcctttcccaatcacctggaatttgttagttccaatagacagctataagggctcttgtagatgagtgtttaaggatttacattgctatctcagcttcagcgagaacaagaaaaatgtcattttttgcccccacagcccaatatgtgcagcgaaaatgtttgttttttctcccgtcaaaatacaatttttgccaggataaggggaccctaggtcagggtggggttaaacggcttggggtatcatatctagaaggaaattgtggatccctggacaattagcgcgcattaaactcacagctctttggtgagctttcctcgttgtctttctgtaggtgcacgtggagccagccaggtatctggggggacctgggaagcattccctcttattgtggtggtagggcctgcaatggggaaactccagcatataaaatttaataaagttatattataatattattattacgactcccgcctatctcataattaaattctgcgatattcccagctctggggtattattggttgttatctccatgatgtataacccgatgtttgatttagcgtgactattgtaaatcacagtggaatgaaagataagagtaaatggggacaggtggtagaagtgacttgcttgactttctgggtggggtggatacatacatatagctcatagagatgtcatgctaaggtatatagacaactctatgacacaacttggatatggtggataaagtgtgacggcctactaactgagaccacaaaggattcaattgtgtaccttaagaacatggaatacttatgctgtaatgttttgtgcatgcaggaatcaccctttcagtagatgtcgaattttacagctttcgaactagcaatccattaccaaataagtatcgcgggtgcgcatttgtgggaagagatgggcattataccataaaccacaggttgtaaaaagtgtttagattttggctgcttccagcgattccatcaggactcaagatatttcctttccagtacaactactaaatagcaaaagatagcgaagagatggaagagagaggccctagtgtccgcctcatgtgcaattacggtatgtttgtgagagacactccgctatgatctactaccttcacagatgttcctaacattatatatacaaacgtgcaatatcccgatctctgatcacacaatcccctgtcctacatccaatcaagttcctttcctccgtgtaatccacttgacatcttagcttctatcccgggagtcggagcgggaccgggggcacattgccccaggctctcttatctgacaatattttcgtccagaatcaagagactttgtccgtcccttctaaactccgcccccttcaatcatcctgccttgcatgaagctcaggaggttgagtgcttagatgctccacaatggtttgctcaatcggcagcggctcctgagccagaagctctgatggcttagagttggttaaagattttaatttcccagctgaaaatccttttggggtcgagctttgctcgctcgtgatccagaccttctccgttcgttatattactataccatcctactgatatcagaatggcctaacttagccaaaaaacccctctgagaccactaggagtaactgctagcaatgttagccacaatccttaatttccagacccttaccgagacccgttgtagcattggatggctgctcctttgttctggagttccatcttttaaacagctttcccgagagctgcatacgccgcatcaaacgccgtagtcctccttgtccattcacctggatgcacaagcggatagccgaacatgggacatccgccggatcggctggtagttttgcaaaagaatttggctttcctggttggcagggcagcgggtgaagctaccatacttctgaattcacatttcgcctaacggcctcctggccattggatatccccccaggacggaaaggacagcctcgggatggtcaggcttaaggtaccggtatggtttcccagcccgccacacagatttatggagatcatattaaattcccaatcagaaaaaactatcttgatccacatacggtatctgatcgcaaagaattattgttcagggctcagataccatacctctctatcaaagaagcaatgtacgagtacaagagaggtggggttgggggacgggcgctgtaacagaacccacatttcctagctcaccctgtttcattaatttctttgttcggaacttctcccgaatgtcaatcagatcttcatcaataccttgactcgttttctaccgtactccagcatctccgaccgtaaaaaaaaattcgcggatctttttcaagataatactattctcgggccctaatagggttgagttgtacgtgacggcttcttggcgtcactctattttttgggtcttggcagtccccctcgtgactagaggcctacaaaggagatccatcctttcccccttctttcctccaccgacaaccgtcaagttcaacagaaacattcggcctttcaacttgcacctctccactcacgtcgtttccttttttctgccactatgactatgacttcttctttgtccattctcaatatggccgttgaagccgggcctcttattcaccaacgttatgatggaatcggtcttgccgagtatattcccttgctcgccaacggaacctttttctttgatgggccggatggagtctccctccaagtccgccatctccttccagcagactggaagcccgtcaatggaccacttacggtcttgttcgattccgccccccataccattattgggactcatttttctatccgcaatcatcccatttataagcttaacgatggttctatctttgtcgtcttggacccggccaccaagcacggtttcgagtttaccgatcaatctttcgtggacccccagcttgaggtctccggagaaaaccgtttcttgcgtctcgttaccaatgaccaagacttcgcctaccatggttctagtcttagcatgattcatgacaagggcgttaagcgccctgccaatgcgtattgatacaattctaccaatgtaccagtttcgtgtgctcatctagtcataggttcattttattcagaacggacaaaatgaagcccctcaaggctcagtacccggagatgggcaacaacgatatctgtacgtggctctcatttcccccttatttagatctctattcttggtaccatttgcctaacgttttaatagccaagattctcggtcgcatgtggcagaattcctcggatgaggtcaaagatgtttataaggtaaaacatctcacctaactgttagttagaatcactaacacattaataggcaagggcccatcagcttgcggtttcccataagttgatcaacccggattacaaatactccccgcgccgctctcgcgaaatcatgaggagggactccgggaataaaggtccttccccccgtatcgaccttcaccgtgctggcgttgcgaagcgtggaaaaaagaggatcgttacccaccttgggttcctcactcaacgtttcgcgactcaccccactgttaacaatctcggtgatatcatcgaggaagccgaaaaggtcttgaaggcctttggtatggtcaattgagtgatctcaacggagggactcatcagtacctgggtggtatactttcaatgggagggatgatcttgtctatcaaaggtcattatctcagtcgaataaaaaaatttatatgacctaaataaataaaaaatatgtttaaaaaaaaaaaaaaaaataaggggtctgaacactatatcatacagtaaaatatcaagttgtgaagcagaagagagtcgactaccggtatgggaagaggcagtgagagtttaattctgactatcttagtctggaaagttcgtacagtacttgcacatatgttatgttatggtggtgctatcacctgagaaacacgccaaaaattcgggacctatggtgccgtgacttagtttgcgcgcaaatatgtactgtacatggtccgacataaaagagtgttacggagcaggggtggtggggttgattttggccatcagcgttatcgcacagattgtttgaaaacacggctcgcattgctcaacaagaacatggagagagctatttttttaattttgataaatgtattggactagaggccaagaatctgatcaattggttatactatatttgttcacagttgactgaggccctgtttggatacaacggtccctttctgtttccccgtacgaggtgaccgttgcagaacgggagggcaatcacttttatatgagaaacagttccaaaacgagacggaaccgttccatccaaacaaggcctgggagaagaggaaattgtgatcttctcaggtttaagtttcttggatgtgtcgctgctactgaaaattttggtatataggtatccgttttatatctgactccctccacacagaccccagtggagtgaatacccggagcactttgctcatgaaagtcgcccttgggcatcaccccttccagtcaaggagatctcatgagacgagctcggaatgtgcttattgtcaccatacggacatgaacctccctggagatttgcttttacagtctggcgtttttttgtttcccgagtactttagagtttggaactagttctattgctttgacaaggtctagtttattataacttcttgccctcgcgttttttctcttgcacaactaatagacatataagtgttcttcagatagaaggaaatggcgtacgatagcgcaaggattcctaagaccattgtgagcaactatgtaaagacttatgacaggtccccacgaatagtgcgatatcatacttagcagcagagaaaccatcaagaaggtagactacagtgttgaaaatcacgaagcttccactgtttagagtatgaggcgatggttgtttggcaatgttttagaaacacaatggtggaacatgctaaaggaacgagtcaacgaaagagtcaacgggtaagtaagagtacgtaggaactagcgttagtgcaaggagaagtgtttgggatgttttaggagttagcactagggatgccggaagtaacgggttacactccggaggtctagtcgacaaccaggtgtcgcactatgggagataggaaactggagaagatgcacgaaggtatcgggactgtctggaacagcgatagcctaggagaaacgtttcggaggatgttcgaaggagttgtgtggatggaactgtgtggcatcatgaagaaaagatggtatggaattctgcgaggcagatggacgaagaaaatgtttgcaaaggaacagcgcgtgtatatataggacacctccggcagcatagaacaaacaagaatcgcagacactctatacctatcatagccttattatttgatacagcacatattcagaggccacggaactcctgagacataagggtagggataaccaccgcacaccagaagaacaatcagaataaaacagagccattttataacaaatactacatgacaaagggataccagaatctattgatgcgcagaggcccatgacagaaagcataggatcccgatttcggtacccgtagtttaagggtgtagaacatgccctgggtcttagtctattttctagggaactgcaattatactataataatattattatgataaatatacttgtcctctggtgtatgtcacatccaccctgcaagcttaaccgctttgggtgctcatcataggggaggcccactagttccaagagccagtcggatattatacaatagaatctatagatcggagatcttaccaatttggctgcggttcgggtaagtctgggacattctgttaagattatacccactaccgaatccggacagagtccagatggccgcttagccactatccgaagggccctaggtcaaagctatcaggtgttctagcacagctgcgcatgctcaggaaatcaaactctgaagataccgtaaaaacccttgccttcgctaccccaccgggttgctaatctccagattcagacatagcgtttaagcaggaatagtgctttcctgacacggtacagcctaagtcctctggcattgttggttctcatgaactagttttcgcaaccctgttaacctgtcagacgtatgatagcatgtcgtgcgctgatgcggcgaccaacagggatattggagacctacggagatatagaatcgaaaggatttttccagtattggtactcgtaatgtcgtgacgtttgtgaacacgaccgggcttgcattagtgtacattagtgtgaatacaagggggtaacagacgagaaatgaacttaatgtagactagttagtaaataggggtacttcagtgaataggggtacttgagtatatgaagttcgtgatcaatagtagcaatacagtgaatccaatatccagcaaaaatgccaactccacgaggtcctacaacttctttgagcaagcgaggtcagattttgggttatgcaattctcgatgggcgacagaaaatgacattgagggagatatcgaaaaagacaggaatacttgaaagtacttgttcgaatattatccgaactgcacgcgagcgagcgagtgtgaatggcatccaagatctctgcgtaactgagaatctaaagccattgcctaccgcactcaaaggatcaaatcaggctcttaccgaggaagaaaagggtcatcttgttgaaacagcactgaaggatgctgagcactgtcgaatgacctttacgcagcttgctgaagctggtatgtattttatattaaaattccctataatacataaatgtaactaaaaaaaaaaaaaaaaaaagccaatttagatatcagtcggcagactgtatctaagatactagctgaaaataaaatccatcgccgaaagccaacaacaaaaccctcactcaatgcagctcaacaggcagcccggcttgccttttgctatgagcatcggaactacgattggcactctgtaatttttacagacgagtcttattttgaaactggcaaccttcgccagcgccgtgcccgtggtgttttacgccgggctggtgaggcctaccggcctcaaaacattcagcgaaagtttgctcaaggagcaacggtcatgttttggggggctattttgtatggtaaatcaggtataatttatttattattattattgttattgtttattactatttctctttctcttactatttttattatttttaatgtacatataggctaatataatatattaaaaaaggaaatgaactaccatatcatttatatgcttcaccatatgaaacagcgggacagcggagagagtcatcaattatcctacagcgagaatacgagttcgagttagcagaaaatactcgcgctactgtacttggacatgtagtcccatttccaacacctgaattaaaggtgcggaaggtagagagaaagggagggatagattggtatatctatcgtgaaagaattttaaatccccttctttaccctttcacccagcaagcaatacaggactttcctgatcggaatattaaaataatggaggataatgcgcctgcgcatatccaccattatcataatattccgcgagagcgtttggggctacggaagctagtctggccggccaactcccccgacttgaatcccatagagactatatggactgagcttaaagataagctgatggatcaaattggtccacggatgactgctcgtgatatccgccatgtacttgaacaagtaagctttaccttcccttttattcttttactactaacaatttacataggagtggagaaactatccacgagaacgtgtgaaccaccatatagaatctatgtcttcacgaatggaagcctgtattgcagataatggtggaaacaactttaatttttaatattaaattttgtatttctttctttctcttttgattccatgagtgctgtattcaccatatataattcgtaatatattatcatacctttatatccccctccctagtttcgccaagcataagcctcatatgcacgctaatgcaagcccggtcgtgttcacaaacgtcacgacattacgagtagatggctgttagactcccttgcctccctccttttgctacgcacgatgaagaatagaacaatatcggtaggttatcatagatcaaacgacgactttgcttggaattgagaaatagagaaatagcatggctcgggcccctgcgcccgtcagccacccagaccggaccagggttgggatttgaaaggagatttccctgggtcagtccggcagcattggtatcatacccaaagtcccagctgtgtcagcctggggttactgattcggcgctcgaatctcccccaataacccctcccttgcagcatctcgcccaaagctagtagcatagggccaaaatgctatgtatcataataatattattatgata

SEQ ID NO: 49 - DNA, 8730 bp, Tuber indicum
  misc_feature (3897)..(3897): n is a, c, g, or t
ttcggcttgcatgatatcagcgaatgtagctcggggactaaatctgatagttacactaaacgttggataccacgctcgttcagagctagctccttctccgctcgaggtagaggattaagttcacaggcctctgaatggcatctgatgatgttccgggcaacttctctcggggcatctgtgtcgctagcaattgtatgataacttttgccttctgcatgaagctcaatcattcgtactctctcatcgcttgcggccattctccgcttgcctgttgcttgaccttgaggtatggtgtatgatgggaggtgttggctcccataaagaagtcgtggctaagatactagtgtcgggttactaaataacctctactgctgagaccgttgcgaaagctgggtaggttctctctacatattctagtagatattgacgctgatttgcgggaggaaaaaaaggaattttatgttttttttggttttaaagataccgagggcgtttcattgatcctagatacataagtttaatttaagtctacgcgatctataatacgctgagaatgcctcccaactatgcccttaatttccgccgctcccacacagtcatactgtgatacagtaacttcagtttttttttgtacccactagcctctttaggccttctcgggaggtcatatcggttcacaatccagtggaggattctccaataggaggctgtttagatcatgttagaattgggcgctggtggaatatgttgttcggggaacttctcacaaaaacgctgtatccaccagaagtgaaagtgttcatgtgcatctcggtctcgggctggaagtgcgggtcgagttgagagtctccattctcctcagcgctagcgatgtcgaaagcattgaggatgttggcggcttcctcctcggtgagatcgagctgatggttgctggaaacgtaaacacgggtgatggtgaatctggacaccgcggatgcgggcttggcacgagccttccttcttgcagcagccttatcggccgcttccttgtcgatgggggccttggggacaaagccagcttcacgcaagagcttctcaggggtagtagggtgaccgtgtttcgccaattcggtctcgataaattcttcgagcacgagaccagggtcaccacgatccctagcagcagtataaagagaggcaatgttttgccacatatacttattggtctcagcgacccagacatcacgagtgagattggaaatttcggcttgagtgagcccataaccgttacagtacttagaaatccaggctaatagatggttagaaatagaggcaaaatagggatgtgatttacttacatctctgcgcaacgtagggattcaatgcacgaaggtgctgagagggggggatgcgggccaaggcctcatcaccattgacgataacagccggaatagtggcatcctgggccgtccacggctgtccaggtttatagtatccgcaatacaaacgtagaaccgacacaacataagggacttcggggtcgagggggactcgccctcgatgacggacggttgattgaattgcaccaagccacttgtcgtagggcaagagttcgccgctgacagtgaaagtgacaccaccggggggttgaacgataatgggggagttctcgtgttggacccagatgcacccgatcctgtcttgcatgagaatgtacccacggtccgaaaggtacaaggcgcatcccaaggtagcattacgcataatgaggcgtctctcgagcctgttctcccactccaaaaccttaggggggggggagaggttaacgaccatgctcagattgacaggacgagacgacattatgaaaggcggggggggtggagacgggaaaagaaatttgaaccgacaaaaggaggattgcgtatgggcgtttcaatgagtggagattggcctcaatggctgttgatggaagaaaggagggtggaaggtctggcctcttttgtatgctacccgtcgaagtgggatgccaaggcccaaaaaaaagagtgacgccaagaagccgtcacgtacaactcagccctactagggtctatcataagatttttcccagagcaggtagagagcccttaggtttctgggattcctcgcggaaagatcggggaacatttttgtatttacggtcagagttgctcgaatagaaagacgagtcaaggtatttgatgaagatctgatcgacattcgggagaagtttaaaacaaaggaattagtgggggtgagtgggaaatgtgggttctgttaaagtatctgtcccccaatcttaactacccctcttgtacattgcctctttggtaaaggggtatggtatctgaaccttgaacaataattccttgcgataatatatatcgatcaggacagtttgctgactggaaaatttaatatagtctccataaaaccgtgtgaccgactcggactgggaaatcataccttaagtctggccatccggaagccgctctttctgctatggggtaatatcaaatagccaagaggccatcaggcgaaatgagaattcagaaatatgatagccctcccctgccaaccaagagacaaatacttcggcaataccaccacccgatctggtggatgtctccatgttcggctatccgcttgagtatccaggggaatagacaaggaagactaccacatttgatgcggtgtgtgcggctgccgagaaaactgtttgaaagatggaactccagaacaaaggattaactggtcaatgcaacacctggtcgctgcaagggtctgaagattaaaggattgtaactagtattgttggaggttactcctagcaatttcagaggggtttttggtaaagatagactacagtcgcaccccgctgtcccggacctcgctctcccggatcccccgttgtccgggacgaaattctctgagctctgaatttttttgcaccagaacgtaccccgctgtccaggacagattctgcggattttggccatccgggacagcgaggtgcgactgtatactgatatcagtaggatggtatggcactacaatgaaagaaagtggtctggaccatgagctcaaaaactccaaagagaatttccggccgggaaattataatatttaaccggtttaaaaccgtgagatcctctggcccaggaccagcggccgattgagagagctgttgtgagagcatcttagcactcaaccattagaactccatgataggtaggatgataaaaggggaatgggtttagaatgggtgcacaaagcttcttgattctggattaagatatgactacataagggagtctgaaacaatgtacactctatctccgcttggacttataggataaaaatggacatgttaagtgaattataatgaggaaaaaagcctgattggttgtagggatcgattgtatgatcaggggtgggagtactacaagtttgtctatatagtattgggaataactgtaaaaatagttccagaatatggctgagtatttcacaaacatgcattagttgaatatgaggtagatactagagtcaccttgttcagtcccttggctgtctttgaatatctggaaaatataccaggaaagaaagtgggaaaaacaatatcttcaacttctttggcaaactctttggaagtagcaaaacctaacctctccttacagcctgagcctgtagcttgatatggtactcatttactctttcatccgcatagctatgcacttgcaatatctcttccaactgtttgtttaaaaaccatgaggtttgacatcaaatgaaggagtaatttccaggtgcacaaaaaatatattgtgtgaatacctacaattgagcccaaatcgatttagtaaattgaaacccatcaagtagttggtaatctatctnatgaagttttttgagctgaagggaaaagagaaatgttaggcaaatttttttgtgacccttagctggtcggctgtcaatgcttgatattgtcatccaaaccaccacagattgtattgctgggatgttgacatgttgcagtacgacatctcctaaggcgatatgcatatcagtgattaccttatcgtactaagaaagacaagacagtcatttctactacttgcttttttattctcttacttcatattctaccatgatttacaggggtcatgctaaattaaatattgtgtgataccttgtgaaaaatgtccaccaaagccacagagctgtgaatatcgcaaattgtagggtgagagggaaatcacaatgcataactttataataattataaacaccacaactgctccactggggtgccttatgctaggactttcctccatgcgaccctttttgcatcataaaacaaataataacagattttacaaaagaaaaaataaaaaccaatgaggatgccccaccgccacattaaaggagaaagtagttcttagatcttgtctccctcaattcacttagtgcacctgtggatagaaacttaggtgaagatagagaaagaggtatcatgagttttaatgtacattattcattgcaatttaattccgggcacactaagttatttatttatttattcaatccctactgcaatccaagctactcttgttcttgccaagactatatcttgggggcggggaacaaactattttcattgcaccttttgggcgctacgagcaaaaaattgaccattaaattttcatcaatttaaaatcctaaatacaagtatcatacacatctactcaagtacaagaatccttgtaactattggaacaaaacaaattctgggactcttgtcggacactacgttaacgctagtaagtaacaaaaaatcttttaatattagaagtgtgtgtgacatccacttagattttgcgtgacatccctgccgctaccagaccttagtactcgagtagtagaataagaggagtagcgaccttcaacgacagccaagccatcaaaaagggtatcgtactcgagtgttcaaatagaaaatctaccttcctgtgataccaggtgaggtcattgtggtggttcaatacaacaatcaagtcctgcgattacgcataacaagtgtgagtactcgagaagagcgcgaatacacggctgcctggcccgcgtgggtctaacagatctgtccttgctctatttatttgttttgtttttcctaagcgcagcactttttaccctatgaggtatttataataatattatagatattctggaggaggttcccaaaaatatatcggaaatgcacctaggggcagtggtactaggattcccaccgcacactatctgctagtggatatgtctaggtttctagtttccataaacaccagacgagaaaggcttaccaaaacgacagagggggttgggtctgggagatgtctacggtgcgacgatagaacggataaaagcgcagggcggggataaatcgagactcgggatggcaactttgatgtggatcagtcatgcgcagaggctgctcgggacggacgagctctcccacgccctggcagttgagctgagctccaccaacttcaacgctagcaacatctcttcgatgtcgatactgctgggttgtttctaagggcttattacggtggacaaggaagcatcagccgtgcaactggcccactttactctccaagagtacctttccgctcatcccgatattttctgcagaccccatgcggcgatggcagagatctgcttgacctatctgaattcccggcaaatcaaggacctttcgaccgacccatctcctaacaccccgggtgcaccctttctagaatattgttctgtgcactggggaatccatgcgaaaacggaactctgggattgcaggagatcgcttgcgctgaagctattctaggagtttgatggccacatatccacagaatttcttttagcgcaagtggcgaatttaagtcccaaggggcgtgatacctggcctccgttcagtgggctgtactgcgcatcgttcttcggaatcgtggagattatggctgctttaacatacgtgggatgktgggataccggggaaggaggttgtttggggtaaccgccgcttgcacgggctgttcataacgggcatgggggggtggtggaaatgctattcgggttggaaaaggtccatccggacgagctggataaccgcgaccggacaccgctctcacatgttactgcctgtcggcatgaagaagtggtgaaaatattgctcctgtttgagcgggaagaggatagccacaaaaagccggacaatgacggtcaaacaacactctctttcgctgctcggtttggacatgaggaagtggtgggaatactacttgggtgggaagatgttaatcccaatgagctagataatgacggccaaacaccgctctcgaatgctgcttggaggggtcatgagagagttgcgaagatattactcgggcgggaagaggtcaacctcgaagagccagacgagctcggccgggcgccgctcttgcatgccgctggtcatggacatgagggagtagtgaaaatttttctcgggcgggaagaggtcaacctcgggaagctagacaattactcccaaacaccgcttttgcatgcgtttggtctggatatgaagaggtggtgagagtgctactcatacggggagtgtcgataccggcgaaccggacatcagcggccaaataccgctctcgcatgctgncgctcatgggcacgaggaggtggtagaaatgttactcatgtgggaagaggtcaaacccgacaggccagatgatttcggccaaacacttgctgcttgggatggacatggggggatgatgaagtactactcggccgggaagatgtcaaaccggacaggccagacgatagcggtaaaaccctgctcttgttggcctcttgggataggaatgagggagcggtgaaaatactacttgggcgcgaagttgtcggccccgacgaaccggataatgacgaccaaatacccctctcgttcgcaactgggaatgaccatcagaaggcggtggcccacaaagcgttaacccctagcgcggtttaaggtttataaaaagcaacctcttcgaagggatcaccttccttttcttacagagtactttgctcgtgggggtcgacctagagcatcaccctttccagtcgaaggagatcccatgagacgagtctggaatgcgcttatatgccaccatgcggacgtgaacctctctggggatttgcttttatagtcgggcgtgggttttttttctgagtacggtagagtttggaactagttctattgccgtgacaaagtctagtttagtttattacaccttgcagtcctcatcctatcatatttgatttcctccagccgtacattcttgccctcacgttttttctctcccttgcgcaattgataagcatatagagtgttgttcatatagaaggaaagtggtgtatgagaggggaagggttcttaagaccaatgtgagcagctatgtaaggactgaatgataggtccccacgaatagtgcgatatcatacttagcagcagagaaaccatcaggaaggtaaactacagcacatattcagatgccacagacctgagacataaggatagggataaacactgtacacgagaaggacaactagaataaaacagagccattttataacaaatactacatgacaaagggctaccagaatctattgatacgcagaggctcatgagagaaagtataagatcccaatttcggtacccgtagtttaagagggtgaaacatgccctagatcttagtatattttctagggaactgctcttataatatgatatgaaaaatatacttgtcctctagcgtgtgtcacatccaccctgcaagactaacatcttcgggtgctcaccataggcgaggcccactagttccaggagccagtcagatattatacaatagaatctatagatctgaggtcctaccgatttggctgtgattcgggtgagtctgggacattctgttaagattatacccactaccgaagcctgacagagtccagatggccgcctggccaatatccgaaggatcctaggccaaagctatcaggtgttctagcacagctgcacatgctcaggaaatcaaactctgaagataccgtaaaacaccatgccgttcgctaccccaccaggtcgctgatatccatattcagacataagcgcttaagcaggaatagtgctttcctgatacggtacagcctaagtcttgtggcattgttggctctcatgaactcgcaaccctgttaacttgttggacgtatgatagcatgtcgtgcgctgatgcggcgacctacagggatattggagacctacggaaatattgaatcgaaaggatttttacagtattggtacatggccgttagactctcttgccctccctccttttgctacccacgatggaaaatagaacaataccggtaggtcatatcaaatgacgactctgcttggaatagagagatagaaaaacagcatggctcgggccactgctccctttcagccacccggaccgggccagagttgggatttgaaagaagatttccctggatcagcccggcagcactgtacctaaagccacacgctatgacagcctagggttactcactcggcactcgaacctcccccaataacccctcccttacagcatctcgcccaaaactagtatcatagggctaaaatgctatgtatcatatgac

SEQ ID NO: 50 - DNA, 9016 bp, Tuber indicum
tccgcccggccgagccggcgcggtgtccttcacggcggggcgtgggctcggagaacgggtgtttgtctatcgagtgatgttttgtgatcttactgattttagttatcaatacatccacatatacgaggagcttcggcttgcatgatatcagcgaatgtagctcggggactaaatctgatagttacactaaacgttggataccacgctcgttcagagctagctccttctccgctcgaggtagaggattaagttcacaggcctctgaatggcatctgatgatgttccgggcaacttctctcggggcatctgtgtcgctagcaattgtatgataacttttgccttctgcatgaagctcaatcattcgtactctctcatcgcttgcggccattctccgcttgcctgttgcttgaccttgaggtatggtgtatgatgggaggtgttggctcccataaagaagtcgtggctaagatactagtgtcgggttactaaataacctctactgctgagaccgttgcgaaagctgggtaggttctctctacatattctagtagatattgacgctgatttgcgggaggaaaaaaaggaattttatgttttttttggttttaaagataccgagggcgtttcattgatcctagatacataagtttaatttaagtctacgcgatctataatacgctgagaatgcctcccaactatgcccttaatttccgccgctcccacacagtcatactgtgatacagtaacttcagtttttttttgtacccactagcctctttaggccttctcgggaggtcatatcggttcacaatccagtggaggattctccaataggaggctgtttagatcatgttagaattgggcgctggtggaatatgttgttcggggaacttctcacaaaaacgctgtatccaccagaagtgaaagtgttcatgtgcatctcggtctcgggctggaagtgcgggtcgagttgagagtctccattctcctcagcgctagcgatgtcgaaagcattgaggatgttggcggcttcctcctcggtgagatcgagctgatggttgctggaaacgtaaacacgggtgatggtgaatctggacaccgcggatgcgggcttggcacgagccttccttcttgcagcagccttatcggccgcttccttgtcgatgggggccttggggacaaagccagcttcacgcaagagcttctcaggggtagtagggtgaccgtgtttcgccaattcggtctcgataaattcttcgagcacgagaccagggtcaccacgatccctagcagcagtataaagagaggcaatgttttgccacatatacttattggtctcagcgacccagacatcacgagtgagattggaaatttcggcttgagtgagcccataaccgttacagtacttagaaatccaggctaatagatggttagaaatagaggcaaaatagggatgtgatttacttacatctctgcgcaacgtagggattcaatgcacgaaggtgctgagagggggggatgcgggccaaggcctcatcaccattgacgataacagccggaatagtggcatcctgggccgtccacggttgtccaggtttatagtatccgcaatacaaacgtagaaccgacacaacataagggacttcggggtcgagggggactcgccctcgatgacggacggttgattgaattgcaccaagccacttgtcgtagggcaagagttcgccgctgacagtgaaagtgacaccaccggggggttgaacgatgatgggggagttctcgtgttggacccagatgcacccgatcctgtcttgcatgagaatgtacccacggtccgaaaggtacaaggcgcatcccaaggtagcattacgcataatgaggcgtctctcgagcctgttctcccactccaaaaccttaggggggggggagaggttaacgaccatgctcagattgacaggacgagacgacattatgaaaggcggggggggtggagacgggaaaagaaatttgaaccgacaaaaggaggattgcgtatgggcgtttcaatgagtggagattggcctcagtggctgttgatggaagaaaggagggtggaaggtctggcctcttttgtatgctacccgtcgaagtgggatgccaaggcccaaaaaaaagagtgacgccaagaagccgtcacgtacaactcagccctactagggtctatcataagatttttcccagagcaggtagagagcccttaggtttctaagattcctcgcggaaagatcggggaacatttttgtatttacggtcagagttgctcgaatagaaagacgagtcaaggtatttgatgaagatctgatcgacattcgggagaagtttaaaacaaaggaattagtgggggtgagtgggaaatgtgggttctgttaaagtatctgtcccccaatcttaactacccctcttgtacattgcctctttggtaaaggggtatggtatctgaaccttgaacaataattccttgcgataatatatatcgatcaggacagtttgctgactggaaaatttaatatagtctccataaaaccgtgtgaccgactcggactgggaaatcataccttaagtctggccatccggaagccgctctttctgctatggggtaatatcaaatagccaagaggccatcaggcgaaatgagaattcagaaatatgatagccctcccctgccaaccaagagacaaatacttcggcaataccaccacccgatctggtggatgtctccatgttcggctatccgcttgagtatccaggggaatagacaaggaagactaccacatttgatgcggtgtgtgcggctgccgagaaaactgtttgaaagatggaactccagaacaaaggattaactggtcaatgcaacacctggtcgctgcaagggtctgaagattaaaggattgtaactagtattgttggaggttactcctagcaatttcagaggggtttttggtaaagatagactacagtcgcaccccgctgtcccggacctcgctctcccggatcccccgttgtccgggacgaaattctctgagctctgaatttttttgcaccagaacgtaccccgctgtccagggcagattctgcggattttggccatccgggacagcgaggtgcgactgtatactgatatcagtaggatggtatggcactacaatgaaagaaagtggtctggaccatgagctcaaaaactccaaagagaatttccggccgggaaattataatatttaaccggtttaaaaccgtgagatcctctggcccaggaccagcggccgattgagagagctgttgtgagagcatcttagcactcaaccattagaactccatgataggtaggatgataaaaggggaatggggtttagaatgggtgcacaaagcttcttgattctggattaagatatgactacataagggagtctgaaacaatgtacactctatctccgcttggacttataggataaaaatagacatgttaagtgaattataatgaggaaaaaagcctgattggttgtagggatcgattgtatgatcaggggtgggagtactacaagtttgtctatatagtattgggaataactgtaaaaatagtttcagaatatggctgagtatttcacaaacatgcattagttgaatatgaggtagatactagagtcaccttgttcagtcccttggctgtctttgaatatctggaaaatataccaggaaagaaagtgggaaaaacaatatcttcaacttctttggcaaactgtttggaagtagcaaaacctaacctctccttacagcctgagcctgtagcttgatatggtactcatttactctttcataaataaataaacaaataaacccgtcggacggggggggggggctggctggctggctggctggactgggctggcggttgtgtatgtgtgagatttgctattttaccttccacagtaccatcatcatcatacttgcttacttttttacttttaacttcatccgcatagctatgcacttgcaatatctcttccaactgtttgtttaaaaaccatgaggtttgacatcaaatgaaggagtaatttccaggtgcacaaaaaatatattgtgtgaatacctacaattgagcccaaatcgatttagtaaattgaaacccatcaagtagttggtaatctatcttatgaagttttttgagctgaagggaaaagagaaatgttaggcaaatttttttgtgacccttagctggtcggctgtcaatgcttgatattgtcatccaaaccaccacagattgtattgctgggatgttgacatgttgcagtacgacatctcctaaggcgatatgcatatcagagattaccttatcgtactaagaaagacaagacagtcatttctactacttgcttttttattctcttacttcatattctaccatgatttacaggggtcatgctaaattaaatattgtgtgataccttgtgaaaaatgtccaccaaagctatagagctgtgaatatcgcaaattgtagggtgagagggaaatcacaatgcataactttataataattataaacaccacaactgctccactggggtgccttatgctaggactttcctccatgcgaccctttttgcatcataaaacaaataataacagattttacaaaagaaaaaataaaaaccaatgaggatgccccaccgccacattaaaggagaaagtagttcttagatcttgtctctctcaattcacttagtgcacctgtggatagaaacttaggtgaagatagagaaagaggtatcatgagttttaatgtacattattcattgcaatttaattccgggcacactaagttatttatttatttattcaatccctactgcaatccaagctactcttgttcttgccaagactatatcttgggggcggggaacaaactattttcattgcaccttttgggcgctacgagcaaaaaattgaccatcaaattttcgtcaatttaaaatcctaaatacaagtatcatacacatctactcaagtacaagaatccttgtaactattggaacaaaacaaattctgggactcttgtcggacactacgttaacgctagtaagtaacaaaaaatctttttaatattagaagtgtgtgtgacatccacttagattttgcgtgacatccctgccgctaccagaccttagtactcgagtagtagaataagaggagtagcgaccttcaacgacagccaagccatcaaaaagggtatcgtactcgagtgttcaaatagaaaatctaccttcctgtgataccaggtgaggtcattgtggtggttcaatacaacaatcaagtcctgcgattacgcataacaagtgtgagtactcgagaagagcgcgaatacacggctgcctggcccgcgtgggtctaacagatctgtccttgctctatttatttgttttgtttttcctaagcgcagcactttttaccctatgaggtatttataataatattatagatattctggaggaggttcccaaaaatatatcggaaatgcacctaggggcagtggtactaggattcccaccgcacactatctgctagtggatatgtctaggtttctagtttccataaacaccagacgagaaaggcttaccaaaacgacagagggggttgggtctgggagatgtctacggtgcgacgatagaacggataaaagcgcagggcggggataaatcgagactcgggatggcaactttgatgtggatcagtcatgcgcagaggctgctcgggacggacgagctctcccacgccctggcagttgagctgagctccaccaacttcaacgctagcaacatctcttcgatgtcgatactgctgggttgtttctaagggcttattacggtggacaaggaagcatcagccgtgcaactggcccactttactctccaagagtacctttccgctcatcccgatattttctgcagaccccatgcggcgatggtagagatctgcttgacctatctgaattcccggcaaatcaaggacctttcgaccgacccatctcctgacaccccgggtgcaccctttctagaatattgttctgtgcactggggaatccatgcgaaaacggaactctgggattgtaggagatcgcttgcgctgaagctattctaggagtttgatggccacatatccacagaatttcttttagcgcaagtggcgaatttaagtcccaaggggcgtgatacctggcctccgttcagtgggctgtactgcgcatcgttcttcggaatcgtcgagattatggctgctttaacatacgtgggatgttgggataccggggaaggaggttgtttggggtaaccgccgcttgcacgggctgttcataacgggcatggggaggtggtggaaatgctattcgggttggaaaaggtccatccggacgagctggataaccgcgaccggacaccgctctcacatgttactgcctgtcggcatgaagaagtggtgaaaatattgctcctgtttgagcgggaagaggatagccacaaaaatccggacaatgacggtcaaacaacactctctttcgctgctcggtttggacatgaggaagtggtgggaatactacttgggtgggaagatgttaatcccaatgagctagataatgacggccaaacaccgctctcgaatgctgcttggaggggtcatgagagagttgcgaagatattactcgggcgggaagaggtcaacctcgaagagccagacgagctcggccgggcgccgctcttgcatgccgctggtcatggacatgagggagtagtgaaaatttttctcgggcgggaagaggtcaacctcaggaagctagacaattactcccaaacaccgcttttgcatgcgtttggtctggatatgaagaggtggtgagagtgctactcatacggggagtgtcgataccggcgaaccggacatcagcggccaaataccgctctcgcatgctgccgctcatgggcacgaggaggtggtagaaatgttactcatgtgggaagaggtcaaacccgacaggccagatgatttcggccaaacacttgctgcttgggatggacatggggggatgatgaagtactactcggccgggaagatgtcaaaccggacaggccagacgatagcggtaaaaccctgctcttgttggcctcttgggataggaatgagggagcggtgaaaatactacttgggcgcgaagttgtcggccccgacgaaccggataatgacgaccaaatacccctctcgttcgcaactgggaatggccatcagaaggcggtggcccacaaagcgttaacccctagcgcggtttaaggtttataaaaaacaacctcttcgaaggcatcaccttccttttcttacagagtactttgctcgtaggggtcgacctagagcatcaccctttccagtcgaaggagatcccatgagacgagtctggaatgcgcttatatgccaccatgcggacgtgaacctctctggggatttgcttttatagtcgggcgtgggttttttttctgagtacggtagagtttggaactagttctattgccgtgacaaagtctagtttagtttattacaccttgcagtcctcatcctatcatatttgatttcctccagccgtacattcttgccctcacgttttttctctcccttgcgcaattgataagcatatagagtgttgttcatatagaaggaaagtggtgtatgagaggggaagggttcttaagaccaatgtgagcagctatgtaaggactgaatgataggtccccacgaatagtgcgatatcatacttagcagcagagaaaccatcaggaaggtaaactacagcacatattcagatgccacagacctgagacataaggatagggataaacaccgtacacgagaaggacaactagaataaaacagagccattttataacaaatactacatgacaaagggctaccagaatctattgatacgcagaggctcatgagagaaagtataagatcccaatttcggtacccgtagtttaagagggtgaaacatgccctagatcttagtatattttctagggaactgctcttataatatgatatgaaaaatatacttgtcctctagcgtgtgtcacatccaccctgcaagactaacatcttcgggtgctcaccataggcgaggcccactagttccaggagccagtcagatattatacaatagaatctatagatctgaggtcctaccgatttggctgtgattcgggtgagtctgggacattctgttaagattatacccactaccgaagcctgacagagtccagatggccgcctggccaatatccgaaggatcctaggccaaagctatcaggtgttctagcacagctgcacatgctcaggaaatcaaactctgaagataccgtaaaacaccatgccgttcgctaccccaccaggtcgctgatatccatattcagacataagcgcttaagcaggaatagtgctttcctgacacggtacagcctaagtcttgtggcattgttggctctcatgaactcgcaaccctgttaacttgtcggacgtatgatagcatgtcgtgcgctgatgcggcgacctacagggatattggagacctacggaaatatcgaatcgaaaggatttttacagtattggtacatggccgttagactctcttgccctccctccttttgctacccacgatggaaaatagaacaataccggtaggtcatatcaaatgacgactctgcttggaatagagagatagaaaaacagcatggctcgggccactgctccctttcagccacccggatcgggccagagttgggatttgaaagaagatttccctggatcagcccggcagcactgtacctaaagccacacgctgtgacagcctagggttactcactcggcactcgaacctcccccaataacccctcccttacagcatctcgcccaaaactagtatcatagggctaaaatactatgtatcata

SEQ ID NO: 51 - DNA, 9062 bp, Tuber indicum
atccgcttttcgccctccgcccggctgagccggcgcgctgtccttcacggcggggcgtgggctcggggaacgggtgtttttctatcgagtgatgttttgtgatcttactggttttggctatcaatacatccacatatacgaggagcttcggcttgcatgatatcagcgaatgtagctcggggacgtaatctgataattacactaaacgttggataccacgcaggttcagagctagctccttctccgctcgaggtagaggattaagttcaccggcctctgaatgacatctgatgatgttccgggcaacttctctcggggcatctgtgtcgctagcaattgtatgataacttttgccttctgcatgaagctcaatcattcgtactctctcatcgcttgcggccattcgccgcttgcctgttgcttgaccttgaggtatggtgtataatgggaggtgttggctcccataaagaaatcgtggctaagatactagtgacgggttactaaatagcctctactgctgagaccgttgcgaaagctgagtaggttctctctacatattctagtagatcttgacgctgatttgtggaaggaaaaaaagaaattttatgttttaagagattttatgttttaagaaattttatgttttaagaaattttatgctttaagaaattttattttttaagaaattttatgctttaagaaattttatgttttaagaaattttatgtttttaagaaatttcatgttttaagaaattttatgttttttttggttttaaagatactgagggcgtttcattgatcctaggtacataagtttaatttaagtctacacgatctataatacgctgagaatgcctcccagctatgcccttgatttccgccgctcccacacagtcatactgtaatacagtaacttcagtttttttttgtacctgctagcctctttaggctgtctcgggaagtcatataggttcacaatccagtggaggattctccaatagtaggctgtttagatcatgttagaattgggggctggcggaatatgctgttcggggaacttctcacaaaaacgctgtatccaccagaagtgaaagtgttcatgtgcatctcgttctcgggctggaagtgcgggtcgagttgagagtctccattctcctcagcgctagtgatgtcaaaagcatggaggatgttggcggcttcctcctcggtgagatcgagctgacggttgctggaaacgtaaacacgggtgatagtgaatctggacactgcgggtgcgggcttggcacgagccttccttcttgcagcagccttatcggccgcttccttgtcgatgggggccttggggacaaagccagcttcacgcaagagcttctcaggggtagtagggtgaccatgtttcgccaattcggtctcgataaattcttcgagcacgagaccagggtcaccacgatccctagcagcagtataaagagaggcgatgttttgccacatatacttattggtctcagcgacccagacgccacgagtgagattggaaatttcggcttgagtgagcccataaccgttacagtacttagaaatccaggctaatagatggttagaaatagaggcaaaatagggatgtgatttacttacatctctgagcaacgtagggattcaatgcacgaaggtgctgagaggggggaatacgggccaaggcctcatcaccattgacgataacagccggaatagtggcatcctgggcggtccacggctgtccaggtctatagtatccgcaatacaaacgcagaaccgacacaacataagggacttcggggtcgagggggactcgcccttgatgacggacggttgattgaattgcaccgagccacttgtcatagggcaagagttcgccgctgacagtgaaagtgacaccaccggggggttgaacgataatgggggagttctcatgttggacccagatgcacccgatcctgtcttgcatgaggatgtacccacggtccgaaaggtacaaggcgcatcccaaggtagcattgcgcataatgaggcgtctctcgagcctgttctcccactccaaaaccttagggggggggagaggttaacgaccatgctcagattgacaggacgagacgacattatgaaaggcgggggggggggtggagacgggaaaagaaatttgaaccgacaaaaggaggattgcgtatgggcgtttcaatgagtggagattagcctcagtggctgttgatgttgatggaagaaaggagggtggaaggtctggcctcagttgtatgctacctcacgaagtgggatgccaaggcccaaaaaaaagagtgacgccaagaagccgtcatgtacaactcagccctactagggtctatcatagtacatacaataataataataatattattatgataaataatattattattatgatatcataatatttttcccagagcaggtagagaacccttaggtttctaagattcctggcggaaagattggggaacatttttgtatttacggtcagagttgctcgaatagaaaaacgagtaaaggtatttgatgaagatctgattgacattcgggagaagtttagaacaaaggaattagtggggatgagctgggaaatgtgggttctgttacagtatctgtcacccaatcttaactacccctcttgtacattgcctctttggtagggggggatggtatctgaggcttgaacaataattccttgcgataatatatatcgatcaggacagtttgctgactggaaagtctaatatatactatatagtcttcataaaaccgtgtgaccgactgggaaatcataccttaagtctggccatccggaagccgctctttctgctatgggggaatatcaaatagccaagaggccatcaggtgaaatgagaattcagaaatatgatagccttcccctgccaaccaagagacaaatgtttcggcaataccaccacctgatctggtggatgtctccatgttcggctatccgcttgagtatccaggggaatagacaaggaagactaccacattttatgcagtgtgtgcggctgccgagaaaactgtttgaaagatggaactccagaacaaaggattaactggtcaacgcaacacctggttgatgcaagggtctgaagattaaaggattgtaactagtattgttggacgttacttctagcaatttcagagtggttttgggtaaagatagactatactgatattggtaggatggtatagcactacaacgaaagaaagtggtctggacaatgagcacaaaaactctaaagagaatttccgggtgggaaattagaatatttaactagtttaaaaccataagattctctatcccaagacctgttgaaaatcacgaagcttccatggtttggatgatgaggtgtaggttgtttggccttggtttggaaacgcaatggcggaataagctaaggaaagggtcggcgtgtgggtgagaactagcagttggtgtaaggagaagtgtttgggatgttttaggagttagcactagggatactggaagtaacaggttatgctccggaggtccagtcgacaaccaggtgtcgcactataggagataggaaactggagaacaaccacaaaagtatcagggctgtctggaatagcgatagcttaggggaaacgttttggaggatgtttgaaggagttgtgtgaaaggaacggtgtggcaacatgaagaaaagatggtatggaattctgcgaggcagatggatgaagaaaatatttgcaaaggaatagcttctgtatatagaggacacctccggcagcatagaacaaacaagaatcgcagacactctctacctatcatagccttattatttgaagaccagcagctgattgagagagctgttgtgagagcatctaagcactcaacctgcagaactccatgataggtaggatgataaaaggggaatggggtttagaatgggtatataaagcctcttgattctggattaggatatggctagataatgttgttttttgtcacatctcttccaactatttatttaaaaaccacgagattggacattaaaatttaatgaaggagtgatttcctcatgcacaaaatatatattgtgtgaatacctacaattgagactgcataaacacatgagcctaaatcaatttagtaaattaaaatccatcaagtagttggtgatctagcttatgaagtttgtgaagttttttgagctgaagggaaaagagaaaggttaggcaaatttttttgtgacccttagctggttggctgtcaatgcttgatattgtcatccaaaccaccacagattgtattgtggggatgttgacatgttgcagtacgacatctcctaagaccgtatgcatataagagattactttatcctactaaaagagacaagacagtcgtttctactacttgcttctgtattctcttacctcatattcaaccataatttacaagagtcatgccaaatcaaatgttgtgtgatacctggtgaaaaatgtccacctaagcctcagctgtgaatatcgcaaattgtaaggtgagaggggaatcacaatgcataactttatattataaacaccacaactgctccactggggtaccttatactaggactttcctacatgcaactctttttgcatcataaaacaaataagaacagattttacaaaagaaaaaaaaaaaactagtgaggatgccctaccgcgacgttaaaggagaaagtagttctcagatcttgtctccctcagctcgcttagtgtacctgtggacagaaactcatgtgaagatagagaaagaggtatcatgaattttaatgcagattattcattgcaatttaattccgggcatactaatttatttattcatttactcaatccctactgcaatccaagctactcttgtccttgccaaggctatcttgagcgccgtgaacaaacttattttcattgcaccttttgggcgctatgagcaaaagattgactatttcatcaatttaaaatcctaaatacaaatatcatacacatctactcaagtactgtacaagaatccttgtaactattggaacaaaataaattctgggactcttgtcggacactacgttaacgctagtaagtaacaataaatcttttaaaattactagtgtgtgacatccacttagattttgcgtgacatccctgccgctactagaccttagtactcgagtaatagaataataggagtcgcgaccttcaacgacaagggtatcgtactcgagtattcaaatagaaaatctaccttcctatgataccaggtgaggtctttgctgtggttcaatacgacaatcaagtcccgcgattacgcataacaagtgtgagtactcgagaagagtgcgaatacacggctgcctggcctgcgtgggtctaacagatctatccttgctctatttatttgttttgtttttcctgagcgcagcactttttaccctatgaggtatttataataatattatagatattctggaggaggttcccaaaaatatatcggaaatgcacttaggggcaatggtactaggattcccaccgcacactatccgctaatagatatatctaggtttctagtttccataaacaccagatgagaaaggcttaccaaaacgacagagggggttgggtctgggggatgcctacggtgcgacgatagaacggataaaagcgcagggcggggataaatcgagactcgggatggcaactttgatgtggatcagtcatgcgcagaggctgctcgggacggacgagctctcccacgccctggcagttgagctgagctccaccgacttcaacgctagcaacatctcttcgatgtcgacactgctgggttacttctaagggcttattacggtggacaaggaagcatcagccgtgcaactggcccaatttactctccaagagtacttttccgctcatcccgatattttctgcagaccccatggggcaatggcagagatatacttgacctatctgaattcccggcaaatcaaggacctttcgaccgacccatctcctgacaccccggatgcaccctttctagaatattgttctgtgcactggggaatccatgcgaaaacggaactctcggattgcaggagatcgcttgcgctgaagctattctaggagtttgatggccacatatccacagaatttcttttagcgcaagtggagaatttaagtcccaaggggcgtgatacctttcctccgttcagtgggctgtactgcgcatcgttcttcggaaccgtcgaggttatggctgctttaacatacgtgggatgttgggatactcggggtggaacaggtccatcccgacgagctggataaccgcgaccggacaccgctctcacatgttgctgcctgtgggcatggggggggtggtgaaaatactgttcctgtttgagcaggaagaggatagccacgaaaagccggacaatgacggtcaagcaccactctctttcgctgcttggtttggacatgaagaagtggtgagaatactacttgggcgggaagatgtaaatcccaatgagccagataatgacggccaaacaccgctctcgaatgctacttggaggggtcatgagagagttgcgaagatattactcgggcgggatgaggtcaacctcgaagagccagatgagctcggccgggcgccgctcttgcatgccgctggtcatggacatgagggagtagtgaaaatttttctcgggcgggaaggggtcaacctcgaaaagctagacaattactcccgaacaccgcttttgcatgcgtttggtctggatatgaagaggtggtgagagtgctactcatacggagggtgtcgatcctggcgaaccggacagcagcggccaaataccgctctcgcgtgctgccgctcatgggcacgaggaggtggtaaaaatgttactcatgtgggaagaggtcaaacccgacacgccagatgatttcggccaaacacttgctgcttgggatggacatggggggataatgaagtactactcggccgggaagatgtcaaaccggacagcggtaaaaccctgctctcgtggcctcttgggatgggaatgagagagtggtgaatatactacttgggcgcgaagttatcggccccgacgaaccggataatgacgaccaaacacccctctcgttcgcagctgggaatgaccatcagaaggtggtggccctgctacagtcttacaaatcgttaactcctagcgcggtttaaggtttataaaaagcagcctcttcgaaggcatcaccttccttttcttacagagtactttgctcgtgggggtcgtcctagggcatcaccctttccagtcgaaggagatcccatgagacgagtccggaatgcgcttatatgccaccatgcggacgtgaacctccctggggatttgcttttatagtcgggtgtgggttttttttctgaatacggtagaggttggaactagttctattgccgtgacaaagcctagtttagtttattacaccttgcagtcctcatcctatcatatttgatttcctccagccgtacattcttgccttcacgttttttctctcccttgcgcaattgataagcatatagagtattgttcatatagaaggaaagtggtgtacgagaggggaagggttcttaaggccaatgtgagcagctatgtaagggctgaatgatagatcctcacgaatagtgcgatatcatacttagcagcagagaaaccatcaagaaggtaaactacagcacatattcagaggccacagacctgagacataaggatagggataaacaccgtacacaagaagaacaactagaataaaacagagtcattttataaaatactacatggcaaagggctaccagaatctattgatacgcagaggctcatgagagaaagtataagatcccaatttcggtactcgtagtttaagagggtgaaacaggccctagatcttagcatattttctaggggactgctcttataataatattatgatattattaggaaaaatatacttgtcctttagcgtatgtcacatccaccctgcaagcttaacctctttgggtgctcaccataggcaaggcccactagttccaggacccagtcagatattatacaatagaatctatagatctgaggtcctaccgatttggctgtggttcgggtgagtctgggacattctgttaagattatacccactacggaagcaggacagagtccagatggccgcctggcctggctggccaatatccgaagggtcctaggccaaagctatcaggtgttctagcacagctgcgcatgctcaggaaatcaaactctgaagataccgtataacaccttgccattcgctaccccaccaggtcgctaatctccatattcagacataagtgcttaagcaggaatagtactttcctgacacggtacagcctaagtcttgtggcattgttggctctcatgaactagttttcgcaaccctgtaaacttgtcggacgtatgatagcatgtcgtgcgctgatgcggcgacctacagggatattggagacctacggaaatatcgaatcgaaaggatttttacagtattggtacatggccgttagactctcttgcactccccccttttgctacccacgatggaaaagagaacaataccggtaggtcagatcaaacgacgactctgcttggaatagagagatagagaaacagcatggctcgggccactgcccccgtcagccacccgggccgggccagagttgggatttgaaagaagatttccctccggtagcactgtacctaaagccacacgctgtgctagcctagggttactcactcggcactcgaacctccccaaataacccctcccttacagcatctcgcccaaaactagtatcatagggctaaaatactatgtatcatatgacaccacacgatatcgttgtcccatgcaaaaagaccgcaaacccaaacctaattgaaggttgtgatcccactacaaaagtcaggggat

SEQ ID NO: 52 - DNA, 9136 bp, Tuber borchii
  misc_feature (534)..(534): n is a, c, g, or t
gatacccctacataggtttgtgcgtttgtttttttcaacatgcccttaccattctcaaccaataccaactttatcataaaacggagttcacaaggctaaagaggaaccrattttaaaacagttttgtcctaggtttttctccgaggactttgttgttggggccagttagttattagactgggttgcggcgagagtcgatttcaagcgccctgcaccgcagagcatcgaagaatagcccaattaagctgaggcgtgaaaccaatacctgcatgaaagtggttaaaattttgtggcgcagtgtagcacaaaatgacaggacagataagatagtctggcacctaactagtcctatgactttagtctacactacgccacaaaatattaatcactttcatgcacccccgagcacagcactagtacagtactagtacttgtacttgggtgtgagggtaagtggtcggaaagtaacccaattctcggtgcaaattctaagggatacaggaaggcattgagggagattgaagataatttganaanaagatagtgggggagagaaaggagataaagaaggaaaggagggatggagcaaacatctaaattggatatataaagcctgtagtagtataaatatactcgaaaagttttaaaaaaaaaaaaaaagtgatcggaaaaaaaaagtggtttttccgatcactttttgctccaggctggtactcgggtatggatgtaaaaatcgagttctgtaagtaaccctgtcgttagaatgaggtactgagttggctagaatagaagaaaagatcggcaagtcagggtcaccaagtacccggaccaaggctgggcagatgcaacatggcagtgatggtcctttctttttctttttctgctttctatatttgagtctgtaaagtttgatagggaattgagctattaggggtttggaatttagaagactttcaataagagcagtaacaactacaagcagcttccgcaatgattaagatgcaaccayyccaggtttttagctccttycaaggtctagctttttccatgacgccaattgcacgattcccgagattccttgttcatatccctgctktgctcgatttttacatccctacccgagtactaggggctttcaaagtcaggaatgggggccaacggtaaaatgatcgaacactcaaagccacgcattagttctgcgttagggcgacatattaactgtagccaactgtatcaaagcatttcaagtgacataatagccggtcccctcctattnnatcgcgcatagtsggaaaagcaagtgtgacctacactgtaaaaaaaaaagccgacgtgtcacacctgcttgagcgtcacacttgcttgagcgtcacacttgcttttcccgactatgagcgattgcgaaataggaggggaaccggctattagtatcattctaaattatttcggtttttggggtagagaaacggtgaacaaaagagacaggatcgcacgtacagtaagaatgattacaggtgaatcacgaggctaggggagtaatttcagttagtcgcagcgttaaacccccctaccaggttcatggttcctcccatgcagtctacgaaatttaatggaagagaccccctgctgtgtctacattggagaaaacctcaggtgaaaaaaatcaaacgcaaacaaatactgacaaaggtcatacaagtgaaagcttcctcgcctccttactaaaaaaggtggaaaaataccccacctttaagcgtgatcaatagcgcgagtcacagcccaccaacaggcctctcgaacagatgctgattagtaggccccctgtgaggaaacgggtagtagtcagcacagcagcaacagttcgtccaagtaaaccacttgaaagtttcttatttaggtttaactgttcgagtagaggacctgctcgagtgaactatgaggcctaatcgtaggatttaaaagtaccagctatgttatcccaaataggaaggcagatcttctactcaagtacggtacaagtagtattgtatttgtatcccactctcattcctacaacctccagtgggcagggatgtcaagcaaaatctaagcaaacatcacaagcaacttgaatggagttaatttacacttttgagcgaatacagcgcaagagaactcgcaactcctttttagcgagagtttcacgcatcttatcgctaagcgcgctaggaaaaaatcaggcgaaataaggtcttgggatcggagcttttttttattgtagtgacaggtcttcaatagaatagcctaatgacgttgtttaaacgagatctgttaagatgatcatgttgtaacactggtgtgggttttgggggctgaggggtagcttcacccggtatcactctgtattttgtttgattttgcgtgacccaaccaccttactgaccttcctaaataggaaggcaggcttttctactctttttggtgcttggctgtccttgaagtttctgtaatactgctggctcgttccgctactaatccttggcagtgcgtagggatgtcacacagagcttaagcaaacctcctacacttctaatatgatactaaagaatttttacttgctaaagctgctgtctaagtaattagggtgtttaagtaaaatttaagaattacgcttccctttcatctcacaatttgtgatgcccccagctctagggtgttggatgttttcgttgggtgtcaatcgatgtttgatttcgcgtgaccgctgcaaatcaaggtggaatgggagacaaaaagtatatgggacgagtgtggaggtgacttgcttgactttcctttgcgggataagcagatcttttatacggtatgtaaaactcctaggggtatcatacatataggcatctctttagtgatgcgctggatggcgagatccaatagcgatggccaccagctagtgatcgcaaagtacttccccagtgtttttcttttttctcgctagaacaaacttcacaagcttcataaaccagagcgccaacaacttagcgaagctcaattcaccggatgaatttaggcctctgcctttgcgccggctcaattatgtaatttgagaacattagatactcgcgctgtgtatgttttatgcgagcagaaatcatcccctcgtcatagcccccaagtaaacaaactgtcgtaagaactcgaataactgacctcccgcaaagacgtcccaaatttcctctcttggataggggggcgagcgcacagcgccaactggcctcctcagcctgagcttaagacagaataaaaagaaaaagtaccagaccttcccagcagaggcaattcaaacatcctagcgtgaggttcaattgttcgagtccttatggtgctgtaaaagaagtgtcgcgggtgggcggttgtgggaaagaaagagtactcaagtcgccggattgaagatgctcccgtcttagtagtgacgcactaaaaaaacgtatcttcaatccagcgacgcatactgtagagtgctataggccgcaagttaaaggaggagtttagattctttgttccttcggaatggtctaggaaaggatttgaagacttttccttttcaagtatagcatccaaatagcaaaaagggcagcgaggagattggccaaggcagctctaaaatccgcttgatatgcagcccccgcatagttgtgggagactgtctgctataatctacgagtagaactttcccagatattcctaagactacatatataaaggcgcagtatcctgtttctggtcatacgactcactcttctgcaactgatcaaacttctttcctccatataattagcttgaaatctcggtttctatcctggtggtccaagcgggggcgtcgggcacattgcttctggctctcctatctggcgatatcttcatccagaatcaggagacttagtgcgcccctttcaaatcctaccctccttccgtcatcctccctggtatgaatctttggaggttggtcaaacactctcacagcggctctttcaactggccgctggccctgggcaagaggctctgacggtttagaaccggttagggattctaatttctctgccgaaaatccttcttaggggcgttcactcactcacaatccaaggcttcgccttgtgctatgatactatgatcccaccaatcttagcatggtctaaccagaaggccctctgaaactatcacgtgcaaccggcaacagcgctagccacaattccttaatctccagacctttcgctgtccggttgttgcatcgggcacctgctcctttgttctggagttccatctttcaaatcgcgctctctgcgcccgcacacgccgcctcaaatagggcagcccttctgacctattcgcctggatgcgcaaggagtagctgaacgtggggacatatagcggatcggttggtagtgttatcgaatcattcagcccccgaccggttggggagaaaactacttttgaattcttatttcgcgtgacagcttcttggttattggatattccctcagaatggaaaaaacggcttccgaagtttgagatatgatattccggtctgccataccgtattgtggaggttattcctattacatttccaattagcaaaatatcctaatttatatatcgaatcacaactaattattcctctagggggctctaccaaagtagtaaagtacaggactgacttgggggacggataatagaacccgcatttctcagttcatcctgtttcactaattcctttgttcgaagctcctcctgaatattgaaccatcattaactatcccgacttgttttccgcatgagcatccttggccgcaagaaggaaaacattggttgatattttcgccgagaatcttagaaacctaagggctctctccctgccctagtggaaagctcaccatacatactatgatattttcacgataatactatgctcagaccctagtagggcttgctggagttgtacgtgacttcttcttagggtcacccttttttgggcgcacccacccccttcgtgactagtagtctatataagagatcccaactgcccctcttctctcttccatcaacaaccactcagcagaaacaacatccatacagcaatcctccctccgccggttgatttttcaaattcatctttcctccccatttttatctttcatcatgtcagcgagaccggtcaatctcactatggtggtcactacctccccgcctccaaagctctttgagtgggagaacaaggttcagaggcgcctcgttatgcgtaatgccactttgggttgcgccttgtatctctcggaccgtgggtatatcctcatgcaggacagaatcggttgtatmtgggtccagcacgaaaactcccccgtcattgttcaacctccgggtggtattacttttaacgtcagtggtgagctcttgccttacgacaagtggctcggtgcaatccagtcaaccgtccgccatcaggggcgagttcccctcgatcctgaagtcccctatgttgtttctgttctacgtctttattgcggatactatcagcccggacaaccgtggacggcccaagatagcgctatccccgccgccatcatcaacggtgatcagacccttgctcgtgttccacgttctcagcaattacgtgctttgaatccctatgttgcgcagagatgtaagtaaatcacactcctacagtccctaggcaaaagtgttcgtgaatttgcttagtcagatgtctcacattttcacgaacacttgctgtattttgtccctggtactaattaaatctccatccatcagcttggatctctaaatactgcaacggttatgggctcacacaggctgaaatttccaatcttactcgcgaagtctgggttgcggagcccaacaagtatatgtggcaaaacatcgcctctctttatactgctgctagggatcgcagcgagcctggtcttgtgctagaagacttcattgagactgaattggcgaaacacggccaccctagtaccccagaaaagctgctccgtgaagctgggtttgtccccaagactccaatcgacaaggaagccgccgataaggctgccgcaagaaggaaagctcgcgccacgcccaaattgtctaacttcgctctcacccgcgtttacttgggtaacgaccaccagctcagccttaccgaggatgaagctgccgatattcttcgggctttcgatggatatgaaccgaacttgggtgacggacactcgcaaatcggccctcatgtccagcacttgctggcgatggaaatgcaagacaagtttacccacacttccgttggttccaatggcttcgtgagtgataccgtgaatagtatgatttacttgttgctcaattctaacatttacccgaacagcatactatgggagaatcctccacaggattgtaaccggatatgatccccttggagatctgaggaggctgcaggtgcgaaaaaaaaaaaatggtggaaaaactgtgtacgacgggaagcagggtatagctagaaggcatactcggcgggttatggatcaatgtagacttaaattaagcttatctgtctgggaccaatagaaagcccttagtaactttaaatccagatatcaatgctttcttccataaatcagcgttcgcgacttcgatgtactaggccgttttttttttaagtattcggatatggaaaatataagtaatctagtcatggtataccggtacagtaagagcttgagcaattaaaccgtgtgcgaggtagtccaaaatttcctcctctagaaaatgagagaaaaacacactaaaaaacctgccctccgagacaaataaaaagaaaaataacaccacaccttactaggagagccaactccaacatcccagcacatagttcaattaccggatgccagtgggctttcaatgccccgaatgcatcgtctaacggtattccagcatcgcgagattctggcactgtaagtacaagtacagaagaaagacaccatgatagtcaagcgctcgcaagcggccaggtcggcctgcaaatagccggtaggctttcagtccggccaggccaaagtgagacagacctgggcttcctaggccaggctttgggaaagccggcaggcttcatgggtccctggcggcaccggtaccgtaaaaacttgaataattggacggtgtgctatgtcgctgggttttcctctttcagtaaggtcaagaaattttcgtttttttttccgtcccggaaggggaaaggggctgtgtttttgctgtgcgttttgctccctttccagagaagggaattctggactacatagcagaccgtccaattattcaagtttttacggtaggctttcggtatattccatgtgactagtcaatattttcgtaatgtgtgggtgatatctgcagatatttgaagataccgccagatatctgaagatgccgccagatatctgaaggtgcctgtaggtttctgcttattattcaggcttgggccacaccattcgatttaaaacatattctgcttggcttcagatcttctagagtttagcgagaaagtagtatcttatttttcattcactttcaccctttttctcctaattgacaaactaactactgtaactagcggtaaagacagaagcacataaacttgaacgaagtcgtcctcccattctcccatccatcgcaccacatatcctttctctgcgacctgtatacccctctccctttcatggacacctaaactaagttagcaaaagaaccgacatgcctttcaaagaactcccacggctcataacctccacgccgagcgtccaaccttcagcacgtctgactccttcgagatcgtactgcatctggcggtgcgtccatccaatttggcaatacagctcggagtagacgcggtcggccccagcgcacctggtgggggcggccgggggttgttctcatataccgcccggggtgggcgtgaagggatagctctgggtataggaggatgctacccacgggagtgaaagcgcgacttagaggaaaacaattgcatggcagagcgagaaagacacaggggaaaggtagcactagaagagtgcaattacgaatttgaatgagtaggatgtaaggaggccgctatgcgttgtgagagttttcaatgaactttgttagcacaagagtctgtaggggggaatgggagacaggggcaagagggagagaacgcgtgcaatagtccctagagcactggttggtagatgggaagctcgtggtttatgtgtactcatgtctttacctctaattagttgatttatttattgaataagggcatacaagcaggagtcaagttgaatcagaagaactttcccccaattgtgctgtgtgttggggatgaatcttccatgcttggtgttggtttgtgcttggtgtttcgtttgtgcttggtgtttcgtttcattctccagctgaatcacatcttatcactcgaaagcctgcagatatctgcagtccggccaggcccggtcagatgagcttcagaagccgggctggccgcttgcgaacactggtctcactggggcccccagactaatgcgaacaccgcctagagtatgatacccaatgccagacgctctgccacccggggggtcactaaggggctgtttggagtcacaactttggggccgcaaaccattgtttgtaactacaggtcttatttggcagatgggaattacaaacaatggtttacaacaaccagcagcaatttttttgctgtttatgaaatagggcacagggttctttcaaccatggtttgtatcatagttaaccaaacttcacaaagttgtcacgccctaactctctacccaaccctatcattgccccacacaaaaaaccgcaaaaccaaatctaattgaagggcgtcaccacctataaaaatccgaggcctcacactccctcataatcctatatcccccctgtaccaaaactctcttcttctcaacacccactccagagtactcaacctcacaacaccctccgcatattggtcagccagtctgccaatatcagtattcgacttacagttgacctctccacacatacgcttgttttctttgctaaaatgcccccaagtatccccccggct

SEQ ID NO: 53 - DNA, 1156 bp, Tuber borchii
atgcgtaatgccactttgggttgcgccttgtatctctcggaccgtgggtatatcctcatgcaggacagaatcggttgtatmtgggtccagcacgaaaactcccccgtcattgttcaacctccgggtggtattacttttaacgtcagtggtgagctcttgccttacgacaagtggctcggtgcaatccagtcaaccgtccgccatcaggggcgagttcccctcgatcctgaagtcccctatgttgtttctgttctacgtctttattgcggatactatcagcccggacaaccgtggacggcccaagatagcgctatccccgccgccatcatcaacggtgatcagacccttgctcgtgttccacgttctcagcaattacgtgctttgaatccctatgttgcgcagagatgtaagtaaatcacactcctacagtccctaggcaaaagtgttcgtgaatttgcttagtcagatgtctcacattttcacgaacacttgctgtattttgtccctggtactaattaaatctccatccatcagcttggatctctaaatactgcaacggttatgggctcacacaggctgaaatttccaatcttactcgcgaagtctgggttgcggagcccaacaagtatatgtggcaaaacatcgcctctctttatactgctgctagggatcgcagcgagcctggtcttgtgctagaagacttcattgagactgaattggcgaaacacggccaccctagtaccccagaaaagctgctccgtgaagctgggtttgtccccaagactccaatcgacaaggaagccgccgataaggctgccgcaagaaggaaagctcgcgccacgcccaaattgtctaacttcgctctcacccgcgtttacttgggtaacgaccaccagctcagccttaccgaggatgaagctgccgatattcttcgggctttcgatggatatgaaccgaacttgggtgacggacactcgcaaatcggccctcatgtccagcacttgctggcgatggaaatgcaagacaagtttacccacacttccgttggttccaatggcttcgtgagtgataccgtgaatagtatgatttacttgttgctcaattctaacatttacccgaacagcatactatgggagaatcctccacaggattgtaa

SEQ ID NO: 54 - DNA, 9381 bp, Tuber borchii
  misc_feature (7267)..(7267): n is a, c, g, or t
ctgttactcatcaggtatggtgccgggacttgaaatcaaccctcatgggtaattcattttgcttaataatacacacaaaatagtggggcttggcaaaagtgtcttcaaaaaccatcctacatgggtaattcaaaattttactatctccgctaatattgctctgattgaaaattagaaaacatagaattagagaggagggtcttctgagcacttctatgaagccatatcctattaaagatgtggttgtggttgaaaactttcgaagattgttcttgtgttggtctttgggtgatttcaagtgcccggcaccataccgaagaaagtccaattgagccgccgagagtttgtgaaaccgctcttgctcttgtgtggcactgttttttttttgttaagtccttgatttattcgtagctaactaacaggaacatcatggcaagcaccccaaggtatggcctaaaaaccctatataacaaaagaaacttcttcgtcccctaaataacaaataaacgataaaacatcactatacaatccttaactaaagcaaagctttcatatgatcatgaagccaaaaaggcatctctcgtaaaaggaaaaacccccatcttcaccgggaaatttatccttccctccttcacataagacataaagtcctccatcaatttccttcccacctttcccccttcccacaatttagactctggtctgatgtcgaaagctcgcgggtccttattctctctcgccctggccattgctgggcattccattatatgcgctggtgtttccaggctcgaaagacacctcccacaggtggtgtcccgatccagattcgaacaatccatctgagctccgaggtaactcttgttcgacctcatttggtgtagccttgacgccatgaacttagggagtgtcataaaaaaccaacattccctatatggcggcctgtatccgtagtagggggggtactcccatcctacccatttctgatgcatctctcttctcaccctttctctcgctttgaaatgaaccttcttgtgtggcactgtactcgtatcatcctagtactagtgcggccgatagataaaatagtcacgtgggtctgttaaggcgcttgacaccctatatatagtttatctatcgcgcaggagggagggaggaggaaaggaagaacggaaaacgtgtaggtaaaaattcacagctaaataaacaacggagtatgataggatgaaaactacaagctataaactaggcttcccacagcaataggaccacttccccccagggaccaaactctactgtgacttgtaatcagacttggaaatcctcacctggcataaaagcgaatccctagcgagtttcgcgtgtgcatgtgacatatacgtgcattccccgggcccatctcatcggattccctccagctgcaaaggatgacgtccatggacaatttctataagtgacatactccgaaagggaaggatggcagcgatttcgacgaggcggtgtctcctacacctcaaactgccccaggggttgccgctttgcgcgactgtagcagcgctgccactctgttatggctgcccctagcagcgaacgagagcggtgtttggccgaaattatttggtttttcggggttaacatcttcccgcatgagcggtattttgaccacttcttcatgcccataccaagcagcgaatgagagcagtgtttggccaccattatccggcctgtcgaggttgaccccttcccatccgagtagtattttcaccacttcctcatgcccattccaagcggcgtacgagagtggtgtcctgccacgattatcttgttcgtcagagttaacatcttcttgcctatgccttctgctcgggaatgaatctcggggttttatctgcttcctctattaatgacaagtttaggcgcatttcacaatccaagggagcactactgggctagtagccatatccccaactctagaagatggtgaagcaccaaccaatatggggtgccaagttaaactggtttacgagcgatggataaataaagcggtgtcaccgtgagaggtacttttttgtcgcataatataagggcgtgagggcctaaggataattgtcagagtttgccgcgtacgggacgctcgggcactcagtccatgggtttcttaggctgcctggcgtagggcgtccgctcagccaaaaaaatgacggactatgacccacaattcgtcccgccctactgctcacaacttgatgtttatcttatggtttggtgttcggatttctcactctcattttaagaaattttttgtttatttggaccgtacaagtttatttaagtgaacaaatgaccctttacaaacagctagatcatccctcccattaaaagtaaatcacccaagtaacccataagctccccccattgagatcattcaattaatcaaaccgaaagatttcagaaccctctccgcttcctcgataatatcgcccaaggcgtttgcgctgggctgggaggcgaagcggtcggtgagggctccaagatgggtagaagctctcttcttcgcacgcttcgcgaccccagcacggtgaaggtcggcacgggtcgaagggcccttgttggcgcacgccctcctcataatttcatgagagcggcgcggggtgtatttatagttgggattgaccaacttgtgagaagccgcgagttggcgggcccttgcctattaatgtgttagtgattccaactaacaaacagtcaagtgagatgttatactttataaacatctttggcttcgtccggggaatcctgccacatgcgaccgagaatttttgctattgaaactattagggaaatggtactaagagcggagagtgaaaaaggggagaatgagaaccacatacaaatatcgttgttaccaaccccaggatactgagccttgaccgcattaacattctccgttctaaacaagatgaatctatgattgtatcagcacatgaaatcggtacgttacagtcgcaccccgctataacgaaggccaaaaattgggaattttgttagttatagcgaggttttggaagaaattccttatagcaagggccacccgcataggagcaggagtgtaaattcccagagggttcattcgtaatagcgaggtttcccttataagcggtccattatagcggggcacgactgtagtaggattgtacttacgcattagcggggcgcttaacacccttatcgcggatgaaactaagattagcgccatgagaggcaaagtcttgatcgttgacagcaagacgcaaaaaacggttttcgccggtaatctcgtgctggggatcctcgaaggaatgatcggtgacgttgcaattcatgtcggtagtagcgggatcgaacacaagaagaatagaaccatcgttaagcttgtaaatcgggcgattgcggatagtaaggtgggtcccaataatggtgtggagggccgaatcaaacgagacgataagaggtccagaggcaggcttccagttgatcggaagcatatggtggatctggagctgcgctccatccgatccatcgaaataaaacttgttgctggcgagcatagaaagatactcacagaggtcgactccatcgtagcgttggtgaataagaggcccggcctcgacggccatctcgagagcagagagggagctcatattgaagttaatgaaaataaaatgagttggaaaatatgccgtgataggagcactaccagtaggaaggttgaatgttgcgatgttgaatttgttgttggtcgtgttggttgttggtggaagaaagaagagggacagttgggatctcttatatagactactggtcacgaagggggtgcgcccaaacaagtctgaccctaaaaagaggtcaggtacaaattcaacaagccctacaagggtctaagcatagtattatcgtgaaaatatcatagtatgtatggtgagctttccactagggcagtgagagaacccttaggtttctaagattcttggcgaaaatatcaacgaatgttttccttcttgcagccaaggacgctcatgaggaaaacaagtcgagatggttaatgatgatcggtttaatattcgggaggaacttcgaacaaaggaattggtgaaacaggatgaactgagaaatgcgggttctattatccatccccaaagtcagtcctgtgctttgctgctctggtaagccccctagaggaataacctcggacggaagccgttctttccattctgagggagtatcatatccaatagccaagaagctgccacgcgaaataagaattgaaaagtagttttctccccaaccggtcgggggctggatgattcgataatactaccaaccgatctggtagatgtccccacgttcagctaccccttgcgcatccaggcgaatagaggagggccgccctacccgaggcggtgtgcgcgggcgccgagagggcggtttgaaagatggaactccagaacaaaggagcaggtgcccgatgcaacaacagggcagcaaaggctctggagcttaaggaattgtgactagagctgttggcggttgcacgtgataatttcggaagttgttctggctagaccgtgctgatattagtgggatcatagtaccatagcacaattgtgagtgggtaaactgagcacccctgagaaggattttcagcagagaaattagaatccccaaccagttctaaaccgtcagagccccttgcccagggccagcagccagctgaaggagccgctgtgagaatgtttgaacaccaacctccaaagcttcatacaagggaggatgacggaaggagggtaggatttaaaaggagcgcgctaagtctcttgattctggatgaagatatcgccagataggagaaccagaagcaatgtgcccgacgcccccgcttagaccaccaggatagaaatcgaaatttcaagctaattatatggaggaaagaagcttgatcggctgtggaagagtgagccgtatgaccagagacaggacactgcgcctttatatatgcagtctaagggacatctgcgaaggtgctagattatccggttccagcactctagtcctgttacggaacgatattagtcattaagcactgctagctgcccctcaatctggccttagtgtgctgaaaccgaattttactatttcacaccgcctgggatagcagactgtctcccatcactatgcggaggctgcatatcaagcggatattagagcccctcgctgccctttttgctatttggatgctataccggaaaaggaaaagtcttcaaatcttttcccagacccttccgaagtaacgaagaatctgaactcctcctttagctcgcggcctatagcactctactgtttccgggctttcccgtcaggtgttcttttgcgggcagttggcaattgggtttgctgagcttccggtgaaggtagccagcagatgaaagtgaaagaaagcgaagagagataactgaagaggatgaagatgccttgatggtcgtgattgagatattctcaagtcacgaagcaaggacggatgaagaatgatggaatgcgttctttcagaagaagaaagatcccacgatgctcattgtttctaggaggagttgtttcctcgtttaaataacatgaggactgctaactgagctacctagttctttattgcgtaaaagcatacgcaatgttcaccaaagtctatgaaaaactcgctgagccatccggacgcacgacactacaatacttttgtttagtgcgtcactactaaaacgggagtatcttcaatccggcgacttgagtactctttctttcccacaaccgcccatcctcgacacttcttctacaacaccgtaagaactcgaacaattgaacctcaagctaggatgttggaattgcctctgctggtaaggtctggctctttttcttttttgtctgtcttgagctcaggctgaggaggtcagttttccctgagcactcgcccccctatttaagagaggaaacttgggacgtctttgcgcgaggtcaattattcgagttcttacggcagtttgtttacttggaggctatggcgaggggatggtttctgctcgcataaaacatacacagcgcaagtatccaatgttctcaagttacataattaagcgggtgtaaaggcaggggcccgaattcatccggtgaattgagcttcgctaagttgttggtgctctggtttatgaagtttgcgaagtttgttcttctagcgagaaaaagaaaaccactggggaagtactttgcgatcaccagctggcggccatcgctattggatatcgccatctgacgtatcacaggtcgtgttaaagagatgcctatatgtatatgacacccctaggagtcttgagtaccttacttaagagatctgcttatcccgcaaagaaagtcaagcaagtcacccccaccactcgtcccttatacattttgtctcccattccaccttgatttgcagcggtcacgcgaaatcgaacatcgattgacacccagcgaaagcatctatcaacaccgcctagcactgaaggcatcacaaattatgagatgaaaaggaaagcgtaattcttgaattttacttaaacaccaaagctacttcgctaatgatgcctagccaccacaaataaaagaaacccctagactccctcccctgcccttagagcacttgtagaaacgatgcgtcaagctcgccaaagagatgtcacgagttttaatgtatggtattggtccaaaagggttgtaattctagacgctctaaccatctaccgtaaaacctcaaataaaagaaccgtgtgctatgtagtcgagaattacccacttgagaaaaggggctaaaaacacagcaaaaactgagctgctcaggtcgcctgagagtcataaaaaaacaaaaaaaacaccagaccttaccagaggaggcaattactcggatagggtcaaaaagggcggaaaactttcggtggcgaaacaaggatagaggcgctaatcgatcaggaggaagtagaaaaaatggggggtgttataggatgccattaacctggagatgttgcagtgtagtcaccattgtattccatctatgggtcttattaagaatctgatcatataaggccaaattaataccaaaggagaaggaatcagggcttttggaatagttctttaagagactcatataaatgcataggttcaattgcacattggcaatttcatcatcatttaaatggtcaagtatcatactagnatacttatttcttcaacctggatttctggtgtccgccaaggcaagaatttttccactgttgcaacctacttcctaactaccccacctgatcctgctgatctttttttccgccctttttgaccctatccgagtaattgcctcctctggtaaggtctggtgttttttttgtttttttattgtacttctcaggcgacctgagcagctcagtttttgctgtgtttttagccccttttctcaagtgggtaattctcggactacatagcacacggttcttttatttgaggttttacggtattcatctgcctcaatcccagcccaagttgctcttgtcttggccaaaatcctaggcgtgacttgctctaattgcttggacaccagctagcaagtaaaaaattctttagtatgatatcatattagcagtgtgggaggtttgcttaggctntgcgtgacctccctacgcactgccagggattagtagcagaacgagccaacattaccagcgcggctgatagaaaacatagtcacgtgagtttgataaggcgcttaacagcaagaaggttcaccaaattttttttattaggttcacgtgactattttacctgccggccgcgcccgtacagaaacttcaaggacaaccaagcacccaaaagagtagaaaatctgccttcctatttaggaaggccagtaaggtggttgggccacgcaaaatcaaacaaaatgccgagtgataccgcgtgaagctgcccctcagcgccccaaaacccatacgagtgttacaacatgataatcctaatacaacgccattacgctattctgttgaatatgtcttgtcactacaataaaaaacagctccgatcctaggactttatttcgcctggtttgttcccagcgcacttagcgacaagatacgtgaggctctcacttaaaaggagttgcgagttctcttgcgctgtattcgcccaaaagtgtaaattagctccattcaagttgcttgtgatgtttgcttagagcctgtttggatacaacagttccgttccgttcccccatacaaatggcccttttccttccgtttcaaaacggaaagaacgctcttttcacagtagaaatggtcgcccgacggaacggccccatttttgccaaaaaggttcttaagctttctagctctcaccatcgagtatgacagtcaaagacattgttggtcaatgggagcagaagttcacgtgacctacttagcactcggcgacctgagtacagaaacacgaaagtaacaagtttacaaactagacaaaagtacaatgccagtttaaaagtctacctgtaggatttgtatggaaattttggcagaagggcacttttgtgggttcacgcaagctataactacagttataccttctaaatctcgatacgtctacagaaattgttagatctcccacctagaaaaatataggtctgtttaaactagtgcccatgccgtgaggccacagaaaaaaaacagggctccccatacaaaacacatggctaatgaccaaatatatcaccaaactaggctcgggtagggtgctctgtcggggttgggaatggagaaaaaaaggggtcagcaagatcctatggggcagtgagcagacggttcgtgtgagtggaaaataatgagttctggcagatgcaaacaattctgagtggatactaaacacattatcttaccgacaataagagatcccgagcggatgattaagaattacccttagtgaaaactagctttatagagattacttgaagctccctgaagccctccagaatatctgagttggagtttaaagcagacactaatatacttggtccgggcccctaatttctacaactccctctttgatggggcccggctagcaattttcgttttaatttaaattcacaactctctagcataggagggagaaaatatgaagtaaaactgcgggaaatcttatagtatcataccagagatgcacaaatatttaataaaactatctaaagacacaacccaggtggtagtaaactccggcaccccccaactgatagaggatagttatcaagggctactgggaatagcagaattgaacgaccccggccccaacagg

SEQ ID NO: 55 - DNA, 1255 bp, Tuber borchii
atgagctccctctctgctctcgagatggccgtcgaggccgggcctcttattcaccaacgctacgatggagtcgacctctgtgagtatctttctatgctcgccagcaacaagttttatttcgatggatcggatggagcgcagctccagatccaccatatgcttccgatcaactggaagcctgcctctggacctcttatcgtctcgtttgattcggccctccacaccattattgggacccaccttactatccgcaatcgcccgatttacaagcttaacgatggttctattcttcttgtgttcgatcccgctactaccgacatgaattgcaacgtcaccgatcattccttcgaggatccccagcacgagattaccggcgaaaaccgttttttgcgtcttgctgtcaacgatcaagactttgcctctcatggcgctaatcttagtttcatccgcgataagggtgttaagcgccccgctaatgcgtaagtacaatcctactacagtcgtgccccgctataatggaccgcttataagggaaacctcgctattacgaatgaaccctctgggaatttacactcctgctcctatgcgggtggcccttgctataaggaatttcttccaaaacctcgctataactaacaaaattcccaatttttggccttcgttatagcggggtgcgactgtaacgtaccgatttcatgtgctgatacaatcatagattcatcttgtttagaacggagaatgttaatgcggtcaaggctcagtatcctggggttggtaacaacgatatttgtatgtggttctcattctcccctttttcactctccgctcttagtaccatttccctaatagtttcaatagcaaaaattctcggtcgcatgtggcaggattccccggacgaagccaaagatgtttataaagtataacatctcacttgactgtttgttagttggaatcactaacacattaataggcaagggcccgccaactcgcggcttctcacaagttggtcaatcccaactataaatacaccccgcgccgctctcatgaaattatgaggagggcgtgcgccaacaagggcccttcgacccgtgccgaccttcaccgtgctggggtcgcgaagcgtgcgaagaagagagcttctacccatcttggagccctcaccgaccgcttcgcctcccagcccagcgcaaacgccttgggcgatattatcgaggaagcggagagggttctgaaatctttcggtttgattaattga

SEQ ID NO: 56 - DNA, 14624 bp, Tuber borchii
  misc_feature (358)..(358): n is a, c, g, or t
agactctcgtccatggcatccggrgtctcrycctcgctcagtctagyacgaargtatctratratatcatctttkctggggcctatagakasrcttatcacymgtccggsragacrcttttcgryctcwgccsgaatatgkggccttcsgstyayraatattygtgtkccsggggacttctcaaggattygtttywsygaatcaaggakcttraytckgtgsactrcyrcacattcgtcmagggcgtcaatgcacatraatrtgsgctgygaggasgtgatrgcctgcaacatsttcacgatatcaggaagtcgcrgtgycckyccayccatrgccttttyctsctcttgaaaggmtngtgatatttcctcagggatcctttccattccgctaacgacctgtttcagcaaggaacccagcacactcattgcagactgctcctttcgggccgcgaagtcaaagtagaaacatgtaacggcggtatttttttcgacggtctgatcgcttagcttatccattaccagcgaactgacatcgtgacctgtcagcaccccgcgctctctttgcttcccggagaatagtctttgttaccttataaacgtcttgccgacccccggacctccatagcaaaacaagactgctttatcgccttcgccttcgccactacagtcataccagcttctgaattcttctgtttgcatgagccactcccccacattgtcgacacgccgctcttgaacatcccgatgccgtgacctaggcgacaggggcgacagccagcttaggagttgagactgatcatcggcaatagtacagttgttccacaccttattataagagttcgtggtattgaggcagttggtgctatgactgatggtatattgagacatcttccgttctttgctgcttggggtgttgtagrggaggagttatcgagtatcgcctgttgtcgacattcccgggagccgaagaaaagtggggtctagcgaaagaaaaagcgggtctgctggcgcttaagcaacgttgcagatacccctacataggtttgtgcgtttgtttttttcaacatgcccttaccattctcaaccaataccaactttatcataaaacggagttcacaaggctaaagaggaaccrattttaaaacagttttgtcctaggtttttctccgaggactttgttgttggggccagttagttattagactgggttgcggcgagagtcgatttcaagcgccctgcaccgcagagcatcgaagaatagcccaattaagctgaggcgtgaaaccaatacctgcatgaaagtggttaaaattttgtggcgcagtgtagcacaaaatgacaggacagataagatagtctggcacctaactagtcctatgactttagtctacactacgccacaaaatattaatcactttcatgcacccccgagcacagcactagtacagtactagtacttgtacttgggtgtgagggtaagtggtcggaaagtaacccaattctcggtgcaaattctaagggatacaggaaggcattgagggagattgaagataatttganaanaagatagtgggggagagaaaggagataaagaaggaaaggagggatggagcaaacatctaaattggatatataaagcctgtagtagtataaatatactcgaaaagttttaaaaaaaaaaaaaaagtgatcggaaaaaaaaagtggtttttccgatcactttttgctccaggctggtactcgggtatggatgtaaaaatcgagttctgtaagtaaccctgtcgttagaatgaggtactgagttggctagaatagaagaaaagatcggcaagtcagggtcaccaagtacccggaccaaggctgggcagatgcaacatggcagtgatggtcctttctttttctttttctgctttctatatttgagtctgtaaagtttgatagggaattgagctattaggggtttggaatttagaagactttcaataagagcagtaacaactacaagcagcttccgcaatgattaagatgcaaccayyccaggtttttagctccttycaaggtctagctttttccatgacgccaattgcacgattcccgagattccttgttcatatccctgctktgctcgatttttacatccctacccgagtactaggggctttcaaagtcaggaatgggggccaacggtaaaatgatcgaacactcaaagccacgcattagttctgcgttagggcgacatattaactgtagccaactgtatcaaagcatttcaagtgacataatagccggtcccctcctattnnatcgcgcatagtsggaaaagcaagtgtgacctacactgtaaaaaaaaaagccgacgtgtcacacctgcttgagcgtcacacttgcttgagcgtcacacttgcttttcccgactatgagcgattgcgaaataggaggggaaccggctattagtatcattctaaattatttcggtttttggggtagagaaacggtgaacaaaagagacaggatcgcacgtacagtaagaatgattacaggtgaatcacgaggctaggggagtaatttcagttagtcgcagcgttaaacccccctaccaggttcatggttcctcccatgcagtctacgaaatttaatggaagagaccccctgctgtgtctacattggagaaaacctcaggtgaaaaaaatcaaacgcaaacaaatactgacaaaggtcatacaagtgaaagcttcctcgcctccttactaaaaaaggtggaaaaataccccacctttaagcgtgatcaatagcgcgagtcacagcccaccaacaggcctctcgaacagatgctgattagtaggccccctgtgaggaaacgggtagtagtcagcacagcagcaacagttcgtccaagtaaaccacttgaaagtttcttatttaggtttaactgttcgagtagaggacctgctcgagtgaactatgaggcctaatcgtaggatttaaaagtaccagctatgttatcccaaataggaaggcagatcttctactcaagtacggtacaagtagtattgtatttgtatcccactctcattcctacaacctccagtgggcagggatgtcaagcaaaatctaagcaaacatcacaagcaacttgaatggagttaatttacacttttgagcgaatacagcgcaagagaactcgcaactcctttttagcgagagtttcacgcatcttatcgctaagcgcgctaggaaaaaatcaggcgaaataaggtcttgggatcggagcttttttttattgtagtgacaggtcttcaatagaatagcctaatgacgttgtttaaacgagatctgttaagatgatcatgttgtaacactggtgtgggttttgggggctgaggggtagcttcacccggtatcactctgtattttgtttgattttgcgtgacccaaccaccttactgaccttcctaaataggaaggcaggcttttctactctttttggtgcttggctgtccttgaagtttctgtaatactgctggctcgttccgctactaatccttggcagtgcgtagggatgtcacacagagcttaagcaaacctcctacacttctaatatgatactaaagaatttttacttgctaaagctgctgtctaagtaattagggtgtttaagtaaaatttaagaattacgcttccctttcatctcacaatttgtgatgcccccagctctagggtgttggatgttttcgttgggtgtcaatcgatgtttgatttcgcgtgaccgctgcaaatcaaggtggaatgggagacaaaaagtatatgggacgagtgtggaggtgacttgcttgactttcctttgcgggataagcagatcttttatacggtatgtaaaactcctaggggtatcatacatataggcatctctttagtgatgcgctggatggcgagatccaatagcgatggccaccagctagtgatcgcaaagtacttccccagtgtttttcttttttctcgctagaacaaacttcacaagcttcataaaccagagcgccaacaacttagcgaagctcaattcaccggatgaatttaggcctctgcctttgcgccggctcaattatgtaatttgagaacattagatactcgcgctgtgtatgttttatgcgagcagaaatcatcccctcgtcatagcccccaagtaaacaaactgtcgtaagaactcgaataactgacctcccgcaaagacgtcccaaatttcctctcttggataggggggcgagcgcacagcgccaactggcctcctcagcctgagcttaagacagaataaaaagaaaaagtaccagaccttcccagcagaggcaattcaaacatcctagcgtgaggttcaattgttcgagtccttatggtgctgtaaaagaagtgtcgcgggtgggcggttgtgggaaagaaagagtactcaagtcgccggattgaagatgctcccgtcttagtagtgacgcactaaaaaaacgtatcttcaatccagcgacgcatactgtagagtgctataggccgcaagttaaaggaggagtttagattctttgttccttcggaatggtctaggaaaggatttgaagacttttccttttcaagtatagcatccaaatagcaaaaagggcagcgaggagattggccaaggcagctctaaaatccgcttgatatgcagcccccgcatagttgtgggagactgtctgctataatctacgagtagaactttcccagatattcctaagactacatatataaaggcgcagtatcctgtttctggtcatacgactcactcttctgcaactgatcaaacttctttcctccatataattagcttgaaatctcggtttctatcctggtggtccaagcgggggcgtcgggcacattgcttctggctctcctatctggcgatatcttcatccagaatcaggagacttagtgcgcccctttcaaatcctaccctccttccgtcatcctccctggtatgaatctttggaggttggtcaaacactctcacagcggctctttcaactggccgctggccctgggcaagaggctctgacggtttagaaccggttagggattctaatttctctgccgaaaatccttcttaggggcgttcactcactcacaatccaaggcttcgccttgtgctatgatactatgatcccaccaatcttagcatggtctaaccagaaggccctctgaaactatcacgtgcaaccggcaacagcgctagccacaattccttaatctccagacctttcgctgtccggttgttgcatcgggcacctgctcctttgttctggagttccatctttcaaatcgcgctctctgcgcccgcacacgccgcctcaaatagggcagcccttctgacctattcgcctggatgcgcaaggagtagctgaacgtggggacatatagcggatcggttggtagtgttatcgaatcattcagcccccgaccggttggggagaaaactacttttgaattcttatttcgcgtgacagcttcttggttattggatattccctcagaatggaaaaaacggcttccgaagtttgagatatgatattccggtctgccataccgtattgtggaggttattcctattacatttccaattagcaaaatatcctaatttatatatcgaatcacaactaattattcctctagggggctctaccaaagtagtaaagtacaggactgacttgggggacggataatagaacccgcatttctcagttcatcctgtttcactaattcctttgttcgaagctcctcctgaatattgaaccatcattaactatcccgacttgttttccgcatgagcatccttggccgcaagaaggaaaacattggttgatattttcgccgagaatcttagaaacctaagggctctctccctgccctagtggaaagctcaccatacatactatgatattttcacgataatactatgctcagaccctagtagggcttgctggagttgtacgtgacttcttcttagggtcacccttttttgggcgcacccacccccttcgtgactagtagtctatataagagatcccaactgcccctcttctctcttccatcaacaaccactcagcagaaacaacatccatacagcaatcctccctccgccggttgatttttcaaattcatctttcctccccatttttatctttcatcatgtcagcgagaccggtcaatctcactatggtggtcactacctccccgcctccaaagctctttgagtgggagaacaaggttcagaggcgcctcgttatgcgtaatgccactttgggttgcgccttgtatctctcggaccgtgggtatatcctcatgcaggacagaatcggttgtatmtgggtccagcacgaaaactcccccgtcattgttcaacctccgggtggtattacttttaacgtcagtggtgagctcttgccttacgacaagtggctcggtgcaatccagtcaaccgtccgccatcaggggcgagttcccctcgatcctgaagtcccctatgttgtttctgttctacgtctttattgcggatactatcagcccggacaaccgtggacggcccaagatagcgctatccccgccgccatcatcaacggtgatcagacccttgctcgtgttccacgttctcagcaattacgtgctttgaatccctatgttgcgcagagatgtaagtaaatcacactcctacagtccctaggcaaaagtgttcgtgaatttgcttagtcagatgtctcacattttcacgaacacttgctgtattttgtccctggtactaattaaatctccatccatcagcttggatctctaaatactgcaacggttatgggctcacacaggctgaaatttccaatcttactcgcgaagtctgggttgcggagcccaacaagtatatgtggcaaaacatcgcctctctttatactgctgctagggatcgcagcgagcctggtcttgtgctagaagacttcattgagactgaattggcgaaacacggccaccctagtaccccagaaaagctgctccgtgaagctgggtttgtccccaagactccaatcgacaaggaagccgccgataaggctgccgcaagaaggaaagctcgcgccacgcccaaattgtctaacttcgctctcacccgcgtttacttgggtaacgaccaccagctcagccttaccgaggatgaagctgccgatattcttcgggctttcgatggatatgaaccgaacttgggtgacggacactcgcaaatcggccctcatgtccagcacttgctggcgatggaaatgcaagacaagtttacccacacttccgttggttccaatggcttcgtgagtgataccgtgaatagtatgatttacttgttgctcaattctaacatttacccgaacagcatactatgggagaatcctccacaggattgtaaccggatatgatccccttggagatctgaggaggctgcaggtgcgaaaaaaaaaaaatggtggaaaaactgtgtacgacgggaagcagggtatagctagaaggcatactcggcgggttatggatcaatgtagacttaaattaagcttatctgtctgggaccaatagaaagcccttagtaactttaaatccagatatcaatgctttcttccataaatcagcgttcgcgacttcgatgtactaggccgttttttttttaagtattcggatatggaaaatataagtaatctagtcatggtataccggtacagtaagagcttgagcaattaaaccgtgtgcgaggtagtccaaaatttcctcctctagaaaatgagagaaaaacacactaaaaaacctgccctccgagacaaataaaaagaaaaataacaccacaccttactaggagagccaactccaacatcccagcacatagttcaattaccggatgccagtgggctttcaatgccccgaatgcatcgtctaacggtattccagcatcgcgagattctggcactgtaagtacaagtacagaagaaagacaccatgatagtcaagcgctcgcaagcggccaggtcggcctgcaaatagccggtaggctttcagtccggccaggccaaagtgagacagacctgggcttcctaggccaggctttgggaaagccggcaggcttcatgggtccctggcggcaccggtaccgtaaaaacttgaataattggacggtgtgctatgtcgctgggttttcctctttcagtaaggtcaagaaattttcgtttttttttccgtcccggaaggggaaaggggctgtgtttttgctgtgcgttttgctccctttccagagaagggaattctggactacatagcagaccgtccaattattcaagtttttacggtaggctttcggtatattccatgtgactagtcaatattttcgtaatgtgtgggtgatatctgcagatatttgaagataccgccagatatctgaagatgccgccagatatctgaaggtgcctgtaggtttctgcttattattcaggcttgggccacaccattcgatttaaaacatattctgcttggcttcagatcttctagagtttagcgagaaagtagtatcttatttttcattcactttcaccctttttctcctaattgacaaactaactactgtaactagcggtaaagacagaagcacataaacttgaacgaagtcgtcctcccattctcccatccatcgcaccacatatcctttctctgcgacctgtatacccctctccctttcatggacacctaaactaagttagcaaaagaaccgacatgcctttcaaagaactcccacggctcataacctccacgccgagcgtccaaccttcagcacgtctgactccttcgagatcgtactgcatctggcggtgcgtccatccaatttggcaatacagctcggagtagacgcggtcggccccagcgcacctggtgggggcggccgggggttgttctcatataccgcccggggtgggcgtgaagggatagctctgggtataggaggatgctacccacgggagtgaaagcgcgacttagaggaaaacaattgcatggcagagcgagaaagacacaggggaaaggtagcactagaagagtgcaattacgaatttgaatgagtaggatgtaaggaggccgctatgcgttgtgagagttttcaatgaactttgttagcacaagagtctgtaggggggaatgggagacaggggcaagagggagagaacgcgtgcaatagtccctagagcactggttggtagatgggaagctcgtggtttatgtgtactcatgtctttacctctaattagttgatttatttattgaataagggcatacaagcaggagtcaagttgaatcagaagaactttcccccaattgtgctgtgtgttggggatgaatcttccatgcttggtgttggtttgtgcttggtgtttcgtttgtgcttggtgtttcgtttcattctccagctgaatcacatcttatcactcgaaagcctgcagatatctgcagtccggccaggcccggtcagatgagcttcagaagccgggctggccgcttgcgaacactggtctcactggggcccccagactaatgcgaacaccgcctagagtatgatacccaatgccagacgctctgccacccggggggtcactaaggggctgtttggagtcacaactttggggccgcaaaccattgtttgtaactacaggtcttatttggcagatgggaattacaaacaatggtttacaacaaccagcagcaatttttttgctgtttatgaaatagggcacagggttctttcaaccatggtttgtatcatagttaaccaaacttcacaaagttgtcacgccctaactctctacccaaccctatcattgccccacacaaaaaaccgcaaaaccaaatctaattgaagggcgtcaccacctataaaaatccgaggcctcacactccctcataatcctatatcccccctgtaccaaaactctcttcttctcaacacccactccagagtactcaacctcacaacaccctccgcatattggtcagccagtctgccaatatcagtattcgacttacagttgacctctccacacatacgcttgttttctttgctaaaatgcccccaagtatccccccggctgaaaaaagaaatgaacttaataataataattaacacaatcatagacgaggacatcaggctccgccgggaggccctgtttcgggccggaaggttgcttgctaacgaaatggaagccaccgcctggaaccctggcaacttaatggttggaatggccattcataagaacccaagcattagcacggttaaagctgttgagtcggcccgagacttgcttcgtgaagtcactgtgagtgacaatagattacgcaagatggatcccatcaatgatctccaaccgctggcgaatatccataacacacttttacagattggaggattagtttccttgaaacaacgcctctatgaggcacttcaagatatcaagctgcgcctccaggaactccaaagatatacgggagacaacattggccaggcagctgcagaagttcatgacttcatctgtttcgtgacttttgtcaacttgatccatccgcaacaatgcatattaacctcggaatccatcaaggatgccgagatcattgcctccaagattccgttttttgaggctcgtcaggcctttgtcgaactccgccaagcttaccaactgcagactcctgttccggggtatcgtctaagcaaagccaggcagttgcctgcgatcactagatgcattatggagcagtggtataaagattacctttctcacgcggccgcaactggaacagtaagcaaaagaataccttttctagatcagcgctcgtacaatgtttccctacgtcgcgagcacccggaaaggagagcactggctacaaaatgctttgaactacggtttaaaacaataatataagcacgaaagacaagccggtaagtgttgagtgctcgctacgccgggggctcgcaaagtagggaaacatagtatctaataagccccaccccgcctcatactgacattaacgtatagaaaattatcccattccagatcgtgttcggatggtggagtcagttcaattcccttattcaggtttgctagcactaccagctgacaataaacgatcttgggttatagttaattaccaacgcaagtttattagtagcgctcaagagaaaactactccgatcgtgcaagaacttatcgaccaagcctatatctcgaaggccaagattccagaggatgaagacgagcaaccatggatgcaggaagataaggaagcttttaaggccgccacttctctggctatgtctatgaccaaactatgcaaaaatatccctctacctaagcatgttaaaccagaccctcagagagaagaacaattgccaattcacacccagttcaatgcccgtcctcaaacccccccgatcacccgagagctccagaagctgctggctcaaatttatggcctttttgaaggaaattcgtgcgcagcttttactatcatgaccaaagcgtattataccaaagaggaaaactattttagattctcggcccaggaccagaaaacgtcttctgtcaagcacttgaagtgagttattgcccaagtctcaagatttgttctacatcaaagctaattttcaacagcgccgcgacctactttttcaattcgctccgccaggttagggttgtggttctgaaggatcagtctggcaggagtgctggggaaggcaaggttgaggttccgctccttgacgcagttagtgcggtctgtcgggaccccaccatcacagacgaagagcatctggcagccattacctgcaagagtttaccacaagttggcatgcattgtaggttcccattatcactagtttcggatatgtgctaatctcagaatgtaatcctggcaatagtccttgttagatatcgcgggcccgttggccggggccgttaaattttactattcccagtagcacttgacaacgtcctctatcaattaaaatgtgccaaagtctactactatccgagctatgtctttagataatctgaaatctttgtactcacttgtgaattcaaatgagagctcaaaggaagcagtgaaaccatctgtaagggaatcatctaggtcagccccttagactagtggtgttatagaacgttagagtttggagaaatagacatttccaatcatattcatactcttgctctagcccgtgggccttcgtgggccgtgtcatgctgtagaggaccggcccacggcaggcactggcaatgaagacagcaaaacctcccatatgagacaaaatccaggatatattactacgaggactacaaaaaaaaaaaacgccctctagagccagctttgtctatccacctacctaccaataacgcccccaacatccgggtcctccctcaatgcggccttaacctcctcctcaccaatcaaaaacctaattctccccccgccgccgccccgcttctccacactgagaactccagattcaaccaaccccttgaccgcggcttctattccgccaactcttggcccaccgccaagctctttttccgcaacgccctgatttcgaacgatcctttccgcttcatcaaggacatcacggagaacaccttcgccgactccggagcgacgggtacggagtaatagagccgtgatgaagatcttggaggaaagggaagtggttttcaggaacacttgcaaaggggaagaggtagcttcctggattgcctgtttaacaacggcaatggttactttcccaccggctttgaggccgccgccgcgcagtggggagacgcccccgcccttccgcctggacggcgaagacggtacttgctcgtctcctccctcgttggaggagtattcgacaatctcgactgcgcgcctgcatatatctagggctcttctagcatcgccagatacagcagcgacttttctactagcgaactgaacggcgtctgggtccacaatgttaccaggtacaccgctgaggcgggactgaattatcgtcattagctgcgaatgggtgtagccaggaaatgtaatcctagtaagacctgttaaacaatttagctgcatttctcaagagttaagggtgtctgtgccaacctaatctcgacgagatcttattgctcaaggttctctcgggcaagtccatggtattcgcaaccgccaaaacaataagcctagaataccgtcccccgggccaattgaagaaattgtacatgactccttggccccgcgtaactaattgatcaagttcgtccatcaagacgacacaaggaacccttcgaggactgggggtgctgaactctgactccaaaagtcccaaggcttgtgaggggcttacacgatcacctttaatcgcctcccaaagaagagagtacgcctgatggggatccgtgaccttcattccattgatctcaacaaaggtaaagtcgtccaattcttccaaggtcactaagtactgcagttgtgaaataacctcacgaactgtagcagtttttccagttccaggagttccggagatgtaaatacacgatccggttccctcggcaatagcagcagaaaggtgagtgtacacctctgtaaactcattctcacgacaaggcaaagaaggtggaagggtagacacgtgcagccggtggcgtgcaagtgcataaggggttgacaagtgagtacctggggacagtgtacgagttgaaagaggagtaaagagcagaggtttcttcaccactattctagagagatgttagcaattatacagaccaatcgacagattgtctaacctcttatgtgtaggagtactgtatttcttcgagacgcttccaggagtccgtctagttggagtagtaagagcagatgtcttccttttcttacttggagtgcggggagctttagtccgtcgactaaaattcgggtcatcctccaccacatcgtccaactctgcaatttcaccctcatccaacatatcccgatcccattcctgatctgtctcgtgatcctcatcacttgctttgcgccttttcggtctccgtacgccttgcccgacagatttgttccctctccgcttaaccaccttagtctcattatacaccatctccccaatggcggcaatatcagtgtccctccctttccaaaattcctcccaaataaactcaggtgtataagttgcagttctaggattacacccccgtcggcaaacaaaaaccttcccatactccttggatttcttctgcacgccgttgggatattcttcgttatatttctgaagggaaagaacctttgctcggccatttatcgacggcagagggatcaggttataggaggggttaacgtaaagttcattctattcccaaagtcagcacactgaacgtgtaaaagaaacaagcaaacaaaccggtaaataatccatccgcttcttttccttattcttaacctcgaactcagtggaaaaccacatgacatttaccatcatttcaaccccacccccatcctcaatctttccctcttcgtcaaactcacatatcatcccgacat

SEQ ID NO: 57 - DNA, 11180 bp, Tuber borchii
  misc_feature (8629)..(8629): n is a, c, g, or t
aagcttgacagagctcgcccgcttgtaacggccgctctgcatgactgatccacattaaagctgccatcccgagtaccgatttattcccaccctgcgcctttatccgttctatcgtcgcttcgtaagcatctcccaaccccaatccatcggtgattttgctgagcctttctcgcctctgatcgattgtcggttcttgcaagattgcctcaatatttaaggaaacgagaagaaacctagatatatttgttagcagagaatgtggggtaggactcccagtaccattgccctacgtacatttccgatatacccccgggaatcctctctagaatgtcggcctctagactctcgtccatagcatctagagtttcgtcctcgctcagcctagcacgaaggtatctaatgatatcatctttgttggggcctatagacacacttatcactcgtccggaaagacgcttttcgatctcagcacaaacatggggccttccggttatgaatacacgtgtgcccggggacttccccaggatctgttccagtgaatcaaggattttgatccgatggactgccgcacattcgtccagggcgtcaatgcacataaatgtgtgttgcgaggaggtgacggcctgcagcatcttcacgatctctggaagtcccggcgactttccaccaatggccgttttctgctcttgaaaggctcgtgatatttcttctggaatcctttccattccgctaacgacttgttttagcaaggaacccagcacactcgtcgcggaatgctccttgcgggccgcgaagtcgaaataaaaacatgtaacagtggggccttgtcctttagtctgatcgcacagcgtgtccactaccagcgaactaacaccgcggcttgttagcactgctgctcttcttccccgaagaatggtttttgttacctgataaatgttttgccgacccctggatctccataacaaagcaagactgccttatccccttcaccttctccactacaatcataccagcttttaaattgctcggtctgtataacccattcccccacgttatcgacgcggcgctcctgaacatcccgatgccgtgacctgggctctagggatgacaaccagcttaggagttgggaccgatcatcgacaatagtatagttgttccacacattattaaaagagttcgtattattgaaggagttgttactgtcattgatggtatattgagacatcttccgctctttaccgcttgcaatgttgaagaggagttatcgagtaacttacggctggttgatggtattcctgggaatcgaagaaaagcatggtgcagctcaaaagcggggcttgctggcgttaaagcagccttgcactgttactcatcaggtatggtgccgggacttgaaatcaaccctcatgggtaattcattttgcttaataatacacacaaaatagtggggcttggcaaaagtgtcttcaaaaaccatcctacatgggtaattcaaaattttactatctccgctaatattgctctgattgaaaattagaaaacatagaattagagaggagggtcttctgagcacttctatgaagccatatcctattaaagatgtggttgtggttgaaaactttcgaagattgttcttgtgttggtctttgggtgatttcaagtgcccggcaccataccgaagaaagtccaattgagccgccgagagtttgtgaaaccgctcttgctcttgtgtggcactgttttttttttgttaagtccttgatttattcgtagctaactaacaggaacatcatggcaagcaccccaaggtatggcctaaaaaccctatataacaaaagaaacttcttcgtcccctaaataacaaataaacgataaaacatcactatacaatccttaactaaagcaaagctttcatatgatcatgaagccaaaaaggcatctctcgtaaaaggaaaaacccccatcttcaccgggaaatttatccttccctccttcacataagacataaagtcctccatcaatttccttcccacctttcccccttcccacaatttagactctggtctgatgtcgaaagctcgcgggtccttattctctctcgccctggccattgctgggcattccattatatgcgctggtgtttccaggctcgaaagacacctcccacaggtggtgtcccgatccagattcgaacaatccatctgagctccgaggtaactcttgttcgacctcatttggtgtagccttgacgccatgaacttagggagtgtcataaaaaaccaacattccctatatggcggcctgtatccgtagtagggggggtactcccatcctacccatttctgatgcatctctcttctcaccctttctctcgctttgaaatgaaccttcttgtgtggcactgtactcgtatcatcctagtactagtgcggccgatagataaaatagtcacgtgggtctgttaaggcgcttgacaccctatatatagtttatctatcgcgcaggagggagggaggaggaaaggaagaacggaaaacgtgtaggtaaaaattcacagctaaataaacaacggagtatgataggatgaaaactacaagctataaactaggcttcccacagcaataggaccacttccccccagggaccaaactctactgtgacttgtaatcagacttggaaatcctcacctggcataaaagcgaatccctagcgagtttcgcgtgtgcatgtgacatatacgtgcattccccgggcccatctcatcggattccctccagctgcaaaggatgacgtccatggacaatttctataagtgacatactccgaaagggaaggatggcagcgatttcgacgaggcggtgtctcctacacctcaaactgccccaggggttgccgctttgcgcgactgtagcagcgctgccactctgttatggctgcccctagcagcgaacgagagcggtgtttggccgaaattatttggtttttcggggttaacatcttcccgcatgagcggtattttgaccacttcttcatgcccataccaagcagcgaatgagagcagtgtttggccaccattatccggcctgtcgaggttgaccccttcccatccgagtagtattttcaccacttcctcatgcccattccaagcggcgtacgagagtggtgtcctgccacgattatcttgttcgtcagagttaacatcttcttgcctatgccttctgctcgggaatgaatctcggggttttatctgcttcctctattaatgacaagtttaggcgcatttcacaatccaagggagcactactgggctagtagccatatccccaactctagaagatggtgaagcaccaaccaatatggggtgccaagttaaactggtttacgagcgatggataaataaagcggtgtcaccgtgagaggtacttttttgtcgcataatataagggcgtgagggcctaaggataattgtcagagtttgccgcgtacgggacgctcgggcactcagtccatgggtttcttaggctgcctggcgtagggcgtccgctcagccaaaaaaatgacggactatgacccacaattcgtcccgccctactgctcacaacttgatgtttatcttatggtttggtgttcggatttctcactctcattttaagaaattttttgtttatttggaccgtacaagtttatttaagtgaacaaatgaccctttacaaacagctagatcatccctcccattaaaagtaaatcacccaagtaacccataagctccccccattgagatcattcaattaatcaaaccgaaagatttcagaaccctctccgcttcctcgataatatcgcccaaggcgtttgcgctgggctgggaggcgaagcggtcggtgagggctccaagatgggtagaagctctcttcttcgcacgcttcgcgaccccagcacggtgaaggtcggcacgggtcgaagggcccttgttggcgcacgccctcctcataatttcatgagagcggcgcggggtgtatttatagttgggattgaccaacttgtgagaagccgcgagttggcgggcccttgcctattaatgtgttagtgattccaactaacaaacagtcaagtgagatgttatactttataaacatctttggcttcgtccggggaatcctgccacatgcgaccgagaatttttgctattgaaactattagggaaatggtactaagagcggagagtgaaaaaggggagaatgagaaccacatacaaatatcgttgttaccaaccccaggatactgagccttgaccgcattaacattctccgttctaaacaagatgaatctatgattgtatcagcacatgaaatcggtacgttacagtcgcaccccgctataacgaaggccaaaaattgggaattttgttagttatagcgaggttttggaagaaattccttatagcaagggccacccgcataggagcaggagtgtaaattcccagagggttcattcgtaatagcgaggtttcccttataagcggtccattatagcggggcacgactgtagtaggattgtacttacgcattagcggggcgcttaacacccttatcgcggatgaaactaagattagcgccatgagaggcaaagtcttgatcgttgacagcaagacgcaaaaaacggttttcgccggtaatctcgtgctggggatcctcgaaggaatgatcggtgacgttgcaattcatgtcggtagtagcgggatcgaacacaagaagaatagaaccatcgttaagcttgtaaatcgggcgattgcggatagtaaggtgggtcccaataatggtgtggagggccgaatcaaacgagacgataagaggtccagaggcaggcttccagttgatcggaagcatatggtggatctggagctgcgctccatccgatccatcgaaataaaacttgttgctggcgagcatagaaagatactcacagaggtcgactccatcgtagcgttggtgaataagaggcccggcctcgacggccatctcgagagcagagagggagctcatattgaagttaatgaaaataaaatgagttggaaaatatgccgtgataggagcactaccagtaggaaggttgaatgttgcgatgttgaatttgttgttggtcgtgttggttgttggtggaagaaagaagagggacagttgggatctcttatatagactactggtcacgaagggggtgcgcccaaacaagtctgaccctaaaaagaggtcaggtacaaattcaacaagccctacaagggtctaagcatagtattatcgtgaaaatatcatagtatgtatggtgagctttccactagggcagtgagagaacccttaggtttctaagattcttggcgaaaatatcaacgaatgttttccttcttgcagccaaggacgctcatgaggaaaacaagtcgagatggttaatgatgatcggtttaatattcgggaggaacttcgaacaaaggaattggtgaaacaggatgaactgagaaatgcgggttctattatccatccccaaagtcagtcctgtgctttgctgctctggtaagccccctagaggaataacctcggacggaagccgttctttccattctgagggagtatcatatccaatagccaagaagctgccacgcgaaataagaattgaaaagtagttttctccccaaccggtcgggggctggatgattcgataatactaccaaccgatctggtagatgtccccacgttcagctaccccttgcgcatccaggcgaatagaggagggccgccctacccgaggcggtgtgcgcgggcgccgagagggcggtttgaaagatggaactccagaacaaaggagcaggtgcccgatgcaacaacagggcagcaaaggctctggagcttaaggaattgtgactagagctgttggcggttgcacgtgataatttcggaagttgttctggctagaccgtgctgatattagtgggatcatagtaccatagcacaattgtgagtgggtaaactgagcacccctgagaaggattttcagcagagaaattagaatccccaaccagttctaaaccgtcagagccccttgcccagggccagcagccagctgaaggagccgctgtgagaatgtttgaacaccaacctccaaagcttcatacaagggaggatgacggaaggagggtaggatttaaaaggagcgcgctaagtctcttgattctggatgaagatatcgccagataggagaaccagaagcaatgtgcccgacgcccccgcttagaccaccaggatagaaatcgaaatttcaagctaattatatggaggaaagaagcttgatcggctgtggaagagtgagccgtatgaccagagacaggacactgcgcctttatatatgcagtctaagggacatctgcgaaggtgctagattatccggttccagcactctagtcctgttacggaacgatattagtcattaagcactgctagctgcccctcaatctggccttagtgtgctgaaaccgaattttactatttcacaccgcctgggatagcagactgtctcccatcactatgcggaggctgcatatcaagcggatattagagcccctcgctgccctttttgctatttggatgctataccggaaaaggaaaagtcttcaaatcttttcccagacccttccgaagtaacgaagaatctgaactcctcctttagctcgcggcctatagcactctactgtttccgggctttcccgtcaggtgttcttttgcgggcagttggcaattgggtttgctgagcttccggtgaaggtagccagcagatgaaagtgaaagaaagcgaagagagataactgaagaggatgaagatgccttgatggtcgtgattgagatattctcaagtcacgaagcaaggacggatgaagaatgatggaatgcgttctttcagaagaagaaagatcccacgatgctcattgtttctaggaggagttgtttcctcgtttaaataacatgaggactgctaactgagctacctagttctttattgcgtaaaagcatacgcaatgttcaccaaagtctatgaaaaactcgctgagccatccggacgcacgacactacaatacttttgtttagtgcgtcactactaaaacgggagtatcttcaatccggcgacttgagtactctttctttcccacaaccgcccatcctcgacacttcttctacaacaccgtaagaactcgaacaattgaacctcaagctaggatgttggaattgcctctgctggtaaggtctggctctttttcttttttgtctgtcttgagctcaggctgaggaggtcagttttccctgagcactcgcccccctatttaagagaggaaacttgggacgtctttgcgcgaggtcaattattcgagttcttacggcagtttgtttacttggaggctatggcgaggggatggtttctgctcgcataaaacatacacagcgcaagtatccaatgttctcaagttacataattaagcgggtgtaaaggcaggggcccgaattcatccggtgaattgagcttcgctaagttgttggtgctctggtttatgaagtttgcgaagtttgttcttctagcgagaaaaagaaaaccactggggaagtactttgcgatcaccagctggcggccatcgctattggatatcgccatctgacgtatcacaggtcgtgttaaagagatgcctatatgtatatgacacccctaggagtcttgagtaccttacttaagagatctgcttatcccgcaaagaaagtcaagcaagtcacccccaccactcgtcccttatacattttgtctcccattccaccttgatttgcagcggtcacgcgaaatcgaacatcgattgacacccagcgaaagcatctatcaacaccgcctagcactgaaggcatcacaaattatgagatgaaaaggaaagcgtaattcttgaattttacttaaacaccaaagctacttcgctaatgatgcctagccaccacaaataaaagaaacccctagactccctcccctgcccttagagcacttgtagaaacgatgcgtcaagctcgccaaagagatgtcacgagttttaatgtatggtattggtccaaaagggttgtaattctagacgctctaaccatctaccgtaaaacctcaaataaaagaaccgtgtgctatgtagtcgagaattacccacttgagaaaaggggctaaaaacacagcaaaaactgagctgctcaggtcgcctgagagtcataaaaaaacaaaaaaaacaccagaccttaccagaggaggcaattactcggatagggtcaaaaagggcggaaaactttcggtggcgaaacaaggatagaggcgctaatcgatcaggaggaagtagaaaaaatggggggtgttataggatgccattaacctggagatgttgcagtgtagtcaccattgtattccatctatgggtcttattaagaatctgatcatataaggccaaattaataccaaaggagaaggaatcagggcttttggaatagttctttaagagactcatataaatgcataggttcaattgcacattggcaatttcatcatcatttaaatggtcaagtatcatactagnatacttatttcttcaacctggatttctggtgtccgccaaggcaagaatttttccactgttgcaacctacttcctaactaccccacctgatcctgctgatctttttttccgccctttttgaccctatccgagtaattgcctcctctggtaaggtctggtgttttttttgtttttttattgtacttctcaggcgacctgagcagctcagtttttgctgtgtttttagccccttttctcaagtgggtaattctcggactacatagcacacggttcttttatttgaggttttacggtattcatctgcctcaatcccagcccaagttgctcttgtcttggccaaaatcctaggcgtgacttgctctaattgcttggacaccagctagcaagtaaaaaattctttagtatgatatcatattagcagtgtgggaggtttgcttaggctntgcgtgacctccctacgcactgccagggattagtagcagaacgagccaacattaccagcgcggctgatagaaaacatagtcacgtgagtttgataaggcgcttaacagcaagaaggttcaccaaattttttttattaggttcacgtgactattttacctgccggccgcgcccgtacagaaacttcaaggacaaccaagcacccaaaagagtagaaaatctgccttcctatttaggaaggccagtaaggtggttgggccacgcaaaatcaaacaaaatgccgagtgataccgcgtgaagctgcccctcagcgccccaaaacccatacgagtgttacaacatgataatcctaatacaacgccattacgctattctgttgaatatgtcttgtcactacaataaaaaacagctccgatcctaggactttatttcgcctggtttgttcccagcgcacttagcgacaagatacgtgaggctctcacttaaaaggagttgcgagttctcttgcgctgtattcgcccaaaagtgtaaattagctccattcaagttgcttgtgatgtttgcttagagcctgtttggatacaacagttccgttccgttcccccatacaaatggcccttttccttccgtttcaaaacggaaagaacgctcttttcacagtagaaatggtcgcccgacggaacggccccatttttgccaaaaaggttcttaagctttctagctctcaccatcgagtatgacagtcaaagacattgttggtcaatgggagcagaagttcacgtgacctacttagcactcggcgacctgagtacagaaacacgaaagtaacaagtttacaaactagacaaaagtacaatgccagtttaaaagtctacctgtaggatttgtatggaaattttggcagaagggcacttttgtgggttcacgcaagctataactacagttataccttctaaatctcgatacgtctacagaaattgttagatctcccacctagaaaaatataggtctgtttaaactagtgcccatgccgtgaggccacagaaaaaaaacagggctccccatacaaaacacatggctaatgaccaaatatatcaccaaactaggctcgggtagggtgctctgtcggggttgggaatggagaaaaaaaggggtcagcaagatcctatggggcagtgagcagacggttcgtgtgagtggaaaataatgagttctggcagatgcaaacaattctgagtggatactaaacacattatcttaccgacaataagagatcccgagcggatgattaagaattacccttagtgaaaactagctttatagagattacttgaagctccctgaagccctccagaatatctgagttggagtttaaagcagacactaatatacttggtccgggcccctaatttctacaactccctctttgatggggcccggctagcaattttcgttttaatttaaattcacaactctctagcataggagggagaaaatatgaagtaaaactgcgggaaatcttatagtatcataccagagatgcacaaatatttaataaaactatctaaagacacaacccaggtggtagtaaactccggcaccccccaactgatagaggatagttatcaagggctactgggaatagcagaattgaacgaccccggccccaacaggtccgcgatatctgacaaggactgttgccaatattacattctgagattagcacatgtcagaaacaagcgataacgaaaacatacaatgcatgccaacttataccaaactcttgcaggtaatcgctgccagatgctcttagtctgtaatggtgggatcccggcagacctcactaacagcgtcaaggagaggaatcgcaactttctcttgcccagcaccctcaccaaaccggtccttgagaaccacagccctcacctggcggaacgaattgaaaaagtacgtcgcagcactatttgagaattagctttcacctaaagaaactttgaagcttgaaagatagcttgcttcaggtgctttacagaggacattttctggtcccgggcagagaacctaaagtagttttcctctttagtataatacgctttggtcatgatagtaaa

SEQ ID NO: 58 - DNA, 1067 bp, Tuber magnatum
atgtcttccctctctgtccttcagatggctgttgaagccggacctcttattcaccaacgttaccacggagtcaacctcagtcaatacctttccatgtttgccaataaaactttttccttcaatggatcggatggagctcagctcagtgttctccatatgcttccggccgactggaagcctgcctctggaccccttagcgtttcgtttgattcggccccccataccgtcatcgggactcaccatactatccgcagccgccctgtttataagcttagcgacggatccatcctcctcgtcttcgaccctgtcgttaccgacgtcgattttcagctcactgatcgctctttcgaggacccccagcatgagattaccggcctaactcgttttctacgtcttgctacagatgaacaagacttcgcctctcacggtgctagtcttagccttattcgcgctaagggtgtcaagcgccccgccaatgcgtaagtacaactttcctgacctacaggcttcatgcgctcatacagccacagrtttatcctgttcaggaccgagaatatgaggactatcaagactcggtatcctcagatgggcaacaatgacatttgtatgcagctctcatcccttttattaataagctcttcattcttagtatcatttcactaatagtttcaatagcaaaaattctyggtcgcatgtggcagaattcctcgaacgaggcgaaagaggtttataaggtataccacctaacctaactgttagttgaaatagctaacacattaataggcaagggcccgtcaactcgcgatttcgcacaagttggtgaatccgaactacaaatacaccccgcgccgttctcatgaaattaggaggagggcctatgcgagaaagggtctttcgaactatgtcgatctttactctcctggagctgaaagagatgccagagagagggctacaacccaccttgggacccccactgcacagtttgcctccgagccgagcaccgtcaccctcggtgaaattatcgaggaagcggagaaggtcttgggttcctttggtctgattaattg

SEQ ID NO: 59 - DNA, 4790 bp, Tuber magnatum
caagttgtatgcaagcagaaatcatcccttcgcttgatgtccaattccacacctttcaaacgaaaaaaccgttaccttataagtatgatatcacgggtgggagcttgtggtaaggaggagagtctgccagcactaccataagaaagtcgccggaaaaggtgtttagattttcgttacttcgaagagtccggtaaaggacctgaaggcttttacttctccagcattactcacgggcagcaaaagacagcgaaggggttggccaagggggctctaggatcagcttcgtacgcaactactgtatgtttgtgagagatattccagagtattctagatccaccctcacaaacaccctcgcaatattcatagacgtgtagtatccccgcctctgatcataccactcacttttccacgaaacaagacaatcggtactctttcttccgtatattttcctcgatgcatcggtttctatcctgggagcccgagcgggagcatcgggcccttttcttcaaacacccctatctgacaatatcttcgcacagaatcaggagacatagtgcgccccttttaagtcccactcatcctgccaagcatgaagttctcggggttgagctctcaaatactctcacagcgactttcccaattgtcccctagtcctgagcgagaaatcctggtggtttagaactggttagagattgcaatttctccctggaacccctccccggggggttttcacactcacaatccagagctcttcctttcgctataccactacaccatcccactagcctcggtatggtctaaccaagaacctctccgaaaccatcacgagcaaccgccagcaatgctaaccacaattccttaatctccaggccctttcgctcgcgggctactgcatagacctgctcctttgtccaggagttccatctttcaaatagcgtccttggcagccgcaccacccatcgaatacggtagttaacattatctatccgcccgcatgcaccagcgggtagccgagcgtggggacatccatcggatcggttggtagtgctgccgaaatattcatcttatttgattggccgaggagaaagccacttctgaatccgtgtttcgcgtgacgggttctggggtattggatatcccagaatttagagaagagcttccgaatggctgggcttgaggaatcgttttccagtccgccataccgccttatggaggttatctatataggattttccaattagcatgctaccttaatccaaataccgaatcgaaaggcaatattgttcaagagagcctagataccttacctctctgccaaagaagcaaagtacgggagaagtaggattgaggagacggataacagaacccacatttctcggctcatcctgtttcactaattcttttgttcgaaactcctctcgaatatcaaacagatcttcattaaccaccttgacttttttctctttgggcatctcgggctgttggagaaaactttttcgccgatcttttcgccaagcatcttaaaaacctaagggtcgccctcttgccctgggaaaaatcttaccacaccatactatgatatggtgttttcacggcaatactatgctcagaccctggttagggttgctggagttgtacgtgacggcttcttggtgtcacttcttttggggagtcttggcgtccccccccttcgtgacgggaggtctatataagaggtcccaactcccctcctcctttcttccaccaacaaccgacaactcccacaaggttagattcacagaaacatccaaccttcctacaggcaattttcctatcacggcatatttgtcaccctctatcttcaccacctcagcaatgtcttccctctctgtccttcagatggctgttgaagccggacctcttattcaccaacgttaccacggagtcaacctcagtcaatacctttccatgtttgccaataaaactttttccttcaatggatcggatggagctcagctcagtgttctccatatgcttccggccgactggaagcctgcctctggaccccttagcgtttcgtttgattcggccccccataccgtcatcgggactcaccatactatccgcagccgccctgtttataagcttagcgacggatccatcctcctcgtcttcgaccctgtcgttaccgacgtcgattttcagctcactgatcgctctttcgaggacccccagcatgagattaccggcctaactcgttttctacgtcttgctacagatgaacaagacttcgcctctcacggtgctagtcttagccttattcgcgctaagggtgtcaagcgccccgccaatgcgtaagtacaactttcctgacctacaggcttcatgcgctcatacagccacagrtttatcctgttcaggaccgagaatatgaggactatcaagactcggtatcctcagatgggcaacaatgacatttgtatgcagctctcatcccttttattaataagctcttcattcttagtatcatttcactaatagtttcaatagcaaaaattctyggtcgcatgtggcagaattcctcgaacgaggcgaaagaggtttataaggtataccacctaacctaactgttagttgaaatagctaacacattaataggcaagggcccgtcaactcgcgatttcgcacaagttggtgaatccgaactacaaatacaccccgcgccgttctcatgaaattaggaggagggcctatgcgagaaagggtctttcgaactatgtcgatctttactctcctggagctgaaagagatgccagagagagggctacaacccaccttgggacccccactgcacagtttgcctccgagccgagcaccgtcaccctcggtgaaattatcgaggaagcggagaaggtcttgggttcctttggtctgattaattgagtgatcttagtggagggggcttatgggttacttggatgatatacttttatgggattgatgatcttgtttgctcgtgatgggtcgttagctcagttaggtaaatttgtatgatccaaataaacaaaaattatcttaaaaatgagcccgagaaatccaaacaccaagtcgtgggcaaggtaggagcgagtagtgtatggcaaggggcagtgggaacttaaccattcgagtatggatacagcaacccagaaagtttatacagtacccgcgcggccgaatttaagcccgacaccaccccttataatcgtcaccagccgaccaataagaggtggaactcagatggaaacttagtaaacacatacggtataccagatagaaaaatgtccaattacatgcatcctaaactatcgtatacctgtatcatacacagaaaatttgaaatcaaacacttcctataagaattaccacccccaaattaggtgtcaagccccaattcagttatataagtatatcatttacccattaaagttatcgtttcggccatgaagaataacaagctcagattcagcagttgaggcatcacaacctattcttttcaatggtttatcgagttacatcccccagcttggcaaggccatttctgcataattattcgttggctgatcactaccataagaagtggtgtccggctttaattcggccgcgagtatgacctagtttgagtggaaatcccctctggaggtcaaggtgtcgcgtcccccgcattcccacacattactaaaccgggtgttgtgggtaagtctgtcgaactcactttaattggctccagggcctgctcgaccacttagggaacagtattgctccggccactgtgccaagagttcgacagacttttttccttcaccaaaaattagcgtctatggcctgttcagttgcataactcgcgttgcacacacgttaattcaaccaaccagaccagagttcgacagacttttgcgcacaccctaagtacaccggccgccggccggtaggttattcttagtaatggcatcagaaaaggcggccacgaatcgatgcccagtatgggaggcaccagccacgccccggccagctaacacactatcatactcatcgggatcaaaggtctgtccccgaggtatagtagccagtggaagcacggcaaccactcccctccgccagccttgacgataggagaattgtgttgataggtatcgcttataaatcaaatatccttagaaaacaagattactagtagcgctgagagaagcaagctgcaatctttacaaggtgttgtggctaggttgtttgtgcataggtgaaagtcggggacagactatttttttgccgaatacaatagaaaccggcgcagttcggaatgggctagagaagcactggaaaaatcgtgggtatagtgcacggccagtagtggcgagaccaggcttaccccataatcagacctcaaatttaggccaaccaaaccatgagaataaacaccggccgcaccctaatcactagcggtagtgcgacttttgtgagtttatggtatggcggaggaacagaggggtgtatcatacgagtaaaccaggtggcaaccatgaatgcagccgctaatacgactaaaaatgcgcgaggtgcgccaggtgttggaggtcgagtgtgtgtattgagagattgaagagaaagtaaagaccgaggaatataggggggaataagaagctatatattggaaacgaagagcgttgatttttctcaccgtcctatagttacatgcgccagagacagccattcttatatccgtagaaaaccgcttgtgaaccaagtggtgaaaagagaagctccgagctgagcat

SEQ ID NO: 60 - DNA, 8221 bp, Tuber magnatum
caagttgtatgcaagcagaaatcatcccttcgcttgatgtccaattccacacctttcaaacgaaaaaaccgttaccttataagtatgatatcacgggtgggagcttgtggtaaggaggagagtctgccagcactaccataagaaagtcgccggaaaaggtgtttagattttcgttacttcgaagagtccggtaaaggacctgaaggcttttacttctccagcattactcacgggcagcaaaagacagcgaaggggttggccaagggggctctaggatcagcttcgtacgcaactactgtatgtttgtgagagatattccagagtattctagatccaccctcacaaacaccctcgcaatattcatagacgtgtagtatccccgcctctgatcataccactcacttttccacgaaacaagacaatcggtactctttcttccgtatattttcctcgatgcatcggtttctatcctgggagcccgagcgggagcatcgggcccttttcttcaaacacccctatctgacaatatcttcgcacagaatcaggagacatagtgcgccccttttaagtcccactcatcctgccaagcatgaagttctcggggttgagctctcaaatactctcacagcgactttcccaattgtcccctagtcctgagcgagaaatcctggtggtttagaactggttagagattgcaatttctccctggaacccctccccggggggttttcacactcacaatccagagctcttcctttcgctataccactacaccatcccactagcctcggtatggtctaaccaagaacctctccgaaaccatcacgagcaaccgccagcaatgctaaccacaattccttaatctccaggccctttcgctcgcgggctactgcatagacctgctcctttgtccaggagttccatctttcaaatagcgtccttggcagccgcaccacccatcgaatacggtagttaacattatctatccgcccgcatgcaccagcgggtagccgagcgtggggacatccatcggatcggttggtagtgctgccgaaatattcatcttatttgattggccgaggagaaagccacttctgaatccgtgtttcgcgtgacgggttctggggtattggatatcccagaatttagagaagagcttccgaatggctgggcttgaggaatcgttttccagtccgccataccgccttatggaggttatctatataggattttccaattagcatgctaccttaatccaaataccgaatcgaaaggcaatattgttcaagagagcctagataccttacctctctgccaaagaagcaaagtacgggagaagtaggattgaggagacggataacagaacccacatttctcggctcatcctgtttcactaattcttttgttcgaaactcctctcgaatatcaaacagatcttcattaaccaccttgacttttttctctttgggcatctcgggctgttggagaaaactttttcgccgatcttttcgccaagcatcttaaaaacctaagggtcgccctcttgccctgggaaaaatcttaccacaccatactatgatatggtgttttcacggcaatactatgctcagaccctggttagggttgctggagttgtacgtgacggcttcttggtgtcacttcttttggggagtcttggcgtccccccccttcgtgacgggaggtctatataagaggtcccaactcccctcctcctttcttccaccaacaaccgacaactcccacaaggttagattcacagaaacatccaaccttcctacaggcaattttcctatcacggcatatttgtcaccctctatcttcaccacctcagcaatgtcttccctctctgtccttcagatggctgttgaagccggacctcttattcaccaacgttaccacggagtcaacctcagtcaatacctttccatgtttgccaataaaactttttccttcaatggatcggatggagctcagctcagtgttctccatatgcttccggccgactggaagcctgcctctggaccccttagcgtttcgtttgattcggccccccataccgtcatcgggactcaccatactatccgcagccgccctgtttataagcttagcgacggatccatcctcctcgtcttcgaccctgtcgttaccgacgtcgattttcagctcactgatcgctctttcgaggacccccagcatgagattaccggcctaactcgttttctacgtcttgctacagatgaacaagacttcgcctctcacggtgctagtcttagccttattcgcgctaagggtgtcaagcgccccgccaatgcgtaagtacaactttcctgacctacaggcttcatgcgctcatacagccacagrtttatcctgttcaggaccgagaatatgaggactatcaagactcggtatcctcagatgggcaacaatgacatttgtatgcagctctcatcccttttattaataagctcttcattcttagtatcatttcactaatagtttcaatagcaaaaattctyggtcgcatgtggcagaattcctcgaacgaggcgaaagaggtttataaggtataccacctaacctaactgttagttgaaatagctaacacattaataggcaagggcccgtcaactcgcgatttcgcacaagttggtgaatccgaactacaaatacaccccgcgccgttctcatgaaattaggaggagggcctatgcgagaaagggtctttcgaactatgtcgatctttactctcctggagctgaaagagatgccagagagagggctacaacccaccttgggacccccactgcacagtttgcctccgagccgagcaccgtcaccctcggtgaaattatcgaggaagcggagaaggtcttgggttcctttggtctgattaattgagtgatcttagtggagggggcttatgggttacttggatgatatacttttatgggattgatgatcttgtttgctcgtgatgggtcgttagctcagttaggtaaatttgtatgatccaaataaacaaaaattatcttaaaaatgagcccgagaaatccaaacaccaagtcgtgggcaaggtaggagcgagtagtgtatggcaaggggcagtgggaacttaaccattcgagtatggatacagcaacccagaaagtttatacagtacccgcgcggccgaatttaagcccgacaccaccccttataatcgtcaccagccgaccaataagaggtggaactcagatggaaacttagtaaacacatacggtataccagatagaaaaatgtccaattacatgcatcctaaactatcgtatacctgtatcatacacagaaaatttgaaatcaaacacttcctataagaattaccacccccaaattaggtgtcaagccccaattcagttatataagtatatcatttacccattaaagttatcgtttcggccatgaagaataacaagctcagattcagcagttgaggcatcacaacctattcttttcaatggtttatcgagttacatcccccagcttggcaaggccatttctgcataattattcgttggctgatcactaccataagaagtggtgtccggctttaattcggccgcgagtatgacctagtttgagtggaaatcccctctggaggtcaaggtgtcgcgtcccccgcattcccacacattactaaaccgggtgttgtgggtaagtctgtcgaactcactttaattggctccagggcctgctcgaccacttagggaacagtattgctccggccactgtgccaagagttcgacagacttttttccttcaccaaaaattagcgtctatggcctgttcagttgcataactcgcgttgcacacacgttaattcaaccaaccagaccagagttcgacagacttttgcgcacaccctaagtacaccggccgccggccggtaggttattcttagtaatggcatcagaaaaggcggccacgaatcgatgcccagtatgggaggcaccagccacgccccggccagctaacacactatcatactcatcgggatcaaaggtctgtccccgaggtatagtagccagtggaagcacggcaaccactcccctccgccagccttgacgataggagaattgtgttgataggtatcgcttataaatcaaatatccttagaaaacaagattactagtagcgctgagagaagcaagctgcaatctttacaaggtgttgtggctaggttgtttgtgcataggtgaaagtcggggacagactatttttttgccgaatacaatagaaaccggcgcagttcggaatgggctagagaagcactggaaaaatcgtgggtatagtgcacggccagtagtggcgagaccaggcttaccccataatcagacctcaaatttaggccaaccaaaccatgagaataaacaccggccgcaccctaatcactagcggtagtgcgacttttgtgagtttatggtatggcggaggaacagaggggtgtatcatacgagtaaaccaggtggcaaccatgaatgcagccgctaatacgactaaaaatgcgcgaggtgcgccaggtgttggaggtcgagtgtgtgtattgagagattgaagagaaagtaaagaccgaggaatataggggggaataagaagctatatattggaaacgaagagcgttgatttttctcaccgtcctatagttacatgcgccagagacagccattcttatatccgtagaaaaccgcttgtgaaccaagtggtgaaaagagaagctccgagctgagcatactttcgtcctattgaagaaatactggagtacttcagacacaagatatagccaaccattgagtgataatctgtgacattatagatggcaatcccatatgttgtattaagttgcactattataccaagaccccaacactacactaaatttttgcctttctggacccttaattacctcctcctgcactcataaatggaaagaaagaaattgccctttatttttctcccttttgcccacatcccgacagctcttccgcccagtctggacaagcacctgggagcttacccggggactaaaatcacctatttgcttgatttacacttattgcctctcactctatggagtgtatcatctgcattttcaggtgcacttattccatttggttgatagaacagctatagtatccctttctcagtttcctgactaggtacaagataacagaagtgctaattcaataaagatcccagttgtagatgacaagtatagcaagtgtcttctcatgatttgttttccagaaggctgtgattcagagcttgccttacctattcccttaagatcaagatttcttcttcatactatttgagacttatgccatacatttgcctaaaaaaaataggtggaaacaaagggatcttgaatgcctcccattgagatgaagctaagtttacttaagctcctaagaatagacaaagtcctgttggtaggcaaaatttaatacctaaatatacaatactagttgtttgaagttgtactctctttggcatcaaattgacagtaacagtaatgtacctaaaagagcaagtacagagggcaaacaagtaccttctacaaattaaccacaatttttcacattccatgctagatacttactcctctgcactacactctaggaacttttactcaacaatatttggtgcagtattcataactgtttggatattctggtagctttgcagggagactataagaggactatatcggataatactaggattcacaatcaattctatcctgtaactattgagaacttgaatgatttgttttaacaaaatctatagcttggacaatagaaatattattttgttatcttagtatcagaactccaggtgaaacagtagactgctagtacagatttcctttatagaactatatagaataatgagttaatagaaaatcgataaaaagttgaagtggactatgagaatccagaaaatctgactaaccagaagcgattgccagcatgggggcatactgtagctacaaactaagttatagatataatcatatatacatagttttaaagtatcatactaggaattttctagtatttcttttcacatttctctattgttttagctatttaaaatataccataggtatatgctagcagatgtaaaactgccaagagttagagtctgtttggataagactattctattctttttctttatataaaataactattatgtgatggaaaaacaatagtattcatatgtgaaaatgaaactgaatagaacagaacttatgaaagggtcatattcaaacaaactctaaaattatttaacttcttttccagtataagtcatattaatacaaaaattttaatacaagtaccaatcctgtgaaaagttactagaaaataaactattatatactattatacctatcaatagtagtgaacaagtcctgctaacttcgatataatttattgtattatagttgactactaggtaatctctactctctttctacaagttttttgagtttttctactctactctagtaaaagcctcaaacagggtcaaattcttctcaattattaaatattcatctatattaatacctttattactattactttctattatattcagaaaaatatagatcagattatcacaatatattttataatagtatgatacaagtacttaatagtatgttatcacataatactaataataatattatcatacttatagagatatagtatttaggttttataagttctacaagtaagatttgctgcctttagcctctatagaatgtataaaacttagtggctatatttctatacactcttggcattttttggcgcattgtgtaagtcttggcatgtttggtcatgtgactatttctttgaaaagctcttactttataaaagggataaataatgctcacagtctatactaccaaaacttctctcaaacacccaacaccagaacactcgattcgacaacatcctttgcacacttgttagtggatctgctggtatcagtctcaggcccaagattggcctctacacatataagatactttttttttttctttgtcaaaatgcctttaagtatcttctctctaccaggaaatccaaatttaatacagatactaactcaaaaaattagatgaggatattagactccgccgtgaggcactattccgggctggattgatgcttactagagcaatggatgaaactgcctggaaccctggcaatctaatggttggaatggccattcataaaaatccaagcattactgtggttaaagcagttaaatcatctgaagttatacttaatgaagcagctgtgagtgataatatattacgcaagatggatccagtaaatgacctccagccactggcaaatattcacaatacacttttacagcttggagcattaattactataaaacaagaactctatgatgcacttcaaaatatcaaattacgccttcaggaacttcaagcaaatatgaccaacaacattggccaggccatggcagaagttcatgactttatctgctttgtcacatttgttaacttgattcatccacaaaactgcatattaacctctgaatctatcaaaaatgctgagattcttgcttccaaaagtatattttcagaagctcgtcaggcctttgttgaactccgtcaagcttatgacctgcaaactcctgttccaggataccgtctgagcaaagctagagagttacctgcatttactagctgcattatggaacagtggcacagaggttattcttctcatacagctaaaactggaatagtaagtagaaaatacctctcttaacttagtacctatatccaatatactctactgtatattatactaacatcagagcataggaaattattccattccatatcatatttggatggtggagtcagttcaattaccttatttcatctaaacagtatcattaactaacaataaataatcttggtttatagtcaattaccaacgcaagtttattagtagtgctcaagagaaaactacaccaattgtggaaaagcttatcagccaagcctatatctcgaagtccaagatgccagaagatgaggcccagcaaccatggatgcaggaagataaggatgccttcaaggctgcaaccaatctagccatgtctatgactatac

SEQ ID NO: 61 - DNA, 205 bp, Tuber magnatum
tttggagctgaacccaacaagtatatgtggcaaaacatcgcatctctctatactgccgctaggaatcgtgccgagcccggtcttgtgctcgaagagtttatcgagaatgaattggtgaaacacggtcacccaaggacgccggagaagctcttgcgcgaagctgggttcgttcccaaggctccaattgrcaagggcgccgtcgata

SEQ ID NO: 62 - DNA, 1041 bp, Tuber aestivum
atgtcaactctcaccgctctggagatggctatcgaagccgaggcatttattcaccagcgttacggtgggatctcgttccacgagtatctccccttgctcaccgatgggactttttgctttgagggatcggacggagctcagctccaggtttcccatatgcttcctaccaagtggaaacctacccccgagccgctcagggtttctttcaatccgtccccccataccatcgtcgggactcatctcactcttcatgaccgccctgtctacaagcttagcgatgggtccattcttgtcgtttccgaccccaccactagccccaccagtttccagctcacggatatcactttcgatgatcctcagcatgaagtcactaatgaaaaccgttttttgcgctttttcatcaaggctggtcaagacctctcatctcatgagatcagtgttcgcaaaattcaggagaagggtatcaagcgccctgccaatgcgtaagtacaactctaccaacctacaagcttcatacgctcatgcagtcacagattcattttgttccggaccgagaggatgggttccatcaaatcccaatatccggggattggaaacaatgacatctgtatgtagttctcattccctcttattaaaacttcattcctagtatcaccttcactaatagtttcgatagccaaaattcttggtcgtatgtggcgggattccccggacgaggcgaaggaagtgtataaggtatagcatctcactcaactactagttgacctggctaacaaattaataggcaaaggcccgtcaactcgcgatctcccacaagttgatctacccgaactacaaatacaccccgcgccgtccttatgaaattttaaggcgattgaatgggaatgtagatcctgagatcgtaaacgaacccggaaatacggttgcaaatgaccttggggcgctcacccaaaggtttatcgcccatccgaccgccgccactctcaatgaaattatcgcggaggcagggcagatcatgagcgcccttggctatactgagta

SEQ ID NO: 63 - DNA, 5520 bp, Tuber aestivum
cgagacaaaagaagagatgccgcaccttgccgggagagccaacctcaacaccctagcaaacggtgcgattattcaagttcttgcggtactcgagcacccagacactcgcccttgcccacaaccgggagattcgagcgtagtattgtatcactgtaatgaccttcctaaataggaaggtgatttactacacttcagatggcttggctgtcgttaaaggtcctgcattattcgtacaaccggcactctccaactaactctcggttacacgcagggatgtcacgcagaatcctagtaggcatcacgcagactactaattattaaggatttttttatccgctagcctttccgcgaccccgatcgggggaaatcagtttaagcattcttgtagacgtgtactggaaacttcacgtcgatgcccccgcccctgggcgagcaaaaatataatgcgagaaatgaatataggagcctcacgtttttcccggggagccaaacagcgatgataacactataccctatctcactggctctaggtgtctaccctaaagttagaggaacggttaattttggttaacctgcgaaattatacctgcggtagagacatgttttttctcctttaactgctagaatgtagcttcgcccgggacaagtagtctaggtccagactggggactgaggcagatggataaacggatttagtggctataatacgatagaattgcgtctttgaatgaaatgccatagtatatgaaaactatatgaaaactcgtagtactccttttggcaagcttcactcatcattgtgcataaagaataaagtccgaaactagttttaccccccccggcaaggcaacttcggtatccgaataaaatacaggtattaagattcgctcccatcccatgacttgtaaaatcctcagctccgagcgtctccggatgttttcacaatgtatcactcgatattttattttgcgtgattgttgcacagagatggaacgggagacaaaaggattatatgggaggacgagtggaaggaataacttgcccgaacttctaggtgggataaacacaccgctcaagtgcgtatgatataacgatcgtagatgtcaaactgctacttttgaacatctctgatacaatcagtgactgtcagcgaaaacccgaacaggatttactccaacatggttctttttttttcctcccctttcccttccctcctccccagaaggaaaacttggactcgtgtctttacgttagatcaattatataagctaagcgttcagttctcatgttgtatccgagtgggtatcgtcccttcgcttggtgtcgaatttcacagattttaaacgaaggaaccgttagcaagaaagtatcgtggaggagggaggttgcaggaaaaatgagagtgcaccagtactgtgctatgagccactagcaaaaaaaaggagattatatttttattatgatacttcgaagagtctggtaaaggacctaaagacttctccttctttggcatcactcccaaatagaaaaaacactacgaaggaattcaccaagtgggccccgggactggcctcgtattcaattacggtatttttgtaggagatatttcgctataatctagaatccccttcacaaatatacagagacgtgtagtatccctcctctgatgatacaactcgcccttctgcgaaacaaaacaatcaatactctttcccccgtatagttcccttgacaccttggtttctatcccgggagctcaagcgggagcgccgtgcccttttcctctaacgcccttctcttgcgatatcttcatacagactcatgagacccagtgtgctccatttaaatcccactcatcataccaagagtaaagttccgggggttatgccttcaaatacgtccccagcgactttccaaattgccccctagtcctgagcgggaattctaacggcttagagctggttagagattccaattccttcgctgaaaatcctattcaggggcgttcacactcacaatgcaaagctcgtccttccgttataccaccaatatctttcccactggtcccagtatggtctagccgagaacctctctgaaaccatcacgagaaactaccagcaatgctagccacaattccttaatctccagaccctctcgcccatgggctgctgcatagtacttctcctttgtttcggagctccatctttcaaatagcgtccttggcagctgcaccccccccccgaataccgccatactcatagccaaattcgactgcatgcgccagcgggtagccgaacgcagggacatccatcggatcggccgggagtgccgaaggatctatctccccctgatcggcagaggagaaagctacttcgaattcttgttttcgagtgccggcttcttgggtattgtaagcaacggcttctggatggttgggtttaaggtatacttttccagcacgcaataccacattatggaggttatctatataagaatatctcattagaattctatctagatctgtatacagaatcgcaaagcaacattgtcagaggggtcttggatagcacacctctctactcaagaagaaatgcacaggaaaaaaaagtatcgaggggacaaatagcataacccacatttcccagttcatcccccctttgcccaaaactcctcccggataccaaatagatcttcattaaccgccttgactttttctgcgggcctctcggactgtaaagagaaaaaaatttcgccgaacattgcagaagcctaagggccccaccctggcccggggagaatcctaccacaccatactatgatactcccgcaataatactatgctcagaccttggttggggtcgctggggtcgtgcgtgaccggtttttttttttttcgtacgtgaccggtttttggtgtcgccgctttttagggtctcagcgtcccaccttcgtgacgggtggtctatataagaagtccaaactgcccccctcctttccctctccttcttccaccaacaaccacaggagcgaatccccctccactcgatagcaagttccaaccttcctatcaacaaatccccctttggcagcatattctccatcacctcgccagcaatgtcaactctcaccgctctggagatggctatcgaagccgaggcatttattcaccagcgttacggtgggatctcgttccacgagtatctccccttgctcaccgatgggactttttgctttgagggatcggacggagctcagctccaggtttcccatatgcttcctaccaagtggaaacctacccccgagccgctcagggtttctttcaatccgtccccccataccatcgtcgggactcatctcactcttcatgaccgccctgtctacaagcttagcgatgggtccattcttgtcgtttccgaccccaccactagccccaccagtttccagctcacggatatcactttcgatgatcctcagcatgaagtcactaatgaaaaccgttttttgcgctttttcatcaaggctggtcaagacctctcatctcatgagatcagtgttcgcaaaattcaggagaagggtatcaagcgccctgccaatgcgtaagtacaactctaccaacctacaagcttcatacgctcatgcagtcacagattcattttgttccggaccgagaggatgggttccatcaaatcccaatatccggggattggaaacaatgacatctgtatgtagttctcattccctcttattaaaacttcattcctagtatcaccttcactaatagtttcgatagccaaaattcttggtcgtatgtggcgggattccccggacgaggcgaaggaagtgtataaggtatagcatctcactcaactactagttgacctggctaacaaattaataggcaaaggcccgtcaactcgcgatctcccacaagttgatctacccgaactacaaatacaccccgcgccgtccttatgaaattttaaggcgattgaatgggaatgtagatcctgagatcgtaaacgaacccggaaatacggttgcaaatgaccttggggcgctcacccaaaggtttatcgcccatccgaccgccgccactctcaatgaaattatcgcggaggcagggcagatcatgagcgcccttggctatactgagtaagccacctcggcagaggggcttgtgtgttccttgggtgatattcttttaatgggagggagggttgtgtctgttcttcatggatcactatcttagttagataaatttgtatgatccaaataaactaaaattgctctaaaaacgtgcgtgagaaacccaaacaccaaaccatcaattaccatcaagttcagtaggggtgagtgatgtactcgtgcgtccgagccaagaatgggcgcctctttcgcaatagctctcccaaccctgtaaataatagtattaatgctgttttttgagtagtgcatatgtggccactacaacatataataaatatcatgaacactcatacaaacatttccactattatactaaatctgatacattatgatacgcaataattgctcctcttatagtgcttgtaagactttattccttcattgcactattctaaatgttaaccttgaagttaaattttaatcacaggtatatactgaccttcaggagctttatctattcatccaattcaatgctctaaagtcttcttaagtattgaaaatatggtctcacatatgatatcattctcaccaggtgccagtattttgtgctatcatcggagctcacaaacagtacagccactatatcttttatggtctctgggagcttttccgaaaggggcgcccattcctggctcggacgcgtgagtaccctatgggccacagtgccgaacctacaaaaaacccctaatatgcctagacaattatactgtaggatagctcttgctttcaactaatgctaaccaaggggccgctcggcagtactcgggggtgcatgaaagcgacgggcaccccacgacgtatgtggcaccagattatggtgcatgttcaagcactagtcttcagctctctggtcctgaccacaggcagtttctaaatggaaaattacaaaatagtaaaatgaagattaaatgttggtcaaggaagactatagcaatagtgaaaatgaacagtgaaataaaccacaggacagcgcatggtaaaaagagcaaaaaaaataccaagaccacaaatacaaaacaattacacaactcagaagtgggggaaaaactcttcacttcagctaccactctctcaactgttcaaaatgttgtaggatatggtacaatcttaataactctgtatgaatatacaatgtagtagaaagagtgtattgtgggtaagctaacttattacctcctatgataatcaatgagtatagcaactacaagaaacatatttgaacatcctgcttattcaacttaaagataagtagacatcctatattggagtaagggttaagtataccatagtcctttgatcataagcgaatcatct

SEQ ID NO: 64 - DNA, 20279 bp, Tuber aestivum
  misc_feature (8849)..(8868): n is a, c, g, or t
atgtttgggtggagggttgcgaggggctacgggcctggtagtgatcttctccgagggttcatgtttgggtggagggttgcgaggggctacgggcctggtagtgatttctctccgaggatttatgtttgggtggagggttgcgaggggctacgggcctggtagtgatttctagttaggataccgggtgggggtggtgttggcttgccactgggacgcaatgccgtataggcgcacatacccttggggtggagtctagtgtttcacgaataaaatacgctaaccagtgttggaatgtcggcgccatgcgggagatatactgtatcatagtctgacggattgggatatggatcgctccgaagctcgagcaaaccctttgcatgctagtctggaaattaggctgtcatcgaaggaaggaagtgtacctaatatctgggtgataagttgagttggaggaaggtggagtaccaaataatcattttcagttcttggttaccgttggctcgggcttcacacgaggggagtagggattacgaggtttgatgccgctctccattttgctgcagctgctagacggcccgcttggcgctttgctatgactttacaatttctgtggaaatgctacctgagaagacaggcggactgcaggccagagatttacattggtcaggggaaaaagtatccgccttgatggctttcatgtttaatttcagggtgataatagtacaaaggggtcaagcactggaggactacaggagtacctgcccacccgccaaattttcattgctcagatattaatgacaaactctgccatgtatttccgcgcatgccgttaacaccgacaacactactcgagcacatgtctctataaaagtatactattgttagcctggttagcactagatatttcctggcatgcccgccaccgaacaggtccatctggaatgcaggggctttcaaacgggaacacagaggaatggcagggattcatgcaacctaccctgtgatgcggagaaattagccggttaatgcgaaactatgcccccgggtaactctcgagatatcatgactcccatttcatcatccagtagattggccaattcctcggcttgctttttggcctggtgagcctccatatccagcttcttcttttgaagttcaatgctttgaagttgtttggacaaggaatcctcaattttctgcttttccgccttctgcttgtcgagggcggactggacagcacatagaatagatttgacggccagctgcttctcctcatcgcccagaatctcggccatttgcagcgaggcacggatgaacaggaggagtttgtcggccggaacgggtattcgatggacagcgctatggcggaggtcgccgagactctttagcagtaaggggagctgagccgtgccgccaatctgttcgagatgaacagtggatgggctctgctcgaatgtcttggacaactccttcgtccacatggaaagttcggccgagtgagcgcaggtccatcgcttccgagcgagcagctccgggacgtgacgtttggcaaacctgaagcaggcaccttcgagaatgcattgcagcgaggtagtgatgttgtgctggacacagtaggggagaattggagagggatcggtcgggttcggttcgaggggacgggcctcaacgggctcgctaatggcatcaaagaaatcgggactgctactatcgaggtagaatgtggggttagcgccatgattcgaaacgctttgaatccattgttggaaggcaagagacctctttgaagcagtgatcacaggcggtgcatcccgcccaacgggttgggggggagccggggtaacggtgggggccgtagcagggccgctcccgttcatgtatgtggtgctgccgttgaccgagccgttcctatcctccccatccctcaagtcgcgagcgcgggcggcgccttcatcattcaacctctttgcgagtccctgagcgcacagggatgccaaggacagccttatctcctcctcggtgatggtagcgaaaccggacttgaacctgtcgcgcagtatcttctgcagcaacaccttcttccccacaaaggtaccgttatcgccgaagccgacctcgtacgagcgcttggtgatccaggcttccagtttttgtgcaagcgaatggggcatatttctgtttccgtttccgtttctttttctttttctttttttcccttccaatcgattatagatgcggcggggagagaggtgtgagggtatctcaaacttgagctgtgagagagtgtgtatgtatgagagagtgggagtgagagtgagagagagtgtgtggggagtgaattggattgaagtgaaggtgatggtgggtggtggtgggaatgagggactgtaggtaaagtgcagaattgagttcataggtctagattaccatgtcgagtgcgctgtctatacagtacactcgtacaggaagggattgggtatctgtgtgtgtggtgtgttatcatccgagtttgttgttgtcccttcctaatttatcattttattgtatagggaagctgtggtacaagtacggtagggcatgcttcgtttgtgtttttggctttttgcacttgcgggtatggtagggtacttgtacctgtacgagtactgtactcgagtacttttatcgtactcggccgactctttctactttgcggggagcgtactcggtgcaccgtacatgtgataccgctgcttgtacgagtacgagtacagtgctgtactagtatgatacagtaccttagtgctgcatatgctctctatcgtcgcttgcataaccccccaagtctctctcgtgtgatgcttccttccatactatctcatacgagtaccgtatatacagtatcatactgtggtttgtttccggttgaagcaattgaggcttacctaataacttgaaccacatttcgccgcacagctaaatccgggaagaaaggggaaaaaaaaaaaaaatcaacccctccaattcacgcctcccccccccacaatccctacaatccccctccgacgaacccaatgatcgctacccgtcgtgcacttcgatacttacggctgtcaagacggctctcgcttcgagatcctccagtccggtcccgccatcttcacacaccacctccgaaccgttcggaggactcgtcgatccattctcctctcttgttggccccccctgcggtgacgataaaaccccccgtggaagagtatgttcgacagatacgcgagcagtatggggactgcctccccgccggtctgcttgaagagggtgaatacaaactctacgagaggtactatggccgcccgacccgactgctgcagcctggcgagacgcttgaggtgcatcccgaggaatgggaggaacttggggctactatgcacgatgaggatggagcggagatttcggaggaggaggccgagacggagtccgactctgaccatttcccgcccccaaagacgcctggggaggaggacttgtacgcacaggtcgcccagcgaatcgcccaccccgaaggggtaaaaacggaggagggaggtcaggaccacgataaccacttccgtgcaggttctcctaacctcttccgcacacacccgctcacgtccatcggtcgcttcgcaacctatccgttcgccatccaattcccgccatcgatgaagcaccggacaaagtctgtcatcgccaatgtccccacctcgcatctaatagactcggcgcaagccaccctcggcggttcaccattcatacactcgcccatctactcgaagaagcggagtaagcgcgagctgcaaattcccttgaacccgactactgctcaattttcggatatggatgcgcatgtgtttctcacggccttgatgccgggcctatacgcgcagtgcttgagttcgttgaccgagctgcggaggcgattgggcagcgattgggtgctcggtggggatcggggggagcaaggtgttaggaatgtgcttgatgtggggagtgggggcgctggtgtgcttgcttggcggagcattgttgaagcggaagaggagagaaggagggatgaactctccgagaggacggggaagccgttgccagcgggggagaggagcgggctgcgcgctgttgttgtcacggggtctatcgctcttaggcggaaggtatcgcaattgttggacgataccatatttattccccgaatgccggatatctatttcgaaaacctacaccacatgccaggcgtctcgacggagaggaacaaggcccagccgagaaaactctacgatctgatcattgctacaaacagactcctccccatagtgaagtctttctaccgccaccaagtcattgagcaactctggtcgcacctaaatccgaatggcggcgtccttttgatgatcgaaaaaggcacgccaatgggcttcgaggccatcgccagcgcccgctccaccatcctaaggaactacatcaaggacgttggtgaagccttccagccccccccgaccgcaaatgcctacggcgaccccaccgacccctccatcgaaaaggagcccggtgccataatcgccccatgcacgaaccacgaacaatgccccttattcgcctttggccccaccgacggcacccgccgtaaagactactgccgcttcccacaacgctatgtacgccccggctatctgcagaaacttatgaatgtgaagcagtcagacaagaaccacgaggacctcgagtactcatacgttgcctttcgtcgtggcgccgaccaccgcgccgacacgaagaacaagatcaacccagcaatggaggacttcgccccaatccccgtcgatggcgaacccgccacgtcgattctgggcccgtacacgatgccacaactccgcaattacagtctaaccctcccgcgcatcatcgtcccacccatcaaaagccacaagcacgtcacacttgacgtgtgcactccgacaggcttagtcgaacgttggattgtcccttggagcctcggaaaattcgagtacagagacgcgaggaagagtaggtggggtgatctctgggcgctcggagcgaaaacccgcacaaagcggaacataaatattgggggcgatggcggcaaggtcatgtcaaagttggttgccatcaaagacccccatggcgatggggtgagggctattgtcaaggataagacgcctgtctacttgaagaatcggaagaggaagatgaaggagttgaagaaggccacaaggcttgaaaggaaacgagaggctaaggagaggcatgctgaaaaggttaggatgcacaaggaggagaaggaaaagaggcggcaggagaaggaggccaggggggggggggcacgaggaaacggttaggatgcataaaggggggaggggaaagaagctggaaggagtgtaaatgagggaaaagggttgggtggtgactaagagagaaatttcactatgggatcgggatgtaactgtatttcatacctggagcgtaggccgcgggtggatggggtgtatcatatatacggggcttctccattttgtaaattatttatccggttccaaagagcactatccttaaagcctgcttgggtgaggccatcccgttcccttgccccatacaaataaccggttcaccttccgtcacagaaacccagagagccgtttctacaggtaaaaaaaattgtcacaagacaaaatgactatccttcctcccattttttgtcaaacagacccttggtttttattacaatagtggatgggagaaggggtaaaacgcccggcacactcactccacgccccttcctccctcatactctcagcaattaccgaacacagcaaccgcaacttccatcacgcatccgacggttgtgaagggtggggaggaagagggagaggaggaggaatttctgtgaggatccaccacgttcacagggtgtattttccaatgcttttgcgggcccttgtgtgccgatcgttgggcgtgcatggttggagatggtcggttttaacgaccctatacagcacaatcttcgcccacattctggttgatgttttgttcttggtagtccacttctcgatcctcctcgctgccctctctgccgcccgtagcatcctgcgatggctcccgcacgtggccggcgcgggctgacatccagaagcagctccaccatgcccaggctccccctttcggcctggagacctcttcaggcattctcaataggtgtgctcccgccgagcgtggtttgggaaatatgatagatatgatagcacctctcctcgggaagtcgagtgaggtttcgtgattggctgctcgtagttgttggagcaatggatgaatggaagcgagagatttgggctccacgatggcgatttcgtgcaagtcggattttacgggcgtcaattgtgggtcggtcctagttccagggtctctcccgtttgcgggtagttaactatttgtatttgcctatgctgaaggctaggcgggcactgttttgtattattgcgagagcgtcggtgggtacgttttgacgaccctatctggcagttatcactagagagtataatacgttcgaaagggcattctcgaggatcgataaggtgctcatttgtatttatcccggcccgcagatttctgatggattacctgtgagccaactgagaagaatcccccagatataccatccagtgggtatcatcccccctccagtacacaaagtgatgatatgccaacgagggatgatagcaaatcccatgggcgtccacccccagagcagcagccagtgaaaacggtaactcccccagcactaccaatcctgtactgtaccatacccccccccccccctgtacccggggacaatacgcaagcaaagcatcccatatcgaggtagagcctcccttgccagcctccaacgcacggactcttggagcttcgcagaattcccttcgctcttcccggggtacaccacgaagcggcccgccccggcaaaatccaacctttcacgagcagaaacctggcaaacctcgaccaacactacttctatctgtgggctgccgtgggaagctgctccgctcctcgacgccgacagtcaaactattttggcaaacgagaacgacatttgcccccctcccccccgccagcggataacgactacaatctggaacagggatgctgtgataggggagtgtgcagaatgggagcttgagggcaattgcatgtcaatgctacctccgagtgatcgcgtgtggatgggaatagttaagcaagcggaagaggtccggaaggaggaaaacacaagaaatctaatgagtcggtggccaggcggagggtgggcacccatctacccgtagtttatttcaacaccgtaaaaaaagaaacgaatagaagatgaatatgtgtttaagaccttgcagtccgcctccctctgcccactttaactcaccgcgcatgcatacagtacgaaggaggtaggggaaagtctggcgaagtcttggttgctaatcccattcaaattctgtgagccacgttcgccagactttcccttacccccttcttgcattccattccactccgtctagacctccagccaagatccgtgagccaaggtccgtgaagcccacctctctcgtagcccagaggtgggatcccaacgtgttcgcagatctgtgttatatccgagctcctacggcattctagattagctgtccggacagtatcatacggtccatctttgacacgcttaacccactccactgacgagtccgtagcagaagagtctcccgaattgttgcgactctttggtccgtctgtctatggcatctgccatggcccgagttccccgttagttccggtgattgttttttttttttggtcgcgggggtggagggggtttgtccaagctacggtatcgctataccgaagagatgaggaatgactgcagcgacctgataaatagtggatacaagaggtgtagtgcaaatgatacagcaggttagtgaaggcctctaagtatcacgagggcccagcaggagaggcagccagaggggagggtgatagacaacctactcgtccatagcagattaaaagtcatacggaaagtcccagtccatctccagacagacccggtgggaaggtggactatccgattgtgggctttagcctttaagttacagtcagcttcataaggcgattctgagatatggacccatccggtggacgctttctccgggtattaacccaatggcacggtttcagggatatgattaccctcagtgaatcccaattttggatacatgctggggggtaagatgcgaaacgggtagaatagttctgtcaagctaggaatgcaattgcctgttttttcccttccttctaacccggtgtgttaacaagtttgccacacccatccaaacaacaatatgcggttttgtatcgtgcagggaggaggcggagagcccggcggcgaaagggtggataatgatggccgaacaccgctctcgcatgccgcttgagataggcatgaaaaattggtaaagatactacccgggaggggggagatctaccccgaaaaggcagataatgatggcggaataccactctcgcatgccacttgagatggacgtgagggagtggtgaaaatactgccggtagggaagaggttggccccgaaaaggcagataatgatggccgaataccgctctcgcatgctacatgagatggacttgaggaagtggcgaaatactgccgggagggagatgtcggcccgaaaaggctggtaatgatggccgaacactgatcgcatgccgcttgggacagaaatgagggagtagtgaaaatactacccnnnnnnnnnnnnnnnnnnnnaagataatgaaggccgaataccactctcgcacgccgcttgggatggacatgaagaagcggcgaaaacgctgccgggaggtcggccccgaaaagactgataacggtggccagacactgctctcgagtgccgcttgggacggggatggggtggtggtgggaatgctacccgtgcgtgaagatgtctactcggcaagccaaataaagaaggccaaacactgttctcgttcgctccttgggatgggcactggaaactggtgaaaatactacccgggaggaaagagctcgacccccaaaagccagataatgacggccgaataccgcccccacatgccgtttggggtagacatgagggaggtgaaatgccactcggacgggaagatgtcagccccgacaggccggataataatggccaaacatcgctctcgtttgctgccaggtgtagctataataggggggcagcactactacactctcgcaaagcggtagcccctaagttagcacgatataaggcctagaggataccccctcgctgaaatcacccagcttccttttcggagcacactactcacaggacttgcccaccgacagacccattgcagtaaagagaagctcatgaaatagggccgtgaatgcgcatacatgtcgccatacccatgcgagccttccggagattcaggttcacagtcgagtggggttttgcggtttttctgtttgtaaagtatcgtacggtagagtttggtccgtgggagaacagtttctatcgtcgtgaaaaggtctagtgtttagcttgcattttttagcctatcatattgaaacctcctctggctgtaaattcttacccgggcgttttttctcgtgttcctattgcaaatatttttgttcctttgcttccctccggagtagtatcttaccacctcctagcgtctatgcgagcagggtgcaagagcggtattcggccatcattatctgccttttcggggccaacctcttccctcccggcagtgttttcgccacttcctcatgtccatatcaagcggcgtgcgagagcggtattcggccttcattatcttcctcttcggggtagacctctcccctcccgggtagtattttcgccacttcctcatgtccataagtagtaacagatgagagccgcggtgcttccccctatcgtctggtctgtcggccgacctcttcctctccgagtagtatttccactattccctcattcccacaagcagcagaaggtgagagtggtatttgggatctcgcttatcgaggttaaaatccctcacacgagctgtaatttcacccccccttatgcccactccaagctgcgtgcgagagtggtgtttggccgtgattgtgcagttcgtctgattgacattttcccgttcgagtagcattttcatgaccccctcatgtccattccaagtggtattatagagctggcggacggccgtaattatttaacctgcctggttcacatcgtcccgcctgagcagtatcttcaccgccaccccatatacaccccaagtggcacgcgagaggggtgttcggccgcgatttcccggcacgttagggctgacatcctccgattcgagtgatattttcacgactccctcaagtccattccaggaagcgctcaagagcggtattagaccgcaattatctggctcccggggttgacctcgtcacacacgagtagcattctccccactttctcgtgtccatcctaaagagcgagcgagagcggtgcttgttcttcgttatcaggcttgttgggcagacaccttcacgcaggggtagtatttccaccactttttcatgtctgccccaagcggtatgcgagagcagtgtctggcctccattatctgacttgccgaggctaacaacttcccgtccgagcagtaccgtaagagatcgaataaccagaccacatcctatttagtcagaaatttcctttttctggagaaggggcgaaatacacaccgaaaacatggcttccgagacaaaagaagagatgccgcaccttgccgggagagccaacctcaacaccctagcaaacggtgcgattattcaagttcttgcggtactcgagcacccagacactcgcccttgcccacaaccgggagattcgagcgtagtattgtatcactgtaatgaccttcctaaataggaaggtgatttactacacttcagatggcttggctgtcgttaaaggtcctgcattattcgtacaaccggcactctccaactaactctcggttacacgcagggatgtcacgcagaatcctagtaggcatcacgcagactactaattattaaggatttttttatccgctagcctttccgcgaccccgatcgggggaaatcagtttaagcattcttgtagacgtgtactggaaacttcacgtcgatgcccccgcccctgggcgagcaaaaatataatgcgagaaatgaatataggagcctcacgtttttcccggggagccaaacagcgatgataacactataccctatctcactggctctaggtgtctaccctaaagttagaggaacggttaattttggttaacctgcgaaattatacctgcggtagagacatgttttttctcctttaactgctagaatgtagcttcgcccgggacaagtagtctaggtccagactggggactgaggcagatggataaacggatttagtggctataatacgatagaattgcgtctttgaatgaaatgccatagtatatgaaaactatatgaaaactcgtagtactccttttggcaagcttcactcatcattgtgcataaagaataaagtccgaaactagttttaccccccccggcaaggcaacttcggtatccgaataaaatacaggtattaagattcgctcccatcccatgacttgtaaaatcctcagctccgagcgtctccggatgttttcacaatgtatcactcgatattttattttgcgtgattgttgcacagagatggaacgggagacaaaaggattatatgggaggacgagtggaaggaataacttgcccgaacttctaggtgggataaacacaccgctcaagtgcgtatgatataacgatcgtagatgtcaaactgctacttttgaacatctctgatacaatcagtgactgtcagcgaaaacccgaacaggatttactccaacatggttctttttttttcctcccctttcccttccctcctccccagaaggaaaacttggactcgtgtctttacgttagatcaattatataagctaagcgttcagttctcatgttgtatccgagtgggtatcgtcccttcgcttggtgtcgaatttcacagattttaaacgaaggaaccgttagcaagaaagtatcgtggaggagggaggttgcaggaaaaatgagagtgcaccagtactgtgctatgagccactagcaaaaaaaaggagattatatttttattatgatacttcgaagagtctggtaaaggacctaaagacttctccttctttggcatcactcccaaatagaaaaaacactacgaaggaattcaccaagtgggccccgggactggcctcgtattcaattacggtatttttgtaggagatatttcgctataatctagaatccccttcacaaatatacagagacgtgtagtatccctcctctgatgatacaactcgcccttctgcgaaacaaaacaatcaatactctttcccccgtatagttcccttgacaccttggtttctatcccgggagctcaagcgggagcgccgtgcccttttcctctaacgcccttctcttgcgatatcttcatacagactcatgagacccagtgtgctccatttaaatcccactcatcataccaagagtaaagttccgggggttatgccttcaaatacgtccccagcgactttccaaattgccccctagtcctgagcgggaattctaacggcttagagctggttagagattccaattccttcgctgaaaatcctattcaggggcgttcacactcacaatgcaaagctcgtccttccgttataccaccaatatctttcccactggtcccagtatggtctagccgagaacctctctgaaaccatcacgagaaactaccagcaatgctagccacaattccttaatctccagaccctctcgcccatgggctgctgcatagtacttctcctttgtttcggagctccatctttcaaatagcgtccttggcagctgcaccccccccccgaataccgccatactcatagccaaattcgactgcatgcgccagcgggtagccgaacgcagggacatccatcggatcggccgggagtgccgaaggatctatctccccctgatcggcagaggagaaagctacttcgaattcttgttttcgagtgccggcttcttgggtattgtaagcaacggcttctggatggttgggtttaaggtatacttttccagcacgcaataccacattatggaggttatctatataagaatatctcattagaattctatctagatctgtatacagaatcgcaaagcaacattgtcagaggggtcttggatagcacacctctctactcaagaagaaatgcacaggaaaaaaaagtatcgaggggacaaatagcataacccacatttcccagttcatcccccctttgcccaaaactcctcccggataccaaatagatcttcattaaccgccttgactttttctgcgggcctctcggactgtaaagagaaaaaaatttcgccgaacattgcagaagcctaagggccccaccctggcccggggagaatcctaccacaccatactatgatactcccgcaataatactatgctcagaccttggttggggtcgctggggtcgtgcgtgaccggtttttttttttttcgtacgtgaccggtttttggtgtcgccgctttttagggtctcagcgtcccaccttcgtgacgggtggtctatataagaagtccaaactgcccccctcctttccctctccttcttccaccaacaaccacaggagcgaatccccctccactcgatagcaagttccaaccttcctatcaacaaatccccctttggcagcatattctccatcacctcgccagcaatgtcaactctcaccgctctggagatggctatcgaagccgaggcatttattcaccagcgttacggtgggatctcgttccacgagtatctccccttgctcaccgatgggactttttgctttgagggatcggacggagctcagctccaggtttcccatatgcttcctaccaagtggaaacctacccccgagccgctcagggtttctttcaatccgtccccccataccatcgtcgggactcatctcactcttcatgaccgccctgtctacaagcttagcgatgggtccattcttgtcgtttccgaccccaccactagccccaccagtttccagctcacggatatcactttcgatgatcctcagcatgaagtcactaatgaaaaccgttttttgcgctttttcatcaaggctggtcaagacctctcatctcatgagatcagtgttcgcaaaattcaggagaagggtatcaagcgccctgccaatgcgtaagtacaactctaccaacctacaagcttcatacgctcatgcagtcacagattcattttgttccggaccgagaggatgggttccatcaaatcccaatatccggggattggaaacaatgacatctgtatgtagttctcattccctcttattaaaacttcattcctagtatcaccttcactaatagtttcgatagccaaaattcttggtcgtatgtggcgggattccccggacgaggcgaaggaagtgtataaggtatagcatctcactcaactactagttgacctggctaacaaattaataggcaaaggcccgtcaactcgcgatctcccacaagttgatctacccgaactacaaatacaccccgcgccgtccttatgaaattttaaggcgattgaatgggaatgtagatcctgagatcgtaaacgaacccggaaatacggttgcaaatgaccttggggcgctcacccaaaggtttatcgcccatccgaccgccgccactctcaatgaaattatcgcggaggcagggcagatcatgagcgcccttggctatactgagtaagccacctcggcagaggggcttgtgtgttccttgggtgatattcttttaatgggagggagggttgtgtctgttcttcatggatcactatcttagttagataaatttgtatgatccaaataaactaaaattgctctaaaaacgtgcgtgagaaacccaaacaccaaaccatcaattaccatcaagttcagtaggggtgagtgatgtactcgtgcgtccgagccaagaatgggcgcctctttcgcaatagctctcccaaccctgtaaataatagtattaatgctgttttttgagtagtgcatatgtggccactacaacatataataaatatcatgaacactcatacaaacatttccactattatactaaatctgatacattatgatacgcaataattgctcctcttatagtgcttgtaagactttattccttcattgcactattctaaatgttaaccttgaagttaaattttaatcacaggtatatactgaccttcaggagctttatctattcatccaattcaatgctctaaagtcttcttaagtattgaaaatatggtctcacatatgatatcattctcaccaggtgccagtattttgtgctatcatcggagctcacaaacagtacagccactatatcttttatggtctctgggagcttttccgaaaggggcgcccattcctggctcggacgcgtgagtaccctatgggccacagtgccgaacctacaaaaaacccctaatatgcctagacaattatactgtaggatagctcttgctttcaactaatgctaaccaaggggccgctcggcagtactcgggggtgcatgaaagcgacgggcaccccacgacgtatgtggcaccagattatggtgcatgttcaagcactagtcttcagctctctggtcctgaccacaggcagtttctaaatggaaaattacaaaatagtaaaatgaagattaaatgttggtcaaggaagactatagcaatagtgaaaatgaacagtgaaataaaccacaggacagcgcatggtaaaaagagcaaaaaaaataccaagaccacaaatacaaaacaattacacaactcagaagtgggggaaaaactcttcacttcagctaccactctctcaactgttcaaaatgttgtaggatatggtacaatcttaataactctgtatgaatatacaatgtagtagaaagagtgtattgtgggtaagctaacttattacctcctatgataatcaatgagtatagcaactacaagaaacatatttgaacatcctgcttattcaacttaaagataagtagacatcctatattggagtaagggttaagtataccatagtcctttgatcataagcgaatcatctaatatgttctttaatcaacactcaattcctaataaatatccttctatggtatgatttgtcttttggagtcaaatgattctgtagtgccataatatgaaggtatttattatatgtagtttccttttaatagatcacagagtagtaatcactctatggtctatgcattttcatattattgccaactctgttgaagctatacaacctgttaacttgaactctctactacccatatccctatacaaaatcgattttatcatgtgacattatactgtactacaaaatacttataagagaacaatatattatttttcattgaattataaagagtaaaagtttcctgcagccaacttgtttgattcagactggtcatatactgtattgctaactcttagtcatttcacttcatgttttattacaatgcatgatatatactacttatatattgtactaagcctgagactaatataacaggaaatactttggagtttgacttttatacaactctggggcccccaggtctgagttcttagagttttattgtattttatttcatattatagaatatggtatgtttggtatgatataagatattttaggaaatttgtatttattataaagtaccctatcaaatcgttccgtatgatgccctttttacaccaaaaacatgtatttttaccccctctttagacaccatcaatgtctatgaaaatctgctaaaatctcatgctacattatcaaaacccaggataacatgtaaaactttgcagagctctcaaaacctataaaattnnnnnnnnnnnnnnnnnnnngtggagatagcatgagccaagtgcatggtatgtggttgccatgtacctttttctatggagccccatacaaaatattaagtcacttttggacttggtgataccattgtatgctattgactcaatttcatcattttttattcatgcttctatgagggtaaggccaaagtgatttcctatccaatagcaaaaatttagttgtttttaaggaggcagtggtgggtattgtggctccgaaaatgttgatttttagccatatttggtgtaaaaactcatgtcgtatggaacgatttggtagggtactttacttcttcatttactcccaatacagcttataaactatacctgaaaagaaatttcaaaacaattatagaaaaaattaggaaaaaaaatttctataaaagaatatcaaatctttagtacttactagtaagatttatactactttaagacaagacttttgtaactttctgatatatttatctttctaaaatgagatctatcaattatatcttaggtctacaagtcttaaaaataatgcatgaaaatattaaagctttgtttataaaaaaatcttgttgaatattttgtagattattactatatttaatatactattacctggaacctaataggttaaataggaatttatatcagatgattgattataataatattataaaatagtattttaaaaattcagacctaaagtcctcagagttgcacagaaatcaaactctaaagcatctctcattatatcatccttagtgctgtgtataagtagcatgtaccatagtattatttttggagattgaagctaccctattatttatagttctttattattgatttgatttaaaaactataaaacctgttccctatttcaataaaagctttacagaccactagtaaaatttatgcatttgaggatttaaaggtcatagattgaactacctcgtagctatagatagagttgtaattcctaatttttgattcatgcattttttgttgtaatgccctataccctctctggagattttttttttttttaaatggtcatttcagaggcacccatccttgagcagtataagttacagtctaacgctctcacacctctcctctctacaacacacaaccccaaaattctcaatccaacaacaccctttgcacacataaaacacaatttgtttgctttgctaaaatgaactcaagtattatttccatactaaaaaccctaaatttaatagtgatggctaacttagaatcatagtccaagatatccgtctccgtcgtgagagaatgttcaaggctggagtgtttcttgctagagcaatggaagcaactgcatggaaccctggaaacctaatggtcgcaatcgccatccataagaatccaagtatttatgcggttaaagctattgagtcgtcccagagcttgcttaatgaggctgctgtgagtgatgacagattgctcaagatggatcccatcaaagaccgccagccactggcgaatatccagaatacactcctacagattggaggattactcaccttgaaaaaagatctctatgctgcgcttaaaaatatcaaattgcgtcttcaggagctccagggaggtatgtctaacaacattggtcaggctgttgcagaggttcacgactttgtctgtttcgccacatttgtcgacttgatccacccgcaaagctccatcttgaccttccaatccatcaaggaggcggagattcgcgcatccaagatgccataccacgaggctcgccaggcgtttatcgaactccgccaggcttacgacctgcaggaccccgttgagggattccgtctaagcaaagccagggaattgcctgcaattactaagtgcatcatggaacagtggcacagaggttatgtttcccacgcctctgcaactggaaaggtaagtataaaaaaccaccccttctctcagtatttcttatctagcaccccctaccctgcactatactaacatcagcacgtaggaagttattcctttcaagaccgtgtttggatggtggagtcagttcaatccctcatttccgattgatagcgtcgtcagctaacaataaacggcccccgtttacagtcaattatcagcgcaaatttatcagccctccccaagagaaaactacgccggttgtgcaagaacttatcagcaaagcctatatctcgaagaccaagttgcccgaggacgaggaccagcaaccatggatgcaagaagacaaggaagcttttaaggccgtgaccgacctagccatctctatgaccccactttgcaaaaacgtcccctacccggagcatgttaaaccaaacccccagaaaaaaaagcaattgccgatctacacccagttcgaagttgaccctcagaaccaaccgctctgccaagaactccagaagctgttggctcaaatttacgagcttttcgaagaaaattcgtgcaaaacttttactatcatgaccaacactcactatggcaaagtggagaactaccttaagtgctctactcaagaccagttactgtcttcgataaagcacttgaagtaagttaccttccaagcttcaaagttttttattacatcaaagctaatctccaacagtgccgtcacctacttcttcacttcggtccgccaggtcagggttgtagtttccaaggatagaccctgggcgagtcctgagcaccgcaaggttgaagttccgatccttgatgccgttagcaaggtctgtaatgatcccaccattacagacgaagagcatctggcagaaattacctgcaagagcttgcctcaagttggaatgcactgtaagttctcccagtcactagtttcggatatttgctgactgccgaatgtaatacctggaatagtccttatcaactaccgcggtcctattcacggtcctgttcccggtcatgttcacggtcctggtcacggtcctgttcacggtcctgttcccggtcatgttcacggtcatgttcacggtcctggtcacggtcatgttcacggtcatgttcacggtcctggtcacggtcctgttcacggtcctgttcccggtc

(19) United States
(12) Patent Application Publication
(10) Pub. No.: US 2013/0224741 A1
(43) Pub. Date: Aug. 29, 2013

(54) Molecular Method for the Identification of Mating Type Genes of Truffles Species

(71) Applicants: Francis Martin, Champenoux (FR); Claude Murat-Furminieux, Saint Max (FR); Francesco Paolocci, Perugia (IT); Andrea Rubini, Cita Di Castello (IT); Claudia Riccioni, Perugia (IT); Beatrice Belfiori, Perugia (IT); Sergio Arcioni, Perugla (IT)

(72) Inventors: Francis Martin, Champenoux (FR); Claude Murat-Furminieux, Saint Max (FR); Francesco Paolocci, Perugia (IT); Andrea Rubini, Cita Di Castello (IT); Claudia Riccioni, Perugia (IT); Beatrice Belfiori, Perugia (IT); Sergio Arcioni, Perugla (IT)

(21) Appl. No.: 13/820,718

(22) PCT Filed: Sep. 7, 2011
(86) PCT No.: PCT/EP2011/065501

§ 371 Date: May 9, 2013

(30) Foreign Application Priority Data
Sep. 7, 2010 (EP) 10175517.1

(51) Int. Cl. C12Q 1/68

(52) U.S. Cl. CPC C12Q 1/6879; C12Q 1/6895
USPC 435/6.11; 536/23.1; 435/6.12

(57) ABSTRACT

The invention concerns a method for determining the Tuber species and fertility of a truffle sample, said method comprising: - identifying whether said truffle sample comprises a first nucleotide sequence associated with a first mating type idiomorph (MAT1-2); and - identifying whether said truffle sample comprises a second nucleotide sequence associated with a second mating type idiomorph (MAT1-1); the fertility of said truffle sample requiring the presence of both the first and the second nucleotide sequences.

### INVENTION DOMAIN

The invention concerns the setting up of a method for the identification of truffles species and their mating type. The invention is based on the use of mating-specific nucleotide sequences.

An aim of the invention is to control the sexual reproduction of the truffles.

An additional aim of the invention is to distinguish between the truffles species.

The invention concerns the production and the control of truffle inoculated seedlings, with direct impact on the activity and interest of truffles nurseries and agro-food industry.

### STATE OF THE ART AND ENCOUNTED PROBLEM

Truffles are Ascomycete mushrooms belonging to the Tuber genus which live in ectomycorrhizal symbiosis with various trees. These mushrooms form fructifications generally named <<truffles>> which are very appreciated for their organoleptic qualities. Among these truffles species, the Périgord black truffle (Tuber melanosporum Vittad.), the Piémont white truffle (Tuber magnatum Pico) and the Burgundy Truffle (Tuber aestivum Vittad.) are the most appreciated species by the gastronomes and have a large cultural and economic impact. Indeed, their price can vary between 200-400 euro/kg for the Burgundy truffle, 400-1,000 euro/kg for the Périgord truffle and 2,000-6,000 euro/kg for the Piémont white truffle. This very high price is, among other things, the consequence of a high decrease of their production over the last decades. In view to preserve these species and to increase their production, a protocol permitting to inoculate host plantlets, with each of the previously mentioned truffle species has been made to be exploited by truffles nurseries.

This protocol consists to inoculate first young oak, hazel, pin or other host plantlets by mean of spores from several truffles. Each truffle used as spore donor is morphologically controlled to avoid contamination with undesired species. Alternatively, host plants can be inoculated with in vitro grown mycelia.

Then, the development of mycorrhizas on the inoculated plants is controlled 1) to exclude plants that either do not develop mycorrhizas or harbour mycorrhizas from contaminating fungal species according to morphological criteria and 2) to assess whether the percentage of mycorrhization is sufficient. If the level of mycorrhization is sufficient and few contamination are detected the mycorrhized plantlets can be sold. Mycorrhized plantlets means that plantlet roots have been colonized by truffle mycelium and that truffle mycelium and plantlet live in a symbiotic relationship the one with the other.

When these verifications have been met, these mycorrhized plantlets are planted in an orchard. After 5 to 8 years of culturing, it might be obtained truffles of the Tuber species that has been inoculated.

The discrimination of Tuber vs non Tuber mycorrhizas by morphological criteria is often an easy task. However, the discrimination among mycorrhizas of the different Tuber species is sometime impossible, such as the typing of mycorrhizas formed by the Périgord Black truffles from those formed by the Chinese black truffles. Mistakes are possible mainly with the Chinese black truffle Tuber indicum. Indeed this species is morphologically very similar to the Périgord Black truffle and sometimes their morphological discrimination is impossible. T. indicum is a truffle that presents organoleptic qualities lesser pronounced than the ones presented by T. melanosporum.

Moreover, despite advances in the inoculation methodology and cultural improvements, some trees and plantations are still sterile without producing truffles. So the quantity of truffles that can be produced by a truffle plant is unpredictable.

### SUMMARY OF THE INVENTION

The inventors have discovered that truffles, and in particular T. melanosporum and T. magnatum are obligate outcrossing fungi, also known as heterothallic fungi. These mushrooms are self-incompatible and therefore need the presence of a partner of opposite mating type to reproduce themselves.

The inventors have discovered that to ensure sexual reproduction of truffle species, the mating between two sexually compatible mycelia is needed. These two mycelia differ for the mating type genes in their genome.

Mating type genes or MAT genes are the master loci controlling sexual reproduction and development in fungi.

Sexual reproduction in Ascomycetes filamentous fungi is controlled by two different MAT genes that established sexual compatibility: one MAT gene codes for a protein with an alpha box domain (MAT1-1-1), whereas the other encodes a high mobility group (HMG) protein (MAT1-2-1). In heterothallic Ascomycetes, there are strains that have only one of these genes in their genome, the other mating type gene is present in different strains at the same site on the chromosome, namely MAT locus. Because this locus contains different genes and other highly divergent sequence, the two versions of a MAT locus (MAT1-1 and MAT1-2) are not called allelic and are called idiomorphs (Metzemberg & Glass, 1990, Bioessays 12: 53-59).

To gain insight into the truffle reproduction system, the inventors have tried to identify the nucleotide sequences of the MAT locus in each of the most famous truffles species taking advantage from the fact that the genome of the Périgord black truffle has been recently deciphered. Thus, the inventors have first tried to identify, in T. melanosporum, the nucleotide sequences corresponding to the two mating type genes. Indeed, due to the high nucleotide sequence divergences between mating type genes within Ascomycetes, the inventors have not immediately found the nucleotide sequences corresponding to the two mating type genes.

The same study has been made to T. magnatum, T. indicum, T. borchii and T. aestivum.

The inventors have first screened the sequenced T. melanosporum genome for the presence of candidate genes encoding for the protein containing the HMG and the alpha-box domain using as queries mating type genes from other Ascomycetes. Following this approach, the inventors have discovered in the T. melanosporum genome three nucleotides sequences (three genes: GSTUMT00001090001, GSTUMT00000587001 and GSTUMT00008644001) that have homologies with the HMG containing mating type gene of other Ascomycetes. On the contrary, no T. melanosporum genes corresponding to the alpha-box mating type genes of other Ascomycetes has been detected, suggesting that T. melanosporum is indeed a heterothallic species. For each of the three HMG genes identified in T. melanosporum genome, the inventors have designed a specific primer pair to verify their presence in the other T. melanosporum strains. Embracing the hypothesis of heterothallism, the putative HMG- mating type gene cannot be present in all T. melanosporum strain, since some strains should be of the opposite mating type. Indeed, this prediction was confirmed, as one of the three HMG containing genes (GSTUMT00001090001) was not found in all strains tested. Thus, this gene has been identified as the first mating type gene of T. melanosporum, namely TmelMAT1-2-1. The HMG box of this first mating type of T. melanosporum has as nucleotide sequence the nucleotide sequence SEQ ID No 1.

Having mapped the position of TmelMAT1-2-1 on the T. melanosporum sequenced genome, the inventors have then designed new primers on its flanking regions to amplify the entire idiomorphic sequence both from strains with and without the TmelMAT1-2-1 gene. In the light of heterothallism in fact, the 5′ and 3′ regions flanking the two putative idiomorphic structures are expected to be conserved between strains of opposite mating type. Following this approach, the second MAT locus (MAT1-1) has been cloned and then sequenced from a strain that lacked the TmelMAT1-2-1 gene.

Sequence analysis confirmed the identification of the MAT idiomorph (MAT1-1) containing a second mating type gene, namely TmelMAT1-1-1, said second mating type gene encodes for a putative alpha-box containing transcription factor showing appreciable sequence similarity with the alpha box mating type genes from other Ascomycetes. This alpha box specific to the second mating type gene of T. melanosporum has as nucleotide sequence the nucleotide sequence SEQ ID N ^{o}2.

Overall, following the above mentioned approaches both MAT idiomorphic regions of T. melanosporum have been identified. The first mating type idiomorph MAT1-2 (TmelMAT1-2) corresponds to the nucleotide sequence SEQ ID N ^{o}3. The second mating type idiomorph MAT1-1 (TmelMAT1-1) corresponds to the nucleotide sequence SEQ ID N ^{o} 4.

The inventors have determined a first genomic region comprising the first mating type gene MAT1-2-1 (or “the first mating type idiomorph MAT1-2”) and a second genomic region comprising the second mating type gene MAT1-1-1 (or “the second mating type idiomorph MAT1-1”). Each of these genomic regions comprises also flanking regions that are highly conserved.

The first genomic region has as nucleotide sequence the nucleotide sequence SEQ ID N ^{o}5 and the second genomic region has as nucleotide sequence the nucleotide sequence SEQ ID N ^{o}6.

By the way of the above identified nucleotide sequences SEQ ID N ^{o}1, SEQ ID N ^{o}2, SEQ ID N ^{o}3, SEQ ID N ^{o}4, SEQ ID N ^{o}5 and SEQ ID N ^{o}6, the inventors were able to implement an identification method of T. melanosporum. But others nucleotide sequences specifics to the two mating type gene of T. melanosporum can be used.

The invention permits to differentiate truffles species between them. In particularly, the invention permits to differentiate Chinese truffle (Tuber indicum Cook & Massee) with regard to the Périgord Black truffle (T. melanosporum).

The invention permits therefore to avoid contamination of plantlets with T. indicum. The invention permits to ban the presence of T. indicum in the plantlets that will be planted in Europe.

The invention permits also to differentiate other truffles species between them, such as T. melanosporum, T. magnatum, T. aestivum.

By the way of the above identified nucleotide sequences, the inventors were also able to screen mycorrhizal plants and determine whether they sustain T. melanosporum mycorrhizas of one or both mating types on their roots. More precisely, the method is based on the amplification with mating-type T. melanosporum specific primers of DNA isolated from randomly selected pools of T. melanosporum mycorrhizas. If the plants harbouring both mating types, the proportion of each mating type can be measured by performing a quantitative PCR analysis or by analyzing randomly selected single ectomycorrhizas and counting the proportion of mycorrhizas harbouring the first or the second mating type.

Likewise, by the way of the above identified nucleotide sequences, the inventors were able to screen in vitro cultivated T. melanosporum mycelia.

It is also possible to guaranty to the truffle cultivators:
the mating type(s) of strains present on mycorrhizal plantlets. The methods, in fact, permits to check whether a host plant harbours strains of only one or both mating types of a given Tuber species.
host plant selection according to the mating type of the fungus;
the setting up of truffle plantations in which the presence of strains of both mating types of a given Tuber species is spatially and numerically balanced in the ground. This can be achieved by using plants harbouring strains of both mating type in their roots or by outplanting close each other host plants that harbour in their roots strains of opposite mating type to promote, in open field conditions, the contact and mating between sexually compatible mycelia;
to control the presence and the distribution of strains of different mating type in previously realized truffle plantations as well as in natural truffle sites to induce ex novo or rescue the truffle production. These analyses permit, in fact, to test whether in a given truffle field the presence of strains of both mating type is spatially and numerically balanced. If not, in order to favour mating between sexually compatible strains, mycorrhized plants harbouring strains with the mating type resulted to be absent or less abundant can be selected and outplanted;
to control the mating type of mycelia and/or other inocula used for host plant inoculation.

Each mating type gene of a given truffle species can be found with the same method as the one disclosed for obtaining the nucleotide sequence of the TmelMAT1-2-1 and TmelMAT1-1-1 as above indicated.

For a given Tuber species, it is wanted strains that comprise a first nucleotide sequence that hybridizes with the HMG containing mating type gene of other Ascomycetes. Then, from the flanking regions of this first nucleotide sequence, it is wanted primers in view to amplify a nucleotide sequence corresponding to the second mating type gene of this same given Tuber species for the other strains that not contain the first mating type gene.

In an embodiment, each mating type gene of a given truffle species can be identified with the help of parts of nucleotide sequences taken from the first genomic region of T. melanosporum and from the second genomic region of T. melanosporum. Each mating type gene of a given truffle species can also be identified with the help of parts of nucleotide sequences taken from the first mating type gene of T. melanosporum and of the second mating type gene of T. melanosporum. These nucleotide sequences can be used as pairs of primers to amplify respectively a first mating type gene and a second mating type gene of a given truffle species.

It is also possible to use the nucleotide sequences of the HMG specific box of T. melanosporum and of the alpha specific box of T. melanosporum as probes to identify the nucleotide sequences of a first mating type gene and a second mating type gene of a given truffle species.

The invention is used to certify the truffle species, to control fructifications of these species, the identity (and mating type) of their in vitro cultured and/or soil living mycelia and mycorrhizas.

The invention permits also to verify the mating type in plantlets inoculated with T. melanosporum, T. magnatum and T. aestivum which will be sold in the future, and to ensure the presence of mycelia of the two mating types in view to optimize truffles production.

The invention permits to certify the presence of compatible mycelia at the root system level.

The invention also permits to increase fruiting and hence truffle production by controlling a priori the presence of mating types on mycorrhized plants used to set a truffle orchard. Also, as reported above, the invention would allow the rescue of man-made and natural plantations by enriching these sites for the presence of strains harbouring the less abundant (or absent) mating type through the introduction of mycorrhized plants specifically selected for the mating type of their fungal partner.

Thus, it is possible to certify to a truffle cultivator the mating type(s) of the truffle strains on the roots of the host plants.

The invention has as object a method for determining the Tuber species of a truffle sample, said method comprising identifying whether said truffle sample comprises at least one nucleotide sequence chosen among a first nucleotide sequence associated with a first mating type idiomorph (MAT1-2) and a second nucleotide sequence associated with a second mating type idiomorph (MAT1-1); both nucleotide sequence being specific to a given Tuber species.

By truffle sample it is intended that it comprises truffle fructification sample and/or truffle mycelium sample and/or mychorrized plant sample and/or a truffle producing soil sample.

The first nucleotide sequence can be at least one part of the nucleotide sequence SEQ ID N ^{o}5. In an example, the first nucleotide sequence can be the nucleotide sequence SEQ ID N ^{o}5, or a part of the nucleotide sequence SEQ ID N ^{o}5. For example, it can be the nucleotide sequence SEQ ID N ^{o}3 or the nucleotide sequence SEQ ID N ^{o}1.

The second nucleotide sequence can be at least one part of the nucleotide sequence SEQ ID N ^{o}6. In an example, the second nucleotide sequence can be the nucleotide sequence SEQ ID N ^{o}6 or a part of the nucleotide sequence SEQ ID N ^{o}6. For example, it can be the nucleotide sequence SEQ ID N ^{o}4 or the nucleotide sequence SEQ ID N ^{o}2.

The nucleotide sequence SEQ ID N ^{o}5 is a first genomic region of T. melanosporum that comprises the first T. melanosporum mating type gene TmelMAT1-2-1 or SEQ ID N ^{o}3 and the nucleotide sequence SEQ ID N ^{o}6 is a second genomic region of T. melanosporum that comprises the second T. melanosporum mating type gene TmelMAT1-1-1 or SEQ ID N ^{o}4.

The invention provides also that the method further comprises determining the fertility of said truffle sample, using the identification in said truffle sample of both the first nucleotide sequence associated with the first mating type idiomorph (MAT1-2) and the second nucleotide sequence associated with a second mating type idiomorph (MAT1-1), the fertility of said truffle sample requiring the presence of both the first and the second nucleotide sequences.

The first nucleotide sequence may comprises at least one part of the nucleotide sequence SEQ ID N ^{o}5. In an embodiment, the first sequence may also comprises the nucleotide sequence SEQ ID N ^{o}5, or the nucleotide sequence SEQ ID N ^{o}3 or the nucleotide sequence SEQ ID N ^{o}1. The second nucleotide sequence may comprises at least one part of the second nucleotide sequence SEQ ID N ^{o}6. In an embodiment, the second nucleotide sequence may comprises the nucleotide sequence SEQ ID N ^{o}6 or the nucleotide sequence SEQ ID N ^{o}4 or the nucleotide sequence SEQ ID N ^{o}2.

The method may comprises the use of a first pair of oligonucleotide primers to determine the presence of the first mating type idiomorph (MAT1-2) in said truffle sample, each primer of the first pair of oligonucleotide primers comprising from 5 to 50, and preferentially from 10 to 30 successive nucleotides of the first nucleotide sequence. The first nucleotide sequence can be the nucleotide sequence SEQ ID N ^{o}5. In an embodiment, the first nucleotide sequence may comprises the nucleotide sequence SEQ ID N ^{o}3 or the nucleotide sequence SEQ ID N ^{o}1.

A forward primer of this first pair of oligonucleotide primers may comprises the nucleotide sequence SEQ ID N ^{o}7. The nucleotide sequence SEQ ID N ^{o}7 is taken from the nucleotide sequence SEQ ID N ^{o}1. Other specific nucleotide sequences can be used, as the nucleotide sequence SEQ ID N ^{o}11 or the nucleotide sequence SEQ ID N ^{o}13 that are parts of the nucleotide sequence SEQ ID N ^{o}5.

A reverse primer of this first pair of oligonucleotide primers may comprises the nucleotide sequence SEQ ID N ^{o}8. The nucleotide sequence SEQ ID N ^{o}8 is taken from the flanking region of the nucleotide sequence SEQ ID N ^{o}1. Other specific nucleotide sequences can be used, as the nucleotide sequence SEQ ID N ^{o}12 or the nucleotide sequence SEQ ID N ^{o}14 that are parts of the nucleotide sequence SEQ ID N ^{o}5.

The nucleotide sequence SEQ ID N ^{o}7 and the nucleotide sequence SEQ ID N ^{o}8 serve to amplify a part of the nucleotide sequence SEQ ID N ^{o}1.

The nucleotide sequence SEQ ID N ^{o}11 and the nucleotide sequence SEQ ID N ^{o}12 serve to amplify the nucleotide sequence SEQ ID N ^{o}3.

The nucleotide sequence SEQ ID N ^{o}13 and the nucleotide sequence SEQ ID N ^{o}14 serve to amplify the nucleotide sequence SEQ ID N ^{o}5.

The method may comprises the use of a second pair of oligonucleotide primers to determine the presence of the second mating type idiomorph (MAT1-1) in said truffle sample, each primer of the second pair of oligonucleotide primers comprising from 5 to 50, and preferentially from 10 to 30 successive nucleotides of the second nucleotide sequence. As indicated above, the second nucleotide sequence can be the following nucleotide sequences SEQ ID N ^{o}2, SEQ ID N ^{o}4 or SEQ ID N ^{o}6.

A forward primer of this second pair of oligonucleotide primers may comprises the nucleotide sequence SEQ ID N ^{o}9. The nucleotide sequence SEQ ID N ^{o}9 is taken from the nucleotide sequence SEQ ID N ^{o}2. Other specific nucleotide sequences can be used, as the nucleotide sequence SEQ ID N ^{o}11 or the nucleotide sequence SEQ ID N ^{o}13.

A reverse primer of the second pair of oligonucleotide primers may comprises the nucleotide sequence SEQ ID N ^{o}10. The nucleotide sequence SEQ ID N ^{o}10 is taken from the nucleotide sequence SEQ ID N ^{o}2. Other specific nucleotide sequences can be used, as the nucleotide sequence SEQ ID N ^{o}12 or the nucleotide sequence SEQ ID N ^{o}14.

The nucleotide sequence SEQ ID N ^{o}9 and the nucleotide sequence SEQ ID N ^{o}10 serve to amplify a part of the nucleotide sequence SEQ ID N ^{o}2.

The nucleotide sequence SEQ ID N ^{o}11 and the nucleotide sequence SEQ ID N ^{o}12 serve to amplify the nucleotide sequence SEQ ID N ^{o}4.

The nucleotide sequence SEQ ID N ^{o}13 and the nucleotide sequence SEQ ID N ^{o}14 serve to amplify the nucleotide sequence SEQ ID N ^{o}6.

The detection of the presence of at least one part of the first nucleotide sequence associated with a first mating type idiomorph of a Tuber species and of at least one part of a second nucleotide sequence associated with the second mating type idiomorph of the same Tuber species could be done with the help of usual molecular biology tools wheel known from one of ordinary skill in the art.

For example, the detection of this presence can be done in labelling a genomic marker or a specific primer and in hybridizing the genomic marker or the specific primer to an amplified DNA sequence. The marker or the primer can be at least one part of one of the two nucleotide sequences associated with a mating type idiomorph of T. melanosporum or an other given Tuber species.

The detection of this presence could be done also in labelling cDNA which are able to be detected by at least one part of a first nucleotide sequence associated with a first mating type idiomorph of a Tuber species and by at least one part of a second nucleotide sequence associated with a second mating type idiomorph of the same Tuber species.

The invention has also as object a nucleotide sequence associated with a first mating type idiomorph (MAT1-2) of a Tuber species comprising the nucleotide sequence SEQ ID N ^{o}5. The nucleotide sequence SEQ ID N ^{o}5 is the first genomic region of T. melanosporum that comprises the first mating type gene of T. melanosporum and the HMG specific box of T. melanosporum.

The invention has also as object a nucleotide sequence associated with a second mating type idiomorph (MAT1-1) of a Tuber species comprising the nucleotide sequence SEQ ID N ^{o}6. The nucleotide sequence SEQ ID N ^{o}6 is the second genomic region of T. melanosporum that comprises the second mating type gene of T. melanosporum and the alpha specific box of T. melanosporum.

The invention has also as object a first pair of oligonucleotide primers associated with a first mating type idiomorph (MAT1-2-1) of a Tuber species, wherein each primer comprises from 5 to 50, for preference from 10 to 30 successive nucleotides of the nucleotide sequence SEQ ID N ^{o}5.

In an embodiment, this first pair of oligonucleotides primers comprises a forward primer with as nucleotide sequence the nucleotide sequence SEQ ID N ^{o}7 and a reverse primer with as nucleotide sequence the nucleotide sequence SEQ ID N ^{o}8.

Other forward primers can be used with as nucleotide sequences the nucleotide sequence SEQ ID N^{o} 11 or the nucleotide sequence SEQ ID N ^{o 13}.

Other reverse primers can be used with as nucleotide sequences the nucleotide sequence SEQ ID N ^{o}12 or the nucleotide sequence SEQ ID N ^{o}14.

The invention has also as object a second pair of oligonucleotide primers associated with a second mating type idiomorph (MAT1-1-1) of a Tuber species, wherein each primer comprises from 5 to 50, for preference from 10 to 30 successive nucleotides of the nucleotide sequence SEQ ID N ^{o}6.

In an embodiment, this second pair of oligonucleotides primers comprises a forward primer with as nucleotide sequence the nucleotide sequence SEQ ID N ^{o}9, and a reverse primer with as nucleotide sequence the nucleotide sequence SEQ ID N ^{o}10.

Other forward primers can be used with as nucleotide sequences the nucleotide sequence SEQ ID N ^{o}11 or the nucleotide sequence SEQ ID N ^{o}13.

Other reverse primers can be used with as nucleotide sequences the nucleotide sequence SEQ ID N ^{o}12 or the nucleotide sequence SEQ ID N ^{o}14.

The invention can provides also the development of fast and easy to use a kit to determinate the fertility of a truffle sample, like truffle fructification sample, truffle mycelium sample, mycorrhized plant sample or truffle-producing soil sample; a kit being able to be used even by unspecialized persons. Indeed, it is possible to create a “two in one” kit allowing the identification of the two mating type in a single reaction. The idea is to merge the findings realized by the inventors with the expertise of nanotechnologists to realize such a kit.

The invention has also as object a kit for determining the fertility of a truffle sample, like truffle fructification sample, truffle mycelium sample, mycorrhized plant sample or truffle-producing soil sample, the kit comprising:
the pair of oligonucleotide primers associated with a first mating type idiomorph (MAT1-2-1) of a Tuber species, as indicated above, and
the pair of oligonucleotide primers associated with a second mating type idiomorph (MAT1-1-1) of a Tuber species, as indicated above.

Means for amplifying a nucleotide sequence are also included in this kit. This kit can be use also to identify the truffle species between them.

The nucleotides sequences SEQ ID N ^{o}1, SEQ ID N ^{o}2, SEQ ID N ^{o}3, SEQ ID N ^{o}4, SEQ ID N ^{o}5, SEQ ID N ^{o}6, SEQ ID N ^{o}7, SEQ ID N ^{o}8, SEQ ID N ^{o}9, SEQ ID N ^{o}10, SEQ ID N ^{o}11, SEQ ID N ^{o}12, SEQ ID N ^{o}13 and SEQ ID N ^{o}14 are nucleotides sequences that are specifics to the species T. melanosporum. But others nucleotides sequences specifics to others Tuber specie could be used.

The same study has been made to T. indicum.

Since T. indicum is an highly polymorphic species (Paolocci et al. 1997 FEMS Microbiology Letters 153: 255-260), some authors regard it as a species-complex (Wang et al., 2006 Mycological Research 110: 1034-1045). The inventors have therefore initially screened a collection of T. indicum ascocarps by performing the RFLP analysis of the ITS region described by Paolocci et al. (1997) FEMS Microbiology Letters 153: 255-260 to sort them into the respective molecular RFLP/ITS classes.

The inventors proved that the ascocarps analyzed can be grouped into three ITS classes according to the restriction patterns produced by the endonuclease RSAI on ITS1/ITS4 (White et al. 1990 Academic Press, New York, pp 315-322) PCR amplicons, hereinafter refereed to as Class A, B1 and B2.

The inventors then used a set of T. indicum ascocarps belonging to each of the three ITS classes for the identification of mating type genes. The inventors used the T. melanosporum MAT primers at low stringent PCR conditions to amplify a portion of T. indicum mating type genes. The T. melanosporum primer pair SEQ ID N o7/SEQ ID N ^{o}8 specific for TmelMAT1-2-1 gene produced a PCR fragment of approximately 500 bp on some T. indicum ascocarps. The T. melanosporum primer pair SEQ ID N ^{o}9/SEQ ID N ^{o}10 specific for MAT1-1-1 gene produced a PCR fragment of approximately 400 bp, on a different set of T. indicum ascocarps. These results were in keeping with the hypothesis of heterothallism in T. indicum. As such, the amplification pattern of the MAT genes from T. indicum ascocarps proved that the gleba of each T. indicum samples produced either a 500 bp- or a 400 bp- long PCR fragments and that these PCR fragments shared a high sequence similarity with HMG-box and α-box sequences of T. melanosporum, respectively.

The inventors have then designed new primers specific to T. indicum HMG-box and α-box sequences. As the 5′ and 3′ regions flanking the two idiomorphs of T. melanosporum were expected to be conserved in T. indicum, which is phylogenetically related to T. melanosporum, the newly designed primers for the T. indicum HMG-box and α-box genes were then combined with reverse primers designed on the 5′ and 3′ regions flanking the two idiomorphs of T. melanosporum to extend sequence information relative to the T. indicum MAT locus. The resulting PCR products from MAT1-2 and MAT1-1 samples were then sequenced. Sequence analysis proved that both T. indicum idiomorphs, along with a portion of their flanking regions, were isolated.

As previously observed in T. melanosporum (Rubini et al., 2011 New Phytologist 189: 710-722), the spores entrapped within the ascocarp can contribute a slight amount of DNA during the process of nucleic acid isolation from ascocarps. As the spores, differently from the gleba which is only formed by uniparental maternal hyphae, contain DNA of both maternal and paternal origin, the use of MAT specific primers allowed, the inventors to amplify and isolate both MAT indiomorphs from a single T. indicum ascocarp. In the light of the high genetic polymorphism of T. indicum this approach has enabled the inventors to identify both idiomorphs from samples belonging to the three distinct ITS classes. Thus, both MAT1-2 and MAT1-1 idiomorphs were amplified and entirely sequenced from the ascocarps TI_CF10, TI_U983 and TI_U986, belonging to the ITS classes A, B1 and B2, respectively.

The first mating type idiomorph MAT1-2 corresponds to the nucleotide sequences SEQ ID N^{o} 34, 35 and 36 for TI_U983 (Tind_B1_MAT1-2), TI_U986 (Tind_B2_MAT1-2) and TI_CF10 (Tind_A_MAT1-2) samples, respectively. The second mating type idiomorph MAT1-1 corresponds to the nucleotide sequences SEQ ID N^{o} 37, 38 and 39 for TI_U983 (Tind_B1 —MAT1-1), TI_U986 (Tind_B2_MAT1-1) and TI_CF10 (Tind_A_MAT1-1) samples, respectively.

Within each idiomorph a single HMG-box (MAT1-2-1) or α-box (MAT1-1-1) containing gene was identified. The first mating type gene MAT1-2-1 corresponds to the nucleotide sequences SEQ ID N^{o} 40, 41 and 42 for TI_U983 (Tind_B1_MAT1-2-1), TI_U986 (Tind_B2_MAT1-2-1) and TI_CF10 (Tind_A_MAT1-2-1), respectively. The second mating type gene MAT1-1-1 corresponds to the nucleotide sequences SEQ ID N^{o} 43, 44 and 45 for TI_U983 (Tind_B1_MAT1-1-1), TI_U986 (Tind_B2 MAT1-1-1) and TI_CF10 (Tind_A_MAT1−1-1), respectively.

The inventors have determined a first genomic region comprising the first mating type idiomorph MAT1-2 which contains the first mating type gene MAT1-2-1 and a second genomic region comprising the second mating type idiomorph MAT1-1 which contains the second mating type gene MAT1-1-1. Each of these genomic regions comprises also conserved flanking sequences.

The first genomic region correspond to the nucleotide sequences SEQ ID N^{O} 46, 47 and 48 for TI_U983, TI_U986 and TI_CF10, respectively. The second genomic region correspond to the nucleotide sequence SEQ ID N^{o} 49, 50 and 51 for TI_U983, TI_U986 and TI_CF10, respectively.

### BRIEF DESCRIPTION OF THE FIGURES

FIGS. 1a and 1b represent schematics representations of Tuber melanosporum mating type locus MAT1-2-1 and Tuber melanosporum mating type locus MAT1-1-1, respectively.

A (SEQ ID N ^{o}3) and B (SEQ ID N ^{o}4) represent respectively the nucleotide sequence for the first mating type idiomorph of T. melanosporum, TmelMAT 1-2, and for the second mating type idiomorph of T. melanosporum, TmelMAT 1-1. The black lines indicate the common flanking regions. C (SEQ ID N ^{o}1) and D (SEQ ID N ^{o}2) arrows represent respectively the nucleotide sequence for the HMG box (mating type gene TmelMAT 1-2-1) and for the alpha box (mating type gene TmelMAT 1-1-1). An additional putative ORF was detected in the MAT1-2 idiomorph (E arrowed box; gene model: GSTUMT00001089001). The F, G and H boxes represent regions sharing sequence similarities between idiomorphs. The 45° grid pattern indicates the opposite orientation of similar sequences within and between idiomorphs. The flanking region downstream the MAT1-1 idiomorph contains a 493 bp long insertion used to design a backward primer to specifically amplify the MAT1-1 idiomorph (I Box). The arrowed boxes J and K indicate the gene models: GSTUMT00001088001 and GSTUMT00001092001, conserved in the regions flanking the idiomorphs.

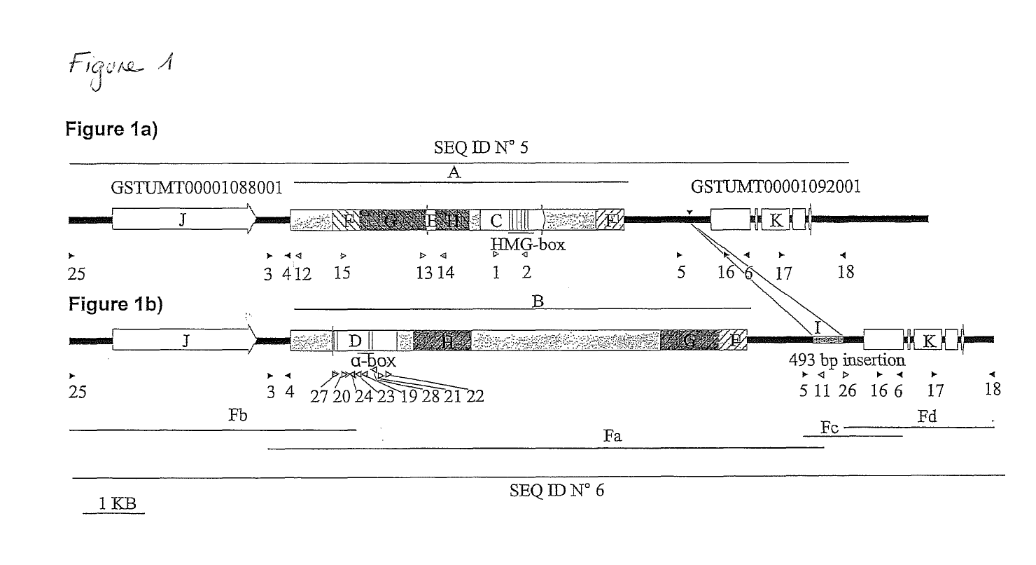

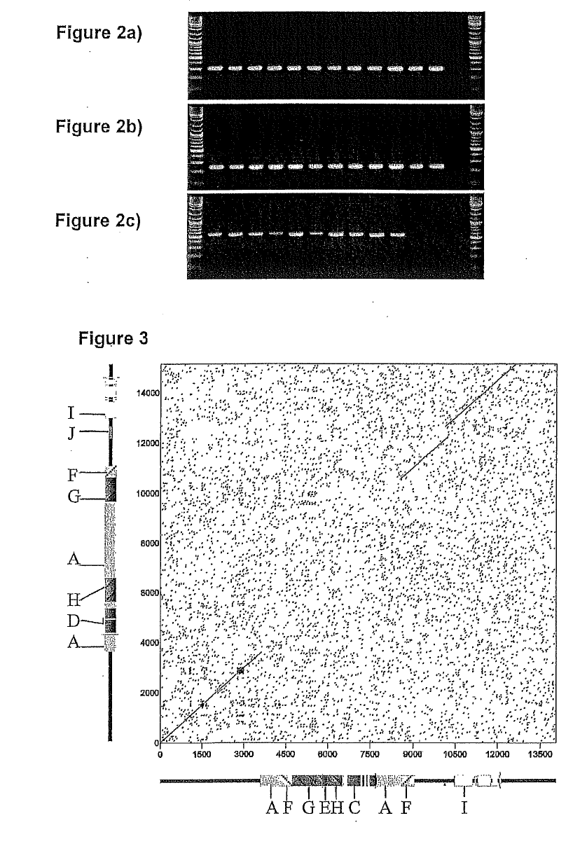

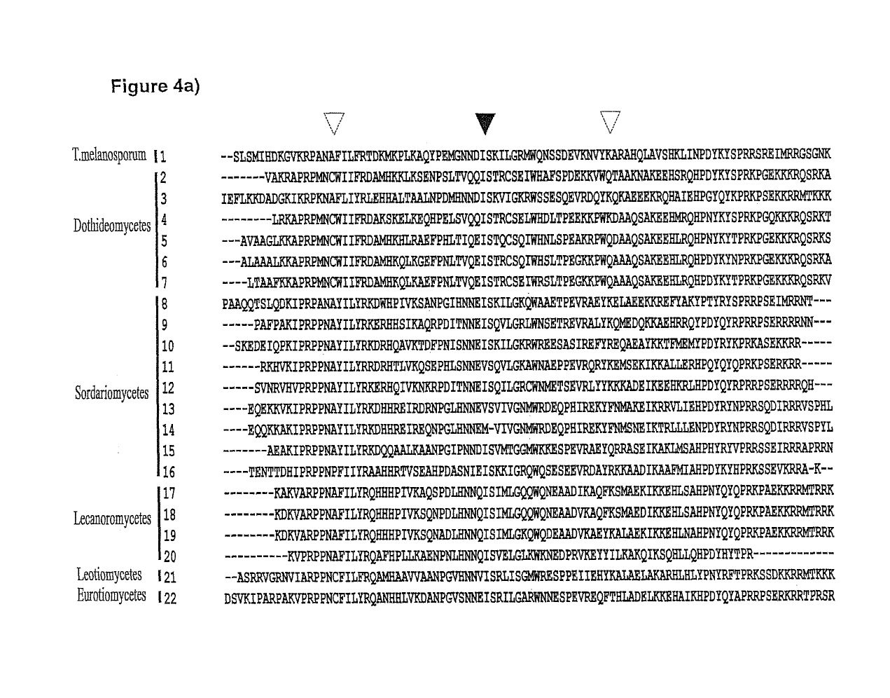

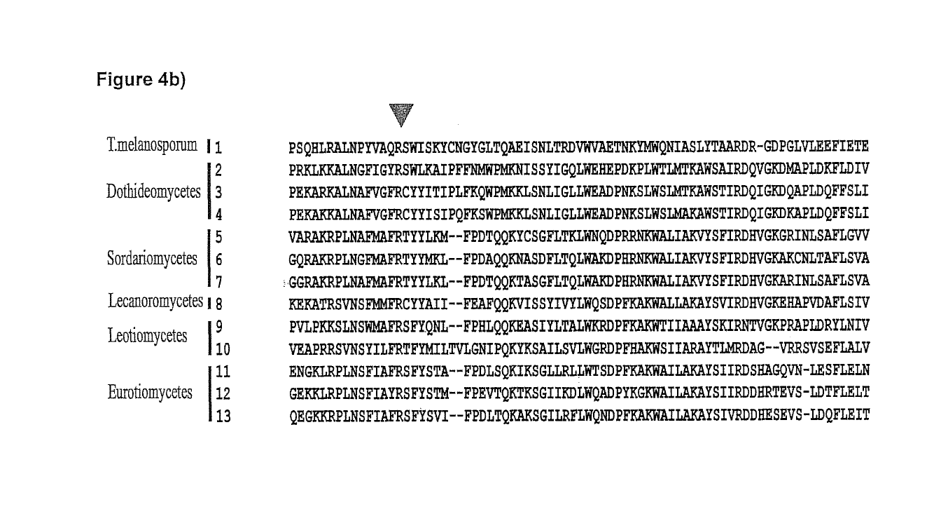

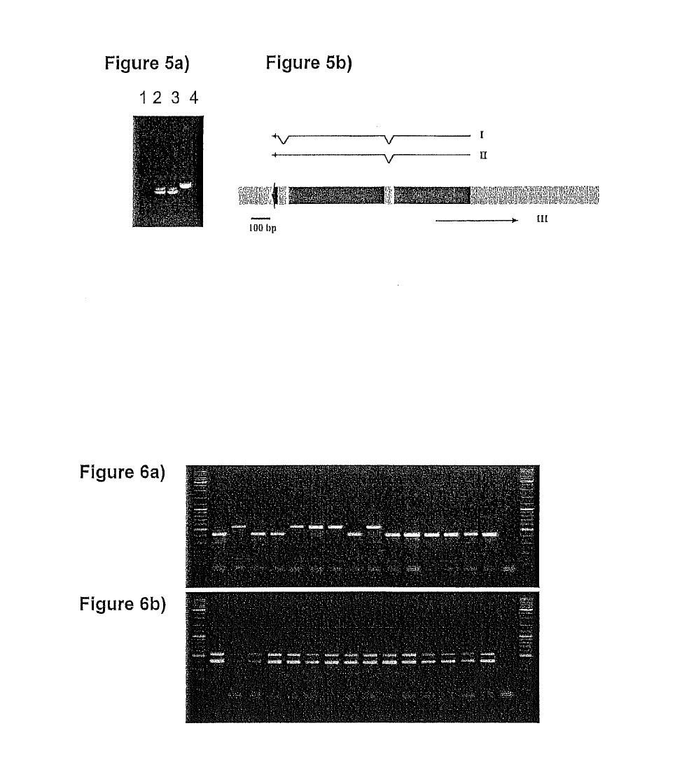

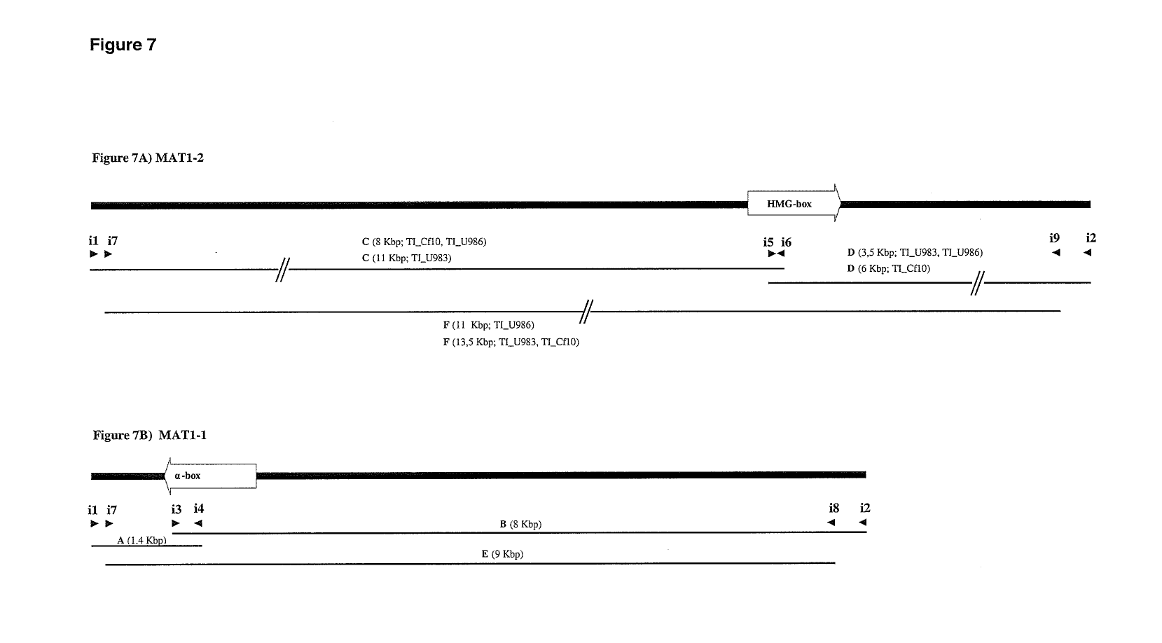

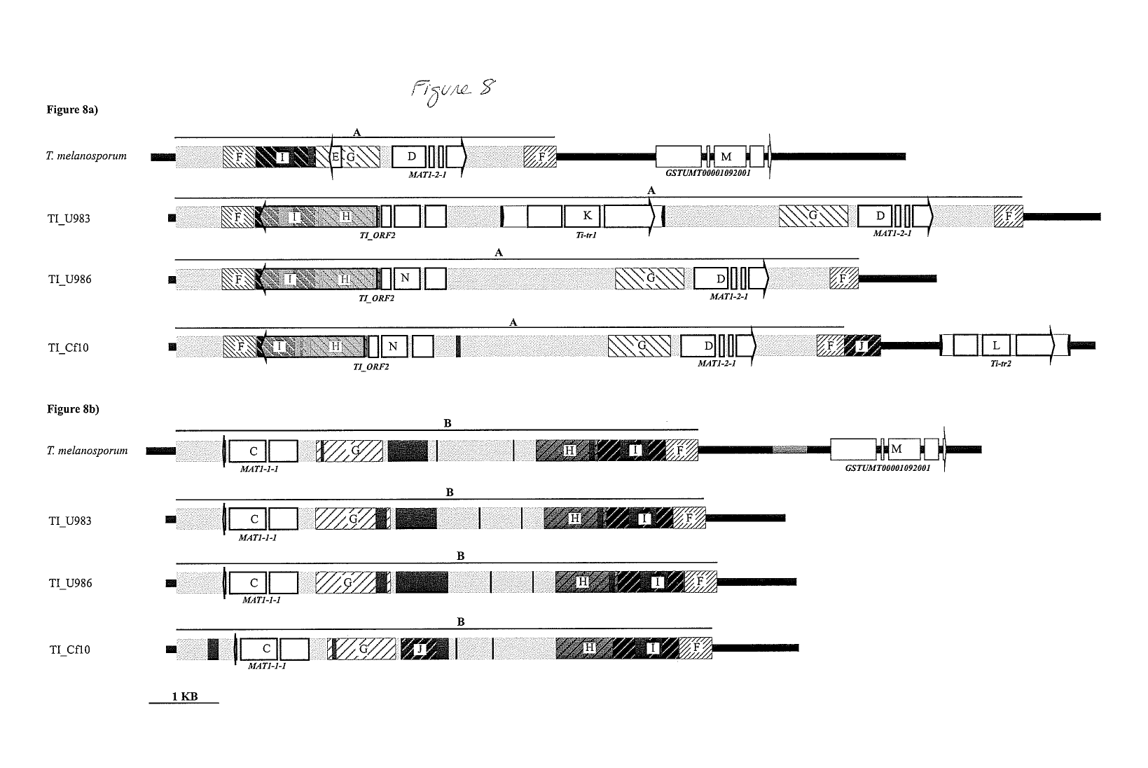

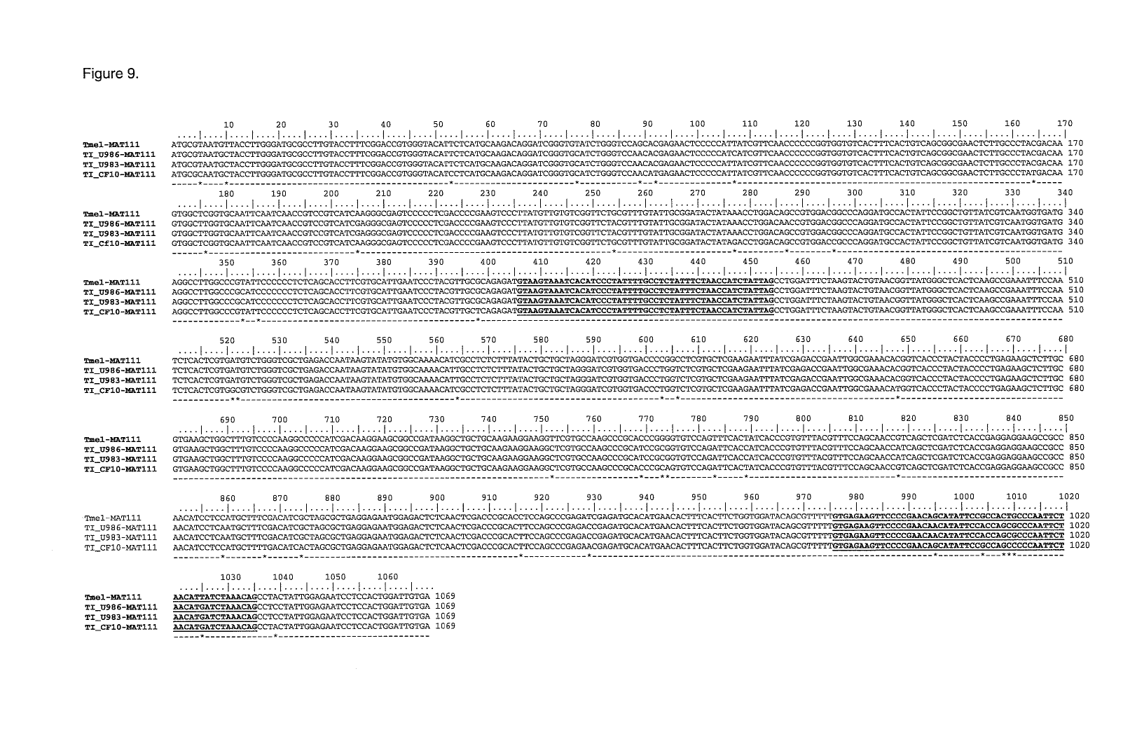

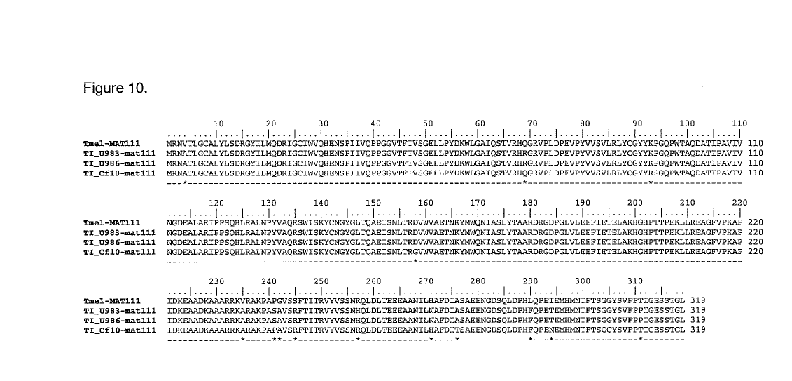

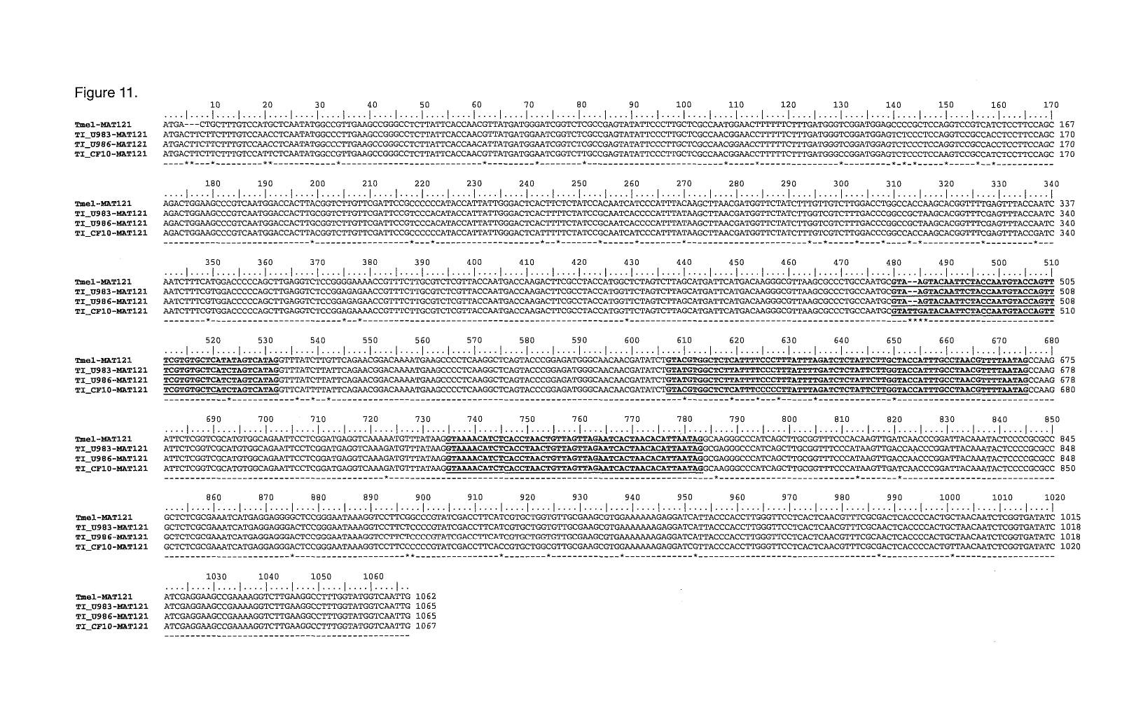

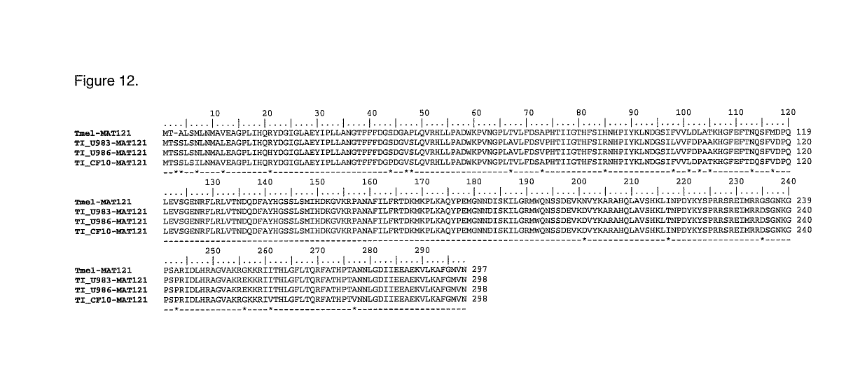

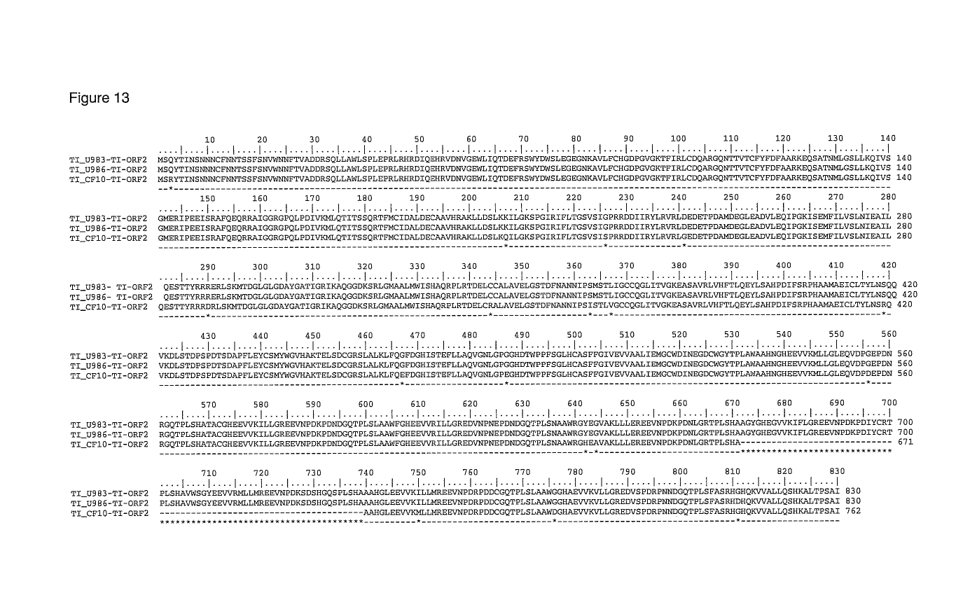

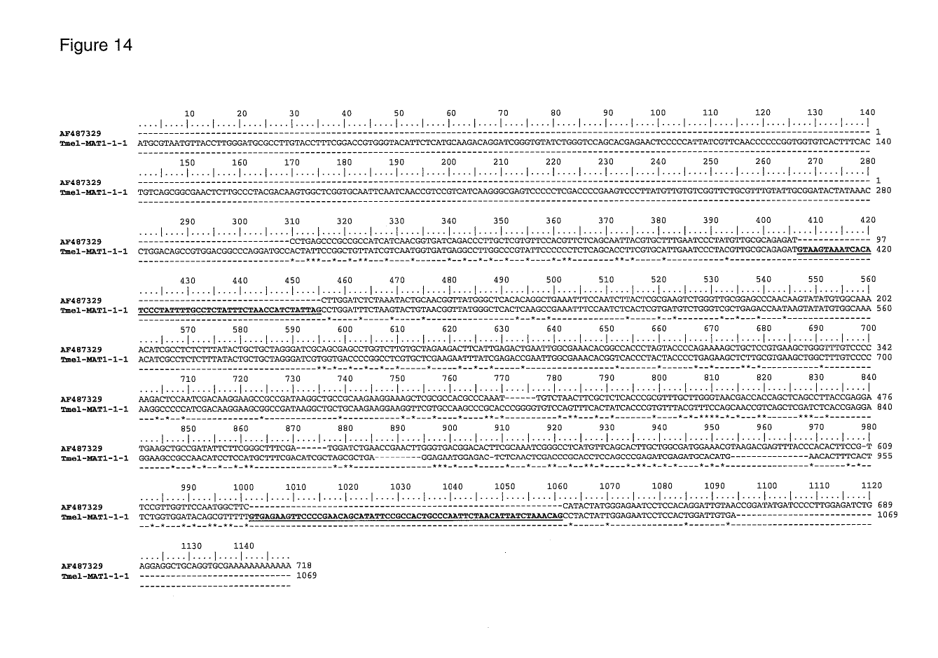

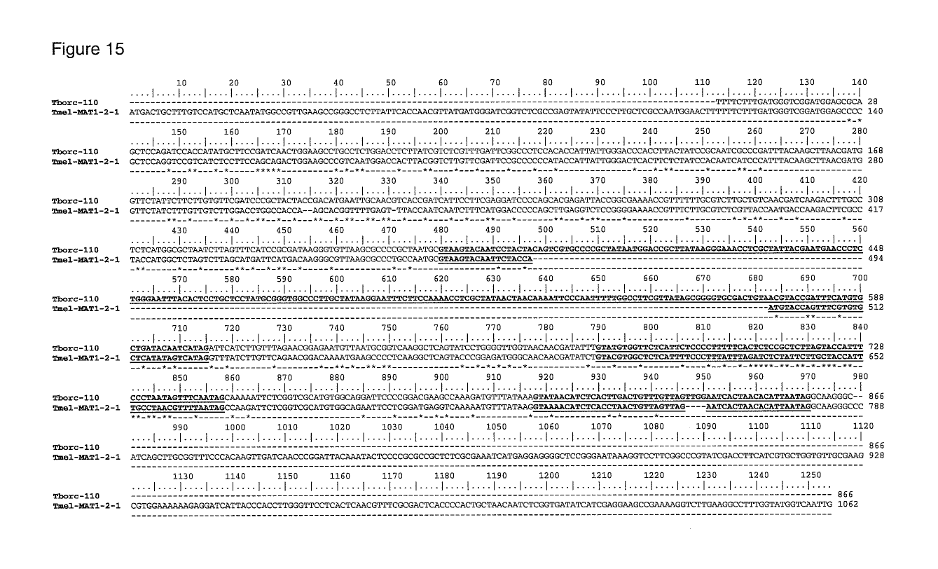

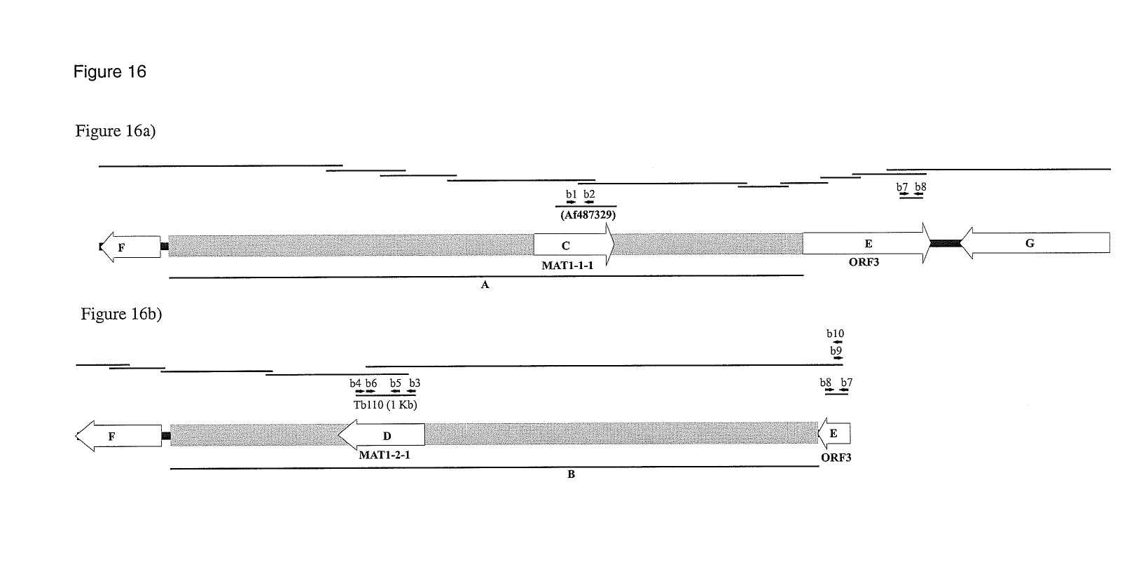

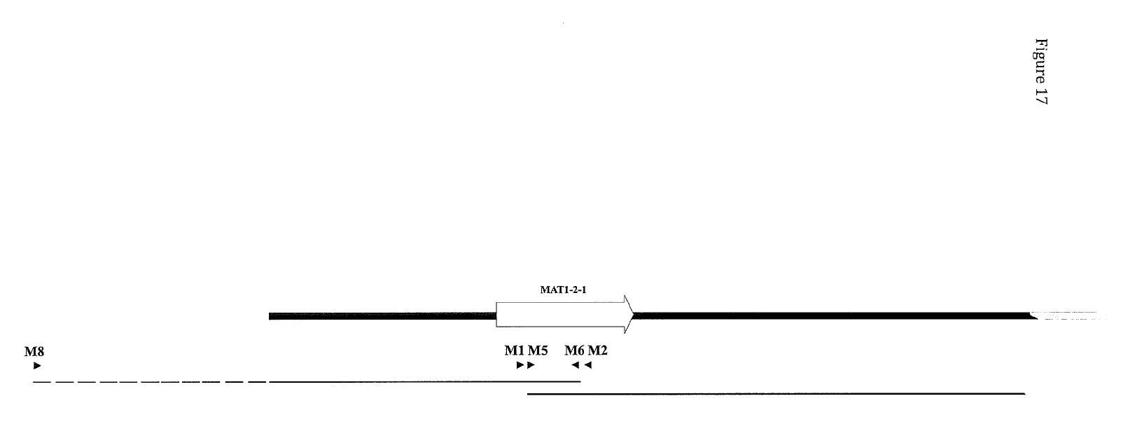

The small black, white and grey arrows indicate the position of common primers or MAT1-1 and MAT1-2 idiomorph-specific primers, respectively. Primers numbers are as in table 1. The black lines at the bottom of the FIG. 1 indicate the PCR amplicons (Fa, Fb, Fc and Fd) assembled to give the entire MAT1-1-1 contig (15.144 bp).

FIGS. 2a, 2b and 2c represent PCR amplification of three HMG-domain encoding genes in T. melanosporum.

PCR amplifications were performed with the primer pair's p7/p8, p9/p10 and p1/p2 designed on the gene models GSTUMT00008644001 (a), GSTUMT00000587001 (b) and GSTUMT00001090001 (c), respectively.

Each PCR was performed with genomic DNA from the following ascocarps: lanes 2-13: me443, me448, me458, me459, me66, me67, me68, me300, me303, me225, me206, me151; lane 14: negative control (no DNA template); lanes 1 and 15: DNA ladder mix (Fermentas).

FIG. 3 represents a dot-plot comparison of MAT1-1 and MAT1-2 DNA sequences highlighting the extent of the idiomorphic region in each mating type and the repeated sequences. The idiomorphic regions are represented as in FIGS. 1a and 1b.

FIGS. 4a and 4b represent amino acid alignment of conserved regions of T. melanosporum MAT proteins with those from others Ascomycetes.

FIG. 4a) represents an amino acid alignment of conserved HMG domain of the following species: 1) Tuber melanosporum; 2) Leptosphaeria maculans (AA037761); 3) Mycosphaerella graminicola (AAL30836); 4) Pyrenophora teres (AAY35017); 5) Cochliobolus sativus (AAF87724); 6) Dendryphiella sp. (AAR83920); 7) Alternaria alternata (BAA75908); 8) Magnaporthe grisea (BAC65094); 9) Gibberella fujikuroi (AAC71056); 10) Ceratocystis eucalypti (AAF00498); 11) Isaria tenuipes (BAC67543); 12) Colletotrichum musae (CAD59615); 13) Sordaria brevicollis (CAB63222); 14) Neurospora crassa (AAA33598); 15) Podospora anserina (CAA45520); 16) Cryphonectria parasitica (AAK83343); 17) Xanthoria flammea (CAI59780); 18) Xanthoria elegans (CAI59778); 19) Xanthoria polycarpa (CAI59768); 20) Cladonia galindezii (AAT48651); 21) Pyrenopeziza brassicae (CAA06843); 22) Penicillium chrysogenum (CAP17333).

FIG. 4b) represents an amino acid alignment of conserved α-box region of the following species: 1) Tuber melanosporum; 2) Phaeosphaeria nodorum (XP_{—}001791062); 3) Stemphylium callistephi (AAR04468); 4) Alternaria brassicicola (AAK85542); 5) Gibberella avenacea (CAD59608); 6) Fusarium poae (CAD59610); 7) Colletotrichum musae (CAD59611); 8) Xanthoria polycarpa (CAI59771); 9) Pyrenopeziza brassicae (CAA06844); 10) Botryotinia fuckeliana (XP_{—}001546438); 11) Ajellomyces capsulatus (ABO26868); 12) Penicillium marneffei (ABC68484); 13) Neosartorya fischeri (XP_{—}001263836).

Ascomycete families are indicated with vertical bars. The position of T. melanosporum specific introns and those conserved in most of the Ascomycetes are indicated by black and white triangles respectively. In the α-box region alignment, the grey triangles indicate the position of an intron conserved in some Ascomycetes but absent in T. melanosporum

FIGS. 5a and 5b represent MAT1-1 gene structure as inferred from RT-PCR and RACE analyses. FIG. 5a) represents PCR amplification with primers p27 and p28; Lane 1 is a 1 Kb DNA ladder (Fermentas). Lanes 2 and 3 are respectively cDNA from MAT1-1 ascocarps me206 and me151. Lane 4 is a genomic DNA from me206. FIG. 5b) represents a schematic representation of MAT1-1 gene structure (dark boxes) and the two different cDNA detected (I and II). The putative antisense transcript (III) is also reported.

FIGS. 6a and 6b represent respectively a multiplex PCR amplification of DNA from ascocarps and a purified asci with mating type specific primers SEQ ID N ^{o}7 (p1); SEQ ID N ^{o}8 (p2); SEQ ID N ^{o}9 (p19); SEQ ID N ^{o}10 (p20).

Lanes 1 and 18: DNA ladder mix (Fermentas); lanes 2-16: ascocarps me442, me443, me444, me445, me448, me458, me459, me69, me66, me71, me222, me182, me226, me334, me335; lane 17: negative control (no DNA template).

FIG. 7 represents the PCR—based strategy followed to isolate the T. indicum MAT1-2 (A) and MAT1-1 (B) idiomorphs.

FIG. 8 represents the T. indicum and T. melanosporum mating type regions. A represents the nucleotide sequences (SEQ ID N^{o} 3, 34, 35 and 36) for the first mating type idiomorph (MAT1-2) of T. melanosporum, and T. indicum TI_U983, TI_U986 and TI_CF10 samples, respectively. B represents the nucleotide sequences (SEQ ID N^{o} 4, 37, 38 and 39) for the second mating type idiomorph (MAT1-1) of T. melanosporum and T. indicum TI_U983, TI_U986 and TI_CF10 samples, respectively. The black lines indicate the common flanking regions. C (SEQ ID N^{o} 1, 40, 41 and 42) and D (SEQ ID N^{o} 2, 43, 44 and 45) boxes represent the nucleotide sequence of the HMG-box (mating type gene MAT1-2-1) and of the α-box (mating type genes MAT1-1-1) of T. melanosporum and T. indicum, respectively. E arrowed box indicates the gene model GSTUMT00001089001. The F, G, H, I and J hatched boxes represent regions sharing sequence similarities between idiomorphs. The 45° grid pattern indicates the opposite orientation of similar sequences within and between idiomorphs. The arrowed boxes K and L indicate the putative transposons Ti_TR1 and TI_TR2, respectively. The arrowed boxes M and N indicates the gene model GSTUMT00001092001 and the TI_ORF2 gene, respectively.

FIG. 9 represents the nucleotide alignment of T. indicum and T. melanosporum MAT1-1-1 genes. Polymorphic nucleotide positions are indicated with asterisks; Introns are shown in bold underlined type.

FIG. 10 represents the amino acid alignment of T. indicum and T. melanosporum MAT1-1-1 deduced proteins. Polymorphic amino acid positions are indicated with asterisks.

FIG. 11 represents the nucleotide alignment of T. indicum and T. melanosporum MAT1-2-1 genes. Polymorphic nucleotide positions are indicated with asterisks; Introns are shown in bold underlined type

FIG. 12 represents the amino acid alignment of T. indicum and T. melanosporum MAT1-2-1 deduced proteins. Polymorphic amino acid positions are indicated with asterisks.

FIG. 13 represents the amino acid alignment of T. indicum TI_ORF2 hypothetical protein. Polymorphic amino acid positions are indicated with asterisks.

FIG. 14 represents the nucleotide alignment of T. melanosporum MAT1-2-1 gene and T. borchii sequence AF487329. Polymorphic nucleotide positions are indicated with asterisks. Introns are shown in bold underlined type. Position and length of introns in T. borchii were desumed on the basis of the alignment of AF487329 cDNA with the genomic sequence of TmelMAT1-2-1

FIG. 15 represents the nucleotide alignment of T. melanosporum MAT1-1-1 gene and T. borchii sequence obtained with primer pair b3/b4. Polymorphic nucleotide positions are indicated with asterisks. Introns are shown in bold underlined type. Position and length of introns in T. borchii were desumed on the basis of the alignment of T. borchii MAT1-1-1 with the genomic and cDNA sequences of TmelMAT1-1-1

FIG. 16 represents the amplification strategy used to sequence the genomic regions containing the MAT1-1 (a) and MAT1-2 (b) idiomorphs of T. borchii.

A represents the MAT1-1 mating type idiomorph whose nucleotide sequence is SEQ ID N ^{o}53. B represents the MAT1-2 mating type idiomorph whose nucleotide sequence is SEQ ID N^{o} 55. The thick black lines indicate the common flanking regions. C (SEQ ID N^{o} 52) and D (SEQ ID N^{o} 54) arrowed boxes represent the α-box containing mating type gene (Tborchii MAT1-1-1) and the HMG-box containing mating type gene (Tborchii MAT1-2-1), respectively. E arrowed boxes indicate genomic regions with homology to T. melanosporum gene model GSTUMT00001092001 (ORF3). The F and G arrowed boxes represent regions with similarity with an ankyrin repeat-containing protein (XP_{—}0021443441) and a origin recognition complex subunit ORC1 (CBX92828), respectively.

The thin lines above the schemes of each idiomorph show the fragments, resulting from a combination of Genome walking, Inverse PCR and Long distance PCR procedures, used to assemble these two genomic regions. The small black arrows represent the primers numbered as in Table 1.

FIG. 17 represents the primers M1, M2, M5 and M6 positioned with Mat1-2-1.

### EXPERIMENTATIONS

### A. Tuber melanosporum

1. Materials and Methods

1.1. PCR Amplification and Sequencing

The primers' sequences and annealing sites on or near the MAT locus are reported in Table 1 and FIG. 1. PCR amplifications were performed in a 50-p1 mixture containing 16 mM (NH₄)₂SO₄, 67 mM Tris-HCl (pH 8.8), 0.01% Tween-20, 2.5 mM MgCl₂, 0.2 mM of each dNTP, 10 nM of each primer, 1 U of EuroTaq polymerase (Euroclone, Milano, Italy) and 20 ng of DNA. DNA comes from ascocarps and pools of spores that were isolated according to Paolocci et. al (2006) (complete the references). PCR reactions were performed in a 9700 PCR system (Applied Biosystems, Foster City, Calif., USA) with the following thermal profile: 2 min at 94° C. followed by 35 cycles of denaturation at 94° C. for 30 s, annealing at 55-60° C. for 30 s, extension at 72° C. for 30 s to 3 min according to the length of the expected amplicon, and a final extension at 72° C. for 7 min. Long-range PCR amplification of the MAT1-1 region (about 10 Kb) was performed using LA Taq DNA polymerase (TaKaRa Bio Inc., Otsu, Japan) and a 2-step thermal profile: 30 s at 94° C., 30-40 cycles of denaturation at 94° C. for 30 s, annealing/extension at 68° C. for 15 min, and a final extension for 7 min at 72° C. The analysis of simple sequence repeat (SSR) loci was performed according to Riccioni et al. (2008) New Phytologist 180: 466-478.

The PCR amplicons were purified using the EuroGold Cycle-Pure PCR purification kit (Euroclone, Milano, Italy) or cloned into the pGEM T-easy vector (Promega, Madison Wis., USA) according to the manufacturer's instructions. Sequences were obtained using the BigDye sequencing kit (Applied Biosystems) according to the manufacturer's protocol. Sequence assembly was performed using Bioedit software v. 7.0.9 (Hall, 1999 Nucleic Acids Symposium. 41: 95-98). Primers were designed with the help of PerlPrimer software v. 1.1.16 (Marshall, 2004 Bioinformatics 20:2471-2472). Sequence comparison and dotplot analysis were carried out using NCBI BLAST and Dotmatcher (EMBOSS Package v. 6.0.1). Gene structure prediction of MAT1-1-1 was performed with FGENESH software (http://linux1.softberry.com) using N. crassa as a reference genome.

1.2. RNA isolation, RT-PCR and RACE analyses

Total RNA was isolated from ascocarps stored at −80° C. according to the protocol described by Chang et al. (1999) Plant Molecular Biology Reports 11: 113-116. Rapid amplification of cDNA ends (RACE) was carried out using the SMART-RACE cDNA Amplification Kit (Clontech, Palo Alto, Calif., USA) according to the manufacturer's protocol. The 5′ and 3′ RACE gene-specific primers were p21 and p23, and the nested primers were p22 and p24, respectively (Table 1, FIG. 1). To confirm the structure of MAT1-1-1 gene, about 5 pg of total RNA isolated from two ascocarps that had gleba with the idiomorph MAT1-1 were reverse-transcribed using the SuperScript® III RT enzyme (Invitrogen, Carlsbad, Calif.) and 200 ng of random primers (Invitrogen, Carlsbad, Calif.) according to the instructions of the enzyme's supplier. First-strand cDNAs were then amplified according to the PCR protocol reported above, using the primers p27 and p28 (Table 1 FIG. 1).

TABLE 1
list of primers used

| Primer | |
|---|---|
| name | Primer sequence 5′-3′ |
| p1 | CAGGTCCGTCATCTCCTTCCAGCAG |
| | (SEQ ID N^{o} 7) |
| p2 | CCACATGCGACCGAGAATCTTGGCTA |
| | (SEQ ID N^{o} 8) |
| p3 | CAATCTCTTCCATCGCCCGTCCAG |
| p4 | TGGTATATGTGGATGTATTGATAACTATAAT |
| | (SEQ ID N^{o} 11) |
| p5 | AGAGATAGAGAAATAGCATGGCTCGG |
| | (SEQ ID N^{o} 12) |
| p6 | AAGTAACCTTTGTGCCATTGCTCCA |
| p7 | CAGCATGGTCTGGTTGGACGTTTTGAT |
| p8 | CTATGCACCCAAGTGTGATTGAGGAC |
| p9 | CCATTCGTTGAGATGCGCCGTTGCTA |
| p10 | AGGCGGTATCAGAGCTTTTCCATGCAG |
| p11 | GCCAACCTCTAGTTGGGATATTTGTTCAGGAC |
| p12 | GTGAATAGACAGACTAGCTTGCCCG |
| p13 | AATCTCTTAATGTCCAGACCCTTACC |
| p14 | TGAATCAAGGTAGTTTGCTGATTGAGG |
| p15 | TATCGTACACCACTTTCCTTCTATCTG |
| p16 | GATCTTCAGCCACTGGCGAATATCC |
| p17 | GTTCCTCCGATCTTCCAAGAATTCCAG |
| p18 | TAATTACATTCCCAGAGCTAGGTGCCC |
| | (SEQ ID N^{o} 14) |
| p19 | CAATCTCACTCGTGATGTCTGGGTC |
| | (SEQ ID N^{o} 9) |
| p20 | TCTCGGGCTGGAGGTGCGGGTCGAGT |
| | (SEQ ID N^{o} 10) |
| p21 | GCCGGAATAGTGGCATCCTGGGCCGTC |
| p22 | GACGGACGGTTGATTGAATTGCACCGAG |
| p23 | GATCGTGGTGACCCCGGCCTCGTGCT |
| p24 | CTTGCGTGAAGCTGGCTTTGTCCCCAAG |
| p25 | GTGTGCGTGCTTGAAGTGAATTGGAGTAA |
| | (SEQ ID N^{o} 13) |
| p26 | ATCCTAACTTACAGCATCTCGCCT |
| p27 | TATCGGCTCACAATCCAGTGGAGGATTC |
| p28 | ACGGCCCAGGATGCCACTATTC |

2. Results

2.1. Searching for Mating Type Genes in T. Melanosporum Genome.

The T. melanosporum genome sequence database “TuberDB” (http://mycor.nancy.inra.fr/IMGC/TuberGenome/) was investigated for the presence of orthologs of Ascomycetes MAT genes in the sequenced strain Mel28 using BLASTN and TBLASTX similarity searches. Orthologs, are homologous genes in different species that are similar to each other because they originated from a common ancestor. Using Ascomycetes HMG-containing sequences as queries, 3 candidate gene models were identified in the Mel28 genome: GSTUMT00001090001 (scaffold 247), GSTUMT00000587001 (scaffold 207) and GSTUMT00008644001 (scaffold 49). BLASTP searches against GenBank revealed that the GSTUMT00001090001 predicted protein was the best match for the MAT1-2-1 gene from Diaporthe sp. (BAE93753, BAE93759), Cryphonectria parasitica (AAK83343), Fusarium sacchari (BAE94382), and other Ascomycetes. The GSTUMT00000587001 predicted protein showed the best BLASTP match with HMG-box containing proteins, especially with a Ste11 transcription factor of S. pombe (CAA77507), whereas the GSTUMT00008644001 predicted protein showed similarity with the HMG-box transcriptional regulator of A. fumigatus (XP_{—}751745.1). Conversely, BLAST searches using α-box- and homeodomain-containing MAT genes from different Ascomycetes as queries did not allow the identification of any significant matches with putative sex genes in the Mel28 genome.

2.2. Selection of Gene Model Harbouring the MAT1-2-1 Gene and Identification of Strains Harbouring the Opposite Mating Type (FIGS. 1, 2a, 2b and 2c).

To assess for the presence and distribution of the three HMG-box-containing genes in different T. melanosporum strains, the primers pairs: p1/p2, p7/p8, and p9/p10, specific to the gene models GSTUMT00001090001, GSTUMT00000587001 and GSTUMT00008644001, respectively, where used to perform a PCR screening on 12 T. melanosporum ascocarps of different geographical origin.

The GSTUMT00000587001 and GSTUMT00008644001 gene-specific amplicons were obtained from all the ascocarps tested, FIGS. 2a and 2b. Conversely, only ten of the twelve samples screened using the primer pair p1/p2 produced the expected amplicon of 550 bp, FIG. 2c.

On the basis of these results the GSTUMT00001090001 was selected as the sex- candidate MAT1-2-1 gene and the two samples (me206 and me151) that did not produce the GSTUMT00001090001-specific amplicon regarded to as strains putatively harbouring the opposite mating type idiomorph (MAT1-1).

The couple of primers p1/p2 can be used to amplify a nucleotide sequence specific to the first mating type gene of T. melanosporum (MAT1-2-1). The primer p1 has, as nucleotide sequence, the nucleotide sequence SEQ ID N ^{o}7 and the primer p2 has, as nucleotide sequence, the nucleotide sequence SEQ ID N ^{o}8.

2.3. Isolation of MAT1-1 idiomorph (FIG. 1, table 1).

Embracing the hypothesis of heterothallism, the 5′ and 3′ regions flanking the two putative idiomorphic structures are expected to be conserved between the sequenced strain (Me128) and strains that lack the MAT1-2-1 genes such as those that form the gleba of truffles me206 and me151.

Thus, on Mel28 strain, a set of primers were designed upstream and downstream the MAT1-2-1 gene to search for genomic regions conserved between strains of different mating type (Table 1, FIGS. 2a, 2b, 2c). To this purpose, on the 5′ MAT1-2-1 flanking region, the primer pairs p3/p12, p13/p14, p15/p14 and p3/p14 (Table 1, FIG. 1) were tested for their capacity to yield amplicons of expected length of about 550, 400, 1700 and 2800 bp, respectively, on all samples screened. These primers pairs produced amplicons only when strains containing the MAT1-2 idiomorph were amplified. Conversely, the primer pair p3/p4 yielded an amplicon of about 370 bp from both MAT1-2 and MAT1-1 strains. On the 3′ MAT1-2-1 flanking region of both the MAT1-2 and MAT1-1 strains, the primer pairs p16/p6 and p17/p18 yielded amplicons of about 400 and 1000 bp, respectively. The primer pair p5/p6 produced an amplification product of about 1200 bp in MAT1-2 strains and a fragment of about 1700 bp in MAT1-1 strains. Overall, these preliminary screenings show that the binding capacity of p3, p4 and of p5, p6 primers was conserved in the upstream and downstream MAT1-2-1 gene regions, respectively, in strains both with and without the MAT1-2 idiomorph.

The PCR fragments obtained from sample me206 with primer pairs p3/p4 and p5/p6 were cloned and sequenced. The alignments showed that that these two fragments were highly similar (about 96% of sequence identity) to the corresponding regions in the Mel28 genome, although the fragment generated by the primer pair p5/p6 (FIG. 1, fragment Fc) showed an insertion of 493 bp in the 3′ region downstream the MAT1-1 idiomorph (FIG. 1b).

On this insertion, the primer p11, specific for MAT1-1 strains, was designed and used in combination with the primer p3 to amplify the genomic region containing the MAT1-1 idiomorph from strain me206.

The amplification with the primer p11 and the primer p3 yielded a fragment of about 9 Kbp (FIG. 1, fragment Fa) whose sequencing confirmed the identification of the MAT1-1 idiomorph. Indeed, BLASTX search against GenBank revealed the presence of about 200 bp region with similarity to the α-box domain that typifies the MAT1-1-1 gene of filamentous Ascomycetes. In particular, BLASTX matches were observed with α-box domains of MAT1-1 proteins of Alternaria brassicae (AAK85543.1), Talaromyces stipitatus (XP_{—}002486084), Aspergillus nidulans (XP_{—}660359), Pyrenophora sp. (CAP08782), and Penicillium marneffei (ABC68484).

The genomic fragment containing the MAT1-1 idiomorph was further extended by PCR at the 5′ and 3′ ends using the primers p24 and p26 coupled with the MAT1-2 primers p25 and p18, respectively. The sequenced fragments Fa, Fb, Fc, and Fd were assembled in a 15,144 bp contig (FIG. 1).

The couple of p19/p20 was then designated and can be used to amplify a nucleotide sequence specific to the second mating type gene of T. melanosporum (MAT1-1-1). The primer p19 has, as nucleotide sequence, the nucleotide sequence SEQ ID N ^{o}9. The primer p20 has, as nucleotide sequence, the nucleotide sequence SEQ ID N ^{o}10.

Mating type specific amplification can be also obtained using any others couples of primers that are specific to the first and/or to the second mating type idiomorph of T. melanosporum.

The entire MAT1-2 idiomorph sequence or SEQ ID N ^{o}3 nucleotide sequence is available in T. melanosporum genome database “TuberDB” (http://mycor.nancy.inra.fr/IMGC/TuberGenome/).

The entire MAT1-1 idiomorph sequence or SEQ ID N ^{o}4 was deposited in GenBank under the accession number HM370280.

2.4. Structure of MAT Idiomorphs.

Sequence alignment and dotplot analyses of the two genomic regions showed that the MAT1-2 and MAT1-1 idiomorphs were about 5550 bp and 7430 bp long, respectively (A in FIG. 1a, B in FIG. 1b and FIG. 3). The MAT1-2 idiomorph has as nucleotide sequence the nucleotide sequence SEQ ID N ^{o}3 and the MAT1-1 idiomorph has as nucleotide sequence the nucleotide sequence SEQ ID N ^{o}4. The sequences flanking these idiomorphs were highly conserved: pairwise sequence alignment showed that the 5′ flanking regions (3603 bp) share 99,1% sequence identity and the 3′ flanking regions (3586 bp), excluding the 493-bp long insertion of MAT1-1, share 91,3% of identity.

Each idiomorph contains only a single MAT gene (HMG or α-box containing gene).

However, blast and dotplot analyses (FIGS. 4 and 3) revealed that the idiomorphic regions were not completely different. In fact, the presence of shared sequences arranged in inverse orientations was observed (FIG. 1). Pairwise alignments showed 960 bp- (FIG. 1: G box) and 950 bp- (FIG. 1: H box) long sequences sharing 76.5% and 71.1% sequence identity, respectively. Additionally, BLASTX analysis revealed a region within the 960-bp sequence (G box) with similarity to conserved ankyrin repeat domains. An EST sequence (SYOAAA61YO16FM1) from Mel28 mycelium relative to GSTUMT00001089001 proved that the transcript of this gene extends its 3′ end into the ankyrin-containing region, but that this last region is not included in the predicted CDS because it is only 57 amino acids long. Furthermore, no ORF could be identified in the corresponding region of the MAT1-1 idiomorph.

Finally, a short repeated sequence of about 460 bp was present in inverse orientation in the MAT1-2 idiomorph (FIG. 1: F boxes). A similar sequence (84% sequence identity) was also present at the extreme 3′ end of the MAT1-1 idiomorph.

2.5. Structure of MAT Genes

The analysis of EST produced by the Tuber Genome Consortium showed that the T. melanosporum MAT1-2-1 gene (TmelMAT1-2-1) consisted of 4 exons since it showed 2 additional introns to the one conserved in most of the Ascomycetes (FIGS. 1a and 4a). The deduced amino acid sequence was 297 residues long and contained a conserved HMG-box motif typical of the MAT1-2-1 genes of Ascomycetes (FIG. 4a).

Gene prediction analysis (performed with FGENESH in the MAT1-1 idiomorph) suggested the presence of 3 exons within the T. melanosporum MAT 1-1-1 gene (TmelMAT1-1-1), FIG. 1b. RACE and RT-PCR analyses were performed on RNA isolated from fruiting bodies harbouring the MAT1-1 idiomorph to confirm the predicted gene structure. Interestingly, these analyses showed that the second intron could be differentially spliced to give putative proteins of either 319 or 399 aminoacids, (FIGS. 5a and 5b). When RACE analysis was performed using the polyA-anchored primer in combination with the primers for 5′ RACE (p21, p22) on the 5′ extended cDNA pools as control, it revealed the presence of an antisense transcript partially overlapping the MAT1-1-1 CDS (FIGS. 5a and 5b).

2.6. MAT genes are expressed both during the vegetative and reproductive phases.

RT-PCR analyses along with the availability of ESTs (http://mycor.nancy.inra.fr/) from fruit body (FB) free living mycelia (FLM) and ectomycorrhizae (ECM) (Martin et al. 2010, Nature 464:1033-1038) allowed the inventors to evaluate transcription of MAT genes at different phases of the T. melanosporum life cycle.

The analysis of EST datasets confirmed that in FLM of the Mel28 strain used for genome sequencing, only the MAT1-2-1 gene was transcribed. On the contrary, both MAT1-1-1 and MAT1-2-1 transcripts were detected in the EST libraries obtained from FB and ECMs.

2.7. MAT Idiomorphs are Equally Distributed Among Truffles within Single Populations.

The presence and distribution of mating type genes in the ascomata collected from natural populations were assessed using a multiplex-PCR approach. To this end, the α-box-specific primer pair p19 (SEQ ID N ^{o}9)/ p20 (SEQ ID N ^{o}10) was designed to produce an amplicon of 421 bp and combined with the HMG box-specific primer pair p1 (SEQ ID N ^{o}7)/ p2 (SEQ ID N ^{o}8) that produced an amplicon of 550 bp. As shown in Table 2, truffles harbouring either the MAT1-2 or the MAT1-1 idiomorphs were identified within each local population.

2.8. All T. melanosporum Truffles Results from Outcrossing.

To test the hypothesis that all T. melanosporum truffles were derived from outcrossing and that T. melanosporum is a “truly” heterothallic spp., the multiplex PCR approach described above was carried out on asci and gleba isolated from single, selected ascocarps.

When screened with SSR and single nucleotide polymorphism (SNP) markers, the asci of most ascocarps did not display any additional alleles with respect to their corresponding gleba (Riccioni et al., 2008, New Phytologist 180: 466-478). Whether these truffles resulted from the crossing of two parents or just from selfing of homothallic strains remained elusive. Multiplex PCR-based mating type screening showed that the gleba of these ascomata had either the MAT1-2- or MAT1-1—specific allele (FIG. 6a). In stark contrast, the pool of spores always presented, with an approximately equal intensity, of both MAT1-2- and MAT1-1-specific amplicons, irrespective of the truffle samples processed, the mating type of the corresponding gleba, and the number of PCR cycles performed (FIG. 6b).

Altogether, these results suggest that the additional mating idiomorph detected in the asci with respect to the surrounding gleba must be contributed by a partner with a different sexual polarity, and that this condition is common to all truffles screened.

TABLE 2
List of truffles whose gleba was genotyped
using mating-specific primers

| Sample | Population | Mating type |
|---|---|---|
| 442 | 2 | α-box |
| 443 | 2 | hmg |
| 444 | 2 | α-box |
| 445 | 2 | α-box |
| 446 | 2 | α-box |
| 448 | 2 | hmg |
| 458 | 2 | hmg |
| 471 | 2 | α-box |
| 478 | 2 | α-box |
| 459 | 2 | hmg |
| 476 | 2 | α-box |
| 482 | 2 | hmg |
| 280 | 2 | α-box |
| 284/287 | 2 | hmg |
| 281 | 2 | α-box |
| 206 | 2 | α-box |
| 151 | 2 | α-box |
| 66 | 6 | hmg |
| 69 | 6 | α-box |
| 67 | 6 | hmg |
| 68 | 6 | hmg |
| 71 | 6 | α-box |
| 70 | 6 | hmg |
| 296 | 7 | hmg |
| 300/303 | 7 | hmg |
| 298 | 7 | α-box |
| 180 | 10 | α-box |
| 224 | 10 | hmg |
| 222/182/226/334/335/227 | 10 | α-box |
| 225 | 10 | hmg |
| 336 | 10 | α-box |
| 337 | 10 | hmg |
| 183 | 10 | α-box |
| 294 | 12 | hmg |
| 367 | 12 | α-box |
| 81 | 12 | hmg |
| 83 | 12 | hmg |

B. Tuber indicum

1. Materials and Methods

1.1. Fungal samples and PCR analysis of ITS T. indicum ascocarps used in this study are reported in Table 3. The genomic DNA was isolated from ascocarps according to Paolocci et al. (2006) Applied and environmental microbiology 72: 2390-2393. The PCR amplification and RFLP analysis of ITS region were performed using ITS1 and ITS4 primers (White et al. 1990 Academic Press, New York, pp 315-322) and RSAI endonuclease according to Paolocci et al. (1997) FEMS Microbiology Letters 153: 255-260.

TABLE 3
List of T. indicum ascocarps

| | ITS/RFLP | |
|---|---|---|
| T. indicum (code) | class | MAT* |
| TI_C69 | A | 1-1 |
| TI_CF11 | A | 1-2 |
| TI_C80 | A | 1-1 |
| TI_D5 | A | 1-1 |
| TI_D9 | A | 1-2 |
| TI_CF10 | A | 1-2 |
| TI_TC37 | A | 1-1 |
| TI_RIBC | B1 | 1-2 |
| TI_C20 | B1 | 1-1 |
| TI_D54 | B1 | 1-1 |
| TI_U983 | B1 | 1-2 |
| TI_F7 | B1 | 1-2 |
| TI_LI2 | B1 | 1-1 |
| TI_LI8 | B2 | 1-2 |
| TI_CF7 | B2 | 1-1 |
| TI_C8 | B2 | 1-2 |
| TI_C18 | B2 | 1-2 |
| TI_C38 | B2 | 1-2 |
| TI_TC61 | B2 | 1-1 |
| TI_U986 | B2 | 1-2 |

*PCR performed with T. melanosporum primers P1/P2 (MAT1-2) and P19/P20 (MAT1-1)

1.2 PCR Amplification and Sequencing of Mating Types

The primers used in this study are reported in Table 4 and FIG. 7. PCR amplification of HMG-box and α-box containing fragments of T. indicum was performed as described above for T. melanosporum except that the annealing temperature was lowered to 50° C.

Long distance PCR amplification of MAT1-1 and MAT1-2 fragments and of the entire idiomorph containing regions was performed using LA-Taq DNA polymerase (TaKaRa Bio Inc., Otsu, Japan) using a 2 step thermal profile: 30 at 94° C. followed by 25-30 cycle of denaturation at 94° C. for 30 s and annealing/extension at 68° C. for 10-15 min according to the length of the amplicons, and final extension of 7 min at 72° C.

In order to amplify both idiomorphs from a single ascocarp, a long distance PCR was performed as described above by using either the MAT1-2 or the MAT1-1 idiomorph specific primer pairs reported above. The number of PCR cycles was increased up to 50.

Sequencing, sequence assembly and analysis and primers design were performed as described above for T. melanosporum.

TABLE 4
List of primers

| Primer | |
|---|---|
| name | Primer sequence 5′-3′ |
| i1 | ACGTGGCAYTCTCTATTGGAGGATTCTG |
| i2 | ATYTTGCGCAGTTTATKGTCGCTCACAG |
| i3 | CCAGAAGTGAAAGTGTTCATGTGCATCTCG |
| i4 | CCCTACTACCCCTGAGAAGCTCTTGCG |
| i5 | ATGGTTCTATCTTGGTCGTCTTTGACCCG |
| i6 | TGGTAGGCGAAGTCTTGGTCATTGGTAAC |
| i7 | TCCTGCCTTAGGCCAATCCGCTTT |
| I8 | TGGACATGGGATCGGGTGGGAATAC |
| I9 | GGGATTGGGTGGGAGTATTGATCCTCG |

2. Results

PCR amplification of the ITS region produced an amplicons of approximately 600 bp in all T. indicum samples. The RFLP analysis of the ITS amplicons allowed the identification of three genetic classes (Table 3) that were named T. indicum A, T. indicum B1 and T. indicum B2 according to Paolocci et al. (1997) FEMS Microbiology Letters 153: 255-260.

2.1 Identification of T. indicum MAT Genes

Amplification of a portion of the mating type genes from T. indicum samples was first performed by using, under low stringent PCR conditions (see M&M), T. melanosporum MAT primers pairs P1/P2 (SEQ ID N^{o} 7/SEQ ID N^{o} 8) and P19/P20 (SEQ ID N^{o} 9/SEQ ID N^{o} 10) specific for MAT1-2 and MAT1-1 genes, respectively (Table 4). The primer pair P1/P2 produced an amplicon (approximately 500 bp) on 11 out of the 20 T. indicum ascocarps analyzed. These 11 ascocarps were then considered MAT1-2. The remaining nine ascocarps were classified as MAT1-1 because a PCR amplicon (about 400 bp) was obtained when amplified with the primer pair P19/P20 (Table 3). The PCR fragments from three MAT1-2 ascocarps (TI_U983, TI_U986 and TI_CF10) and from three MAT1-1 ascocarps (TI_TC61, TI_LI2, and TI_TC37), one representative of each of the three ITS/RFLP classes, were cloned and sequenced. Sequence analysis showed that these PCR fragments shared high similarity with HMG-box and α-box sequences of T. melanosporum, respectively.

2.2. Isolation of T. indicum MAT Idiomorphs.

In order to isolate the T. indicum idiomorphs, two MAT1-2-1 (named i5 and i6) and two MAT1-1-1 (named i3 and i4) primers were then designed and combined with the primers i1 and i2 designed on the 5′ and 3′ flanking regions of T. melanosporum (Table 4, FIG. 7). Using these primers, two fragments, spanning the entire idiomorphic region, were amplified from each of the six T. indicum samples. More specifically, the primer pairs i1/i4 and i2/i3 produced fragments of about 1.3 Kbp and 8 Kbp, respectively, in all three T. indicum MAT1-1 samples (TI_LI2, TI_TC61 and TI_TC37). The primer pair i1/i6 produced a fragment of about 8 Kbp from MAT1-2 samples TI_U986 and TI_CF10 and a fragment of 11 Kbp from TI_U983. The primer pair i2/i5 produced a fragment of 3.5 Kbp from TI_U983 and TI_U986 samples and a fragment of 6.5 Kbp from TI_CF10 sample (FIG. 7). This PCR-based analysis showed a similar orientation of MAT1-2-1 and MAT1-1-1 genes between T. indicum and T. melanosporum and length polymorphism in the MAT1-2-1 idiomorphs of T. indicum samples.

In order to selectively amplify the complete T. indicum regions containing the MAT1-2 and MAT1-1 idiomorphs, the 5′ and 3′ ends of T. indicum PCR amplicons A, B, C and D (FIG. 7) were sequenced and aligned. Then the MAT1-2- and MAT1-1- specific primers i8 and i9 were designed on the 3′ flanking region and the primer i7 common to both idiomorphs on the 5′ flanking region (Table 4, FIG. 7). The flanking regions were identified by comparison with T. melanosporum MAT locus.

The amplification pattern of MAT genes in T. indicum is consistent with a heterothallic organization of the MAT locus because the gleba of different T. indicum ascocarps produced the MAT1-1 or MAT1-2 specific amplicon. However, as previously observed in T. melanosporum (Rubini et al., 2011 New Phytologist 189: 710-722), the spores entrapped within the ascocarp contribute a slight amount of paternal DNA during the process of nucleic acid isolation. By using appropriate PCR conditions and MAT specific primers, DNA contributed from the spores in addition to that contributed by the gleba was PCR amplified enabling the isolation of both mating type sequences from a single ascocarp.

In turn, this approach ensures that both idiomorphs belong indeed to the same biological species. Whether in fact all the genetic variants exhibited by ascocarps showing the T. indicum morphotype belong to the same species is still a matter of debate among mycologists (Wang et al., 2006 Mycological Research 110: 1034-1045; Chen et al., 2011 PLoS ONE 6: e14625). It remains to be elucidate in fact whether or not T. indicum strains of different ITS classes are cross-fertile.

Thus, the T. indicum ascocarps TI_U983, TI_U986 and TI_CF10 were selected and by using the MAT1-1 idiomorph-specific primer pair i7/i8 a 9. Kbp long amplicon was obtained from each of the three ascocarps.

Similarly, by using the MAT1-2 idiomorph-specific primer pair i7/i9, an amplicon of about 13.5 Kbp was obtained from TI_U983 and TI_CF10 samples, whereas a 11 Kbp amplicon was obtained from TI_U986 (FIG. 7, FIG. 8).

These six amplicons were completely sequenced and deposited in GenBank under the accession N^{o}.

3. Structure of T. Indicum MAT Idiomorphs and Comparison with their hortologs from T. melanosporum

Sequence alignment, BLAST and gene prediction analyses were performed to evaluate length and structure of MAT1-1 and MAT1-2 idiomorphs and to identify the putative coding regions. The organization of T. indicum MAT idiomorphs, compared with those of T. melanosporum is reported in FIG. 8.

Blast analysis revealed that, like in T. melanosporum, the T. indicum idiomorphic regions were not completely different each other for the presence of shared sequences arranged in inverse (FIG. 8; “G”, “H”, “I” and “F” hatched boxes) or similar (FIG. 8 “J” hatched box) orientation. Most of these regions were also shared with T. melanosporum. The exceptions were region “J” which is present only in the idiomorphs of T. indicum TI_CF10, and region “H” that was absent in T. melanosporum MAT1-2.

The idiomorph sequences of T. indicum differed each other and from that of T. melanosporum for the presence of some indels ranging from few by to approximately 160 bp. These indels are indicated with grey boxes in FIG. 8.

3.1. T. indicum MAT1-1

T. indicum MAT1-1 idiomorph is 7.583 bp long in TI_U983, 7.741 bp in TI_U986 and 7.666 bp in TI_CF10. The structure and sequence is similar to that of T. melanosporum MAT1-1 which is 7.430 bp long. Gene prediction analysis of MAT1-1 showed the presence of a single gene (FIG. 8b) exhibiting high similarity (97% of sequence identity) with T. melanosporum MAT1-1-1 gene (FIG. 9). The deduced MAT1-1-1 encoded protein is 319 amino acids long in each of the three T. indicum strains. T. indicum TI_U983 and TI_U986 shared identical protein sequences whereas T. indicum TI_CF10 contained nine polymorphic sites with respect to the other two strains. T. indicum MAT1-1-1 proteins of TI_U986 and TI_U983 differed from T. melanosporum MAT1-1-1 for 9 amino acids, whereas TI_CF10 for 7.

3.2. T. indicum MAT1-2

T. indicum MAT1-2 idiomorph is 9.783 bp, 10.096 bp and 12.131 bp long in TI_U986, TI_CF10 and TI_U983, respectively. Compared to T. melanosporum which is only 5.550 bp long, it contains a large insertion (about 4.300 bp in TI_U986 and TI_CF10 and about 6.650 bp in TI_U983) between the “I” and “G” regions (FIG. 8a).

Differently from MAT1-1, where a single ORF was identified, two ORFs were predicted within TI_U986 and TI_CF10 and three ORFs in TI_U983 MAT1-2 idiomorph (FIG. 8a).

In each of the three MAT1-2 amplicons, gene prediction analysis confirmed the presence of an ORF exhibiting high similarity (about 94% of sequence identity) with MAT1-2-1 gene of T. melanosporum (FIG. 8a, FIG. 11). T. indicum MAT1-2-1 gene encoded a hypothetical protein of 298 amino acids with TI_U983 differing from TI_U986 for only one amino acid. Conversely, TI_CF10 differed for thirteen and fourteen amino acids from TI_U983 and TI_U986, respectively . . . T. indicum MAT1-2-1 deduced proteins of TI_U986, TI_U983 and TI_CF10 differed from T. melanosporum MAT1-2-1 for 19, 17 and 13 amino acids, respectively, and for an insertion of a single amino acid in the N-terminal region (FIG. 12).

The second ORF identified in each T. indicum MAT1-2 was named TI_orf2. IT consists of 4 exons and was in reverse orientation respect to MAT1-2-1 (FIG. 8a). The TI_orf2 codified for a hypothetical protein of 830 amino acids in TI_U986 and TI_U983 and 762 amino acids in TI_CF10 (FIG. 13). BlastP analysis against GenBank database showed high similarity (E-value<8e −45) with some uncharacterized Ankyrin containing proteins of ascomycetes (i.e. XP_{—}002487679, XP_{—}002382023, XP_{—}002144344). Searches against the Conserved Domains Database (CCD) at NCBI revealed the presence of a conserved ankyrin domain (ANK) at the C-termini of the predicted amino acidic sequence. Amino acid alignment showed that the length difference between the ORF2 proteins of TI_U986/TI_U983 and TI_CF10 resides in the C-terminal region and consists of a variation on the number of ankyrin repeat units.

The 4^{th} exon of TI_orf2 gene in the MAT1-2 idiomorph spans the regions “H” and “I” (FIG. 8a). A partially similar region (about 85% nucleotide identity) was also present in inverse orientation in MAT1-1 idiomorph of both T. indicum and T. melanosporum. However, the orf2 is not functional in MAT1-1 because the first three exons were completely absent and the region corresponding to the 4^{th} exon contains several stop codons.

A third additional ORF was identified in TI_U983 MAT1-2. In this sample the MAT1-2 idiomorph was longer than those of the other two samples for the presence of an insertion of 2.350 bp. Gene prediction analysis and BlastX searches against GenBank database allowed the identification within this insertion of a putative 1.819 bp gene consisting of three exons and resembling a transposable element, which was named Ti-tr1 (FIG. 8a). A 1.868 bp long insertion resembling a second transposon element, named Ti-tr2, was also identified in the 3′ flanking region of the TI_CF10 MAT1-2 idiomorph (FIG. 8a).

C. Tuber borchii

1. Identification of T. borchii MAT Genes

The inventors used the sequences of T. melanosporum mating type genes MAT1-2-1 and MAT1-1-1 as queries to search for ortholog T. borchii sequences deposited in public databases (GenBank and Expressed Sequence Tags (EST) NCBI databases).

By using the T. melanosporum MAT1-1-1 sequence as a blastn query, the T. borchii sequence AF487329 with 85% sequence identity was identified in the NCBI EST database. Conversely, neither Blastn nor tblastn searches identified any T. borchii sequence with a significant similarity to the T. melanosporum MAT1-2-1 nucleotidic and amino acidic sequences, respectively.

The AF487329 sequence of T. borchii was aligned with the corresponding MAT1-1-1 gene of T. melanosporum (FIG. 14) and T. borchii MAT1-1-1 specific primers b1 and b2 (Table 5, FIG. 16) were designed and used to analyze a set of 5 T. borchii ascocarps and 4 mycelial isolates given in Table 2. The T. borchii MAT1-1-1 specific primers produced the expected amplicon of about 530 bp from two ascocarps and from two of the four in vitro isolated mycelia (Table 6). At the light of a possible heterothallism in this species, the strains that produced the expected 530 bp-long amplicon were regarded to as strains containing the MAT-1-1-1 gene, those that did not as strains harboring the alternative MAT gene MAT1-2-1 (Table 6).

To identify the second mating type gene (MAT1-2-1) in T. borchii, the inventors screened the T. borchii ascocarps and mycelia reported in Table 6 with the PCR primers P1 and P2 specific to the MAT1-2-1 gene of T. melanosporum (Rubini et al., 2011 New Phytologist 189: 710-722). Since these primers did not produce any detectable amplicon, the inventors designed the primer pair b3/b4 on the most conserved HMG-box region of MAT1-2-1 genes of different Tuber spp. and tested it under low stringent PCR condition. This primer pair produced an amplicon of approximately 1 Kbp only on those T. borchii strains that failed to produce the MAT1-1-1 specific amplicon (Table 2). The PCR amplicon from ascocarp TB110 was selected, cloned and sequenced. Blastx search against the T. melanosporum Gene Models database (http://mycor.nancy.inra.fr/IMGC/TuberGenome/blast.php) confirmed a high similarity (68% amino acidic identity) of the TB110 MAT1-2-1 fragment with the MAT1-2-1 predicted protein of T. melanosporum. The alignment of the two above mentioned sequences is given in FIG. 15.

The inventors then designed the primer pair b5/b6 specific to T. borchii MAT1-2-1 (Table 5; FIG. 16). This primer pair produced a band of about 670 bp on the MAT1-2 samples but not on the MAT1-1 samples (Table 6) confirming that T. borchii, like other Tuber spp. is an heterothallic fungus.

Moreover, since the mycelia have been isolated from single ectomycorrhizas, these results proved that the mycelium forming T. borchii mycorrhizas is haploid. The same conclusion was previously drawn for T. magnatum and T. melanosporum mycorrhizas (Paolocci et al., (2006) Applied and Environmental Microbiology 72: 2390-2393; Rubini et al., (2011) New Phytologist 189: 723-735.

2. Identification of T. borchii MAT Idiomorphs

The inventors used the two haploid mycelial strains TB16 and TB15, harboring MAT1-1-1 and MAT1-2-1 genes, respectively, to clone and sequence the corresponding MAT idiomorphs.

To clone the idiomorph containing the MAT1-1-1 gene, the sequence (AF487329) retrieved from GenBank was extended towards the 5′ and 3′ ends following a mixture of genome walking and inverse PCR-based strategies using DNA from TB16 as a target. Genome walking was performed using the Universal GenomeWalker™ Kit (Takara Bio Europe/Clontech Saint-Germain-en-Laye France) according to the supplier's instruction; inverse PCR basically as described in Ochman et al., (1988) Genetics 120: 621-623. Long distance PCR was performed using LA-Taq DNA polymerase (Takara Bio Europe Saint-Germain-en-Laye France). Following these approaches the inventors assembled a 11,4 kb long contig containing the MAT1-1 idiomorph. Within the MAT1-1 idiomorph a single α-box (MAT1-1-1) containing gene was identified (FIG. 16a).

The mating type idiomorph MAT1-1 of T. borchii (TborchiiMAT1-1) corresponds to the nucleotide sequences SEQ ID N^{o} 52. The MAT1-1-1 gene of T. borchii (T.borchiiMAT1-1-1) corresponds to the nucleotide sequences SEQ ID N^{o} 53.

To sequence the genomic region corresponding to the MAT1-2 idiomorph the inventors extended the 1 Kb long fragment resulting from the PCR amplification of TB110 ascocarp with the primer pair b3/b4 toward its 5′ end following the genome walking approach described above and using DNA from TB15 as target. Conversely, the 3′ end of the same idiomorph was cloned using sequence information derived from the TB16 sequence. The inventors have in fact identified within the TB16 contig a sequence corresponding to the T. melanosporum gene model GSTUMT00001092001 denoted as ORF3, which is conserved on the 3′ flanking region of both MAT1-1 and MAT1-2 idiomorphs of T. melanosporum and T. indicum (FIG. 16a). The inventors thought this sequence could also be conserved on the T. borchii MAT1-2 downstream region. Following this rationale, the inventors first designed the primer pair b7/b8 on the ORF3 gene of TB16 (Table 5, FIG. 16a). These primers produced an amplicon of approximately 320 bp not only on TB16, used as control, but also on TB15 as predicted. Sequence analysis confirmed that b7/b8-generated amplicons from TB15 and TB16 have similar sequences (about 90% of sequence identity) and share homology with a portion of the T. melanosporum gene model GSTUMT00001092001. The inventors then designed the primers b9 and b10, specific to TB15 fragment, and used them in combination with the HMG-box forward primer b6 (Table 5, FIG. 16b). Whereas the b6/b9 primer pair did not produce any PCR amplicon on TB15, a 6870 kb long amplicon was obtained using the b6/b10 primer pair on the same target. The inventors interpreted these results to mean that ORF3 sequence is indeed present in the TB15 genomic region downstream the MAT1-2-1 gene but it shows an inverted orientation with respect to the ORF3 sequence detected downstream the T. borchii MAT1-1 idiomorph. All the TB15 sequences obtained following the above reported strategies were assembled on a 14,6 Kb long contig that embraces the MAT1-2 idiomorph and the MAT1-2-1 gene (FIG. 16b)

The mating type idiomorph MAT1-2 of T. borchii (TborchiiMAT1-2) corresponds to the nucleotide sequences SEQ ID N^{o} 54. The MAT1-2-1 gene of T. borchii (T.borchiiMAT1-2-1) corresponds to the nucleotide sequences SEQ ID N^{o} 55.

The inventors have determined a first genomic region comprising the mating type idiomorph MAT1-1 which contains the mating type gene MAT1-1-1 and a second genomic region comprising the mating type idiomorph MAT1-2 which contains the mating type gene MAT1-2-1. Each of these genomic regions comprises also conserved flanking sequences.

The first genomic region correspond to the nucleotide sequences SEQ ID N^{o} 56, and the second genomic region correspond to the nucleotide sequence SEQ ID N^{o} 57.

TABLE 5
List of primers used in this study

| Primer name | Primer sequence 5′-3′ |
|---|---|
| b1 | TCAACGGTGATCAGACCCTTGCTCGTG |
| b2 | GAGCGAAGTTAGACAATTTGGGCGT |
| B3 | TTTCTTTGATGGGTCGGATGGAG |
| B4 | GCCCTTGCCTATTAATGTGTTAGTG |
| B5 | ATTGGGACCCACCTTACTATCCGCAATC |
| B6 | CCGGGGAATCCTGCCACATGCGACCGAGA |
| B7 | TTTACTATCATGACCAAAGCGTAT |
| B8 | TTGCAGGTAATGGCTGCCAGAT |
| B9 | GGGATCCCGGCAGACCTCACTAACAG |
| B10 | GGCTGTGGTTCTCAAGGACCGGTTT |

TABLE 6
list of mycelial strains and ascocarps used in
this study

| code | Species | source | Mating type |
|---|---|---|---|
| Tb6 | T. borchii | mycelium | MAT1-1 |
| Tb7 | T. borchii | mycelium | MAT1-2 |
| Tb15 | T. borchii | mycelium | MAT1-2 |
| Tb16 | T. borchii | mycelium | MAT1-1 |
| tb98 | T. borchii | ascocarp | MAT1-2 |
| tb101 | T. borchii | ascocarp | MAT1-2 |
| tb102 | T. borchii | ascocarp | MAT1-1 |
| tb110 | T. borchii | ascocarp | MAT1-2 |
| tb100 | T. borchii | ascocarp | MAT1-1 |

D. Tuber magnatum

Identification of the Mating Type Genes and of the MAT1-2 Idiomorph in T. magnatum

The inventors used the sequences of both T. melanosporum and T. borchii MAT1-2-1 and MAT1-1-1 genes to design on their conserved HMG-box and α-box regions the primer pairs M1/M2 and M3/M4, respectively (Table 1). The inventors then used these two primer pairs to amplify the DNA isolated from different T. magnatum ascocarps. Those ascocarps that produced an amplicon of about 300 bp with the primers M1/M2 did not yield any amplicon with the primer pair M3/M4. Similarly, ascocarps producing a band of about 500 bp with the primers pair M3/M4 did not yield any amplicon with the primers M1/M2. These results are in keeping with the arrangement of MAT genes shown by heterothallic ascomycetes and among them by the Tuber spp. analysed thus far, in that strains harbouring either the MAT1-2-1 or the MAT1-1-1 genes have been observed. Sequencing of the two T. magnatum products obtained as reported above confirmed the homology with the HMG-box and α-box sequences of the other Tuber spp for the 300 and 500 bp long products, respectively.

The inventors have then designed the primers M5 and M6 (FIG. 17) based on the sequences of T. magnatum HMG-box (MAT1-2-1). The inventors also designed the primers M7 and M8 on the T. melanosporum genes GSTUMT00001092001 and GSTUMT00001088001 present at the 3′ and 5′ extremities, respectively, of both MAT idiomorphs of T. melanosporum. By reasoning that genomic regions flanking the MAT idiomorphs are expected to be conserved among Tuber spp. the inventors used the primer combinations M5/M7 and M6/M8 (Table 7) to clone the entire MAT1-2 idiomorph from T. magnatum. Indeed, using the primer combination M5/M7 the inventors were able to PCR amplify a fragment of about 5600 bp containing the 3′ end of MAT1-2-1 gene and the relative flanking sequence, including a portion of a gene homologous to GSTUMT00001092001 of T. melanosporum (FIG. 17). Conversely, no PCR amplicons were obtained when the primer combination M6/M8 was used. Therefore, to clone the 5′ end of the MAT1-2 idiomorph, the inventors performed a genome walking procedure by using the Universal GenomeWalker™ Kit (Clontech) according to the supplier's instruction and the primer M6 as starting point for walking. This approach has allowed the inventors to clone a PCR fragment of about 2500 bp, whose sequencing proved the successful cloning of the 5′ end of T. magnatum MAT1-2-1 gene along with its upstream sequences. Overall, merging the results of the 5′ and 3′ walking reactions a genomic sequence of about 7700 bp has been identified. This genomic region contains the T. magnatum MAT1-2-1 gene and the MAT1-2 idiomorph. The T. magnatum MAT1-2-1 gene (TmagnMAT1-2-1) corresponds to the nucleotide sequence SEQ ID N^{o} 58.

The mating type idiomorph MAT1-2 (TmagnMAT1-2) corresponds to the nucleotide sequence SEQ ID N^{o} 59.

The inventors have determined the genomic region comprising the first mating type gene MAT1-2-1 and the first mating type idiomorph MAT1-2 of T. magnatum. This genomic region comprises also flanking regions that are highly conserved between Tuber species. The entire genomic region has as nucleotide sequence the nucleotide sequence SEQ ID N^{o} 60.

The inventors have also determined the nucleotide sequence of the conserved α-box region within the MAT1-1 gene. This region has as nucleotide sequence the nucleotide sequence SEQ ID N ^{o}61.

TABLE 7
primers used

| Primer name | Sequence 5′-3′ |
|---|---|
| M1 | GTTYGATTCSGCCCYCCAYACCATTAT |
| M2 | CCGGGGAATCCTGCCACATGCGACCGAGA |
| M3 | CYTTCCTTCTTGCRGCAGCCTTATC |
| M4 | ARTACTGYAACGGTTATGGGCTCAC |
| M5 | ATCGGGACTCACCATACTATCCGCAGC |
| M6 | AAAGGGATGAGAGCTGCATACAAATGTCA |
| M7 | TTGRCGGAGTTCRACAAAKGCCTGACGAGC |
| M8 | CAATCTCTTCCATCGCCCGTCCAG |

E. Tuber aestivum

Identification of the MAT1-2 Idiomorph of Tuber aestivum

The inventors have screened the first assembly of T. aestivum genome for the presence of candidate genes encoding for proteins containing the HMG or the alpha-box domain using the mating type genes of T. melanosporum as queries. Following this approach, the inventors have discovered in the T. aestivum genome one nucleotide sequence on the scaffold 88 that has homology with the HMG containing mating type gene of T. melanosporum (GSTUMT00001090001). On the contrary, no T. aestivum genes corresponding to the alpha-box mating type genes have been detected, suggesting that this is indeed a heterothallic species. The inventors have also identified in the same T. aestivum scaffold the two gene models GSTUMT00001088001 and GSTUMT00001092001 present at the 5′ and 3′ extremities of the T. melanosporum MAT1-2 idiomorph, respectively. This allows the inventors to confirm that this region corresponds to the mating type MAT1-2 of T. aestivum. The MAT1-2-1 gene of T. aestivum (TaestMAT1-2-1), containing the HMG-box region, has as nucleotide sequence SEQ ID N^{o} 62.

Overall, following the above mentioned approaches MAT1-2 idiomorphic region of T. aestivum has been identified. The mating type idiomorph MAT1-2 (TaestMAT1-2) corresponds to the nucleotide sequence SEQ ID N ^{o}63.

The inventors have determined the genomic region comprising the first mating type gene MAT1-2-1 and the first mating type idiomorph MAT1-2. This genomic region comprises also flanking regions that are highly conserved between Tuber species. The genomic region has as nucleotide sequence the nucleotide sequence SEQ ID N ^{o}64.

1. A method for determining the Tuber species of a truffle sample, said method comprising identifying whether said truffle sample comprises at least one nucleotide sequence chosen from a first nucleotide sequence associated with a first mating type idiomorph (MAT1-2) or a second nucleotide sequence associated with a second mating type idiomorph (MAT1-1); both nucleotide sequence being specific to a given Tuber species.

2. The method of claim 1, further comprising determining the fertility of said truffle sample, using the identification in said truffle sample of both the first nucleotide sequence associated with the first mating type idiomorph (MAT1-2) and the second nucleotide sequence associated with a second mating type idiomorph (MAT1-1), the fertility of said truffle sample requiring the presence of both the first and the second nucleotide sequences.

3. The method of claim 1, wherein the first nucleotide sequence comprises a nucleotide sequence selected from SEQ ID N^{o}5, SEQ ID N^{o}46, SEQ ID N^{o}47, SEQ ID N^{o}48 or SEQ ID N^{o}56 and the second nucleotide sequence comprises a nucleotide sequence selected from SEQ ID N^{o}6, SEQ ID N^{o}49, SEQ ID N^{o}50, SEQ ID N^{o}51 or SEQ ID N^{o}57.

4. The method of claim 1, using a first pair of oligonucleotide primers to determine the presence of the first mating type idiomorph (MAT1-2) in said truffle sample, each primer of the first pair of oligonucleotide primers comprising from 5 to 50 successive nucleotides of the first nucleotide sequence.

5. The method of claim 4, wherein a forward primer of the first pair of oligonucleotide primers comprises the nucleotide sequence SEQ ID N^{o}7.

6. The method of claim 4, wherein a reverse primer of the first pair of oligonucleotide primers comprises the nucleotide sequence SEQ ID N^{o}8.

7. The method of claim 1, using a second pair of oligonucleotide primers to determine the presence of the second mating type idiomorph (MAT1-1) in said truffle sample, each primer of the second pair of oligonucleotide primers comprising from 5 to 50, successive nucleotides of the second nucleotide sequence.

8. The method of claim 7, wherein a forward primer of the second pair of oligonucleotide primers comprises the nucleotide sequence SEQ ID N^{o}9.

9. The method of claim 7, wherein a reverse primer of the second pair of oligonucleotide primers comprises the nucleotide sequence SEQ ID N^{o}10.

10. The method of claim 3, wherein each of the nucleotide sequences SEQ ID N^{o}5 and SEQ ID N^{o}6 are specific to Tuber melanosporum.

11. Nucleotide sequence associated with a first mating type idiomorph (MAT1-2) of a Tuber species comprising the nucleotide sequence SEQ ID N^{o}5 or with a second mating type idiomorph (MAT1-1) of a Tuber species comprising the nucleotide sequence SEQ ID N^{o}6.

12. (canceled)

13. Pair of oligonucleotide primers associated with a first mating type idiomorph (MAT1-2-1) of a Tuber species, or with a second mating type idiomorph (MAT1-1) of a Tuber species, wherein each primer associated with the first mating type idiomorph comprises from 5 to 50 successive nucleotides of the nucleotide sequence SEQ ID N^{o}5 and each primer associated with the second mating type idiomorph comprises 5 to 50 successive nucleotides of the nucleotide sequences SEQ ID N^{o}6.

14. Pair of oligonucleotide primers of claim 13 wherein each primer comprises from 10 to 30 successive nucleotides of the nucleotide sequence.

15. Kit for determining the fertility of a truffle sample, like truffle fructification sample, truffle mycelium sample, mycorrhized plant sample or truffle-producing soil sample, the kit comprising:
the pair of oligonucleotide primers associated with the first mating type idiomorph, and
the pair of oligonucleotide primers associated with the second mating type idiomorph, both pairs according to claim 13.

16. The method of claim 2, wherein the first nucleotide sequence comprises a nucleotide sequence selected from SEQ ID N^{o}5, SEQ ID N^{o}46, SEQ ID N^{o}47, SEQ ID N^{o}48 or SEQ ID N^{o}56 and the second nucleotide sequence comprises a nucleotide sequence selected from SEQ ID N^{o}6, SEQ ID N^{o}50, SEQ ID N^{o}51 or SEQ ID N^{o}57.

17. The method of claim 2, wherein the first nucleotide sequence comprises the nucleotide sequence SEQ ID N^{o}5 and the second nucleotide sequence comprises the nucleotide sequence SEQ ID N^{o}6.

18. The method of claim 4, wherein a forward primer of the first pair of oligonucleotide primers comprises the nucleotide sequence SEQ ID N^{o}7 and wherein a reverse primer of the first pair of oligonucleotide primers comprises the nucleotide sequence SEQ ID N^{o}8.

19. The method of claim 7, wherein a forward primer of the second pair of oligonucleotide primers comprises the nucleotide sequence SEQ ID N^{o}9 and wherein a reverse primer of the second pair of oligonucleotide primers comprises the nucleotide sequence SEQ ID N^{o}10.

20. The method of claim 1, using a first pair of oligonucleotide primers to determine the presence of the first mating type idiomorph (MAT1-2) in said truffle sample, and using a second pair of oligonucleotide primers to determine the presence of the second mating type idiomorph (MAT1-1) in said truffle sample; each primer of the first and second pairs of oligonucleotide primers comprising from 5 to 50 successive nucleotides of the first and second nucleotide sequence, respectively.

21. The method of claim 20, wherein a forward primer of the first pair of oligonucleotide primers comprises the nucleotide sequence SEQ ID N^{o}7, wherein a reverse primer of the first pair of oligonucleotide primers comprises the nucleotide sequence SEQ ID N^{o}8, wherein a forward primer of the second pair of oligonucleotide primers comprises the nucleotide sequence SEQ ID N^{o}9 and wherein a reverse primer of the second pair of oligonucleotide primers comprises the nucleotide sequence SEQ ID N^{o}10.
